(19) United States
(12) Patent Application Publication
(10) Pub. No.: US 2024/0382471 A1
(43) Pub. Date: Nov. 21, 2024

(54) GLUCAGON-LIKE PEPTIDE-1 RECEPTOR MODULATORS AND USES THEREOF

(71) Applicant: HEPAGENE THERAPEUTICS (HK) LIMITED, Hong Kong (CN)

(72) Inventors: Xiaodong Xu, Hong Kong (CN); Cheng Mo, Hong Kong (CN); Shijia Chen, Hong Kong (CN)

(73) Assignee: HEPAGENE THERAPEUTICS (HK) LIMITED, Hong Kong (CN)

(21) Appl. No.: 18/580,840

(22) PCT Filed: Jul. 21, 2022
(86) PCT No.: PCT/CN2022/107047

§ 371 Date: Jan. 19, 2024

(30) Foreign Application Priority Data
Jul. 21, 2021 (WO) PCT/CN2021/107693
Feb. 15, 2022 (WO) PCT/CN2022/076288

(51) Int. Cl. A61K 31/444; A61K 31/4184; A61K 31/4439; A61K 31/4995; A61P 3/10; C07D 401/14; C07D 405/06; C07D 405/14

(52) U.S. Cl. CPC A61K 31/444; A61K 31/4184; A61K 31/4439; A61K 31/4995; A61P 3/10; C07D 401/14; C07D 405/06; C07D 405/14

(57) ABSTRACT

The present invention is directed to compounds of formula I or a pharmaceutically acceptable salt thereof, pharmaceutical compositions thereof, and methods related to agonists of glucagon-like peptide-1 receptor (GLP-1R). In particular, the compounds and compositions may be used to treat GLP-1R-related disorders and conditions, including, e.g., obesity, T2DM, NAFLD, NASH as disclosed herein.

### FIELD OF INVENTION

The present invention is directed to compounds, compositions, and methods related to agonists of glucagon-like peptide-1 receptor (GLP-1R). In particular, the compounds and compositions of the present invention may be used to treat GLP-1R-related disorders and conditions, including, e.g., obesity, T2DM, NAFLD, NASH.

### BACKGROUND

The glucagon-like peptide-1 receptor (GLP-1R) belongs to class B G protein-coupled receptors (GPCRs) which is a clinically proved target for type 2 diabetes mellitus (T2DM) and obesity. Multiple peptidic GLP-1R agonists (such as semaglutide, dulaglutide, albiglutide and lixisenatide) are approved for the treatment of T2DM for their efficacious glucose-lowering effect with salient benefits for body weight and cardiovascular event. However, the requirement for injection resulting in the poor patient compliance limits their use. Oral semaglutide is approved by the U.S. Food and Drug Administration (FDA), but its substantial food-drug interactions may potentially generate inconsistent effects in the real-world setting and reduce its efficacy.

Non-alcoholic fatty liver disease (NAFLD) is the hepatic manifestation of metabolic syndrome with a build-up of fat in the liver, which would cause non-alcoholic steatohepatitis (NASH), and even more severe healthy problems. Non-alcoholic fatty liver disease and steatohepatitis are highly associated with obesity and T2DM, and peptidic GLP-1R agonists are evaluated their efficacy for these indications in the clinical trials. There is no approved treatment for NAFLD and NASH worldwide yet.

Therefore, an easily-administered prevention and/or treatment means for T2DM and associated diseases is badly needed to satisfy the unmet clinical need. Lots of small molecular GLP-1R agonists with different active profiles (WO2018/109607A1, WO2019/239319A1, WO2020/263695A1, WO2020/207474A1 and WO2021/018023A1, all of which are incorporated herein by reference in their entity) are reported in the literatures and some of which are in the clinical stage. Given the complex biological functions of GPCRs, it is meaningful to design new GLP-1R agonists with novel scaffold.

### SUMMARY OF THE INVENTION

Disclosed herein are novel compounds as small molecular GLP-1R agonists. As a result, the compounds of the present invention are particularly useful in modulation of GLP-1R and thus in the treatment of GLP-1R-related disorders and conditions.

In one aspect, the present invention is directed to a compound of Formula I

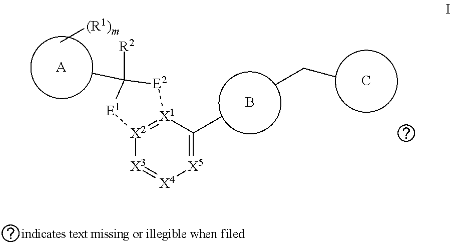

or a pharmaceutically acceptable salt thereof, wherein ring A, ring B, ring C, R¹, R², m, E¹, E², X¹, X², X³, X⁴ and X⁵ are described herein.

In another aspect, the present invention is directed to a pharmaceutical composition comprising the compound of Formula I or a pharmaceutically acceptable salt thereof as described herein, and at least one pharmaceutically acceptable carrier.

In a further aspect, the present invention is directed to a method of treating a GLP-1R-related disorder or condition in a subject in need thereof, comprising administering to the subject a therapeutically effective amount of a compound of Formula I or a pharmaceutically acceptable salt thereof as described herein.

In yet a further aspect, the present invention is directed to a compound of Formula I or a pharmaceutically acceptable salt thereof as described herein for use in treatment of a GLP-1R-related disorder and condition.

In yet a further aspect, the present invention is directed to use of a compound of Formula I or a pharmaceutically acceptable salt thereof as described herein in the manufacture of a medicament for the treatment of a GLP-1R-related disorder or condition.

### DETAILED DESCRIPTION

Reference will now be made in detail to certain embodiments, examples of which are illustrated in the accompanying detailed description. While enumerated embodiments will be described, it will be understood that they are not intended to limit the invention to those embodiments. On the contrary, the invention is intended to cover all alternatives, modifications and equivalents which may be included within the scope of the present invention as defined by the claims.

### Definitions

Definitions of specific functional groups and chemical terms are described in more detail below. For purposes of this disclosure, the chemical elements are identified in accordance with the Periodic Table of the Elements, CAS version, Handbook of Chemistry and Physics, 75th Ed., inside cover, and specific functional groups are generally defined as described therein.

As used herein, the singular forms “a”, “an” and “the” include plural referents unless expressly stated to the contrary.

As used herein, the terms “comprise” and “include” are intended to specify the presence of stated features, integers, components or steps, but they do not preclude the presence or addition of one or more other features, integers, components, steps or groups thereof.

As used herein, the term “substituted”, when refers to a chemical group, means the chemical group has one or more hydrogen atoms that is/are removed and replaced by substituents. The term “substituent”, as used herein, has the ordinary meaning known in the art and refers to a chemical moiety that is covalently attached to, or if appropriate, fused to, a parent group. It is to be understood that substitution at a given atom is limited by valency.

As used herein, the term “Cᵢ₋ⱼ” indicates a range of the carbon atom numbers, wherein i and j are integers and the range of the carbon atoms numbers includes both the endpoints (i.e., i and j) and each integer point in-between, and wherein j is greater than i. For example, the term “C₁₋₃” indicates a range of one to three carbon atoms, including one carbon atom, two carbon atoms and three carbon atoms. In some embodiments, the term “C₁₋₃” indicates 1 to 3 and particularly 1 to 2 carbon atoms.

As used herein, the term “alkyl” refers to a monovalent straight or branched chain, saturated aliphatic hydrocarbon radical. In some embodiments, alkyl groups contain 1 to 3 carbon atoms. Examples of such alkyl group include, but are not limited to, n- and iso-propyl, ethyl and methyl, and particularly, ethyl and methyl.

As used herein, the term “alkoxy” or “—Oalkyl” refers to an alkyl group, as defined above, attached to the parent molecule through an oxygen atom. The term “Cᵢ₋ⱼalkoxy” or “—OCᵢ₋ⱼalkyl” means that the alkyl moiety of the alkoxy group has i to j carbon atoms. In some embodiments, alkoxy groups contain 1 to 3 carbon atoms, and particularly 1 to 2 carbon atoms. Examples of such alkoxy group include, but are not limited to, methoxy, ethoxy and propoxy (e.g., n-propoxy and isopropoxy).

As used herein, the term “aryl” refers to a cyclic aromatic ring system having a specified number of ring-forming atoms. Examples of such aryl group include, but are not limited to, phenyl.

As used herein, the term “heteroaryl” includes a 5- or 6-membered heteroaromatic ring containing 1 to 4 heteroatoms independently selected from N, O and S. Examples of such 5- and 6-membered heteroaryl group include, but are not limited to, pyridyl, pyrrolyl, pyrazinyl, pyrimidinyl, pyridazinyl, triazinyl, thienyl, furanyl, imidazolyl (e.g., imidazol-2-yl, imidazol-3-yl, imidazol-4-yl), pyrazolyl, triazolyl (i.e., 1,2,3-triazolyl or 1,2,4-triazolyl), tetrazolyl, oxazolyl, isooxazolyl, oxadiazolyl (i.e., the 1,2,3-, 1,2,4-, 1,2,5-(furazanyl) or 1,3,4-isomer), oxatriazolyl, thiazolyl, isothiazolyl, and thiadiazolyl.

As used herein, the term “cycloalkyl” refers to a monovalent non-aromatic, saturated monocyclic and polycyclic ring system, in which all the ring atoms are carbon atoms. In some embodiments, the cycloalkyl group may contain 6 to 8 ring-forming carbon atoms. In some embodiments, the cycloalkyl group may be monocyclic ring system. In some embodiments, the cycloalkyl group may be polycyclic (e.g., bicyclic) ring system, which can be arranged as a fused, spiro or bridged ring system. In some embodiments, the cycloalkyl group may be a 6- to 8-membered monocyclic or bicyclic ring system.

As used herein, the term “fused ring” refers to a ring system having two rings sharing two adjacent atoms, the term “spiro ring” refers to a ring system having two rings connected through one single common atom, and the term “bridged ring” refers to a ring system with two rings sharing three or more atoms.

As used herein, the term “cycloalkenyl” refers to a monovalent non-aromatic monocyclic and polycyclic ring system, which contains at least one carbon-carbon double bond. In some embodiments, the cycloalkenyl may contain 6 to 8 ring-forming carbon atoms. In some embodiments, the cycloalkenyl group may be monocyclic ring system. In some embodiments, the cycloalkenyl group may be polycyclic (e.g., bicyclic) ring system, which can be arranged as a fused, spiro or bridged ring system. In some embodiments, the cycloalkenyl group may be a 6- to 8-membered monocyclic or bicyclic ring system.

As used herein, the term “heterocycloalkyl” refers to a monovalent non-aromatic monocyclic and polycyclic ring system, in which at least one (for example, one, two or three, and particularly one or two) ring atoms are heteroatoms independently selected from N, O or S and the remaining ring atoms are carbon atoms. In some embodiments, the heterocycloalkyl group may contain 6 to 8 ring-forming atoms. In some embodiments, the heterocycloalkyl group may contain 4 to 6 ring-forming atoms. In some embodiments, the heterocycloalkyl group may contain one or two ring-forming heteroatoms independently selected from N and O. In some embodiments, the heterocycloalkyl may contain one or two ring-forming N atoms. In some embodiments, the heterocycloalkyl may contain one or two ring-forming O atoms. In some embodiments, the heterocycloalkyl group may be monocyclic ring system. In some embodiments, the heterocycloalkyl group may be polycyclic (e.g., bicyclic) ring system, which can be arranged as a fused, spiro or bridged ring system. In some embodiments, the heterocycloalkyl group may be a 6- to 8-membered monocyclic or bicyclic ring system.

As used herein, the term “heterocycloalkenyl” refers to a monovalent non-aromatic monocyclic and polycyclic ring system, which contains at least one carbon-carbon double bond, and in which at least one (for example, one, two or three, and particularly one or two) ring atoms are heteroatoms independently selected from N, O or S and the remaining ring atoms are carbon atoms. In some embodiments, the heterocycloalkenyl may contain 6 to 8 ring-forming atoms. In some embodiments, the heterocycloalkenyl may contain one or two ring-forming heteroatoms independently selected from N and O. In some embodiments, the heterocycloalkenyl may contain one or two ring-forming N atoms. In some embodiments, the heterocycloalkenyl group may be monocyclic ring system. In some embodiments, the heterocycloalkenyl group may be polycyclic (e.g., bicyclic) ring system, which can be arranged as a fused, spiro or bridged ring system. In some embodiments, the heterocycloalkenyl group may be a 6- to 8-membered monocyclic or bicyclic ring system.

As used herein, the term “alkylene” refers to a bivalent group obtained by removal of a hydrogen atom from an alkyl group as defined above.

As used herein, the term “cycloalkylene” refers to a bivalent group obtained by removal of a hydrogen atom from a cycloalkyl group as defined above.

As used herein, the term “cycloalkenylene” refers to a bivalent group obtained by removal of a hydrogen atom from a cycloalkenyl group as defined above.

As used herein, the term “heterocycloalkylene” refers to a bivalent group obtained by removal of a hydrogen atom from a heterocycloalkyl group as defined above.

As used herein, the term “heterocycloalkenylene” refers to a bivalent group obtained by removal of a hydrogen atom from a heterocycloalkenyl group as defined above.

As used herein, the dashed line “---” in structure formulas as disclosed herein denotes the presence or absence of a bond as valency allows.

As used herein, the wavy line,

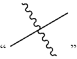

denotes a point of attachment of a substituent to another group.

As used herein, bivalent species are to be read from left to right. For example, regarding the definitions of the formulas described herein, if the specification or claims recite A-B-C and B is defined as

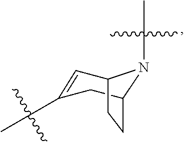 [SMILES: *C1=CC2CCC(C1)N2*]

the resulting group with B replaced is

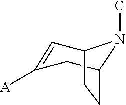

and not

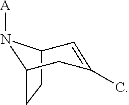

As used herein, the term “halogen” refers to fluoride, chloride, bromide and iodide, particularly fluoride and chloride, and more particularly fluoride.

Where any chiral center in a compound structure is marked as “abs”, it means that the chiral center has only one stereo-configuration (i.e., not a racemate with respect to the chiral center).

When any variable occurs more than one time in any constituent or in Formula I or in any other formula depicting and describing compounds of the present invention, its definition at each occurrence is independent of its definition at every other occurrence. Also, combinations of substituents and/or variables are permissible only if such combinations result in stable compounds.

All ranges cited herein are inclusive, unless expressly stated to the contrary. For example, the term “0 to 3 halogen atoms” may refer to 0 to 3 halogen atoms, 0 to 2 halogen atoms, 0 to 1 halogen atoms, 1 to 2 halogen atoms, 1 to 3 halogen atoms, 2 to 3 halogen atoms, and may include 0, 1, 2 or 3 halogen atoms.

### Compounds

In one aspect, the present invention is directed to a compound of Formula I

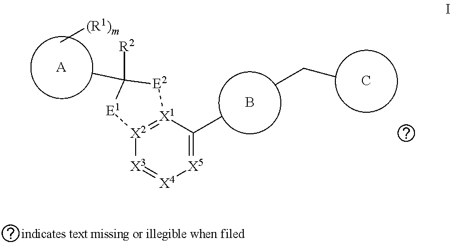

or a pharmaceutically acceptable salt thereof, wherein
ring A is a 5- or 6-membered aryl or heteroaryl group;
each R¹ is independently halogen, —OH, —CN, —C≡CH, —S(O)—C₁₋₃alkyl, —S(O)₂—C₁₋₃alkyl, —P(O)—(C₁₋₃alkyl)₂, —C₃₋₆cycloalkyl, a 3- to 6-membered heterocycloalkyl group, a 5- or 6-membered heteroaryl group, —C₁₋₃alkyl, —C₁₋₃alkylOC₁₋₃alkyl, or —OC₁₋₃alkyl, wherein said alkyl of —C₁₋₃alkyl, —C₁₋₃alkylOC₁₋₃alkyl, and —OC₁₋₃alkyl is substituted with 0 to 5 halogen atoms;
or two R¹ taken together with the carbon atom to which they are attached form a cycloalkyl or heterocyclyl;
m is 0, 1, 2, 3, or 4;
E¹ and E² are independently H, D, halogen (particularly F), O, NH, or CH₂;
X¹ and X² are independently N or CR⁶, R⁶ is independently absent, H, halogen, —C₁₋₃alkyl or —CN;
X³, X⁴ and X⁵ are independently N or CR⁷, wherein R⁷ is independently H, halogen, —C₁₋₃alkyl, —OC₁₋₃alkyl or —CN, wherein said alkyl of —C₁₋₃alkyl and —OC₁₋₃alkyl is substituted with 0 to 3 halogen atoms;
--- denotes the presence or absence of a bond; provided that,
   a) when the --- between E¹ and X² denotes the absence of a bond, then the --- between E² and X¹ denotes the presence of a bond, E¹ is H, D, or halogen (particularly F), and X¹ is C,
   b) when the --- between E² and X¹ denotes the absence of a bond, then the --- between E¹ and X² denotes the presence of a bond, E² is H, D, or halogen (particularly F), and X² is C, and
   c) when the --- between E¹ and X² and the --- between E² and X¹ both denote the presence of a bond, then E¹ and E² are independently O, NH or CH₂, and X¹ and X² are C; R² is independently H, D (i.e., deuterium), halogen (particularly F), or —C₁₋₃alkyl;
ring B is a 6- to 8-membered cycloalkylene, cycloalkenylene, heterocycloalkylene or heterocycloalkenylene group substituted as valency allows with 0 to 3 substituents independently selected from 0 to 3 halogen atoms and 0 to 1 oxo (—O), and may be further substituted by 0, 1 or 2 substituent R, wherein each R is independently H, halogen, —CN or C₁₋₃ alkyl;
ring C is

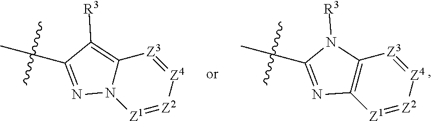

wherein Z¹, Z², Z³ and Z⁴ are independently N, CR⁴ or CR⁸, wherein R⁸ is independently H, —OH, CN, halogen, —C(O) C₁₋₃alkyl, —C(O) C₃₋₆cycloalkyl, —OC₁₋₃alkyl, —C₃₋₆cycloalkyl, or —C₁₋₃alkyl, wherein said alkyl and said cycloalkyl of —C(O) C₁₋₃alkyl, —C(O) C₃₋₆cycloalkyl, —OC₁₋₃alkyl, —C₃₋₆cycloalkyl, and —C₁₋₃alkyl are independently unsubstituted or substituted with one or more substituents selected from D (i.e., deuterium), OH, NH₂, —CN, and halogen; provided that one of Z¹, Z², Z³ and Z⁴ is CR⁴;
R³ is —C₁₋₃alkyl, —C₀₋₃alkylene-C₃₋₆cycloalkyl, or —C₀₋₃alkylene-R⁵, wherein said alkyl may be substituted as valency allows with 0 to 3 substituents independently selected from 0 to 3 halogen atoms and 0 to 1 substituent selected from —C₀₋₁alkylene-CN, —C₀₋₁alkylene-OR⁹, and —N(R¹⁰)₂, and wherein said alkylene and cycloalkyl may be independently substituted as valency allows with 0 to 2 substituents independently selected from 0 to 2 halogen atoms and 0 to 1 substituent selected from —C₀₋₁alkylene-CN, —C₀₋₁alkylene-OR⁹, and —N(R¹⁰)₂;
R⁵ is a 5- or 6-membered heteroaryl group, or a 4- to 6-membered heterocycloalkyl group, wherein said heteroaryl and heterocycloalkyl may be substituted with 0 to 2 substituents as valency allows independently selected from:
   0 to 1 oxo (—O),
   0 to 1 —CN,
   0 to 2 halogen atoms, and
   0 to 2 substituents independently selected from —C₁₋₃alkyl, —OC₁₋₃alkyl and —C₁₋₃alkylene-O—C₁₋₃alkyl wherein the alkyl of —C₁₋₃alkyl and —OC₁₋₃alkyl may be substituted with 0 to 3 substituents as valency allows independently selected from 0 to 3 halogen atoms, 0 to 1 —CN, and 0 to 1 —OR⁹;
each R⁹ is independently H or —C₁₋₃alkyl, wherein-C₁₋₃alkyl may be substituted with 0 to 3 halogen atoms;
each R¹⁰ is independently H or —C₁₋₃alkyl; and
R⁴ is COOH or a carboxylic group surrogate, and particularly, the carboxylic group surrogate is:

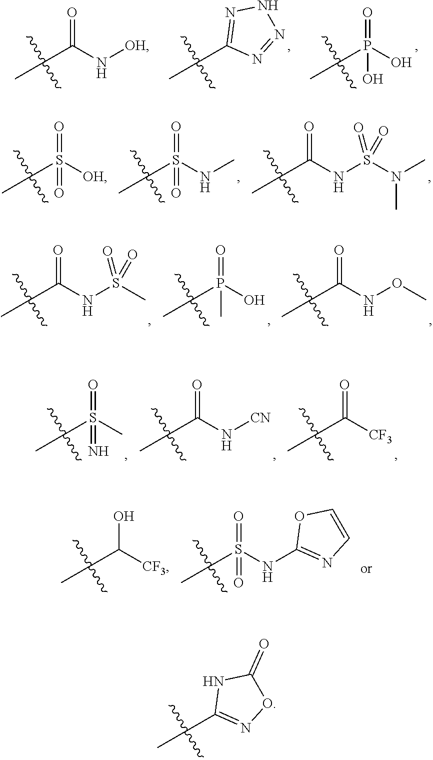

In certain embodiments, the present invention is directed to a compound of Formula I

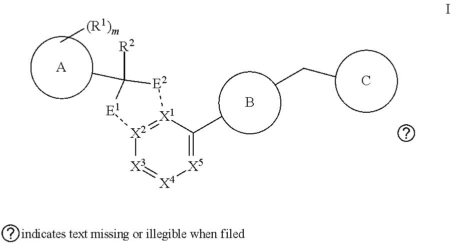

or a pharmaceutically acceptable salt thereof, wherein
ring A is a 5- or 6-membered aryl or heteroaryl group;
each R¹ is independently halogen, —CN, —C≡CH, —S(O)—C₁₋₃alkyl, —S(O)₂—C₁₋₃alkyl, —P(O)—(C₁₋₃alkyl)₂, —C₃₋₆cycloalkyl, a 3- to 6-membered heterocycloalkyl group, a 5- or 6-membered heteroaryl group, —C₁₋₃alkyl, or —OC₁₋₃alkyl, wherein said alkyl of —C₁₋₃alkyl and —OC₁₋₃alkyl is substituted with 0 to 5 halogen atoms;
m is 0, 1, 2 or 3;
E¹ and E² are independently H, O, NH, or CH₂;
X¹ and X² are independently N or CR⁶, R⁶ is independently absent, H, halogen, —C₁₋₃alkyl or —CN;
X³, X⁴ and X⁵ are independently N or CR⁷, wherein R⁷ is independently H, halogen, —C₁₋₃alkyl, —OC₁₋₃alkyl or —CN, wherein said alkyl of —C₁₋₃alkyl and —OC₁₋₃alkyl is substituted with 0 to 3 halogen atoms;
--- denotes the presence or absence of a bond; provided that,
   a) when the --- between E¹ and X² denotes the absence of a bond, then the --- between E² and X¹ denotes the presence of a bond, E¹ is H, and X¹ is C,
   b) when the --- between E² and X¹ denotes the absence of a bond, then the --- between E¹ and X² denotes the presence of a bond, E² is H, and X² is C, and
   c) when the --- between E¹ and X² and the --- between E² and X¹ both denote the presence of a bond, then E¹ and E² are independently O, NH or CH₂, and X¹ and X² are C; R² is independently H or —C₁₋₃alkyl;
ring B is a 6- to 8-membered cycloalkylene, cycloalkenylene, heterocycloalkylene or heterocycloalkenylene group substituted as valency allows with 0 to 3 substituents independently selected from 0 to 3 halogen atoms and 0 to 1 oxo (═O), and may be further substituted by 0, 1 or 2 substituent R, wherein each R is independently H, halogen, —CN or C₁₋₃ alkyl;
ring C is

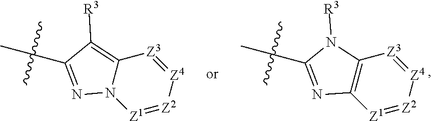

wherein Z¹, Z², Z³ and Z⁴ are independently N, CR⁴ or CR⁸, wherein R⁸ is independently H, CN, halogen, —OC₁₋₃alkyl or —C₁₋₃alkyl, provided that one of Z¹, Z², Z³ and Z⁴ is CR⁴;
R³ is —C₁₋₃alkyl, —C₀₋₃alkylene-C₃₋₆cycloalkyl, or —C₀₋₃alkylene-R⁵, wherein said alkyl may be substituted as valency allows with 0 to 3 substituents independently selected from 0 to 3 halogen atoms and 0 to 1 substituent selected from —C₀₋₁alkylene-CN, —C₀₋₁alkylene-OR⁹, and —N(R¹⁰)₂, and wherein said alkylene and cycloalkyl may be independently substituted as valency allows with 0 to 2 substituents independently selected from 0 to 2 halogen atoms and 0 to 1 substituent selected from —C₀₋₁alkylene-CN, —C₀₋₁alkylene-OR⁹, and —N(R¹⁰)₂;
R⁵ is a 5- or 6-membered heteroaryl group, or a 4- to 6-membered heterocycloalkyl group, wherein said heteroaryl and heterocycloalkyl may be substituted with 0 to 2 substituents as valency allows independently selected from:
   0 to 1 oxo (—O),
   0 to 1 —CN,
   0 to 2 halogen atoms, and
   0 to 2 substituents independently selected from —C₁₋₃alkyl, —OC₁₋₃alkyl and —C₁₋₃alkylene-O—C₁₋₃alkyl wherein the alkyl of —C₁₋₃alkyl and —OC₁₋₃alkyl may be substituted with 0 to 3 substituents as valency allows independently selected from 0 to 3 halogen atoms, 0 to 1 —CN, and 0 to 1 —OR⁹;
each R⁹ is independently H or —C₁₋₃alkyl, wherein-C₁₋₃alkyl may be substituted with 0 to 3 halogen atoms;
each R¹⁰ is independently H or —C₁₋₃alkyl; and
R⁴ is COOH or a carboxylic group surrogate, and particularly, the carboxylic group surrogate is:

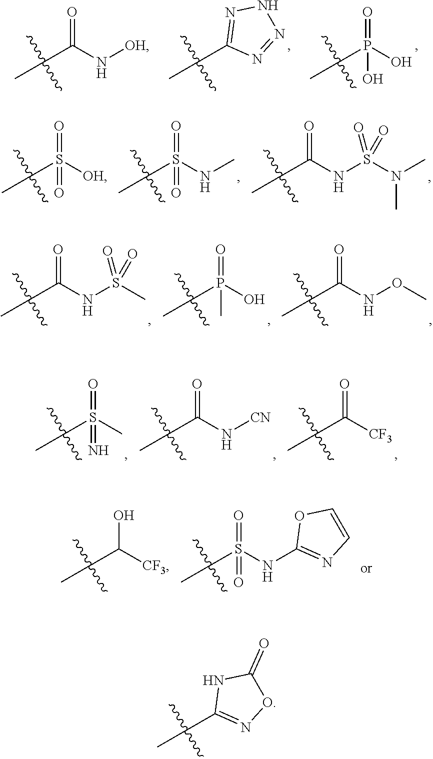

In connection with the compounds of Formula I, the following embodiments may be provided for the purpose of illustration.

In certain embodiments, the --- between E² and X¹ denotes the absence of a bond, the ---between E¹ and X² denotes the presence of a bond, E² is H, D, or halogen (particularly F), and X² is C.

In certain embodiments, the --- between E² and X¹ denotes the absence of a bond, the ---between E¹ and X² denotes the presence of a bond, E² is H, D, or halogen (particularly F), X² is C, and E¹ is O.

In certain embodiments, ring A is phenyl, pyridinyl, or thiophenyl.

In certain embodiments, ring A is phenyl or pyridinyl.

In certain embodiments, each R¹ is independently halogen, —CN, —C≡CH, —C₁₋₃alkyl, —C₁₋₃alkylOC₁₋₃alkyl, or —OC₁₋₃alkyl, wherein said alkyl of —C₁₋₃alkyl and —OC₁₋₃alkyl is substituted with 0 to 5 halogen atoms. In certain embodiments, one R¹ is —OC₁₋₃alkyl, wherein said alkyl of —OC₁₋₃alkyl is substituted with 0 to 5 halogen atoms.

In certain embodiments, each R¹ is independently F, Cl, Br, —CN, —C≡CH, —CH₃, —CF₃, —CH₂OCH₃, —OCH₃, —OCH₂CH₃, —OCH(CH₃)₂, or —OCF₃.

In certain embodiments, each R¹ is independently F, Cl, —CN, —C≡CH, —CH₃, —OCF₃, or —CF₃.

In certain embodiments, each R¹ is independently F, Cl, —CN, —C≡CH, or —CH₃, and m is 0, 1 or 2.

In certain embodiments, m is 1, 2 or 3. In certain embodiments, each R¹ is independently F, Cl, Br, —CN, —C≡CH, —CH₃, —CF₃, —CH₂OCH₃, —OCH₃, —OCH₂CH₃, —OCH(CH₃)₂, or —OCF₃, and m is 1, 2 or 3. In certain embodiments, each R¹ is independently F, Cl, —CN, —C≡CH, or —CH₃, and m is 1, 2 or 3.

In certain embodiments, m is 2 or 3. In certain embodiments, each R¹ is independently halogen, —CN, —C₁₋₃alkyl, or —OC₁₋₃alkyl, wherein said alkyl of —C₁₋₃alkyl and —OC₁₋₃alkyl is substituted with 0 to 5 halogen atoms, and m is 1, 2 or 3, provided that one R¹ is —OC₁₋₃alkyl.

In certain embodiments,

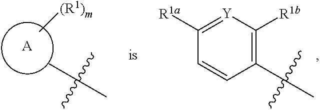

wherein
Y is N or CR¹c;
R^{1a} is H, halogen, —CN, —C≡CH, —C₁₋₃alkyl, —C₁₋₃alkylOC₁₋₃alkyl, or —OC₁₋₃alkyl, wherein said alkyl of —C₁₋₃alkyl and —OC₁₋₃alkyl is substituted with 0 to 5 halogen atoms;
R^{1b} is H, halogen, —C₁₋₃alkyl, or —OC₁₋₃alkyl, wherein said alkyl of —C₁₋₃alkyl and —OC₁₋₃alkyl is substituted with 0 to 5 halogen atoms; and
R^{1c} is H or halogen.

In certain embodiments, R^{1b} is —OC₁₋₃alkyl.

In certain embodiments, R² is H or —CH₃.

In certain embodiments, ring B is

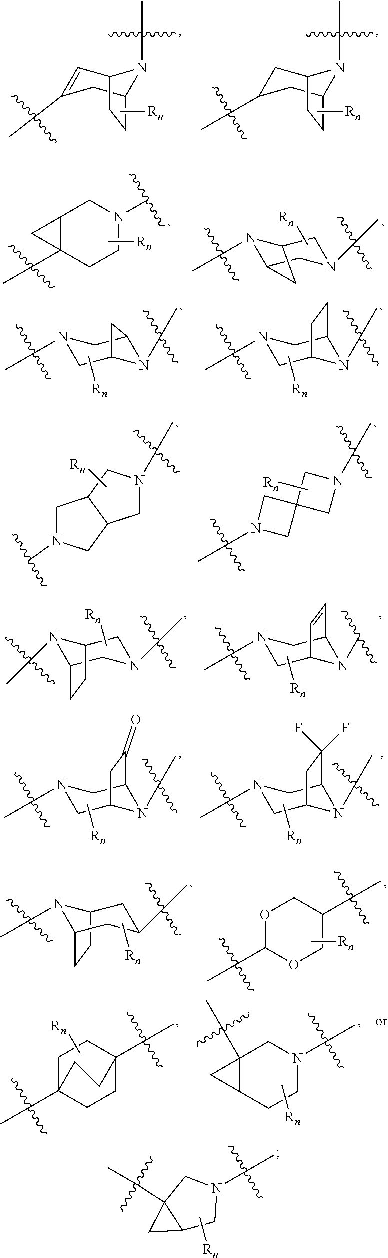

each R is independently H, halogen, —CN, or —C₁₋₃alkyl; and
n is 0, 1 or 2.

In certain embodiments, ring B is:

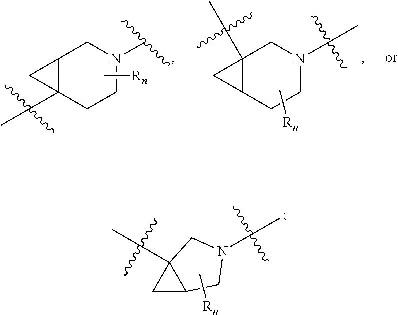

each R is independently H, halogen, —CN, or —C₁₋₃alkyl; and
n is 0, 1 or 2.

In certain embodiments, ring B is:

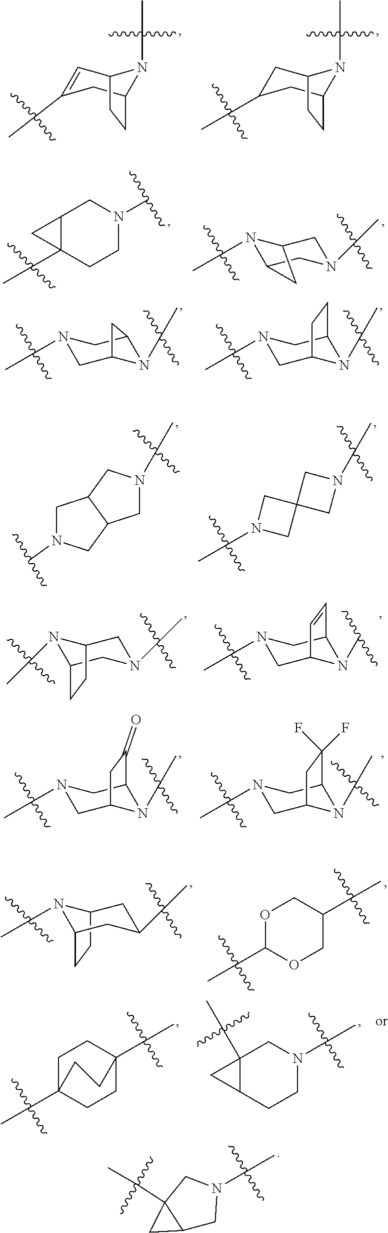

In certain embodiments, ring B is:

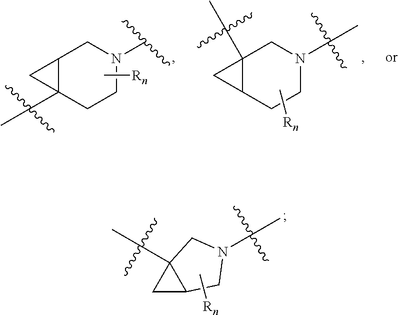

In certain embodiments, in connection with ring C, Z⁴ is CR⁴, wherein R⁴ is as defined above for Formula I. In certain embodiments, Z¹, Z², and Z³ are independently CR⁸, wherein R⁸ is as defined above for Formula I. In certain embodiments, Z¹ is CR⁸, wherein R⁸ is as defined above for Formula I, provided that R⁸ is not H.

In certain embodiments, R³ is —CH₂—R⁵, and R⁵ is a 5- or 6-membered heteroaryl group or a 4- to 6-membered heterocycloalkyl group wherein said heteroaryl and heterocycloalkyl may be substituted with 0 to 2 substituents as valency allows independently selected from:
0 to 1 oxo (═O),
0 to 1 —CN,
0 to 2 halogen atoms, and
0 to 2 substituents independently selected from —C₁₋₃alkyl, —OC₁₋₃alkyl and —C₁₋₃alkylene-O—C₁₋₃alkyl.

In certain embodiments, R³ is —CH₂—R⁵, and R⁵ is oxetan-2-yl, oxazol-2-yl, oxazol-5-yl, thiazol-2-yl, thiazol-5-yl, oxetan-3-yl, azetidin-2-yl, azetidin-3-yl, tetrahydrofuran-2-yl, or tetrahydrofuran-3-yl; particularly, R⁵ is oxetan-2-yl, oxazol-2-yl, azetidin-2-yl, or tetrahydrofuran-2-yl.

In certain embodiments, R³ is —CH₂—R⁵, and R⁵ is imidazolyl, such as imidazole-1-yl, imidazole-2-yl and particularly imidazole-4-yl, which is may be substituted with 0 to 2 substituents as valency allows independently selected from
0 to 1 oxo (═O),
0 to 1 —CN,
0 to 2 halogen atoms, and
0 to 2 substituents independently selected from —C₁₋₃alkyl, —OC₁₋₃alkyl and —C₁₋₃alkylene-O—C₁₋₃alkyl.

In certain embodiments, R³ is —CH₂—R⁵, and R⁵ is

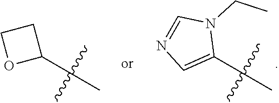

In certain embodiments, R³ is —CH₂CH₂OC₁₋₃alkyl, particularly, —CH₂CH₂OCH₃.

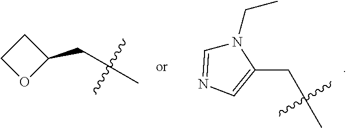

In certain embodiments, R³ is

In certain embodiments, R⁴ is COOH,

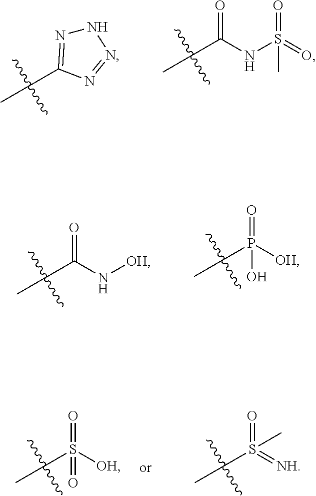

In certain embodiments, the compound has the structure of formula Ia:

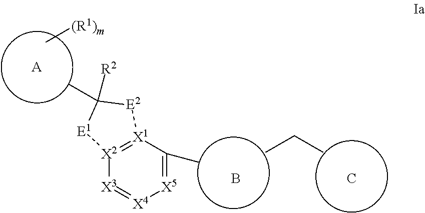

wherein
ring A is phenyl, pyridinyl, or thiophenyl;
each R¹ is independently F, Cl, Br, —CN, —C≡CH, —CH₃, —CF₃, —CH₂OCH₃, —OCH₃, —OCH₂CH₃, or —OCH(CH₃)₂;
m is 0, 1 or 2;
E¹ and E² are independently H, O, NH, or CH₂;
X¹ and X² are independently N or CR⁶, R⁶ is independently absent, H, halogen, —C₁₋₃alkyl or —CN; particularly, R⁶ is independently absent, H, halogen or CH₃;
X³, X⁴ and X⁵ are independently N or CR⁷, wherein R⁷ is independently H, halogen, —C₁₋₃alkyl or —CN; particularly, R⁷ is independently H or CH₃;
--- denotes the presence or absence of a bond; provided that,
   a) when the --- between E¹ and X² denotes the absence of a bond, then the --- between E² and X¹ denotes the presence of a bond, E¹ is H, and X¹ is C,
   b) when the --- between E² and X¹ denotes the absence of a bond, then the --- between E¹ and X² denotes the presence of a bond, E² is H, and X² is C, and
   c) when the --- between E¹ and X² and the --- between E² and X¹ both denote the presence of a bond, then E¹ and E² are independently O, NH or CH₂, and X¹ and X² are C; R² is H or —CH₃;
ring B is

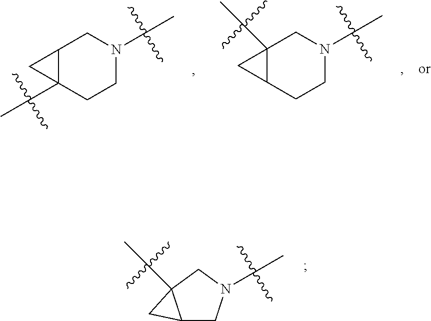 [SMILES: *N1CCC2(*)CC2C1 ; *N1CCC2CC2(*)C1 ; *N1CC2CC2(*)C1]

ring C is

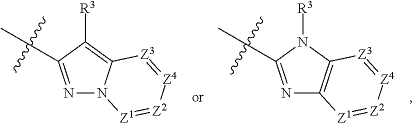

wherein Z¹, Z², Z³ and Z⁴ are independently N, CR⁴ or CR⁸, wherein R⁸ is independently H, halogen, —C₁₋₃alkyl, or —OC₁₋₃alkyl, provided that one of Z¹, Z², Z³ and Z⁴ is CR⁴;
R³ is —CH₂CH₂OC₁₋₃alkyl, particularly, —CH₂CH₂OCH₃; or R³ is —CH₂—R⁵, and R⁵ is oxetan-2-yl, oxazol-2-yl, oxazol-5-yl, thiazol-2-yl, thiazol-5-yl, oxetan-3-yl, azetidin-2-yl, azetidin-3-yl, tetrahydrofuran-2-yl or tetrahydrofuran-3-yl, particularly, oxetan-2-yl, azetidin-2-yl or tetrahydrofuran-2-yl; and
R⁴ is COOH,

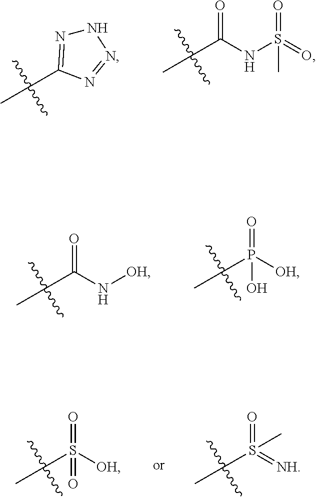

In certain embodiments, the compound has the structure of formula Ia-1 or formula Ia-2:

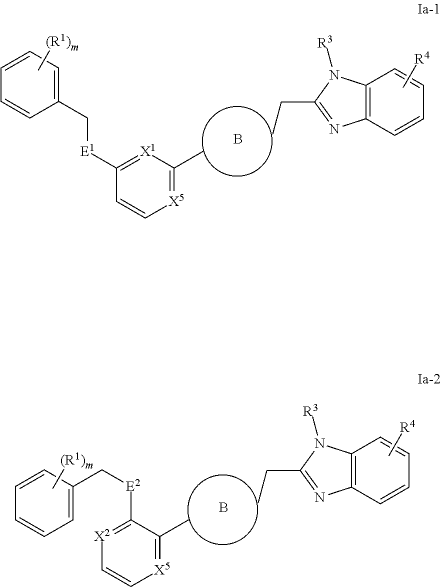

wherein
each R¹ is independently F, Cl, Br, —CN, —C≡CH, —CH₃, —CF₃, —CH₂OCH₃, —OCH₃, —OCH₂CH₃, or —OCH(CH₃)₂;
m is 0, 1 or 2;
E¹ is O, NH, or CH₂;
E² is O, NH, or CH₂;
X¹, X² and X⁵ are independently CH, CCH₃, or N;
ring B is

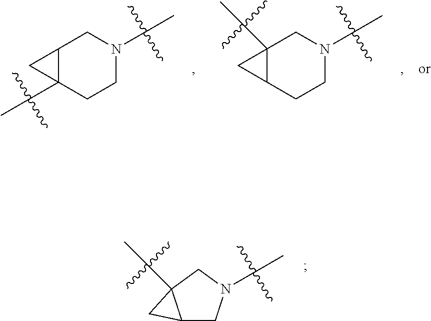 [SMILES: *N1CCC2(*)CC2C1 ; *N1CCC2CC2(*)C1 ; *N1CC2CC2(*)C1]

R³ is —CH₂CH₂OCH₃; or R³ is —CH₂—R⁵, and R⁵ is oxetan-2-yl, oxazol-2-yl, oxazol-5-yl, azetidin-2-yl or tetrahydrofuran-2-yl;
R⁴ is COOH,

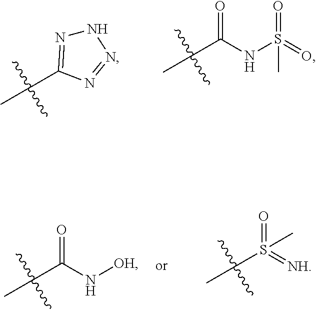

In certain embodiments, the compound has the structure of formula Ia-3:

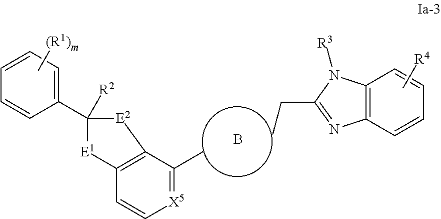

wherein
each R¹ is independently F, Cl, —CN, —C≡CH, or —CH₃;
m is 0, 1 or 2;
E¹ is O, NH, or CH₂;
E² is O, NH, or CH₂;
X⁵ is CH, CCH₃, or N;
R² is H or —CH₃;
ring B is

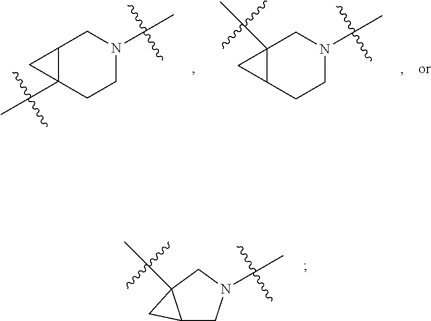 [SMILES: *N1CCC2(*)CC2C1 ; *N1CCC2CC2(*)C1 ; *N1CC2CC2(*)C1]

R³ is —CH₂CH₂OCH₃; or R³ is —CH₂—R⁵, and R⁵ is oxetan-2-yl, oxazol-2-yl, oxazol-5-yl, azetidin-2-yl or tetrahydrofuran-2-yl;
R⁴ is COOH,

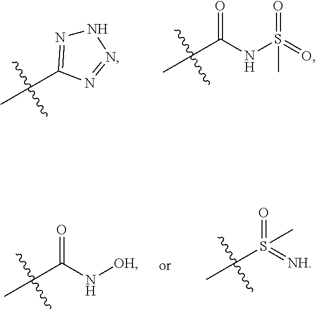

In certain embodiments, the compound has the structure of formula Ia-4:

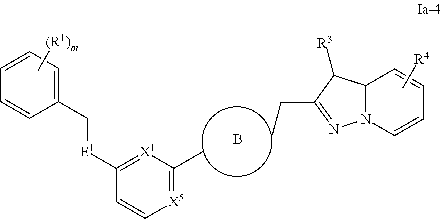

wherein
each R¹ is independently F, Cl, —CN, —C≡CH, or —CH₃;
m is 0, 1 or 2;
E¹ is O, NH, or CH₂;
X¹ and X⁵ are independently CH, CCH₃, or N;
ring B is

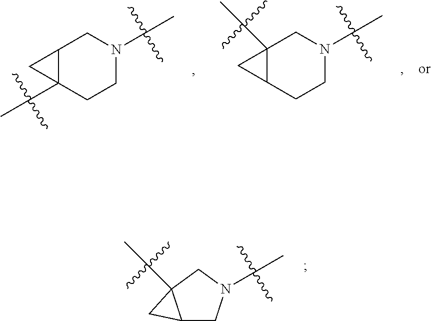 [SMILES: *N1CCC2(*)CC2C1 ; *N1CCC2CC2(*)C1 ; *N1CC2CC2(*)C1]

R³ is —CH₂CH₂OCH₃; or R³ is —CH₂—R⁵, and R⁵ is oxetan-2-yl, oxazol-2-yl, oxazol-5-yl, azetidin-2-yl or tetrahydrofuran-2-yl;
R⁴ is COOH,

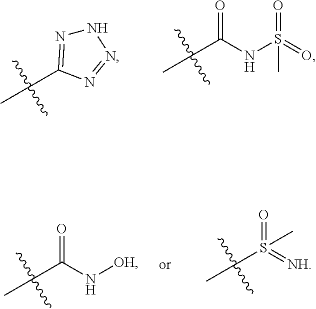

In certain embodiments, the compound has the structure of formula Ib-1 or Ib-2:

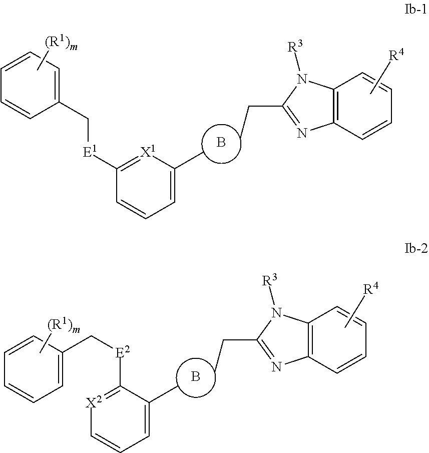

wherein
each R¹ is independently F, Cl, —CN, —C≡CH, or —CH₃;
m is 0, 1 or 2;
E¹ is O, NH, or CH₂;
E² is O, NH, or CH₂;
X¹ and X² is independently CH, CCH₃, or N;
ring B is

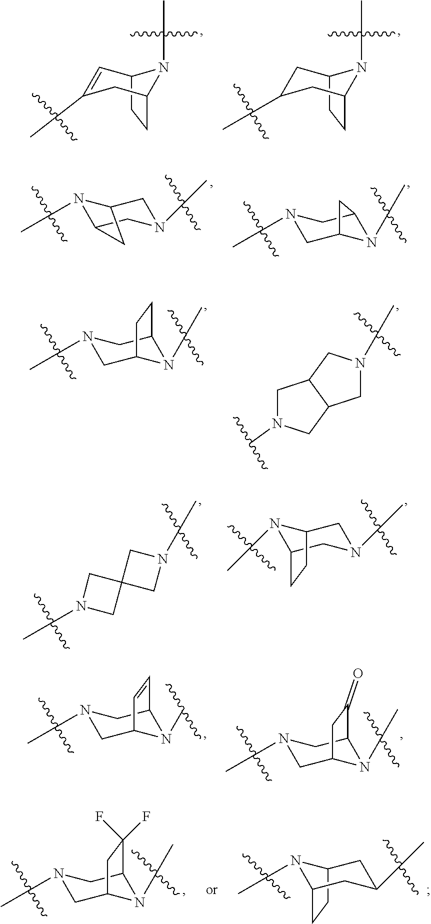

R³ is —CH₂CH₂OCH₃; or R³ is —CH₂—R⁵, and R⁵ is oxetan-2-yl, azetidin-2-yl or tetrahydrofuran-2-yl;
R⁴ is COOH,

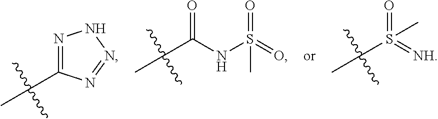

In certain embodiments, the compound has the structure of formula Ic:

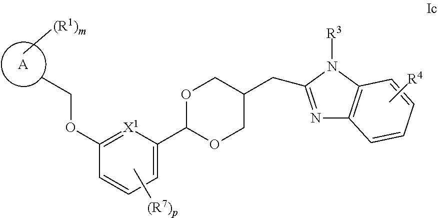

wherein
ring A is phenyl or pyridinyl;
each R¹ is independently F, Cl, —CN, —C≡CH, or —CH₃;
m is 0, 1 or 2;
X¹ is CH, CCH₃, or N;
R⁷ is independently-C₁₋₃alkyl; particularly, R⁶ is CH₃;
p is 0, 1 or 2;
R³ is —CH₂CH₂OCH₃; or R³ is —CH₂—R⁵, and R⁵ is oxetan-2-yl, azetidin-2-yl or tetrahydrofuran-2-yl;
R⁴ is COOH,

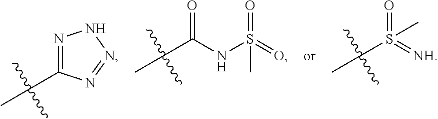

In certain embodiments, the compound has the structure of formula Id:

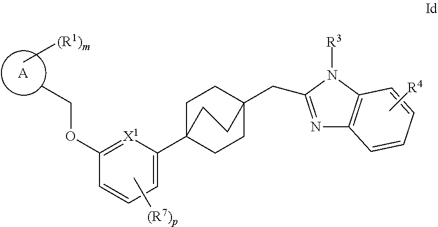

wherein
ring A is phenyl or pyridinyl;
each R¹ is independently F, Cl, —CN, —C≡CH, or —CH₃;
m is 0, 1 or 2;
X¹ is CH, CCH₃, or N;
R⁷ is independently-C₁₋₃alkyl; particularly, R⁶ is CH₃;
p is 0, 1 or 2;
R³ is —CH₂CH₂OCH₃; or R³ is —CH₂—R⁵, and R⁵ is oxetan-2-yl, azetidin-2-yl or tetrahydrofuran-2-yl;
R⁴ is COOH,

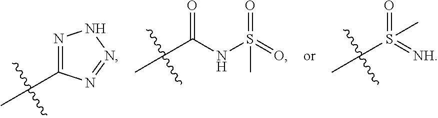

In certain embodiments, ring B is:

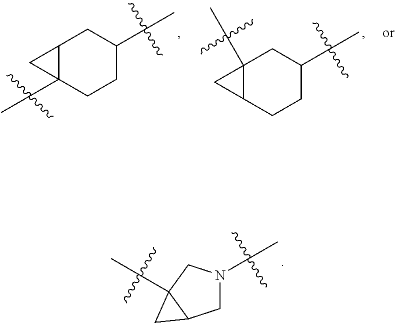 [SMILES: *C1CCC2(*)CC2C1 ; *C1CCC2CC2(*)C1 ; *N1CC2CC2(*)C1]

In certain embodiments, ring C is:

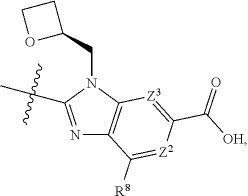

wherein Z² and Z³ are independently N or CH₂, R⁸ has the same meaning as defined in Formula I.

In certain embodiments, R⁸ is independently H, CN, halogen, —C(O) C₁₋₃alkyl, —OC₁₋₃alkyl, —C₃₋₆cycloalkyl, or —C₁₋₃alkyl, wherein said alkyl and said cycloalkyl of —C(O) C₁₋₃alkyl, —OC₁₋₃alkyl, —C₃₋₆cycloalkyl, and —C₁₋₃alkyl are independently unsubstituted or substituted with one or more substituents selected from OH, NH₂, —CN, and halogen.

In certain embodiments,

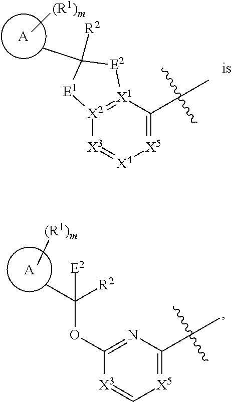

and particularly E² is independently H, D, or halogen.

In certain embodiments, the compound has the structure of formula Ie:

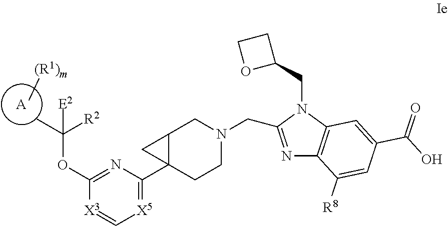

wherein

each R¹ is independently F, Cl, —CN, —C≡CH, —C₁₋₃alkyl, or —OC₁₋₃alkyl, wherein said alkyl of —C₁₋₃alkyl and —OC₁₋₃alkyl is substituted with 0 to 5 halogen atoms;
ring A is phenyl or pyridinyl;
m is 1, 2 or 3;
E² is independently H, D, or halogen;
X³ and X⁵ are independently N or CR⁷, wherein R⁷ is independently H, halogen, —C₁₋₃alkyl, —OC₁₋₃alkyl or —CN, wherein said alkyl of —C₁₋₃alkyl and —OC₁₋₃alkyl is substituted with 0 to 3 halogen atoms;
R² and R⁸ have the same meaning as defined in Formula I.

In certain embodiments,

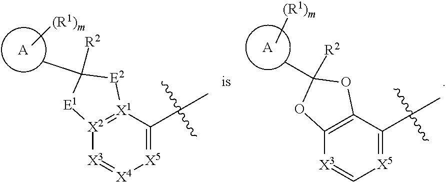

In certain embodiments, the compound has the structure of formula If:

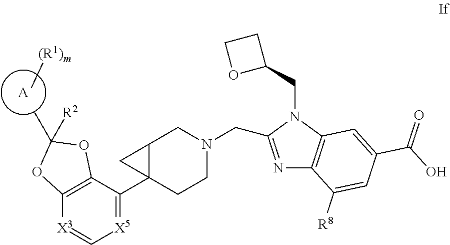

wherein
each R¹ is independently F, Cl, —CN, —C≡CH, —C₁₋₃alkyl, or —OC₁₋₃alkyl, wherein said
alkyl of —C₁₋₃alkyl and —OC₁₋₃alkyl is substituted with 0 to 5 halogen atoms;
ring A is phenyl or pyridinyl;
m is 1, 2 or 3;
X³ and X⁵ are independently N or CR⁷, wherein R⁷ is independently H, halogen, —C₁₋₃alkyl, —OC₁₋₃alkyl or —CN, wherein said alkyl of —C₁₋₃alkyl and —OC₁₋₃alkyl is substituted with 0 to 3 halogen atoms; and
R² and R⁸ have the same meaning as defined in Formula I.

In certain embodiments, R² is H, D, F, Cl, or —CH₃.

In certain embodiments, m is 2 or 3.

In certain embodiments,

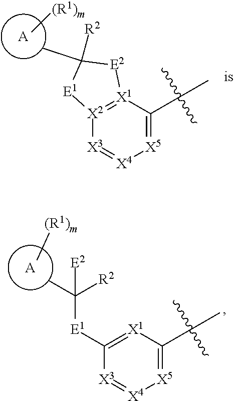

and particularly E² is independently H, D, or halogen. Particularly, E¹ is O.

In certain embodiments, the compound has the structure of formula Ig:

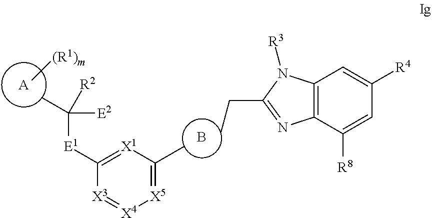

wherein

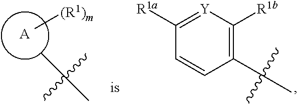

Y is N or CR^{1c};
R^{1a} is H, halogen, —CN, —C≡CH, —C₁₋₃alkyl, —C₁₋₃alkylOC₁₋₃alkyl, or —OC₁₋₃alkyl, wherein said alkyl of —C₁₋₃alkyl and —OC₁₋₃alkyl is substituted with 0 to 5 halogen atoms;
R^{1b} is H, halogen, —C₁₋₃alkyl, or —OC₁₋₃alkyl, wherein said alkyl of —C₁₋₃alkyl and —OC₁₋₃alkyl is substituted with 0 to 5 halogen atoms;
R^{1c} is H or halogen;
E¹ is O;
E² is independently H, D, or halogen;
X¹ is N or CR⁶, wherein R⁶ is H, halogen, —C₁₋₃alkyl, or —CN;
X³, X⁴ and X⁵ are independently N or CR⁷, wherein R⁷ is independently H, halogen, —C₁₋₃alkyl, —OC₁₋₃alkyl or —CN, wherein said alkyl of —C₁₋₃alkyl and —OC₁₋₃alkyl is substituted with 0 to 3 halogen atoms;
ring B is

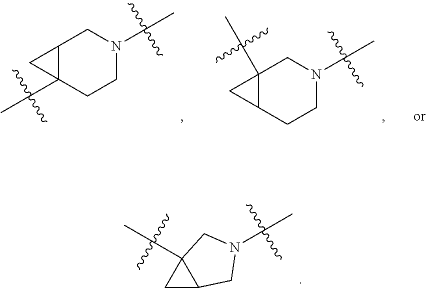 [SMILES: *N1CCC2(*)CC2C1 ; *N1CCC2CC2(*)C1 ; *N1CC2CC2(*)C1]

R³ is —CH₂—R⁵, and R⁵ is a 5- or 6-membered heteroaryl group or a 4- to 6-membered heterocycloalkyl group wherein said heteroaryl and heterocycloalkyl may be substituted with 0 to 2 substituents as valency allows independently selected from:
   0 to 1 oxo (═O),
   0 to 1 —CN,
   0 to 2 halogen atoms, and
   0 to 2 substituents independently selected from —C₁₋₃alkyl, —OC₁₋₃alkyl and —C₁₋₃alkylene-O—C₁₋₃alkyl;
R⁴ is COOH,

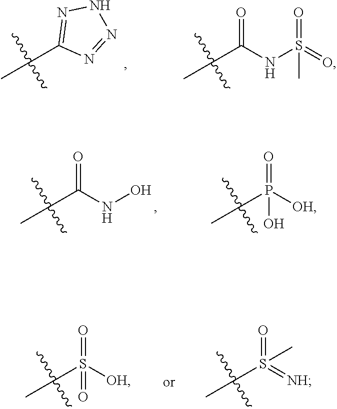

and
R⁸ is independently H, —OH, CN, halogen, —C(O) C₁₋₃alkyl, —C(O) C₃₋₆cycloalkyl, —OC₁₋₃alkyl, —C₃₋₆cycloalkyl, or —C₁₋₃alkyl, wherein said alkyl and said cycloalkyl of —C(O) C₁₋₃alkyl, —C(O) C₃₋₆cycloalkyl, —OC₁₋₃alkyl, —C₃₋₆cycloalkyl, and —C₁₋₃alkyl are independently unsubstituted or substituted with one or more substituents selected from D (i.e., deuterium), OH, NH₂, —CN, and halogen.

In certain embodiments, R⁸ is independently H, CN, F, Cl, —C(O) CH₃, —OCH₃, —OCD₃, —OCH₂CH₃, —OCH(CH₃)₂, —OCF₃, -cyclopropyl, or —CH₃.

In certain embodiments, R^{1b} is —OC₁₋₃alkyl, wherein said alkyl of —OC₁₋₃alkyl is substituted with 0 to 5 halogen atoms.

In certain embodiments, R⁸ is not H.

In certain embodiments, when R^{1b} is —OC₁₋₃alkyl, R⁸ may be independently H, —OH, CN, halogen, —C(O) C₁₋₃alkyl, —C(O) C₃₋₆cycloalkyl, —OC₁₋₃alkyl, —C₃₋₆cycloalkyl, or —C₁₋₃alkyl, wherein said alkyl and said cycloalkyl of —C(O) C₁₋₃alkyl, —C(O) C₃₋₆cycloalkyl, —OC₁₋₃alkyl, —C₃₋₆cycloalkyl, and —C₁₋₃alkyl are independently unsubstituted or substituted with one or more substituents selected from D (i.e., deuterium), OH, NH₂, —CN, and halogen; and when R⁸ is not H, R^{1b} may be H, halogen, —C₁₋₃alkyl, or —OC₁₋₃alkyl, wherein said alkyl of —C₁₋₃alkyl and —OC₁₋₃alkyl is substituted with 0 to 5 halogen atoms.

In certain embodiments, the compound is selected from the group consisting of the following compounds:

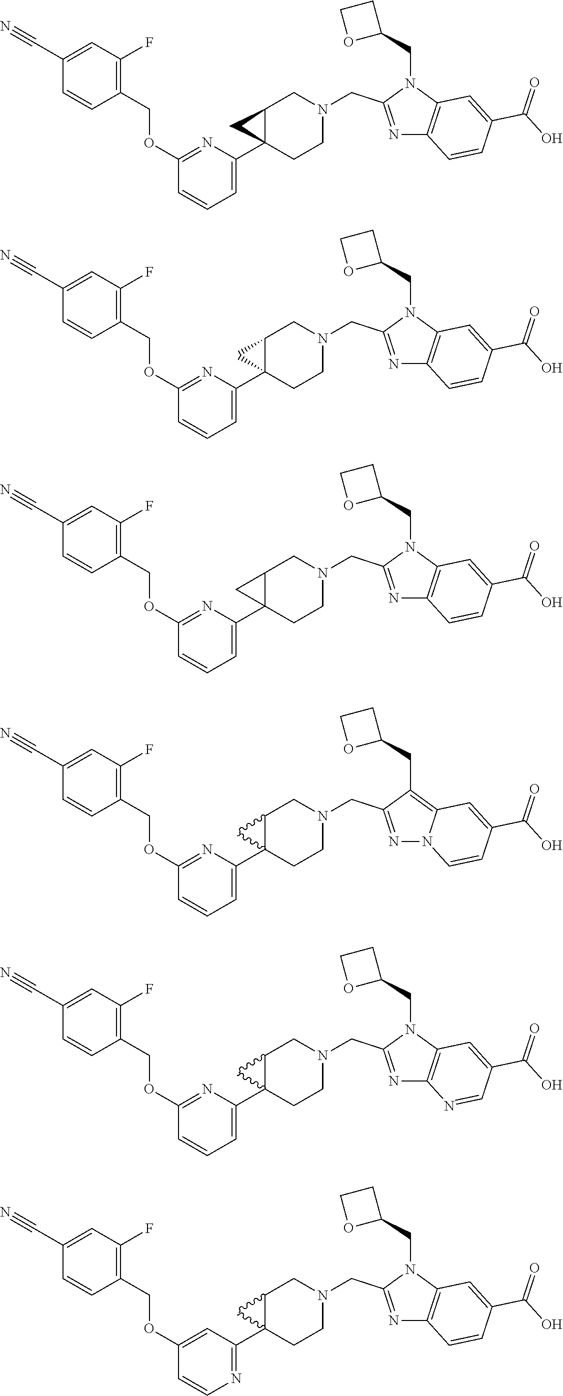

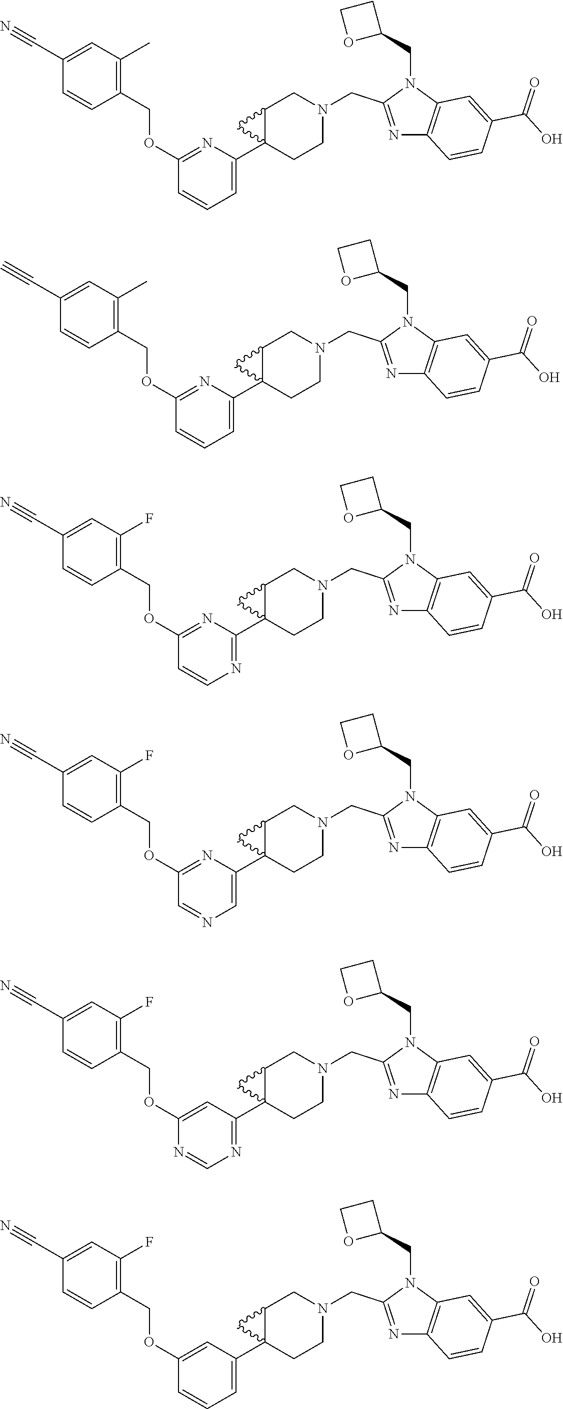

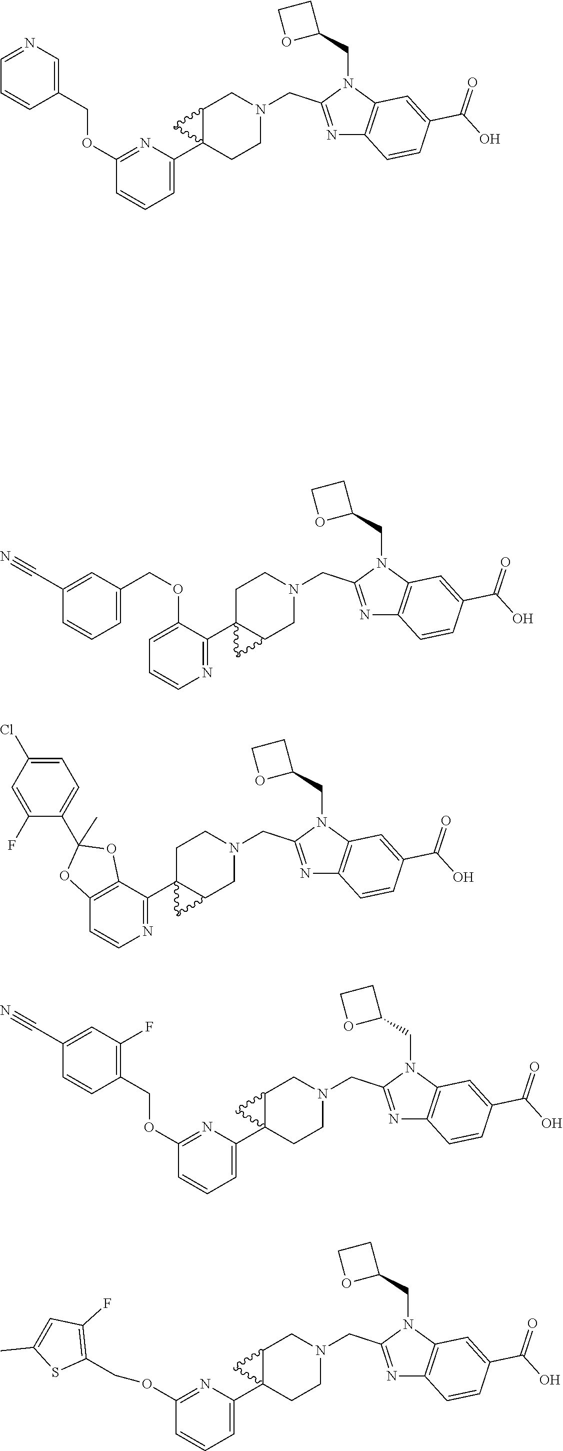

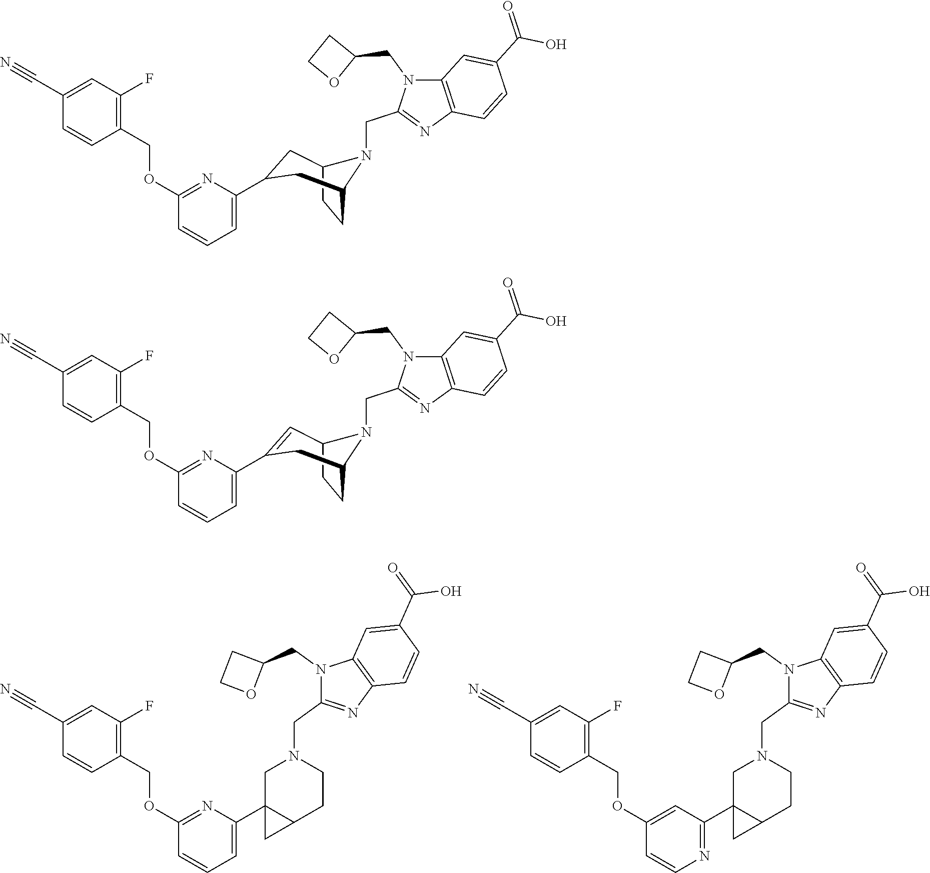

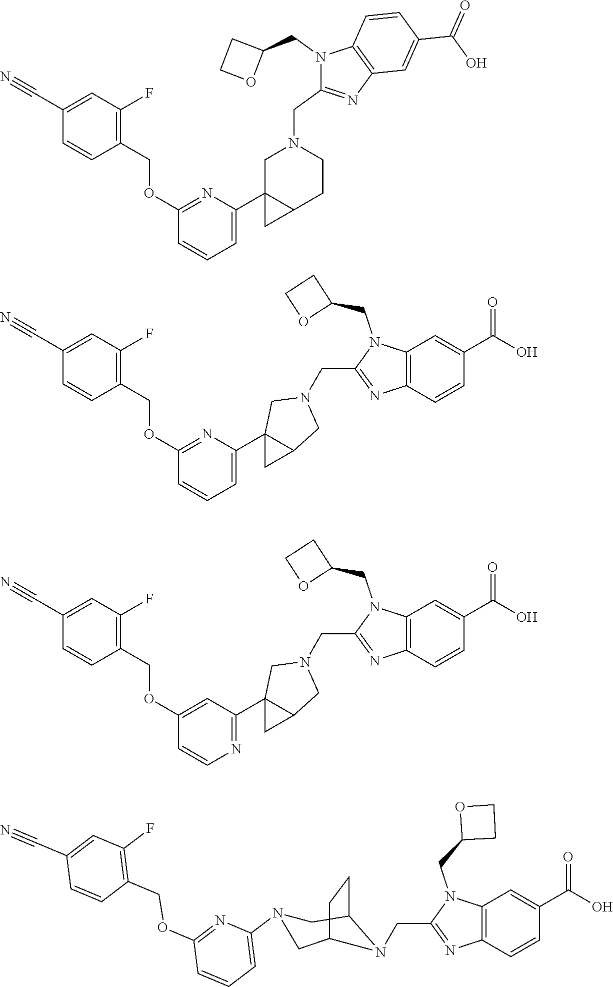 [SMILES: N#Cc1ccc(COc2cccc(C34CC3CCN(Cc3nc5cc(C(=O)O)ccc5n3C[C@@H]3CCO3)C4)n2)c(F)c1 ; N#Cc1ccc(COc2cccc(C34CC3CN(Cc3nc5ccc(C(=O)O)cc5n3C[C@@H]3CCO3)C4)n2)c(F)c1 ; N#Cc1ccc(COc2ccnc(C34CC3CN(Cc3nc5ccc(C(=O)O)cc5n3C[C@@H]3CCO3)C4)c2)c(F)c1 ; N#Cc1ccc(COc2cccc(N3CC4CCC(C3)N4Cc3nc4ccc(C(=O)O)cc4n3C[C@@H]3CCO3)n2)c(F)c1]

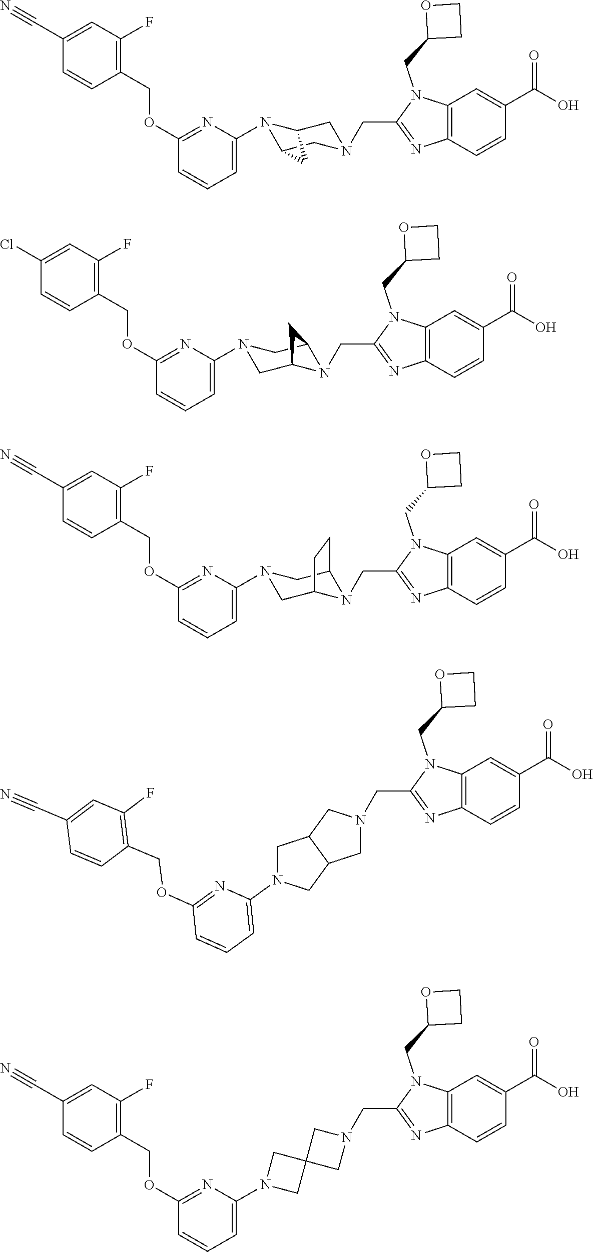 [SMILES: N#Cc1ccc(COc2cccc(N3[C@H]4C[C@H]3CN(Cc3nc5ccc(C(=O)O)cc5n3C[C@@H]3CCO3)C4)n2)c(F)c1 ; O=C(O)c1ccc2nc(CN3[C@@H]4C[C@@H]3CN(c3cccc(OCc5ccc(Cl)cc5F)n3)C4)n(C[C@@H]3CCO3)c2c1 ; N#Cc1ccc(COc2cccc(N3CC4CCC(C3)N4Cc3nc4ccc(C(=O)O)cc4n3C[C@H]3CCO3)n2)c(F)c1 ; N#Cc1ccc(COc2cccc(N3CC4CN(Cc5nc6ccc(C(=O)O)cc6n5C[C@@H]5CCO5)CC4C3)n2)c(F)c1 ; N#Cc1ccc(COc2cccc(N3CC4(CN(Cc5nc6ccc(C(=O)O)cc6n5C[C@@H]5CCO5)C4)C3)n2)c(F)c1]

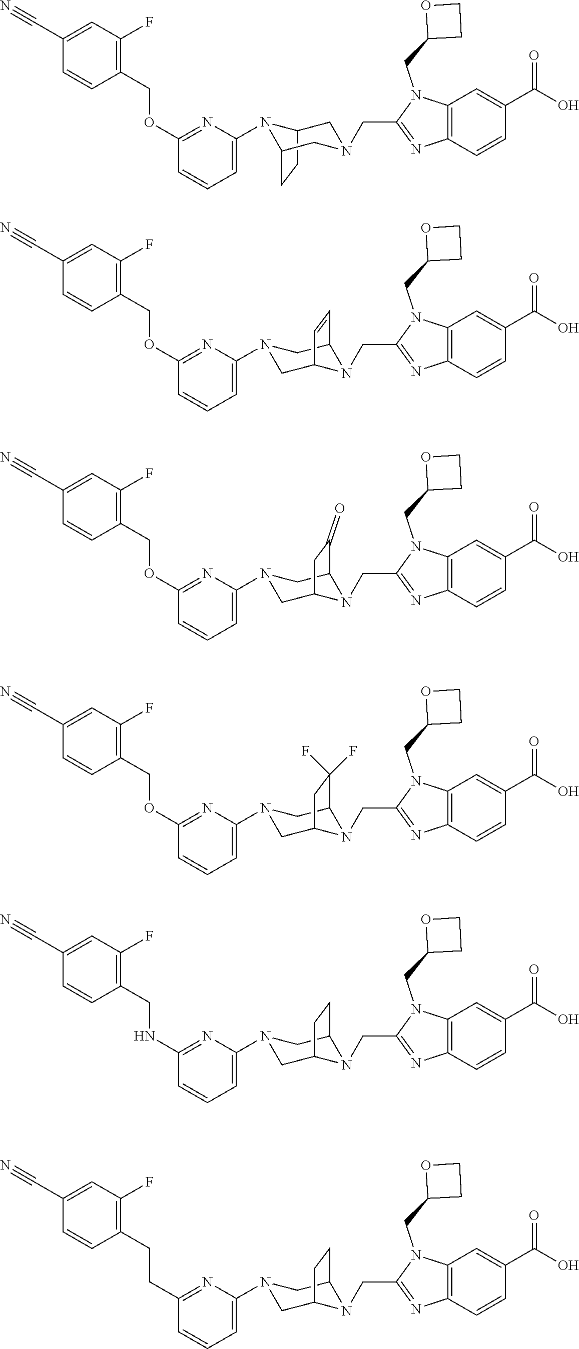 [SMILES: N#Cc1ccc(COc2cccc(N3C4CCC3CN(Cc3nc5ccc(C(=O)O)cc5n3C[C@@H]3CCO3)C4)n2)c(F)c1 ; N#Cc1ccc(COc2cccc(N3CC4C=CC(C3)N4Cc3nc4ccc(C(=O)O)cc4n3C[C@@H]3CCO3)n2)c(F)c1 ; N#Cc1ccc(COc2cccc(N3CC4CC(=O)C(C3)N4Cc3nc4ccc(C(=O)O)cc4n3C[C@@H]3CCO3)n2)c(F)c1 ; N#Cc1ccc(COc2cccc(N3CC4CC(F)(F)C(C3)N4Cc3nc4ccc(C(=O)O)cc4n3C[C@@H]3CCO3)n2)c(F)c1 ; N#Cc1ccc(CNc2cccc(N3CC4CCC(C3)N4Cc3nc4ccc(C(=O)O)cc4n3C[C@@H]3CCO3)n2)c(F)c1 ; N#Cc1ccc(CCc2cccc(N3CC4CCC(C3)N4Cc3nc4ccc(C(=O)O)cc4n3C[C@@H]3CCO3)n2)c(F)c1]

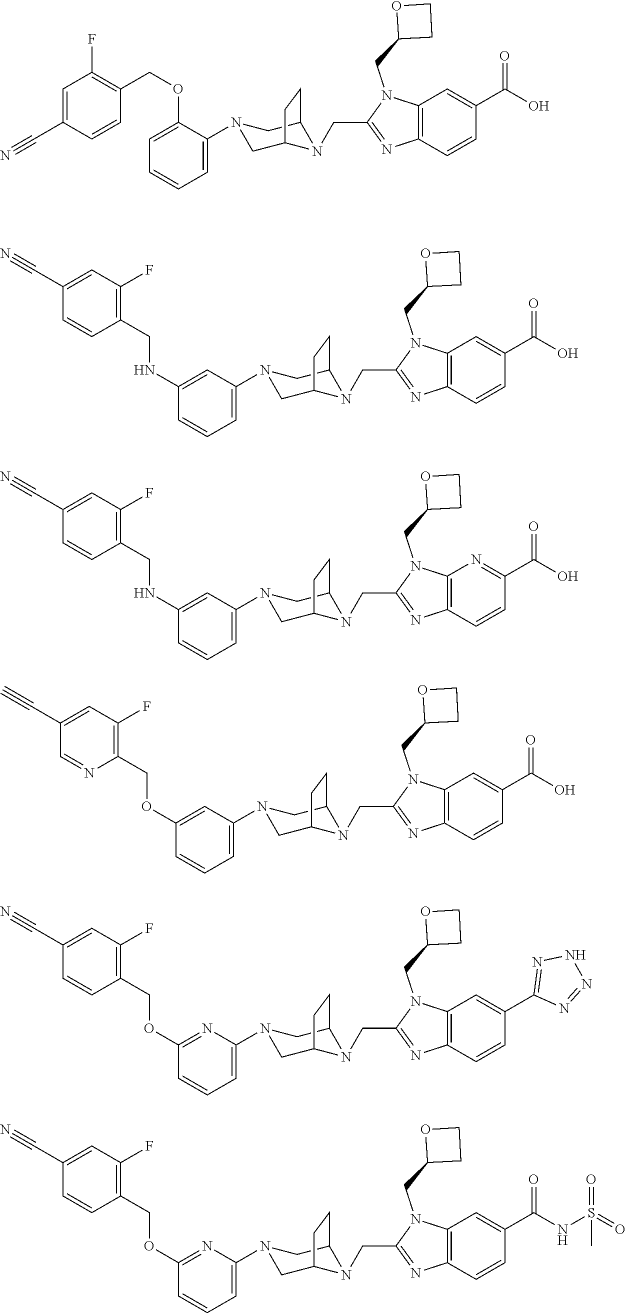 [SMILES: N#Cc1ccc(COc2ccccc2N2CC3CCC(C2)N3Cc2nc3ccc(C(=O)O)cc3n2C[C@@H]2CCO2)c(F)c1 ; N#Cc1ccc(CNc2cccc(N3CC4CCC(C3)N4Cc3nc4ccc(C(=O)O)cc4n3C[C@@H]3CCO3)c2)c(F)c1 ; N#Cc1ccc(CNc2cccc(N3CC4CCC(C3)N4Cc3nc4ccc(C(=O)O)nc4n3C[C@@H]3CCO3)c2)c(F)c1 ; C#Cc1cnc(COc2cccc(N3CC4CCC(C3)N4Cc3nc4ccc(C(=O)O)cc4n3C[C@@H]3CCO3)c2)c(F)c1 ; N#Cc1ccc(COc2cccc(N3CC4CCC(C3)N4Cc3nc4ccc(-c5nn[nH]n5)cc4n3C[C@@H]3CCO3)n2)c(F)c1 ; CS(=O)(=O)NC(=O)c1ccc2nc(CN3C4CCC3CN(c3cccc(OCc5ccc(C#N)cc5F)n3)C4)n(C[C@@H]3CCO3)c2c1]

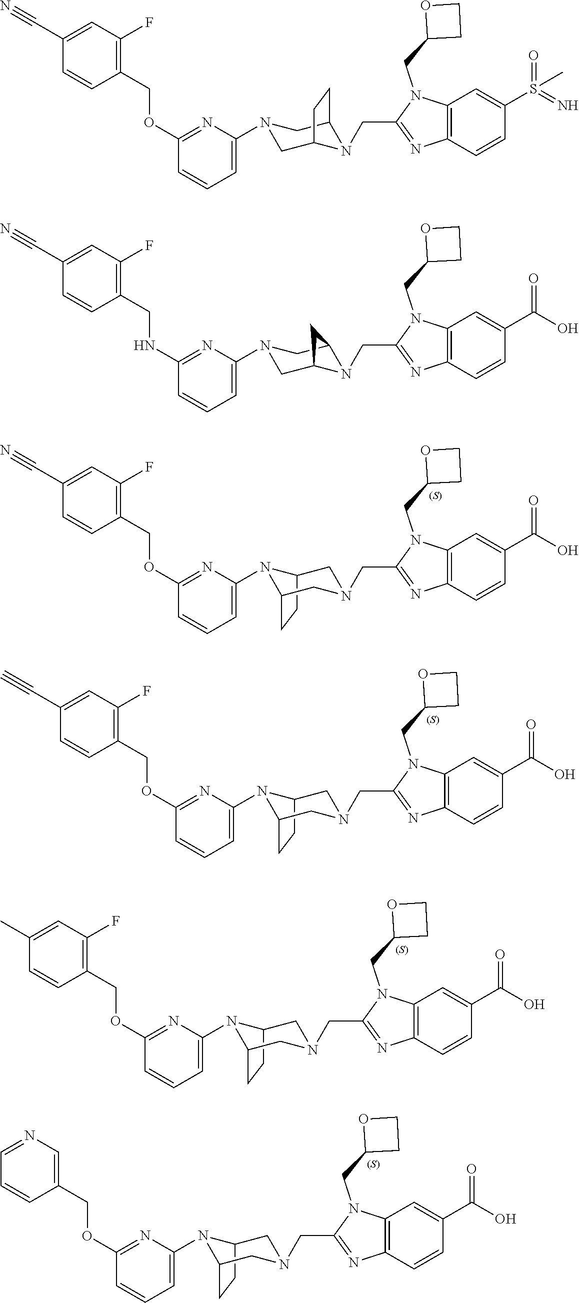

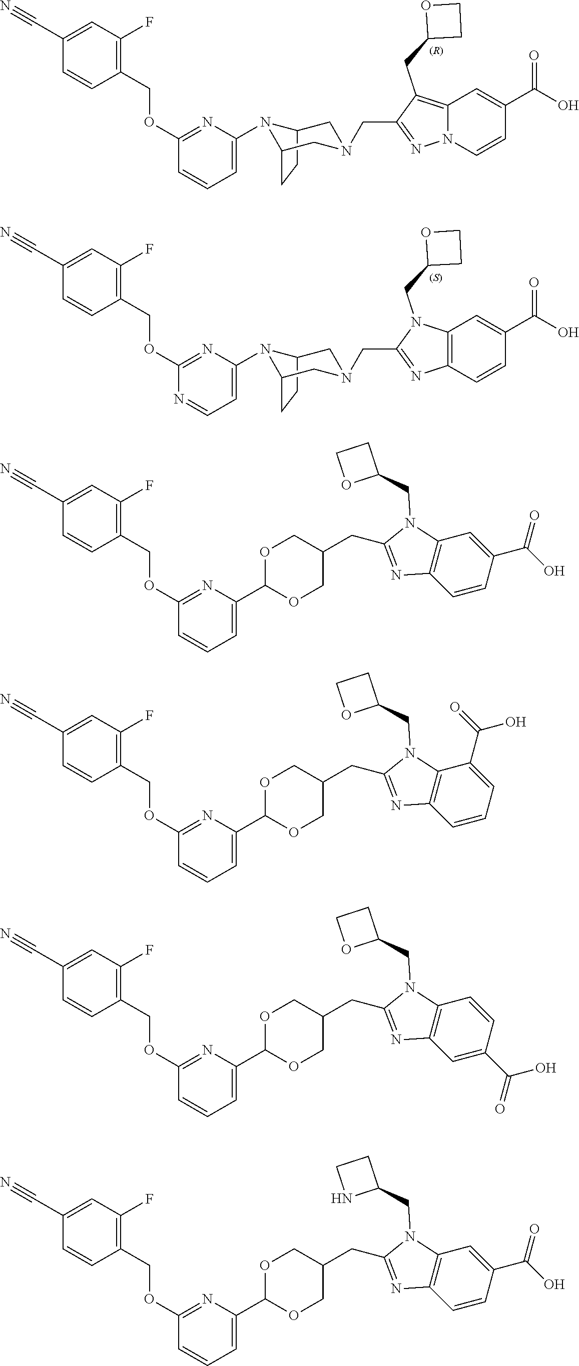

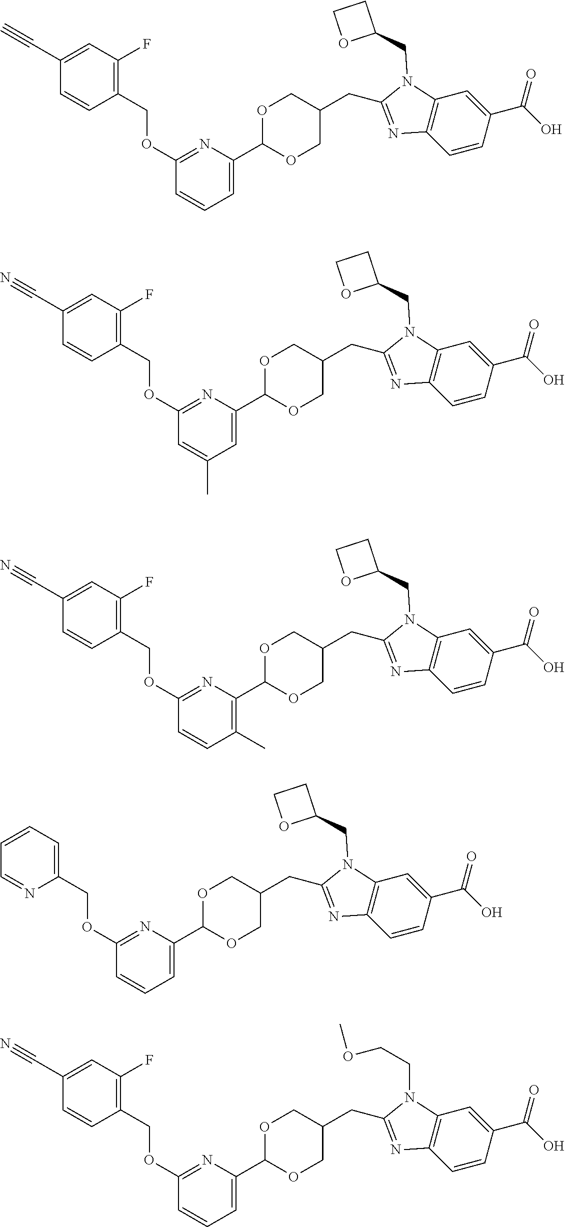 [SMILES: C#Cc1ccc(COc2cccc(C3OCC(Cc4nc5ccc(C(=O)O)cc5n4C[C@@H]4CCO4)CO3)n2)c(F)c1 ; Cc1cc(OCc2ccc(C#N)cc2F)nc(C2OCC(Cc3nc4ccc(C(=O)O)cc4n3C[C@@H]3CCO3)CO2)c1 ; Cc1ccc(OCc2ccc(C#N)cc2F)nc1C1OCC(Cc2nc3ccc(C(=O)O)cc3n2C[C@@H]2CCO2)CO1 ; O=C(O)c1ccc2nc(CC3COC(c4cccc(OCc5ccccn5)n4)OC3)n(C[C@@H]3CCO3)c2c1 ; COCCn1c(CC2COC(c3cccc(OCc4ccc(C#N)cc4F)n3)OC2)nc2ccc(C(=O)O)cc21]

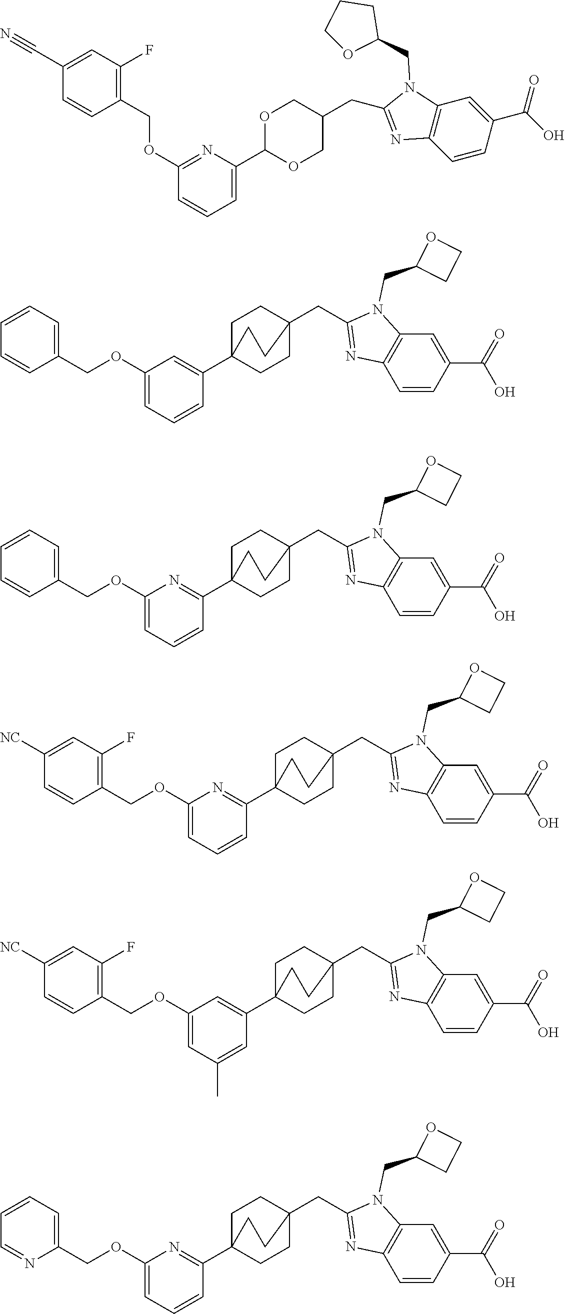 [SMILES: N#Cc1ccc(COc2cccc(C3OCC(Cc4nc5ccc(C(=O)O)cc5n4C[C@@H]4CCCO4)CO3)n2)c(F)c1 ; O=C(O)c1ccc2nc(CC34CCC(c5cccc(OCc6ccccc6)c5)(CC3)CC4)n(C[C@@H]3CCO3)c2c1 ; O=C(O)c1ccc2nc(CC34CCC(c5cccc(OCc6ccccc6)n5)(CC3)CC4)n(C[C@@H]3CCO3)c2c1 ; N#Cc1ccc(COc2cccc(C34CCC(Cc5nc6ccc(C(=O)O)cc6n5C[C@@H]5CCO5)(CC3)CC4)n2)c(F)c1 ; Cc1cc(OCc2ccc(C#N)cc2F)cc(C23CCC(Cc4nc5ccc(C(=O)O)cc5n4C[C@@H]4CCO4)(CC2)CC3)c1 ; O=C(O)c1ccc2nc(CC34CCC(c5cccc(OCc6ccccn6)n5)(CC3)CC4)n(C[C@@H]3CCO3)c2c1]

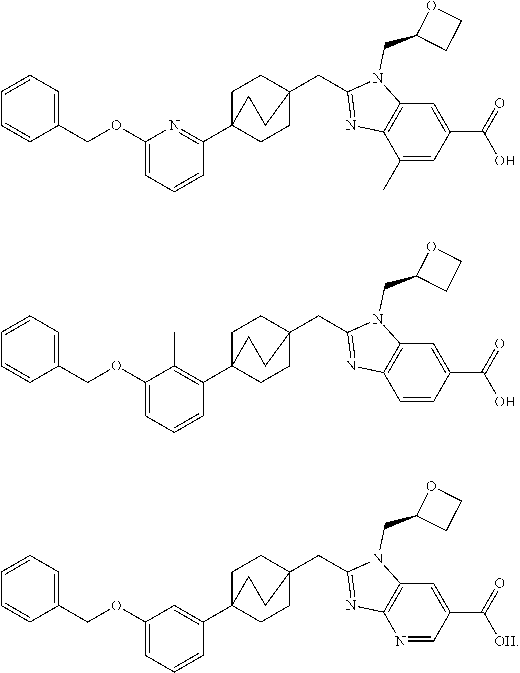 [SMILES: Cc1cc(C(=O)O)cc2c1nc(CC13CCC(c4cccc(OCc5ccccc5)n4)(CC1)CC3)n2C[C@@H]1CCO1 ; Cc1c(OCc2ccccc2)cccc1C12CCC(Cc3nc4ccc(C(=O)O)cc4n3C[C@@H]3CCO3)(CC1)CC2 ; O=C(O)c1cnc2nc(CC34CCC(c5cccc(OCc6ccccc6)c5)(CC3)CC4)n(C[C@@H]3CCO3)c2c1]

In certain embodiments, the compound is selected from the group consisting of the following compounds:

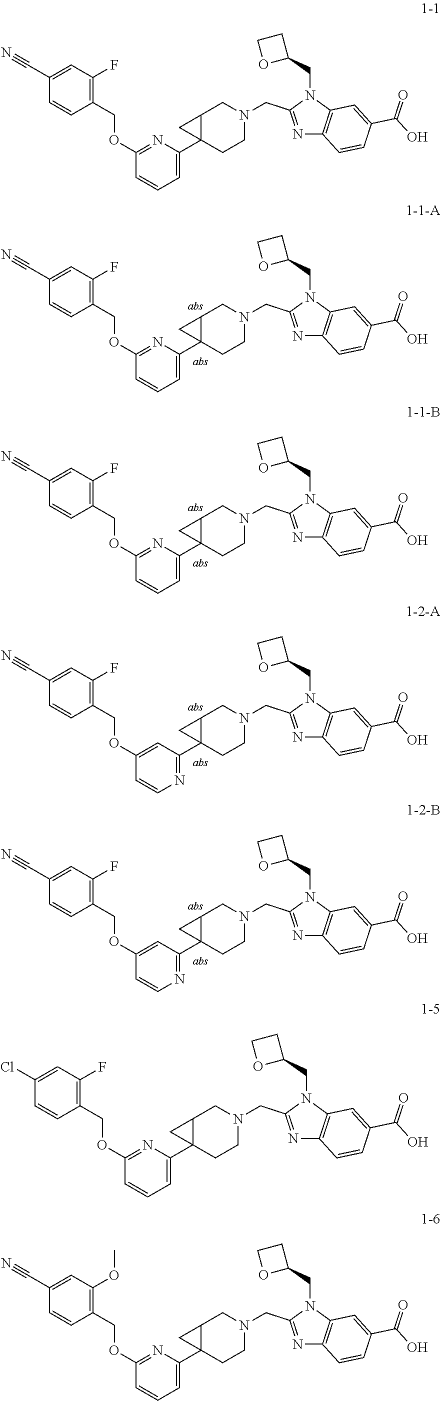

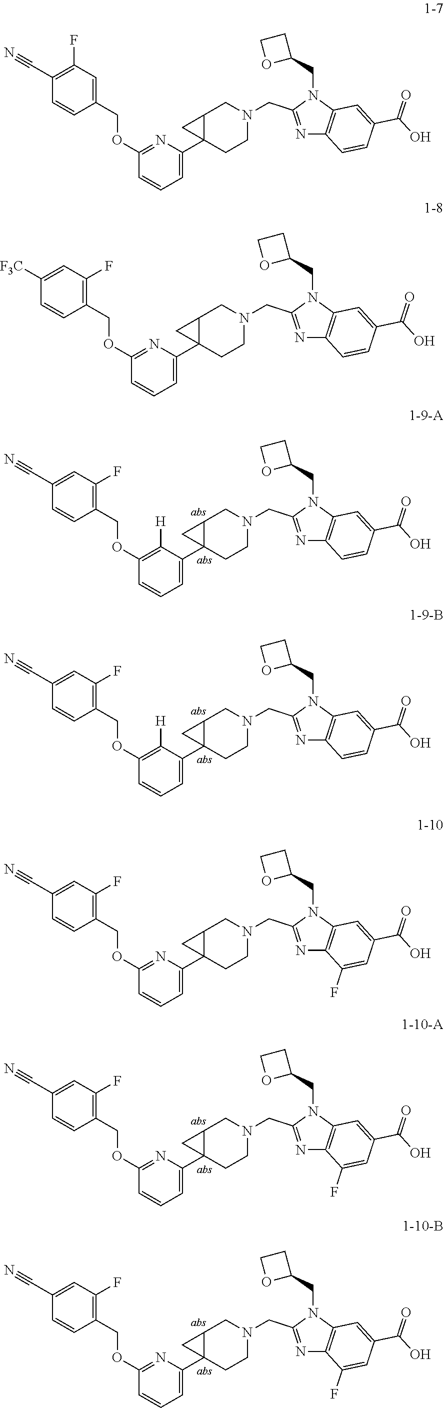

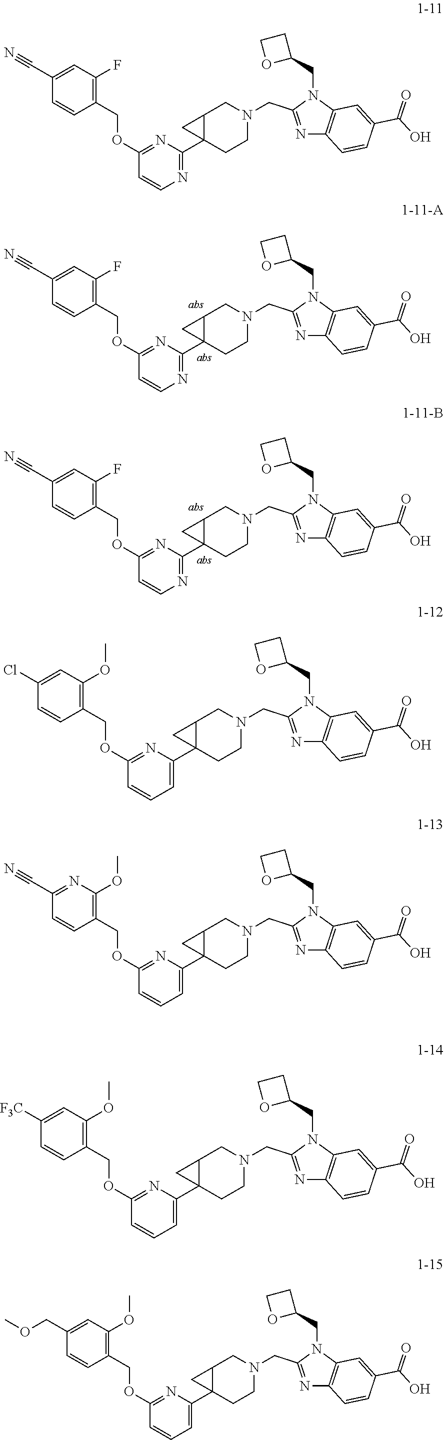

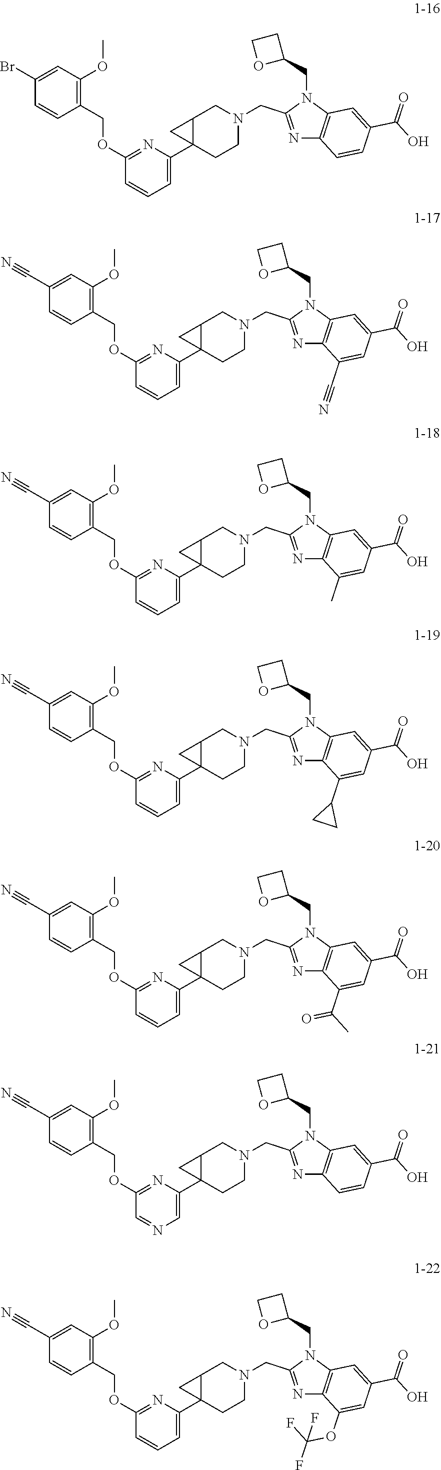

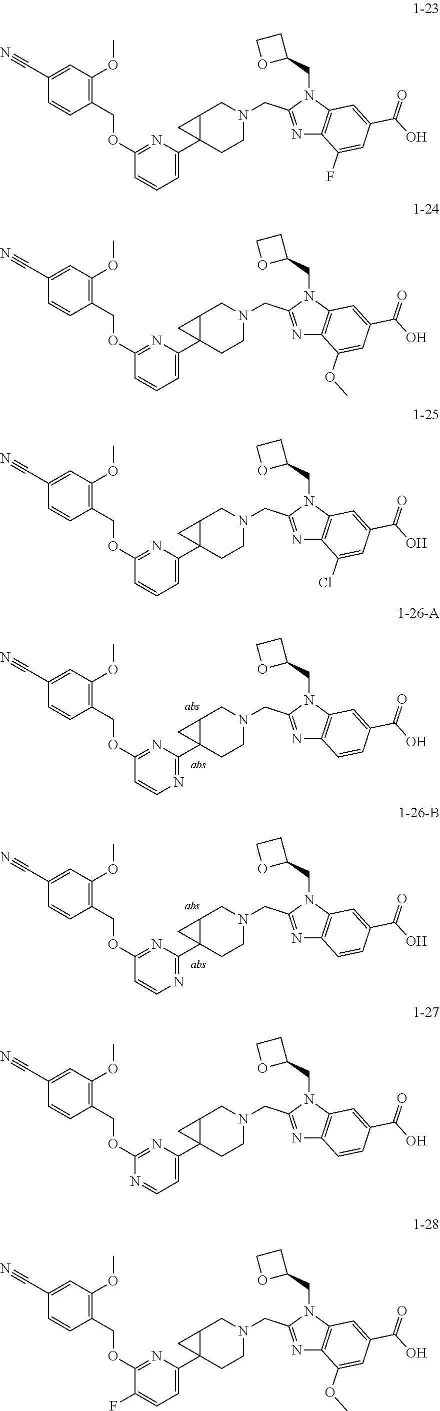

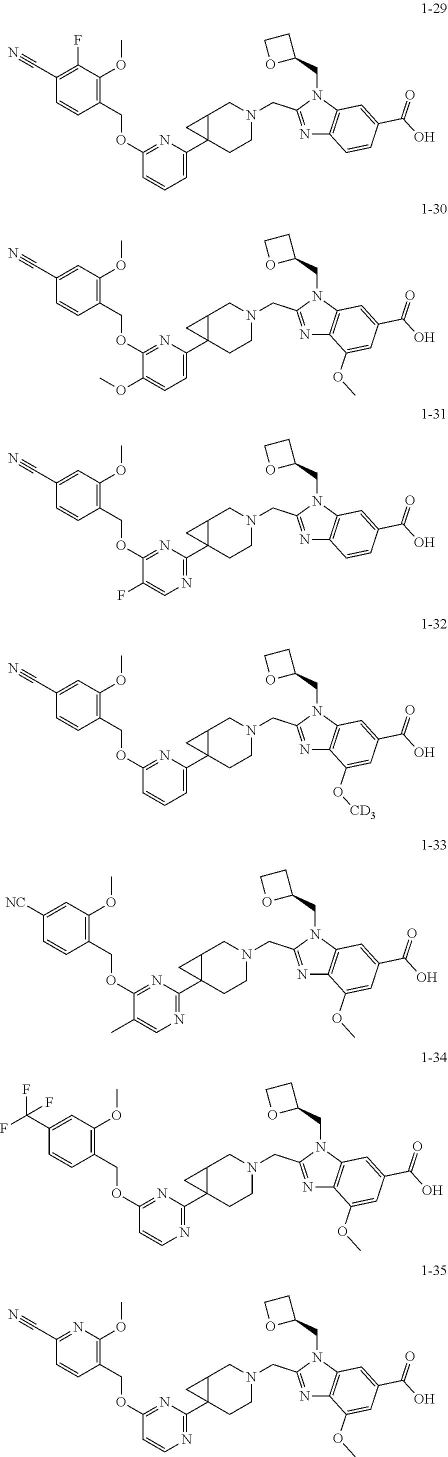

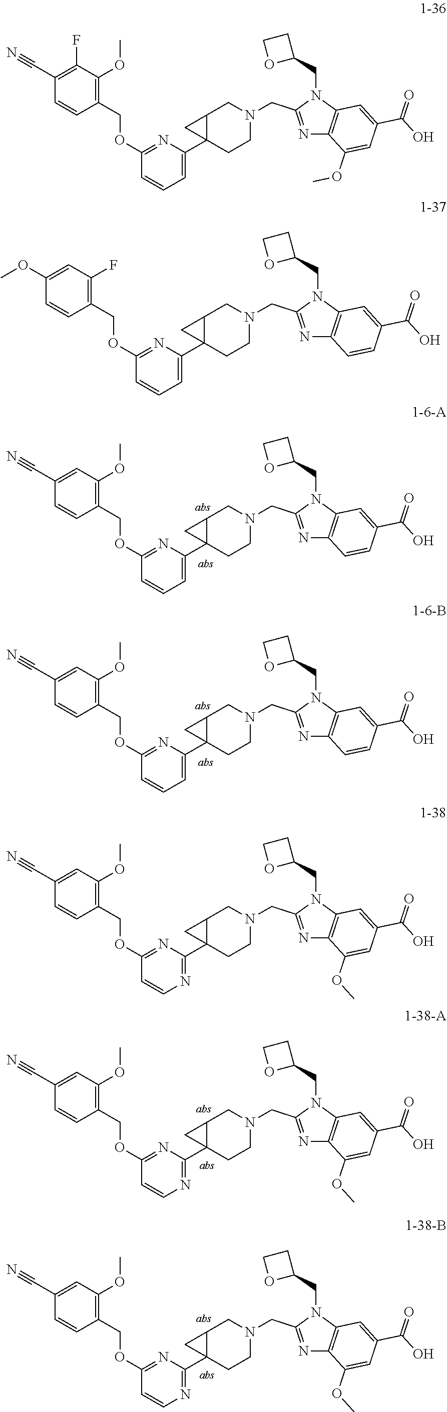

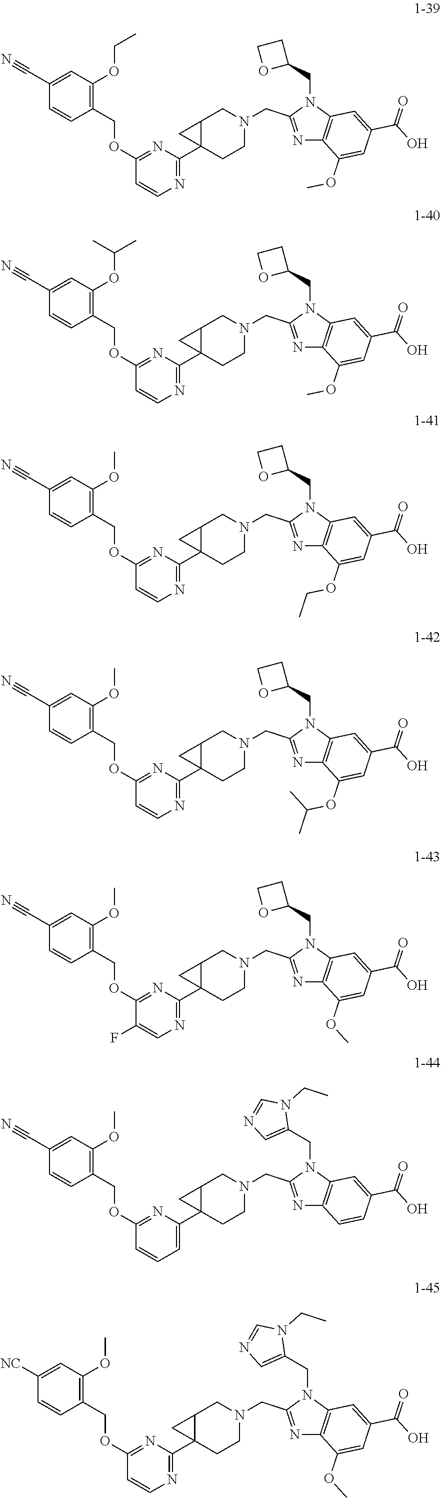

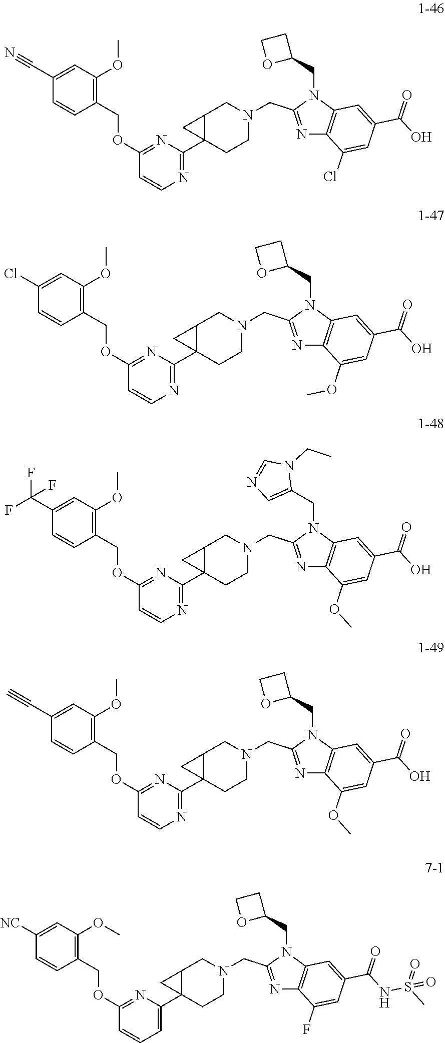 [SMILES: COc1cc(C#N)ccc1COc1ccnc(C23CCN(Cc4nc5c(Cl)cc(C(=O)O)cc5n4C[C@@H]4CCO4)CC2C3)n1 ; COc1cc(Cl)ccc1COc1ccnc(C23CCN(Cc4nc5c(OC)cc(C(=O)O)cc5n4C[C@@H]4CCO4)CC2C3)n1 ; CCn1cncc1Cn1c(CN2CCC3(c4nccc(OCc5ccc(C(F)(F)F)cc5OC)n4)CC3C2)nc2c(OC)cc(C(=O)O)cc21 ; C#Cc1ccc(COc2ccnc(C34CCN(Cc5nc6c(OC)cc(C(=O)O)cc6n5C[C@@H]5CCO5)CC3C4)n2)c(OC)c1 ; COc1cc(C#N)ccc1COc1cccc(C23CCN(Cc4nc5c(F)cc(C(=O)NS(C)(=O)=O)cc5n4C[C@@H]4CCO4)CC2C3)n1]

In certain embodiments, the compound is selected from the group consisting of the following compounds:

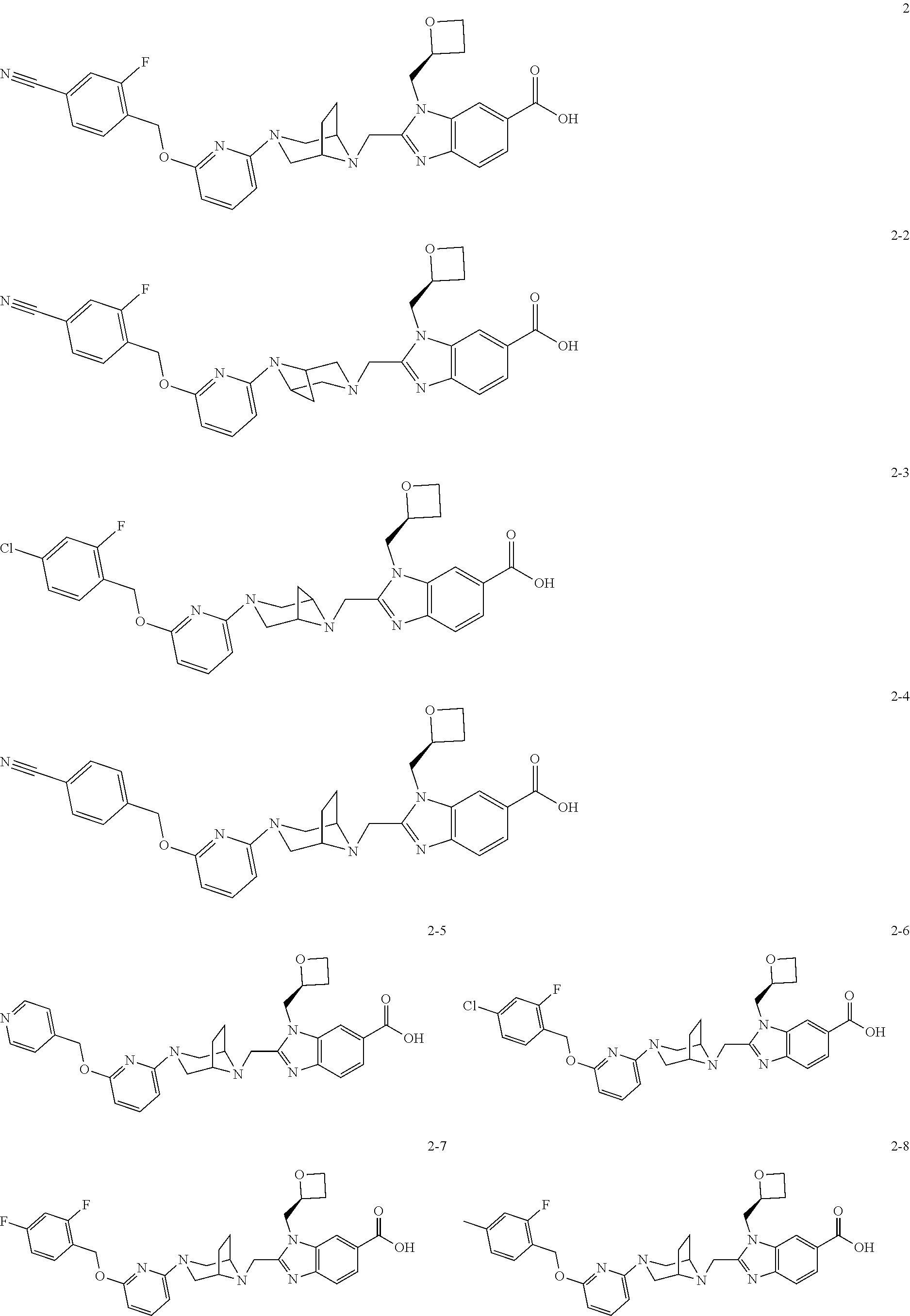

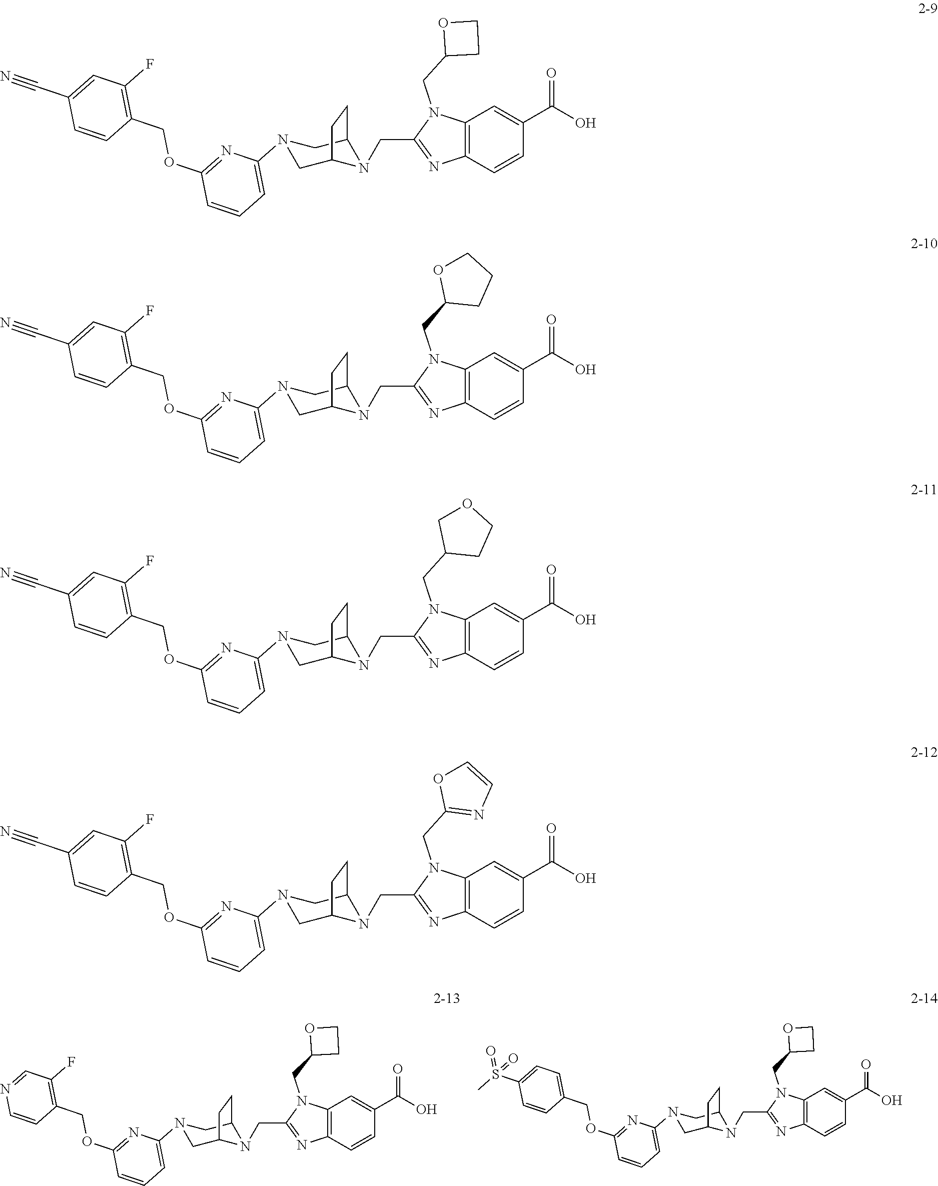 [SMILES: N#Cc1ccc(COc2cccc(N3CC4CCC(C3)N4Cc3nc4ccc(C(=O)O)cc4n3CC3CCO3)n2)c(F)c1 ; N#Cc1ccc(COc2cccc(N3CC4CCC(C3)N4Cc3nc4ccc(C(=O)O)cc4n3C[C@@H]3CCCO3)n2)c(F)c1 ; N#Cc1ccc(COc2cccc(N3CC4CCC(C3)N4Cc3nc4ccc(C(=O)O)cc4n3CC3CCOC3)n2)c(F)c1 ; N#Cc1ccc(COc2cccc(N3CC4CCC(C3)N4Cc3nc4ccc(C(=O)O)cc4n3Cc3ncco3)n2)c(F)c1 ; O=C(O)c1ccc2nc(CN3C4CCC3CN(c3cccc(OCc5ccncc5F)n3)C4)n(C[C@@H]3CCO3)c2c1 ; CS(=O)(=O)c1ccc(COc2cccc(N3CC4CCC(C3)N4Cc3nc4ccc(C(=O)O)cc4n3C[C@@H]3CCO3)n2)cc1]

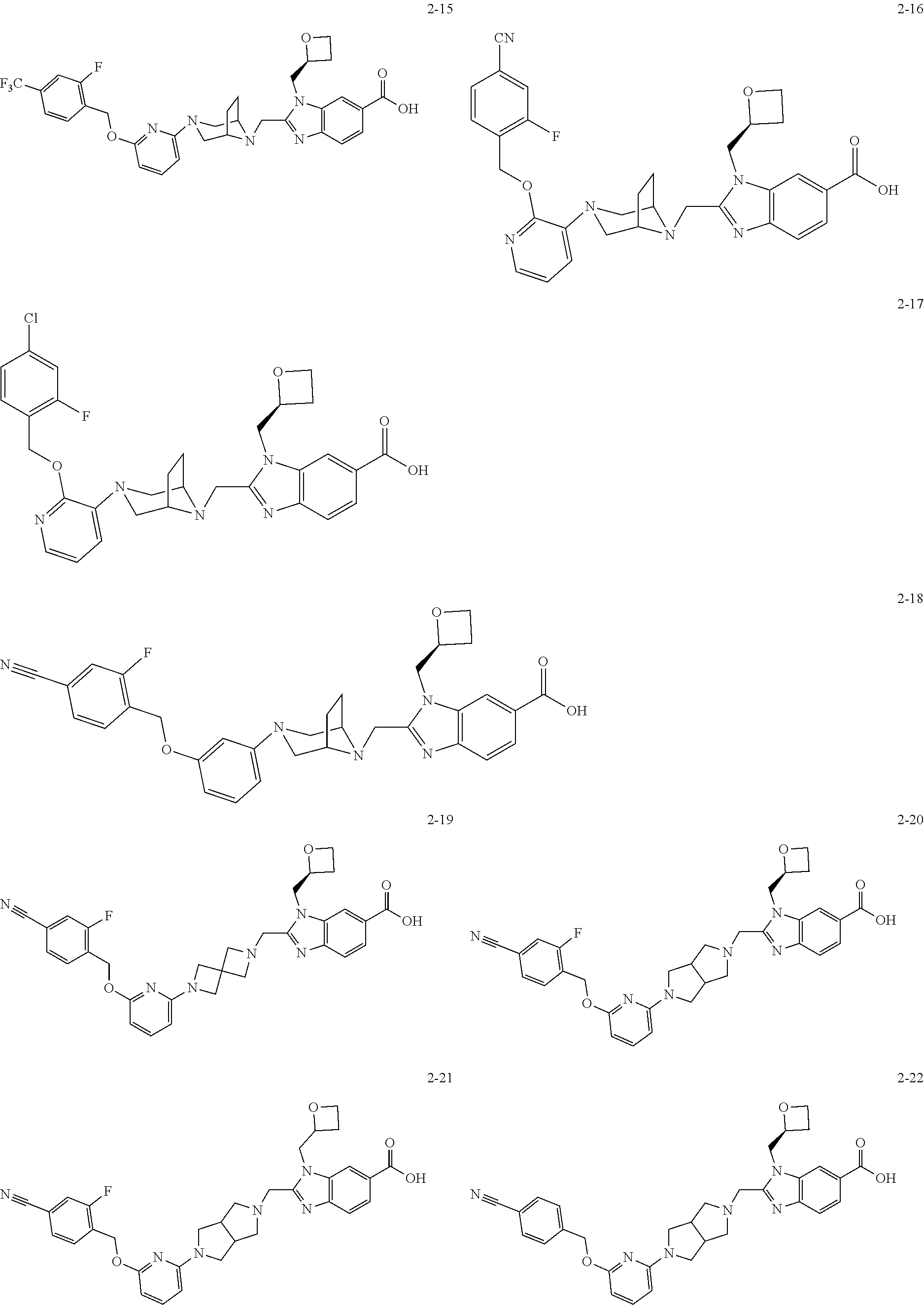

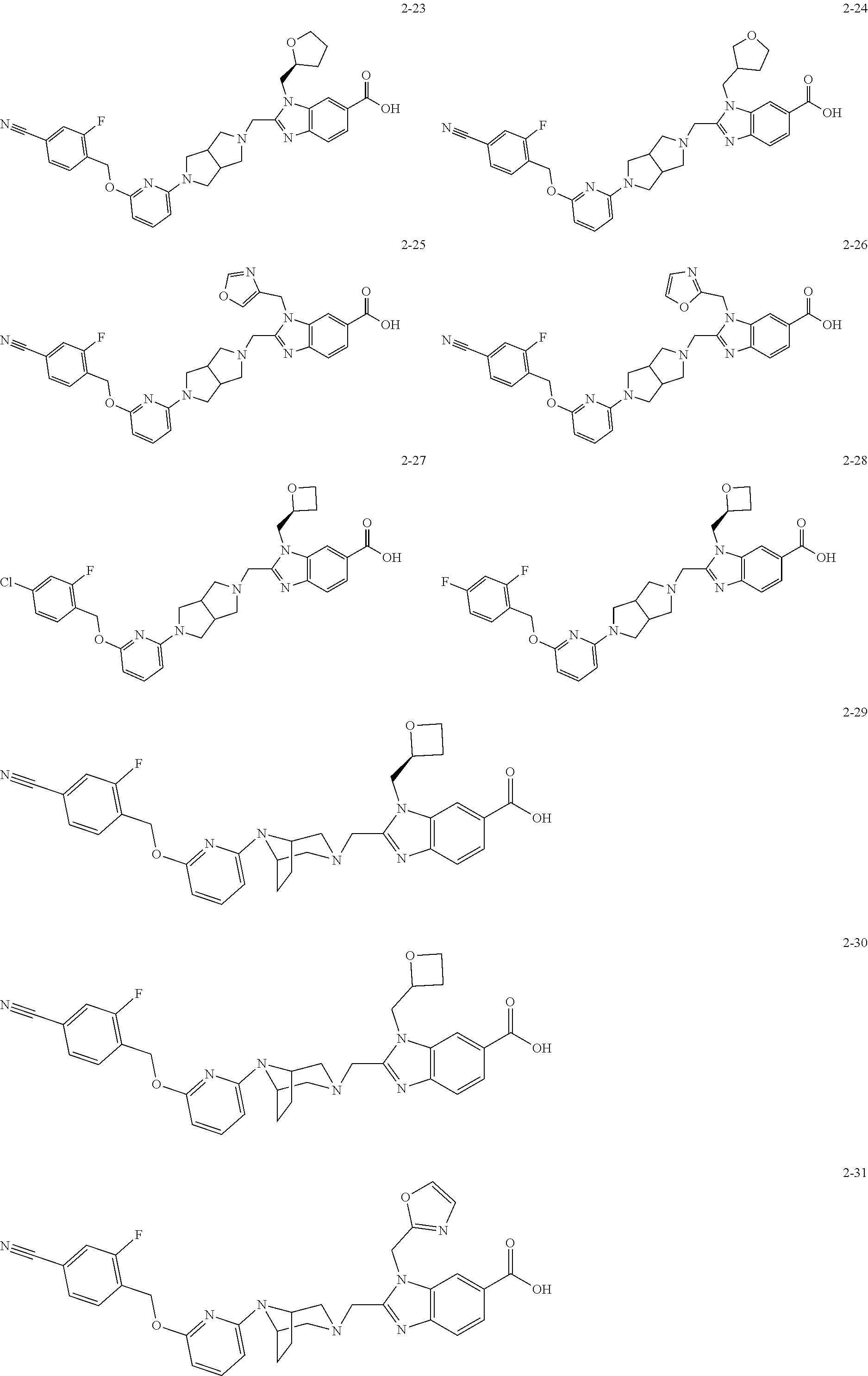

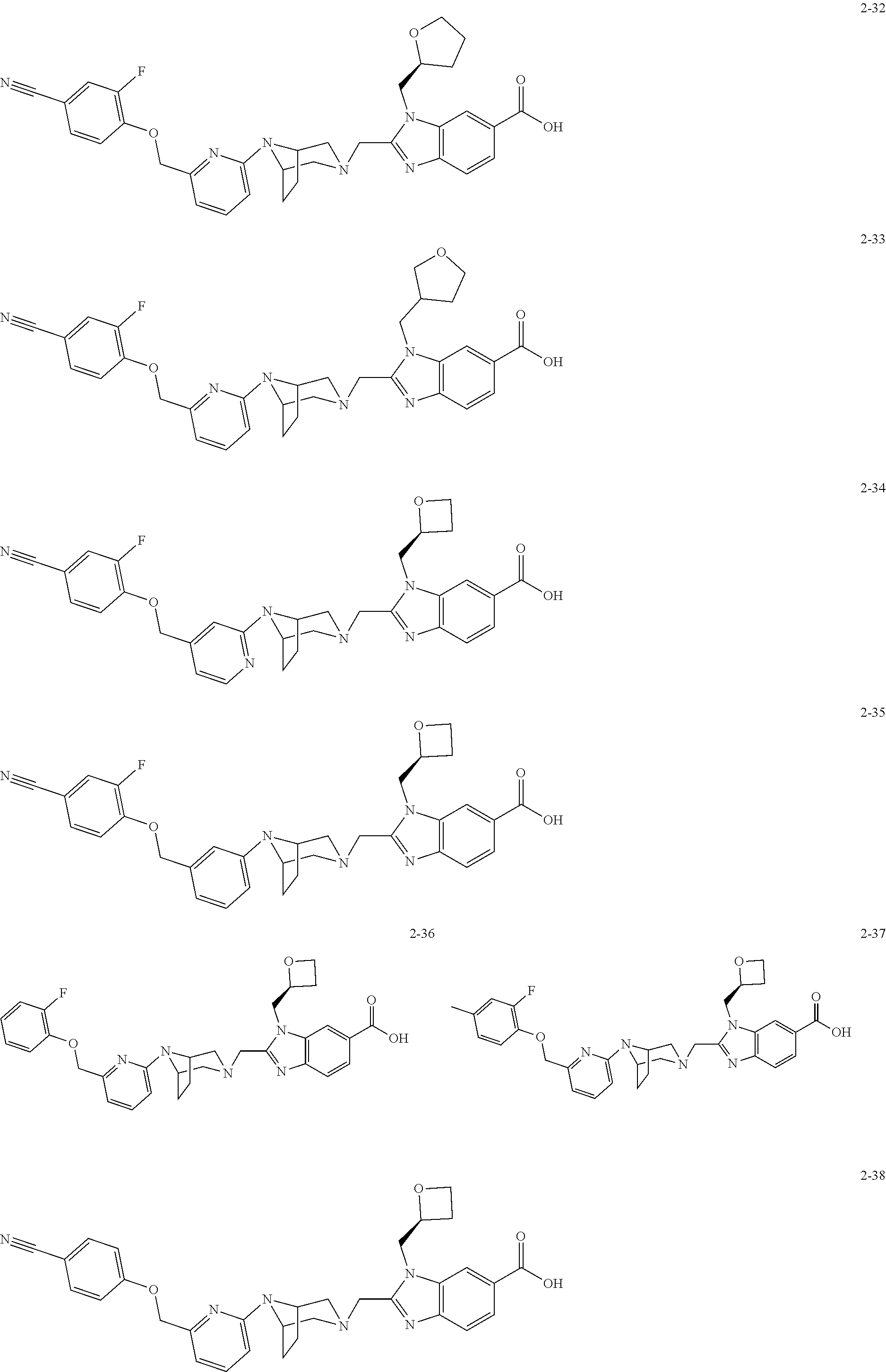

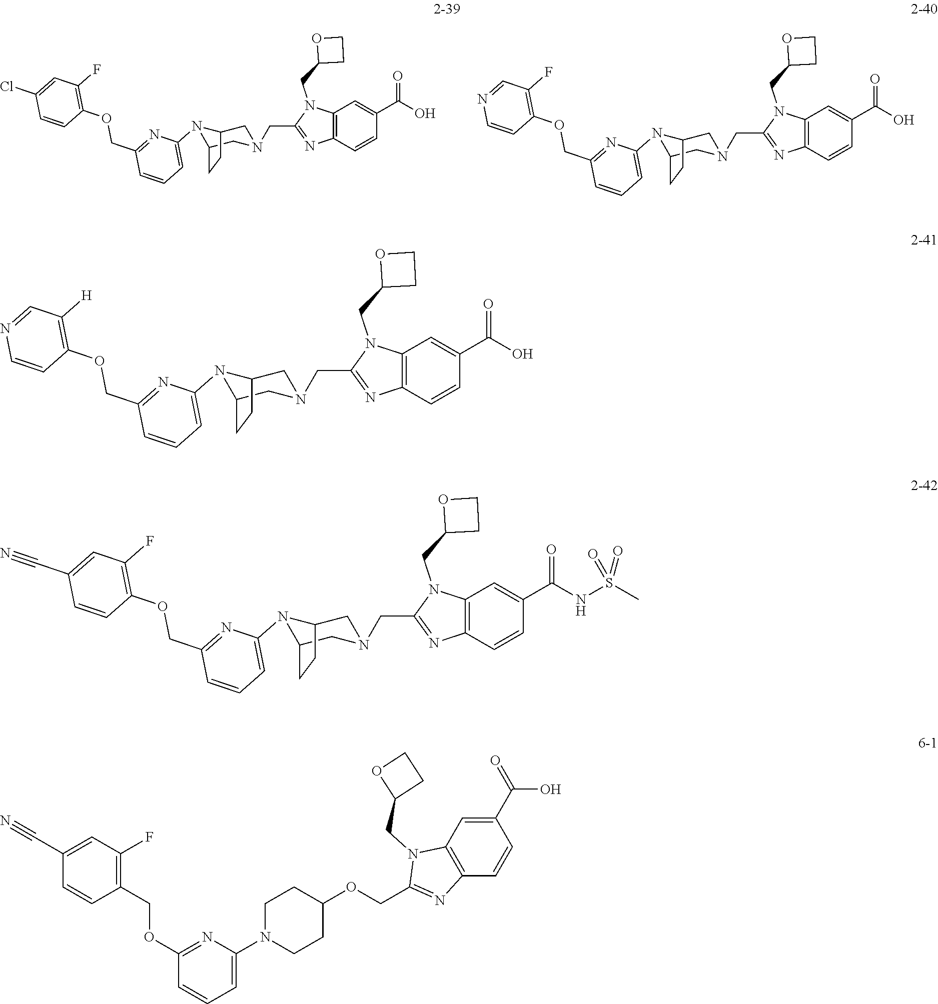 [SMILES: O=C(O)c1ccc2nc(CN3CC4CCC(C3)N4c3cccc(COc4ccc(Cl)cc4F)n3)n(C[C@@H]3CCO3)c2c1 ; O=C(O)c1ccc2nc(CN3CC4CCC(C3)N4c3cccc(COc4ccncc4F)n3)n(C[C@@H]3CCO3)c2c1 ; O=C(O)c1ccc2nc(CN3CC4CCC(C3)N4c3cccc(COc4ccncc4)n3)n(C[C@@H]3CCO3)c2c1 ; CS(=O)(=O)NC(=O)c1ccc2nc(CN3CC4CCC(C3)N4c3cccc(COc4ccc(C#N)cc4F)n3)n(C[C@@H]3CCO3)c2c1 ; N#Cc1ccc(COc2cccc(N3CCC(OCc4nc5ccc(C(=O)O)cc5n4C[C@@H]4CCO4)CC3)n2)c(F)c1]

Compounds provided herein are described with reference to both generic formulae and specific compounds. In addition, compounds of the present invention may exist in a number of different forms or derivatives, all within the scope of the present invention. These include, for example, pharmaceutically acceptable salts, tautomers, stereoisomers, racemic mixtures, regioisomers, prodrugs, solvated forms, different crystal forms or polymorphs, and active metabolites, etc.

As used herein, the term “pharmaceutically acceptable” indicates that the substance or composition is compatible chemically and/or toxicologically, with the other ingredients comprising a formulation, and/or the subjects being treated therewith.

As used herein, the term “pharmaceutically acceptable salt”, unless otherwise indicated, includes salts that retain the biological effectiveness of the free acid/base form of the specified compound and that are not biologically or otherwise undesirable. Contemplated pharmaceutically acceptable salt forms include, but are not limited to, mono, bis, tris, tetrakis, and so on. Pharmaceutically acceptable salts are non-toxic in the amounts and concentrations at which they are administered. The preparation of such salts can facilitate the pharmacological use by altering the physical characteristics of a compound without preventing it from exerting its physiological effect. Useful alterations in physical properties may include, for example, increasing the solubility to facilitate administering higher concentrations of the drug.

Pharmaceutically acceptable salts of the compounds of Formula I include acid addition and base salts.

Suitable acid addition salts are formed from acids which form non-toxic salts. Examples may include, but not limited to, the acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, 1,5-naphathalenedisulfonic acid and xinafoate salts.

Suitable base salts are formed from bases which form non-toxic salts. Examples may include, but not limited to, the aluminium, arginine, benzathine, calcium, choline, diethylamine, bis(2-hydroxyethyl) amine (diolamine), glycine, lysine, magnesium, meglumine, 2-aminoethanol (olamine), potassium, sodium, 2-Amino-2-(hydroxymethyl) propane-1,3-diol (tris or tromethamine) and zinc salts. Hemisalts of acids and bases may also be formed, for example, hemisulfate and hemicalcium salts. For a review on suitable salts, see, Stahl and Wermuth, Handbook of Pharmaceutical Salts: Properties, Selection, and Use (Wiley-VCH, 2002).

Pharmaceutically acceptable salts of the compound of Formula I may be prepared by one or more of three methods: (i) by reacting the compound of Formula I with the desired acid or base; (ii) by removing an acid- or base-labile protecting group from a suitable precursor of the compound of Formula I or by ring-opening a suitable cyclic precursor, for example, a lactone or lactam, using the desired acid or base; or (iii) by converting one salt of the compound of Formula I to another by reaction with an appropriate acid or base or by means of a suitable ion exchange column. The three reactions may be typically carried out in solution. The resulting salt may precipitate out and be collected by filtration or may be recovered by evaporation of the solvent. The degree of ionization in the resulting salt may vary from completely ionized to almost non-ionized.

The compound of Formula I, and pharmaceutically acceptable salts thereof, may exist in unsolvated and solvated forms. As used herein, the term “solvate” refers to a molecular complex comprising the compound of Formula I, or a pharmaceutically acceptable salt thereof, and one or more pharmaceutically acceptable solvent molecules, for example, ethanol. For example, the term “hydrate” is employed when said solvent is water.

A currently accepted classification system for organic hydrates is one that defines isolated site, channel, or metal-ion coordinated hydrates—see, K. R. Morris, Polymorphism in Pharmaceutical Solids (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex may have a well-defined stoichiometry independent of humidity. When, however, the solvent or water is weakly bound, as in channel solvates and hygroscopic compounds, the water/solvent content may be dependent on humidity and drying conditions. In such cases, non-stoichiometry will be the norm. Also included within the scope of the present invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallization, by recrystallization from solvents, or by physically grinding the components together—see, O. Almarsson and M. J. Zaworotko, Chem Commun, 17, 1889-1896 (2004). For a general review of multi-component complexes, see, Haleblian, J Pharm Sci, 64 (8), 1269-1288 (1975).

The compounds of the present invention may exist in a continuum of solid states ranging from fully amorphous to fully crystalline. As used herein, the term “amorphous” refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (“glass transition”). As used herein, the term “crystalline” refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (“melting point”).

The compounds of Formula I may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as “thermotropic” and that resulting from the addition of a second component, such as water or another solvent, is described as “lyotropic”. Compounds that have the potential to form lyotropic mesophases are described as “amphiphilic” and consist of molecules which possess an ionic (such as —COO⁻Na⁺, —COO⁻K⁺) or non-ionic (such as —N—N⁺(CH₃)₃) polar head group. For more information, see, N. H. Hartshorne and A. Stuart, Crystals and the Polarizing Microscope, 4^{th} Edition (Edward Arnold, 1970). The compounds of Formula I may exhibit polymorphism and/or one or more kinds of isomerism (e.g., optical, geometric or tautomeric isomerism). The compounds of Formula I may also be isotopically labelled. Such variation is implicit to the compounds of Formula I defined as they are by reference to their structural features and therefore within the scope of the present invention.

The compounds of Formula I containing one or more asymmetric carbon atoms can exist as two or more stereoisomers. Where a compound of Formula I contains an alkenyl or alkenylene group, geometric cis/trans (or Z/E) isomers are possible. Where structural isomers are interconvertible via a low energy barrier, tautomeric isomerism (“tautomerism”) can occur. This can take the form of proton tautomerism in compounds of Formula I containing, for example, an imino, keto, or oxime group, or so-called valence tautomerism in compounds which contain an aromatic moiety. It follows that a single compound may exhibit more than one type of isomerism.

The pharmaceutically acceptable salts of the compounds of Formula I may also contain a counterion which is optically active (e.g., d-lactate or l-lysine) or racemic (e.g., dl-tartrate or dl-arginine).

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Alternatively, the racemate (or a racemic precursor) may be reacted with a suitable optically active compound, for example, an alcohol, or, in the case where the compound of Formula I contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person. Chiral compounds of Formula I (and chiral precursors thereof) may be obtained in enantiomerically-enriched form using chromatography, typically HPLC, on an asymmetric resin with a mobile phase. Concentration of the eluate affords the enriched mixture. Chiral chromatography using sub- and supercritical fluids may be employed. Methods for chiral chromatography useful in some embodiments of the present invention are known in the art (see, for example, Smith, Roger M., Loughborough University, Loughborough, UK; Chromatographic Science Series (1998), 75 (Supercritical Fluid Chromatography with Packed Columns), pp. 223-249, and references cited therein).

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer. While both of the crystal forms present in a racemic mixture have identical physical properties, they may have different physical properties compared to the true racemate. Racemic mixtures may be separated by conventional techniques known to those skilled in the art—see, e.g., E. L. Eliel and S. H. Wilen, Stereochemistry of Organic Compounds (Wiley, 1994).

It must be emphasized that even if the compounds of Formula I have been drawn herein in a single tautomeric form, all possible tautomeric forms are included within the scope of the present invention.

The present invention is also intended to include all pharmaceutically acceptable isotopically-labelled compounds of Formula I, wherein one or more atoms are replaced by atoms having the same atomic number, but an atomic mass or mass number different from the atomic mass or mass number which predominates in nature. Examples of isotopes suitable for inclusion in the compounds of the present invention include, but not limited to, isotopes of hydrogen, such as ²H (i.e., deuterium (D)) and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S. Certain isotopically-labelled compounds of Formula I, for example those incorporating a radioactive isotope, are useful in drug and/or substrate tissue distribution studies. The radioactive isotopes tritium, i.e., ³H, and carbon-14, i.e., ¹⁴C, may be particularly useful for this purpose in view of their ease of incorporation and ready means of detection. Further, substitution with heavier isotopes such as deuterium, i.e., ²H, can afford certain therapeutic advantages resulting from greater metabolic stability, for example increased in vivo half-life or reduced dosage requirements and, hence, may be utilized in some particular circumstances. Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of Formula I can generally be prepared by conventional techniques known to those skilled in the art or by processes analogous to those described in the accompanying Examples and Synthesis using an appropriate isotopically-labeled reagent in place of the non-labeled reagent previously employed.

Pharmaceutically acceptable solvates in accordance with the invention may include those wherein the solvent of crystallization may be isotopically substituted, e.g., D₂O, d₆-acetone, d₆-DMSO.

One way of carrying out the invention is to administer a compound of Formula I in the form of a prodrug. Thus, certain derivatives of a compound of Formula I which may have little or no pharmacological activity themselves can, when administered into or onto the body, be converted into a compound of Formula I having the desired activity, for example by hydrolytic cleavage, particularly hydrolytic cleavage promoted by an esterase or peptidase enzyme. Such derivatives are referred to as “prodrugs”. Further information on the use of prodrugs may be found in “Pro-drugs as Novel Delivery Systems”, Vol. 14, ACS Symposium Series (T. Higuchi and W. Stella) and “Bioreversible Carriers in Drug Design”, Pergamon Press, 1987 (Ed. E. B. Roche, American Pharmaceutical Association). Reference can also be made to Nature Reviews/Drug Discovery, 2008, 7, 355 and Current Opinion in Drug Discovery and Development, 2007, 10, 550.

Prodrugs in accordance with the invention can, for example, be produced by replacing appropriate functionalities present in the compounds of Formula I with certain moieties known to those skilled in the art as “pro-moieties” as described, for example, in “Design of Prodrugs” by H. Bundgaard (Elsevier, 1985) and Y. M. Choi-Sledeski and C. G. Wermuth, “Designing Prodrugs and Bioprecursors” in Practice of Medicinal Chemistry, (4^{th} Edition), Chapter 28, 657-696 (Elsevier, 2015). Thus, a prodrug in accordance with the invention may include, but not limited to, (a) an ester or amide derivative of a carboxylic acid in a compound of Formula I; (b) an amide, imine, carbamate or amine derivative of an amino group in a compound form Formula I; (c) an oxime or imine derivative of a carbonyl group in a compound of Formula I; or (d) a methyl, primary alcohol or aldehyde group that can be metabolically oxidized to a carboxylic acid in a compound of Formula I.

Certain compounds of Formula I may themselves act as prodrugs of other compounds of Formula I. It is also possible for two compounds of Formula I to be joined together in the form of a prodrug. In certain circumstances, a prodrug of a compound of Formula I may be created by internally linking two functional groups in a compound of Formula I, for instance by forming a lactone.

References to compounds of Formula I are taken to include the compounds themselves and prodrugs thereof. The invention includes such compounds of Formula I as well as pharmaceutically acceptable salts of such compounds and pharmaceutically acceptable solvates of said compounds and salts.

### ADMINISTRATION and Dosing

The compounds of the present invention may be administered in an amount effective to treat the disorders and conditions as described herein. The compounds of the present invention can be administered as compound per se, or alternatively, as a pharmaceutically acceptable salt. For administration and dosing purposes, the compound per se or pharmaceutically acceptable salt thereof will simply be referred to as the compounds of the present invention.

The compounds of the present invention are administered by any suitable route in the form of a pharmaceutical composition adapted to such a route, and in a dose effective for the treatment intended. The compounds of the present invention may be administered orally, rectally, vaginally, parenterally, or topically.

The compounds of the present invention may be administered orally. Oral administration may involve swallowing, so that the compound enters the gastrointestinal tract, or buccal or sublingual administration may be employed by which the compound enters the bloodstream directly from the mouth.

The compounds of the present invention may be administered directly into the bloodstream, into muscle, or into an internal organ. Suitable means for parenteral administration include intravenous, intraarterial, intraperitoneal, intrathecal, intraventricular, intraurethral, intrastemal, intracranial, intramuscular and subcutaneous. Suitable devices for parenteral administration include needle (including microneedle) injectors, needle-free injectors and infusion techniques.

The compounds of the present invention may also be administered topically to the skin or mucosa, that is, dermally or transdermally. In another embodiment, the compounds of the present invention can also be administered intranasally or by inhalation. In another embodiment, the compounds of the present invention may be administered rectally or vaginally. In another embodiment, the compounds of the present invention may also be administered directly to the eye or ear.

The dosage regimen for the compounds of the present invention and/or compositions containing said compounds is based on a variety of factors, including the type, age, weight, sex and medical condition of the patient; the severity of the condition; the route of administration; and the activity of the particular compound employed. Thus, the dosage regimen may vary widely. It is not uncommon that the administration of the compounds of the present invention will be repeated a plurality of times in a day.

### Pharmaceutical Compositions

In some aspect, the present invention is directed to a pharmaceutical composition comprising the compound of Formula I or a pharmaceutically acceptable salt thereof as described herein, and at least one pharmaceutically acceptable carrier.

As used herein, the term “pharmaceutically acceptable carrier” means a carrier or excipient that is useful in preparing a pharmaceutical composition that is generally safe, non-toxic and neither biologically nor otherwise undesirable, and includes carrier or excipient that is acceptable for veterinary use as well as human pharmaceutical use. A “pharmaceutically acceptable carrier” as used herein includes both one and more than one such carrier or excipient. The particular excipient, carrier, or diluent or used will depend upon the means and purpose for which the compounds of the present invention is being applied. Suitable carriers and excipients are well known to those skilled in the art and are described in detail in, e.g., Ansel, Howard C, et al., Ansel's Pharmaceutical Dosage Forms and Drug Delivery Systems. Philadelphia: Lippincott, Williams & Wilkins, 2004; Gennaro, Alfonso R., et al., Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., the compound or pharmaceutical composition as described herein) or aid in the manufacturing of the pharmaceutical product (i.e., medicament).

The compounds of the present invention may be administered by any convenient route appropriate to the condition to be treated. Suitable routes may include oral, parenteral (including subcutaneous, intramuscular, intravenous, intraarterial, intradermal, intrathecal and epidural), transdermal, rectal, nasal, topical (including buccal and sublingual), ocular, vaginal, intraperitoneal, intrapulmonary and intranasal.

The compositions of the present invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The form depends on the intended mode of administration and therapeutic application.

Other modes of administration known in the pharmaceutical art may also be used. Pharmaceutical compositions of the present invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pennsylvania, 1975; Liberman et al., Eds., Pharmaceutical Dosage Forms, Marcel Decker, New York, N.Y., 1980; and Kibbe et al., Eds., Handbook of Pharmaceutical Excipients (3rd Ed.), American Pharmaceutical Association, Washington, 1999.

### Methods of Treatment

In a further aspect, there is provided a method of treating a GLP-1R-related disorder or condition in a subject in need thereof, comprising administering to the subject a therapeutically effective amount of a compound of Formula I or a pharmaceutically acceptable salt thereof, owning to the GLP-1R agonist activity of the compound of the present invention.

As used herein, the term “subject in need thereof” is a subject having a GLP-1R-related disorder or condition, or a subject having an increased risk of developing GLP-1R-related disorder or condition relative to the population at large. The term “subject” includes a warm-blooded animal. In some embodiments, the warm-blooded animal is a mammal. In some embodiments, the warm-blooded animal is a human.

In certain embodiments, the GLP-1R-related disorder or condition is selected from the group consisting of diabetes (T1D and/or T2DM, including pre-diabetes), idiopathic T1D (Type 1b), latent autoimmune diabetes in adults (LADA), early-onset T2DM (EOD), youth-onset atypical diabetes (YOAD), maturity onset diabetes of the young (MODY), malnutrition-related diabetes, gestational diabetes, hyperglycemia, insulin resistance, hepatic insulin resistance, impaired glucose tolerance, diabetic neuropathy, diabetic nephropathy, kidney disease (e.g., acute kidney disorder, tubular dysfunction, proinflammatory changes to the proximal tubules), diabetic retinopathy, adipocyte dysfunction, visceral adipose deposition, sleep apnea, obesity (including hypothalamic obesity and monogenic obesity) and related comorbidities (e.g., osteoarthritis and urine incontinence), eating disorders (including binge eating syndrome, bulimia nervosa, and syndromic obesity such as Prader-Willi and Bardet-Biedl syndromes), weight gain from use of other agents (e.g., from use of steroids and antipsychotics), excessive sugar craving, dyslipidemia (including hyperlipidemia, hypertriglyceridemia, increased total cholesterol, high LDL cholesterol, and low HDL cholesterol), hyperinsulinemia, NAFLD (including related diseases such as steatosis, NASH, fibrosis, cirrhosis, and hepatocellular carcinoma), cardiovascular disease, atherosclerosis (including coronary artery disease), peripheral vascular disease, hypertension, endothelial dysfunction, impaired vascular compliance, congestive heart failure, myocardial infarction (e.g. necrosis and apoptosis), stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, Parkinson's Disease, left ventricular hypertrophy, peripheral arterial disease, macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, prevention or treatment of Polycystic Ovary Syndrome and treatment of addiction (e.g., alcohol and/or drug abuse).

In certain embodiments, the GLP-1R-related disorder or condition is selected from the group consisting of obesity, T2DM, NAFLD and NASH.

The method of treating a GLP-1R-related disorder or condition described herein may be used as a monotherapy. As used herein, the term “monotherapy” refers to the administration of a single active or therapeutic compound to a subject in need thereof. In some embodiments, monotherapy will involve administration of a therapeutically effective amount of one of the compounds of the present invention or a pharmaceutically acceptable salt thereof, to a subject in need of such treatment.

Depending upon the particular disorder or condition to be treated, the method of treating a GLP-1R-related disorder or condition described herein may involve, in addition to administration of the compound of Formula I, combination therapy of one or more additional therapeutic agent(s), for example, a second therapeutic agent having the GLP-1R agonist activity. As used herein, the term “combination therapy” refers to the administration of a combination of multiple active therapeutic agents. In some embodiments, the compound of the present invention may be administered simultaneously, separately or sequentially to treatment with the one or more additional therapeutic agent(s). For example, the additional therapeutic agent(s) may be administered separately from the compound of the present invention, as part of a multiple dosage regimen. Alternatively, the additional therapeutic agent(s) may be part of a single dosage form, mixed with the compound of the present invention in a single composition.

In certain embodiments, the compounds of the present invention may be administered with an anti-diabetic agent including but not limited to a biguanide (e.g., metformin), a sulfonylurea (e.g., tolbutamide, glibenclamide, gliclazide, chlorpropamide, tolazamide, acetohexamide.glyclopyramide, glimepiride, or glipizide), a thiazolidinedione (e.g., pioglitazone, rosiglitazone, or lobeglitazone), a glitazar (e.g., saroglitazar, aleglitazar, muraglitazar or tesaglitazar), a meglitinide (e.g., nateglinide, repaglinide), a dipeptidyl peptidase 4 (DPP-4) inhibitor (e.g., sitagliptin, vildagliptin, saxagliptin, linagliptin, gemigliptin, anagliptin, teneligliptin, alogliptin, trelagliptin, dutogliptin, or omarigliptin), a glitazone (e.g., pioglitazone, rosiglitazone, balaglitazone, rivoglitazone, or lobeglitazone), a sodium-glucose linked transporter 2 (SGLT2) inhibitor (e.g., empagliflozin, canagliflozin, dapagliflozin, ipragliflozin, Ipraglrflozin, tofogliflozin, sergliflozin etabonate, remogliflozin etabonate, or ertugliflozin), an SGLTL1 inhibitor, a GPR40 agonist (FFAR1/FFA1 agonist, e.g. fasiglifam), glucose-dependent insulinotropic peptide (GIP) and analogues thereof, an alpha glucosidase inhibitor (e.g. voglibose, acarbose, or miglitol), or an insulin or an insulin analogue, including the pharmaceutically acceptable salts of the specifically named agents and the pharmaceutically acceptable solvates of said agents and salts.

In another embodiment, the compounds of the present invention are administered with an anti-obesity agent including but not limited to peptide YY or an analogue thereof, a neuropeptide Y receptor type 2 (NPYR2) agonist, a NPYR1 or NPYR5 antagonist, a cannabinoid receptor type 1 (CB1R) antagonist, a lipase inhibitor (e.g., orlistat), a human proislet peptide (HIP), a melanocortin receptor 4 agonist (e.g., setmelanotide), a melanin concentrating hormone receptor 1 antagonist, a famesoid X receptor (FXR) agonist (e.g. obeticholic acid), zonisamide, phentermine (alone or in combination with topiramate), a norepinephrine/dopamine reuptake inhibitor (e.g., buproprion), an opioid receptor antagonist (e.g., naltrexone), a combination of norepinephrine/dopamine reuptake inhibitor and opioid receptor antagonist (e.g., a combination of bupropion and naltrexone), a GDF-15 analog, sibutramine, a cholecystokinin agonist, amylin and analogues thereof (e.g., pramlintide), leptin and analogues thereof (e.g., metroleptin), a serotonergic agent (e.g., lorcaserin), a methionine aminopeptidase 2 (MetAP2) inhibitor (e.g., beloranib or ZGN-1061), phendimetrazine, diethylpropion, benzphetamine, an SGLT2 inhibitor (e.g., empagliflozin, canagliflozin, dapagliflozin, ipragliflozin, Ipragliflozin, tofogliflozin, sergliflozin etabonate, remogliflozin etabonate, or ertugliflozin), an SGLTL1 inhibitor, a dual SGLT2/SGLT1 inhibitor, a fibroblast growth factor receptor (FGFR) modulator, an AMP-activated protein kinase (AMPK) activator, biotin, a MAS receptor modulator, or a glucagon receptor agonist (alone or in combination with another GLP-1 R agonist, e.g., liraglutide, exenatide, dulaglutide, albiglutide, lixisenatide, or semaglutide), including the pharmaceutically acceptable salts of the specifically named agents and the pharmaceutically acceptable solvates of said agents and salts.

In another embodiment, the compounds of the present invention are administered with an agent to treat NASH including but not limited to PF-05221304, an FXR agonist (e.g., obeticholic acid), a PPAR α/δ agonist (e.g., elafibranor), a synthetic fatty acid-bile acid conjugate (e.g., aramchol), a caspase inhibitor (e.g., emricasan), an anti-lysyl oxidase homologue 2 (LOXL2) monoclonal antibody (e.g., simtuzumab), a galectin 3 inhibitor (e.g., GR-MD-02), a MAPK5 inhibitor (e.g., GS-4997), a dual antagonist of chemokine receptor 2 (CCR2) and CCR5 (e.g., cenicriviroc), a fibroblast growth factor 21 (FGF21) agonist (e.g., BMS-986036), a leukothene D4 (LTD4) receptor antagonist (e.g., tipelukast), a niacin analogue (e.g., ARI 3037MO), an ASBT inhibitor (e.g., volixibat), an acetyl-CoA carboxylase (ACC) inhibitor (e.g., NDI 010976), a ketohexokinase (KHK) inhibitor, a diacylglyceryl acyltransferase 2 (DGAT2) inhibitor, a CB1 receptor antagonist, an anti-CB1R antibody, or an apoptosis signal-regulating kinase 1 (ASK1) inhibitor, including the pharmaceutically acceptable salts of the specifically named agents and the pharmaceutically acceptable solvates of said agents and salts.

Therefore, in a further aspect, the present invention is directed to a method of treating a GLP-1R-related disorder or condition in a subject in need thereof, comprising administering to the subject a therapeutically effective amount of a compound of Formula (I) or a pharmaceutically acceptable salt thereof as described herein.

In certain embodiments, there is provided the method as described herein, wherein the GLP-1R-related disorder or condition is selected from the group consisting of obesity, type 2 diabetes mellitus (T2DM), Non-alcoholic fatty liver disease (NAFLD), and non-alcoholic steatohepatitis (NASH).

In a further aspect, the present invention is directed to a compound of Formula I or a pharmaceutically acceptable salt thereof as described herein for use in treatment of a GLP-1R-related disorder and condition.

In certain embodiments, there is provided the compound of Formula I or a pharmaceutically acceptable salt thereof as described herein, wherein the GLP-1R-related disorder or condition is selected from the group consisting of obesity, type 2 diabetes mellitus (T2DM), Non-alcoholic fatty liver disease (NAFLD), and non-alcoholic steatohepatitis (NASH).

In a further aspect, the present invention is directed to use of a compound of Formula I or a pharmaceutically acceptable salt thereof as described herein in the manufacture of a medicament for the treatment of a GLP-1R-related disorder or condition.

In certain embodiments, there is provided the use described herein, wherein the GLP-1R-related disorder or condition is selected from the group consisting of obesity, type 2 diabetes mellitus (T2DM), Non-alcoholic fatty liver disease (NAFLD), and non-alcoholic steatohepatitis (NASH).

### Synthesis

The compounds of the present invention may be prepared by the general and specific methods described below, using the common general knowledge of one skilled in the art of synthetic organic chemistry. Such common general knowledge can be found in standard reference books such as Comprehensive Organic Chemistry, Ed. Barton and Ollis, Elsevier; Comprehensive Organic Transformations: A Guide to Functional Group Preparations, Larock, John Wiley and Sons; and Compendium of Organic Synthetic Methods, Vol. I-XII (published by Wiley-Interscience). The starting materials used herein are commercially available or may be prepared by routine methods known in the art.

In the preparation of the compounds of the present invention, it is noted that some of the preparation methods described herein may require protection of remote functionality. The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see T. W. Greene, Protective Groups in Organic Synthesis, John Wiley & Sons, New York, 1991.

The Schemes described below are intended to provide a general description of the methodology employed in the preparation of the compounds of the present invention. Some of the compounds of the present invention may contain single or multiple chiral centers with the stereochemical designation (R) or(S). It will be apparent to one skilled in the art that all of the synthetic transformations can be conducted in a similar manner whether the materials are enantioenriched or racemic. Moreover, the resolution to the desired optically active material may take place at any desired point in the sequence using well known methods such as described herein and in the chemistry literature.

### EXAMPLES

All reagents and materials are purchased from commercial vendors or may be readily prepared by those skilled in the art. A list of abbreviations for reagents used may be found in Table 1, below.

TABLE 1
Abbreviations
Reagents or Organic Moieties

| Abbreviation | Full Name |
|---|---|
| t-Bu | tert-butyl |
| i-Bu | iso-butyl |
| Boc₂O | di-tert-butyl dicarbonate |
| DMF | dimethylformamide |
| DME | 1,2-dimethoxyethane |
| DCM | dichloromethane |
| DIEA | N,N-Diisopropylethylamine |
| Et | ethyl |
| HATU | 1-[Bis(dimethylamino)methylene]- |
| | 1H-1,2,3-triazolo[4,5- |
| | b]pyridinium 3-Oxide Hexafluorophosphate |
| LDA | Lithium diisopropylamide |
| NMP | 1-Methyl-2-pyrrolidone |
| THF | tetrahydrofuran |
| TFA | trifluoroacetic acid |
| TBS | tert-butyldimethylsilyl |
| p-TsOH | p-toluenesulfonic acid |
| TBAF | tetra-n-butylammonium fluoride |
| TosMIC | toluenesulfonylmethyl isocyanide |
| Tf₂O | trifluoromethanesulfonic anhydride |
| Ph | phenyl |
| Pd(dppf)Cl₂ | [1,l′-bis(diphenylphosphino)ferrocene] |
| | palladium(II) dichloride |

### Example 1. Synthesis of Compounds 1-1, 1-1-A and 1-1-B

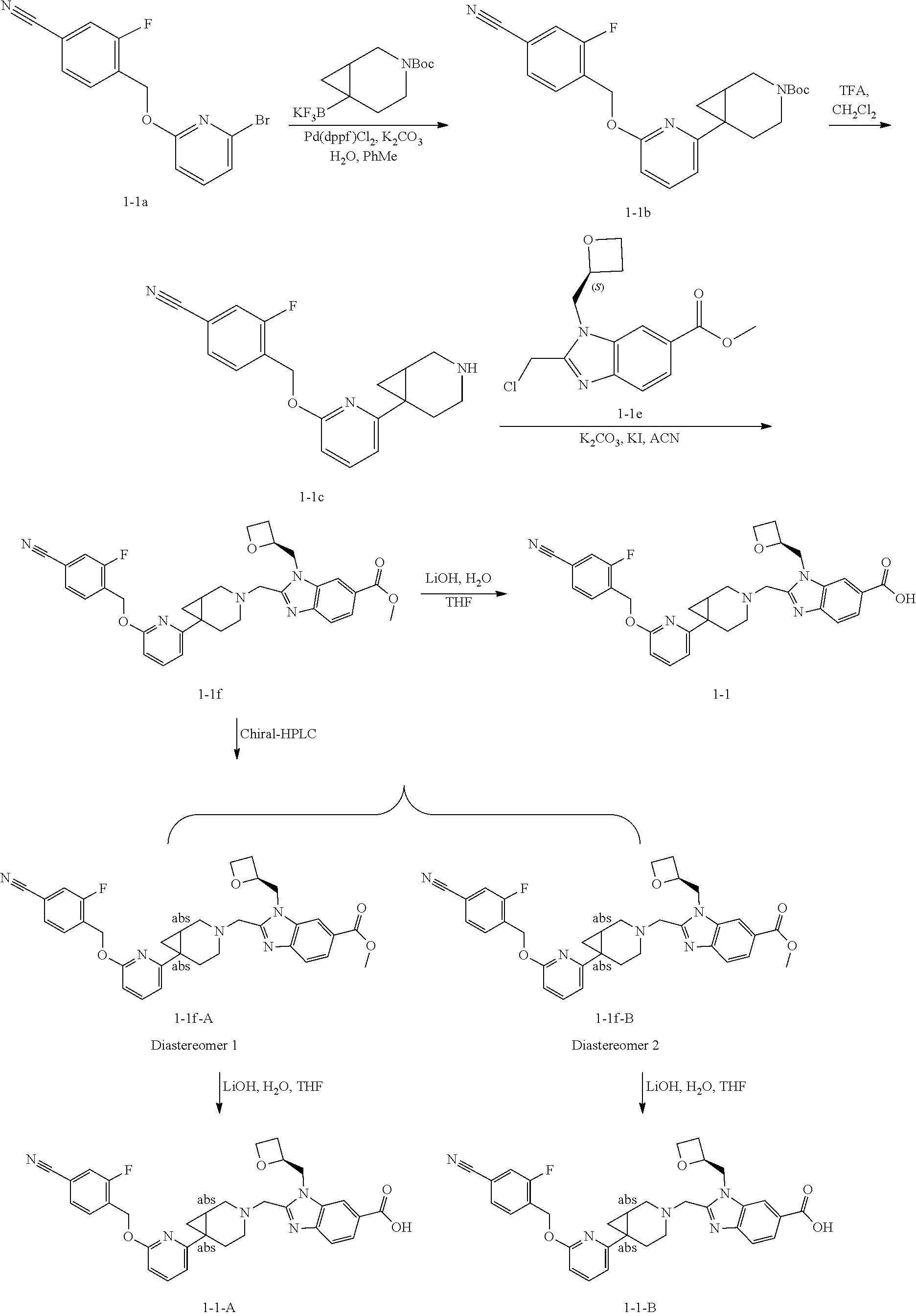

Tert-butyl 6-(6-((4-cyano-2-fluorobenzyl)oxy)pyridin-2-yl)-3-azabicyclo[4.1.0]heptane-3-carboxylate (1-1b): To a solution of 4-{[(6-bromopyridin-2-yl)oxy]methyl}-3-fluorobenzonitrile (200.0 mg, 0.65 mmol) in toluene (10.0 mL) and H₂O (2.0 mL) was added potassium (3-(tert-butoxycarbonyl)-3-azabicyclo[4.1.0]heptan-6-yl)trifluoroborate (217.2 mg, 0.72 mmol), Pd(dppf)Cl₂ (47.7 mg, 0.07 mmol) and K₂CO₃ (135.0 mg, 0.98 mmol) at room temperature under N₂. The resulting mixture was stirred at 100° C. for 16 h under N₂. After the reaction was completed, the resulting mixture was cooled to room temperature and filtered. The filtrate was concentrated under reduced pressure. The residue was purified by flash column chromatography with petroleum ether/ethyl acetate (90/10, v/v) to afford tert-butyl 6-(6-((4-cyano-2-fluorobenzyl)oxy)pyridin-2-yl)-3-azabicyclo[4.1.0]heptane-3-carboxylate (150.0 mg, 47%) as a white solid. LCMS (ESI, m/z): [M+H]⁺=424.2.

4-{[(6-{3-azabicyclo[4.1.0]heptan-6-yl}pyridin-2-yl)oxy]methyl}-3-fluorobenzonitrile (1-1c): To a solution of tert-butyl 6-{6-[(4-cyano-2-fluorophenyl)methoxy]pyridin-2-yl}-3-azabicyclo[4.1.0]heptane-3-carboxylate (140.0 mg, 0.33 mmol) in CH₂Cl₂ (8.0 mL) was added TFA (4.0 mL) at room temperature. The resulting mixture was stirred at room temperature for 1 h. After the reaction was completed, the resulting mixture was concentrated under reduced pressure. The pH value of the residue was adjusted to 7.0 with saturated NaHCO₃(aq.). The mixture was extracted with CH₂Cl₂. The combined organic layer was washed with brine, dried over anhydrous sodium sulfate and filtered. The filtrate was concentrated under vacuum to afford 4-{[(6-{3-azabicyclo[4.1.0]heptan-6-yl}pyridin-2-yl)oxy]methyl}-3-fluorobenzonitrile (100.0 mg, crude) as a white solid. LCMS (ESI, m/z): [M+H]⁺=324.1.

Methyl 2-[(6-{6-[(4-cyano-2-fluorophenyl)methoxy]pyridin-2-yl}-3-azabicyclo[4.1.0]heptan-3-yl)methyl]-3-[(2S)-oxetan-2-ylmethyl]-1,3-benzodiazole-5-carboxylate (1-1f): To a solution of 4-{[(6-{3-azabicyclo[4.1.0]heptan-6-yl}pyridin-2-yl)oxy]methyl}-3-fluorobenzonitrile (140.0 mg, 0.43 mmol) in ACN (5.0 mL) was added methyl 2-(chloromethyl)-3-[(2S)-oxetan-2-ylmethyl]-1,3-benzodiazole-5-carboxylate (127.0 mg, 0.43 mmol), K₂CO₃ (141.4 mg, 1.29 mmol) and KI (36.0 mg, 0.22 mmol) at room temperature. The resulting mixture was stirred at room temperature for 16 h. After the reaction was completed, the resulting mixture was filtered. The filtrate was concentrated under reduced pressure. The residue was purified by flash column chromatography with petroleum ether/ethyl acetate (10/90, v/v) to afford methyl 2-[(6-{6-[(4-cyano-2-fluorophenyl)methoxy]pyridin-2-yl}-3-azabicyclo[4.1.0]heptan-3-yl)methyl]-3-[(2S)-oxetan-2-ylmethyl]-1,3-benzodiazole-5-carboxylate (1-1f) (110.0 mg, 44%) as a white solid. LCMS (ESI, m/z): [M+H]⁺=582.2.

Isolation of methyl 2-((6-(6-((4-cyano-2-fluorobenzyl)oxy)pyridin-2-yl)-3-azabicyclo[4.1.0]heptan-3-yl)methyl)-1-(((S)-oxetan-2-yl)methyl)-1H-benzo[d]imidazole-6-carboxylate (1-1f-A) and methyl 2-((6-(6-((4-cyano-2-fluorobenzyl)oxy)pyridin-2-yl)-3-azabicyclo[4.1.0]heptan-3-yl)methyl)-1-(((S)-oxetan-2-yl)methyl)-1H-benzo[d]imidazole-6-carboxylate (1-1f-B): Separation of 1-1f (100.0 mg, 0.17 mmol) into its component diastereomers was effected using Prep-Chiral-HPLC with the following conditions: Column: CHIRALPAK IG, 2×25 cm, 5 μm; Mobile Phase A: Hex (0.5% 2M NH₃-MeOH)—HPLC, Mobile Phase B: MeOH:DCM=1:1-HPLC; Flow rate: 20 mL/min; Gradient: 50% B to 50% B in 10.5 min; Wave Length: 220/254 nm. The first-eluting diastereomer (Retention time: 4.75 minutes), obtained as a white solid (48.0 mg), was designated as 1-1f-A. LCMS (ESI, m/z): [M+H]⁺=582.0. The second-eluting diastereomer (Retention time: 7.56 minutes), obtained as a white solid (35.5 mg), was designated as 1-1f-B. LCMS (ESI, m/z): [M+H]⁺==582.0.

2-[(6-{6-[(4-cyano-2-fluorophenyl)methoxy]pyridin-2-yl}-3-azabicyclo[4.1.0]heptan-3-yl)methyl]-3-[(2S)-oxetan-2-ylmethyl]-1,3-benzodiazole-5-carboxylic acid (1-1): To a solution of 1-1f (90.0 mg, 0.16 mmol) in THF (3.0 mL) and H₂O (2.0 mL) was added LiOH (111.6 mg, 1.55 mmol) at room temperature. The resulting mixture was stirred at room temperature for 16 h. After the reaction was completed, the pH value of the mixture was adjusted to 5.0 with CH₃COOH. The mixture was extracted with ethyl acetate. The combined organic layer was washed with brine, dried over anhydrous sodium sulfate and filtered. The filtrate was concentrated under vacuum. The residue was purified by Prep-HPLC with the following conditions: (Column: Xselect CSH C18 OBD Column 30×150 mm 5 μm; Mobile Phase A: Water (10 mmol/L NH₄HCO₃), Mobile Phase B: ACN; Flow rate: 60 mL/min;

Gradient: 29% B to 39% B in 8 min; Wave Length: 254 nm) and lyophilized to afford 2-[(6-{6-[(4-cyano-2-fluorophenyl)methoxy]pyridin-2-yl}-3-azabicyclo[4.1.0]heptan-3-yl)methyl]-3-[(2S)-oxetan-2-ylmethyl]-1,3-benzodiazole-5-carboxylic acid (2.5 mg, 3%), which is the mixture of 1-1-A and 1-1-B, as a white solid. LCMS (ESI, m/z): [M+H]⁺=568.3. ¹H NMR (400 MHz, DMSO-d₆): δ 8.22 (s, 1H), 7.90-7.80 (m, 2H), 7.71-7.59 (m, 4H), 6.95 (d, J=7.2 Hz, 1H), 6.66 (d, J=8.4 Hz, 1H), 5.47-5.40 (m, 2H), 5.08-5.04 (m, 1H), 4.79-4.73 (m, 1H), 4.63-4.60 (m, 1H), 4.47-4.43 (m, 1H), 4.40-4.30 (m, 1H), 3.92-3.84 (m, 1H), 3.77-3.69 (m, 1H), 2.82-2.78 (m, 2H), 2.41-2.29 (m, 2H), 1.97-1.94 (m, 1H), 1.70-1.66 (m, 1H), 1.11-1.08 (m, 1H), 0.93-0.90 (m, 1H).

2-((6-(6-((4-cyano-2-fluorobenzyl)oxy)pyridin-2-yl)-3-azabicyclo[4.1.0]heptan-3-yl)methyl)-1-(((S)-oxetan-2-yl)methyl)-1H-benzo[d]imidazole-6-carboxylic acid (1-1-A): To a solution of 1-1f-A (48.0 mg, 0.08 mmol) in THF (1.5 mL) and H₂O (1.0 mL) was added LiOH (9.9 mg, 0.42 mmol) at room temperature. The mixture was stirred at room temperature for 16 h. After the reaction was completed, the pH value of the mixture was adjusted to 4.0 with CH₃COOH. The mixture was extracted with ethyl acetate. The combined organic layer was washed with brine, dried over anhydrous sodium sulfate and filtered. The filtrate was concentrated under vacuum. The residue was purified by Prep-HPLC with the following conditions (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 31% B to 41% B in 8 min; Wave Length: 254 nm) to afford 1-1-A (14.1 mg, 28%) as a white solid. LCMS (ESI, m/z): [M+H]⁺=568.3. ¹H NMR (400 MHZ, DMSO-d₆): δ 8.22 (s, 1H), 7.90-7.81 (m, 2H), 7.71-7.58 (m, 4H), 6.95 (d, J=7.6 Hz, 1H), 6.66 (d, J=8.0 Hz, 1H), 5.44-5.39 (m, 2H), 5.08-5.02 (m, 1H), 4.78-4.72 (m, 1H), 4.63-4.59 (m, 1H), 4.47-4.45 (m, 1H), 4.38-4.36 (m, 1H), 3.91-3.88 (m, 1H), 3.72-3.68 (m, 1H), 2.78-2.76 (m, 2H), 2.68-2.65 (m, 1H), 2.43-2.36 (m, 3H), 2.30-2.25 (m, 1H), 1.97-1.90 (m, 1H), 1.69-1.67 (m, 1H), 1.11-1.08 (m, 1H), 0.93-0.90 (m, 1H).

2-((6-(6-((4-cyano-2-fluorobenzyl)oxy)pyridin-2-yl)-3-azabicyclo[4.1.0]heptan-3-yl)methyl)-1-(((S)-oxetan-2-yl)methyl)-1H-benzo[d]imidazole-6-carboxylic acid (1-1-B): To a solution of 1-1f-B (35.5 mg, 0.06 mmol) in THF (1.5 mL) was added LiOH (7.3 mg, 0.30 mmol) in H₂O (1.0 mL). The mixture was stirred at room temperature for 16 h. After the reaction was completed, the pH value of the mixture was acidified to 4.0 with CH₃COOH. The mixture was extracted with ethyl acetate. The combined organic layer was washed with brine, dried over anhydrous sodium sulfate and filtered. The filtrate was concentrated under vacuum. The residue was purified by Prep-HPLC with the following conditions (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 31% B to 41% B in 8 min; Wave Length: 254 nm) to afford 1-1-B (14.9 mg, 41%) as a white solid. LCMS (ESI, m/z): [M+H]⁺=568.3. ¹H NMR (400 MHZ, DMSO-d₆): δ 12.80 (s, 1H), 8.23 (s, 1H), 7.90-7.81 (m, 2H), 7.70-7.59 (m, 4H), 6.95 (d, J=7.6 Hz, 1H), 6.66 (d, J=8.0 Hz, 1H), 5.47-5.40 (m, 2H), 5.10-5.04 (m, 1H), 4.79-4.73 (m, 1H), 4.63-4.60 (m, 1H), 4.48-4.43 (m, 1H), 4.35-4.30 (m, 1H), 3.87-3.73 (m, 2H), 2.84-2.80 (m, 1H), 2.72-2.62 (m, 3H), 2.42-2.29 (m, 3H), 1.98-1.92 (m, 1H), 1.70-1.65 (m, 1H), 1.15-1.07 (m, 1H), 0.92-0.86 (m, 1H).

Following the synthetic route described above in Example 1 and substituting the appropriate reagents, starting materials and purification methods known to those skilled in the art, the compounds listed below were synthesized.

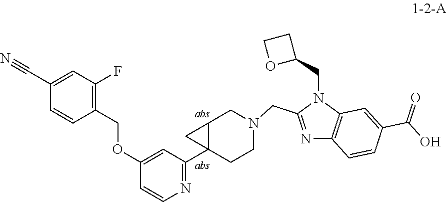 [SMILES: N#Cc1ccc(COc2ccnc(C34CCN(Cc5nc6ccc(C(=O)O)cc6n5C[C@@H]5CCO5)CC3C4)c2)c(F)c1]

2-((6-(4-((4-cyano-2-fluorobenzyl)oxy)pyridin-2-yl)-3-azabicyclo[4.1.0]heptan-3-yl)methyl)-1-(((S)-oxetan-2-yl)methyl)-1H-benzo[d]imidazole-6-carboxylic acid (1-2-A): white solid. LCMS (ESI, m/z): [M+H]⁺=568.1. ¹H NMR (400 MHz, DMSO-d₆): δ 8.27-8.23 (m, 2H), 7.94-7.92 (m, 1H), 7.83-7.76 (m, 3H), 7.59 (d, J=8.8 Hz, 1H), 6.94 (d, J=2.0 Hz, 1H), 6.86-6.84 (m, 1H), 5.33 (s, 2H), 5.09-5.04 (m 1H), 4.76-4.74 (m, 1H), 4.66-4.63 (m, 1H), 4.47-4.45 (m, 1H), 4.34-4.32 (m, 1H), 3.87-3.75 (m, 2H), 2.85-2.80 (m, 1H), 2.75-2.72 (m, 1H), 2.68-2.64 (m, 1H), 2.60-2.56 (m, 1H), 2.46-2.39 (m, 1H), 2.36-2.32 (m, 1H), 2.01-1.97 (m, 1H), 1.77-1.73 (m, 1H), 1.20-1.18 (m, 2H), 0.95-0.92 (m, 1H).

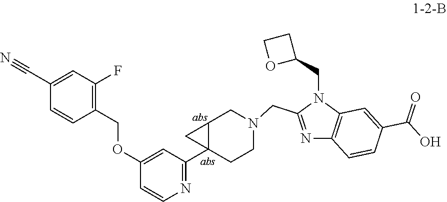 [SMILES: N#Cc1ccc(COc2ccnc(C34CCN(Cc5nc6ccc(C(=O)O)cc6n5C[C@@H]5CCO5)CC3C4)c2)c(F)c1]

2-((6-(4-((4-cyano-2-fluorobenzyl)oxy)pyridin-2-yl)-3-azabicyclo[4.1.0]heptan-3-yl)methyl)-1-(((S)-oxetan-2-yl)methyl)-1H-benzo[d]imidazole-6-carboxylic acid (1-2-B): white solid. LCMS (ESI, m/z): [M+H]⁺=568.4. ¹H NMR (400 MHZ, DMSO-d₆): δ 8.27-8.25 (m, 2H), 7.95-7.92 (m, 1H), 7.82-7.76 (m, 3H), 7.62 (d, J=8.4 Hz, 1H), 6.93 (d, J=2.4 Hz, 1H), 6.87-6.85 (m, 1H), 5.33 (s, 2H), 5.09-5.05 (m, 1H), 4.81-4.75 (m, 1H), 4.65-4.61 (m, 1H), 4.50-4.45 (m, 1H), 4.40-4.35 (m, 1H), 3.94-3.90 (m, 1H), 3.72-3.69 (m, 1H), 2.83-2.78 (m, 2H), 2.71-2.63 (m, 1H), 2.60-2.55 (m, 1H), 2.44-2.38 (m, 1H), 2.31-2.24 (m, 1H), 2.03-1.92 (m, 1H), 1.80-1.74 (m, 1H), 1.24-1.18 (m, 2H), 0.95-0.93 (m, 1H).

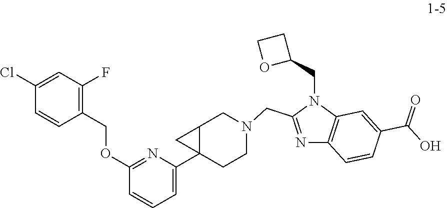 [SMILES: O=C(O)c1ccc2nc(CN3CCC4(c5cccc(OCc6ccc(Cl)cc6F)n5)CC4C3)n(C[C@@H]3CCO3)c2c1]

2-((6-(6-((4-chloro-2-fluorobenzyl)oxy)pyridin-2-yl)-3-azabicyclo[4.1.0]heptan-3-yl)methyl)-1-(((S)-oxetan-2-yl)methyl)-1H-benzo[d]imidazole-6-carboxylic acid (1-5): white solid. LCMS (ESI, m/z): [M+H]⁺=577.4. ¹H NMR (400 MHZ, DMSO-d₆): δ 8.23 (s, 1H), 7.81 (d, J=8.4 Hz, 1H), 7.63-7.60 (m, 2H), 7.53-7.44 (m, 2H), 7.30-7.28 (m, 1H), 6.94 (d, J=7.6 Hz, 1H), 6.61 (d, J=8.0 Hz, 1H), 5.37-5.30 (m, 2H), 5.09-5.03 (m, 1H), 4.80-4.74 (m, 1H), 4.65-4.60 (m, 1H), 4.48-4.40 (m, 2H), 3.93-3.69 (m, 2H), 2.87-2.64 (m, 4H), 2.41-2.29 (m, 3H), 1.98-1.94 (m, 1H), 1.76-1.73 (m, 1H), 1.18-1.14 (m, 1H), 0.95-0.92 (m, 1H).

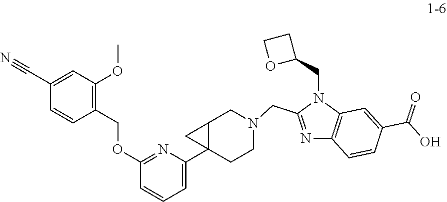 [SMILES: COc1cc(C#N)ccc1COc1cccc(C23CCN(Cc4nc5ccc(C(=O)O)cc5n4C[C@@H]4CCO4)CC2C3)n1]

2-((6-(6-((4-cyano-2-methoxybenzyl)oxy)pyridin-2-yl)-3-azabicyclo[4.1.0]heptan-3-yl)methyl)-1-(((S)-oxetan-2-yl)methyl)-1H-benzo[d]imidazole-6-carboxylic acid (1-6): white solid. LCMS (ESI, m/z): [M+H]⁺=580.3. ¹H NMR (400 MHZ, DMSO-d₆): δ 8.24 (s, 1H), 7.81 (d, J=8.4 Hz, 1H), 7.64-7.60 (m, 2H), 7.51-7.39 (m, 3H), 6.93 (d, J=7.6 Hz, 1H), 6.65 (d, J=8.0 Hz, 1H), 5.38-5.30 (m, 2H), 5.10-5.03 (m, 1H), 4.79-4.74 (m, 1H), 4.64-4.60 (m, 1H), 4.49-4.30 (m, 2H), 3.92-3.84 (m, 4H), 3.77-3.68 (m, 1H), 2.86-2.78 (m, 2H), 2.71-2.64 (m, 2H), 2.39-2.28 (m, 3H), 1.97-1.90 (m, 1H), 1.70-1.64 (m, 1H), 1.10-1.05 (m, 1H), 0.92-0.88 (m, 1H).

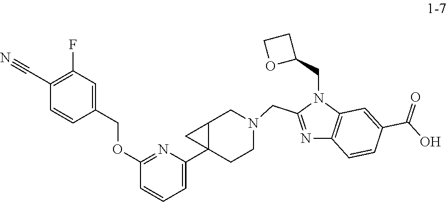 [SMILES: N#Cc1ccc(COc2cccc(C34CCN(Cc5nc6ccc(C(=O)O)cc6n5C[C@@H]5CCO5)CC3C4)n2)cc1F]

2-((6-(6-((4-cyano-3-fluorobenzyl)oxy)pyridin-2-yl)-3-azabicyclo[4.1.0]heptan-3-yl)methyl)-1-(((S)-oxetan-2-yl)methyl)-1H-benzo[d]imidazole-6-carboxylic acid (1-7): white solid. LCMS (ESI, m/z): [M+H]⁺=568.4. ¹H NMR (400 MHZ, DMSO-d₆): δ 8.24 (s, 1H), 7.93-7.90 (m, 1H), 7.82-7.80 (m, 1H), 7.66-7.61 (m, 2H), 7.56-7.53 (m, 1H), 7.43 (d, J=7.6 Hz, 1H), 6.95 (d, J=7.6 Hz, 1H), 6.68 (d, J=8.0 Hz, 1H), 5.44-5.37 (m, 2H), 5.09-5.03 (m, 1H), 4.79-4.74 (m, 1H), 4.64-4.60 (m, 1H), 4.49-4.43 (m, 1H), 4.40-4.31 (m, 1H), 3.92-3.83 (m, 1H), 3.77-3.68 (m, 1H), 2.82-2.77 (m, 2H), 2.71-2.62 (m, 2H), 2.41-2.29 (m, 3H), 1.97-1.91 (m, 1H), 1.70-1.67 (m, 1H), 1.09-1.05 (m, 1H), 0.92-0.89 (m, 1H).

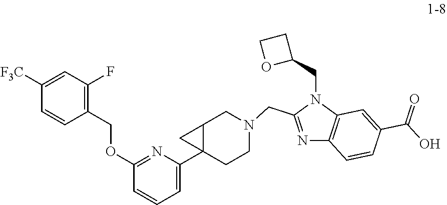 [SMILES: O=C(O)c1ccc2nc(CN3CCC4(c5cccc(OCc6ccc(C(F)(F)F)cc6F)n5)CC4C3)n(C[C@@H]3CCO3)c2c1]

2-{[6-(6-{[2-fluoro-4-(trifluoromethyl)phenyl]methoxy}pyridin-2-yl)-3-azabicyclo[4.1.0]heptan-3-yl]methyl}-3-[(2S)-oxetan-2-ylmethyl]-1,3-benzodiazole-5-carboxylic acid (1-8): white solid. LCMS (ESI, m/z): [M+H]⁺=611.2. ¹H-NMR (400 MHZ, DMSO-d₆): δ 12.76 (s, 1H), 8.25 (s, 1H), 7.82-7.80 (m, 1H), 7.72-7.57 (m, 5H), 6.95 (d, J=7.6 Hz, 1H), 6.66 (d, J=8.0 Hz, 1H), 5.51-5.42 (m, 2H), 5.12-5.01 (m, 1H), 4.80-4.74 (m, 1H), 4.64-4.61 (m, 1H), 4.46-4.42 (m, 1H), 4.41-4.28 (m, 1H) 3.93-3.84 (m, 1H), 3.77-3.68 (m, 1H), 2.77-2.65 (m, 3H), 2.53-2.50 (m, 1H), 2.40-2.36 (m, 2H), 2.34-2.29 (m, 1H), 1.99-1.89 (m, 1H), 1.69-1.67 (m, 1H), 1.12-1.09 (m, 1H), 0.91-0.88 (m, 1H).

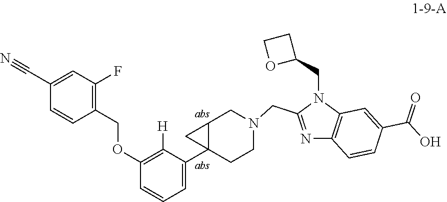 [SMILES: N#Cc1ccc(COc2cccc(C34CCN(Cc5nc6ccc(C(=O)O)cc6n5C[C@@H]5CCO5)CC3C4)c2)c(F)c1]

2-((6-(3-((4-cyano-2-fluorobenzyl)oxy)phenyl)-3-azabicyclo[4.1.0]heptan-3-yl)methyl)-1-(((S)-oxetan-2-yl)methyl)-1H-benzo[d]imidazole-6-carboxylic acid (1-9-A): white solid. LCMS (ESI, m/z): [M+H]⁺=567.2. ¹H NMR (400 MHZ, DMSO-d₆): δ 8.20 (s, 1H), 7.91 (d, J=9.6 Hz, 1H), 7.83-7.73 (m, 3H), 7.54 (d, J=8.0 Hz, 1H), 7.24-7.22 (m, 1H), 6.89-6.83 (m, 3H), 5.22 (s, 2H), 5.11-5.07 (m, 1H), 4.79-4.73 (m, 1H), 4.62-4.59 (m, 1H), 4.49-4.46 (m, 1H), 4.41-4.37 (m, 1H), 3.95-3.92 (m, 1H), 3.70-3.67 (m, 1H), 3.52-3.48 (m, 1H), 2.87-2.76 (m, 2H), 2.34-2.29 (m, 2H), 2.06-2.02 (m, 2H), 1.45-1.41 (m, 1H), 1.28-1.24 (m, 1H), 0.96-0.93 (m, 1H), 0.86-0.83 (m, 1H).

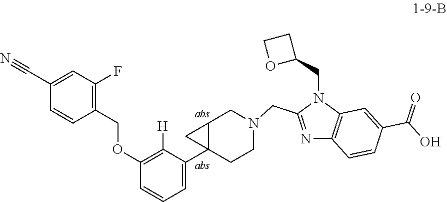 [SMILES: N#Cc1ccc(COc2cccc(C34CCN(Cc5nc6ccc(C(=O)O)cc6n5C[C@@H]5CCO5)CC3C4)c2)c(F)c1]

2-((6-(3-((4-cyano-2-fluorobenzyl)oxy)phenyl)-3-azabicyclo[4.1.0]heptan-3-yl)methyl)-1-(((S)-oxetan-2-yl)methyl)-1H-benzo[d]imidazole-6-carboxylic acid (1-9-B): white solid. LCMS (ESI, m/z): [M+H]⁺=567.2. ¹H NMR (400 MHZ, DMSO-d₆): δ 8.25 (s, 1H), 7.93-7.90 (m, 1H), 7.83-7.75 (m, 3H), 7.61 (d, J=8.0 Hz, 1H), 7.24-7.20 (m, 1H), 6.89-6.83 (m, 3H), 5.22 (s, 2H), 5.13-5.09 (m, 1H), 4.81-4.76 (m, 1H), 4.67-4.63 (m, 1H), 4.50-4.45 (m, 1H), 4.38-4.33 (m, 1H), 3.88-3.77 (m, 2H), 2.90-2.86 (m, 1H), 2.74-2.67 (m, 2H), 2.42-2.38 (m, 2H), 2.34-2.29 (m, 1H), 2.07-2.04 (m, 2H), 1.42-1.39 (m, 1H), 0.95-0.92 (m, 1H), 0.85-0.82 (m, 1H).

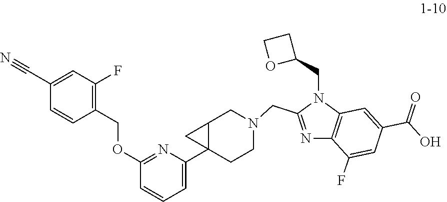 [SMILES: N#Cc1ccc(COc2cccc(C34CCN(Cc5nc6c(F)cc(C(=O)O)cc6n5C[C@@H]5CCO5)CC3C4)n2)c(F)c1]

2-((6-(6-((4-cyano-2-fluorobenzyl)oxy)pyridin-2-yl)-3-azabicyclo[4.1.0]heptan-3-yl)methyl)-4-fluoro-1-(((S)-oxetan-2-yl)methyl)-1H-benzo[d]imidazole-6-carboxylic acid (1-10): white solid. LCMS (ESI, m/z): [M+H]⁺=586.2. ¹H NMR (400 MHZ, CD₃OD): δ 8.04 (s, 1H), 7.66-7.53 (m, 5H), 6.93 (d, J=7.6 Hz, 1H), 6.63 (d, J=8.4 Hz, 1H), 5.52-5.44 (m, 2H), 5.26-5.22 (m, 1H), 4.91-4.89 (m, 1H), 4.73-4.69 (m, 1H), 4.61-4.59 (m, 1H), 4.51-4.40 (m, 1H), 3.99-3.82 (m, 2H), 2.91-2.75 (m, 3H), 2.59-2.39 (m, 4H), 2.08-2.02 (m, 1H), 1.75-1.73 (m, 1H), 1.16-1.14 (m, 1H), 0.97-0.95 (m, 1H).

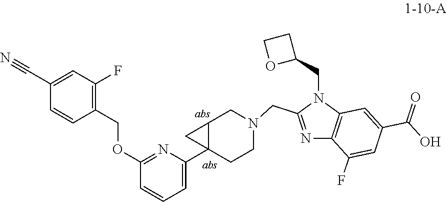 [SMILES: N#Cc1ccc(COc2cccc(C34CCN(Cc5nc6c(F)cc(C(=O)O)cc6n5C[C@@H]5CCO5)CC3C4)n2)c(F)c1]

2-((6-(6-((4-cyano-2-fluorobenzyl)oxy)pyridin-2-yl)-3-azabicyclo[4.1.0]heptan-3-yl)methyl)-4-fluoro-1-(((S)-oxetan-2-yl)methyl)-1H-benzo[d]imidazole-6-carboxylic acid (1-10-A): white solid. LCMS (ESI, m/z): [M+H]⁺=586.3. ¹H NMR (400 MHZ, DMSO-d₆): δ 8.08 (s, 1H), 7.89 (d, J=10.0 Hz, 1H), 7.71-7.61 (m, 3H), 7.52-7.50 (m, 1H), 6.95 (d, J=7.6 Hz, 1H), 6.66 (d, J=8.0 Hz, 1H), 5.44-5.39 (m, 2H), 5.06-5.04 (m, 1H), 4.81-4.77 (m, 1H), 4.65-4.62 (m, 1H), 4.47-4.44 (m, 1H), 4.39-4.36 (m, 1H), 3.93-3.89 (m, 1H), 3.73-3.70 (m, 1H), 2.78 (s, 2H), 2.68-2.64 (m, 1H), 2.42-2.40 (m, 1H), 2.39-2.36 (m, 2H), 2.31-2.28 (m, 1H), 1.96-1.92 (m, 1H), 1.72-1.67 (m, 1H), 1.12-1.09 (m, 1H), 0.93-0.89 (m, 1H).

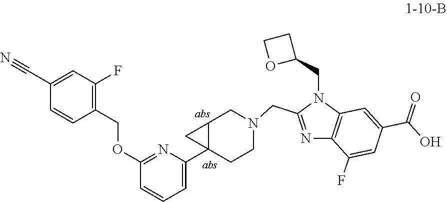 [SMILES: N#Cc1ccc(COc2cccc(C34CCN(Cc5nc6c(F)cc(C(=O)O)cc6n5C[C@@H]5CCO5)CC3C4)n2)c(F)c1]

2-((6-(6-((4-cyano-2-fluorobenzyl)oxy)pyridin-2-yl)-3-azabicyclo[4.1.0]heptan-3-yl)methyl)-4-fluoro-1-(((S)-oxetan-2-yl)methyl)-1H-benzo[d]imidazole-6-carboxylic acid (1-10-B): white solid. LCMS (ESI, m/z): [M+H]⁺=586.4. ¹H NMR (400 MHZ, DMSO-d₆): δ 8.08 (s, 1H), 7.89 (d, J=10.0 Hz, 1H), 7.70-7.61 (m, 3H), 7.53-7.50 (m, 1H), 6.95 (d, J=8.0 Hz, 1H), 6.66 (d, J=8.0 Hz, 1H), 5.44-5.39 (m, 2H), 5.08-5.04 (m, 1H), 4.82-4.76 (m, 1H), 4.66-4.62 (m, 1H), 4.46-4.42 (m, 1H), 4.34-4.32 (m, 1H), 3.88-3.76 (m, 2H), 2.83-2.81 (m, 1H), 2.73-2.63 (m, 2H), 2.41-2.30 (m, 4H), 1.96-1.92 (m, 1H), 1.69-1.67 (m, 1H), 1.11-1.08 (m, 1H), 0.92-0.88 (m, 1H).

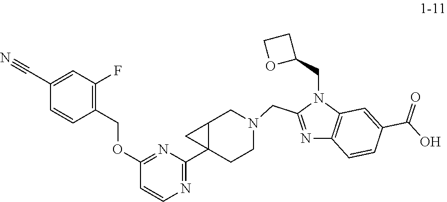 [SMILES: N#Cc1ccc(COc2ccnc(C34CCN(Cc5nc6ccc(C(=O)O)cc6n5C[C@@H]5CCO5)CC3C4)n2)c(F)c1]

2-((6-(4-((4-cyano-2-fluorobenzyl)oxy)pyrimidin-2-yl)-3-azabicyclo[4.1.0]heptan-3-yl)methyl)-1-(((S)-oxetan-2-yl)methyl)-1H-benzo[d]imidazole-6-carboxylic acid (1-11): white solid. LCMS (ESI, m/z): [M+H]⁺=569.2. ¹H NMR (400 MHZ, CD₃OD): δ 8.35 (d, J=5.6 Hz, 1H), 8.29 (s, 1H), 8.02-8.00 (m, 1H), 7.68-7.56 (m, 4H), 6.67 (d, J=5.6 Hz, 1H), 5.55 (s, 2H), 5.23-5.19 (m, 1H), 4.85-4.81 (m, 1H), 4.72-4.57 (m, 2H), 4.50-4.38 (m, 1H), 4.00-3.79 (m, 2H), 3.13-3.09 (m, 1H), 2.94-2.88 (m, 1H), 2.78-2.72 (m, 2H), 2.53-2.49 (m, 2H), 2.28-2.19 (m, 1H), 1.97-1.86 (m, 2H), 1.45-1.40 (m, 1H), 1.13-1.11 (m, 1H).

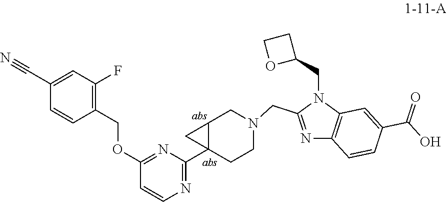 [SMILES: N#Cc1ccc(COc2ccnc(C34CCN(Cc5nc6ccc(C(=O)O)cc6n5C[C@@H]5CCO5)CC3C4)n2)c(F)c1]

2-((6-(4-((4-cyano-2-fluorobenzyl)oxy)pyrimidin-2-yl)-3-azabicyclo[4.1.0]heptan-3-yl)methyl)-1-(((S)-oxetan-2-yl)methyl)-1H-benzo[d]imidazole-6-carboxylic acid (1-11-A): white solid. LCMS (ESI, m/z): [M+H]⁺=569.3. ¹H NMR (400 MHz, CD₃OD): δ 8.36 (d, J=5.6 Hz, 1H), 8.31 (s, 1H), 8.01-7.99 (m, 1H), 7.68-7.57 (m, 4H), 6.68 (d, J=5.6 Hz, 1H), 5.56 (s, 2H), 5.28-5.23 (m, 1H), 4.91-4.88 (m, 1H), 4.75-4.71 (m, 1H), 4.65-4.61 (m, 1H), 4.45-4.40 (m, 1H), 3.96-3.88 (m, 2H), 3.14-3.09 (m, 1H), 2.92-2.89 (m, 1H), 2.80-2.76 (m, 2H), 2.59-2.46 (m, 2H), 2.30-2.24 (m, 1H), 2.01-1.84 (m, 2H), 1.44-1.41 (m, 1H), 1.13-1.11 (m, 1H).

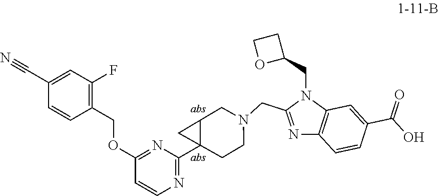 [SMILES: N#Cc1ccc(COc2ccnc(C34CCN(Cc5nc6ccc(C(=O)O)cc6n5C[C@@H]5CCO5)CC3C4)n2)c(F)c1]

2-((6-(4-((4-cyano-2-fluorobenzyl)oxy)pyrimidin-2-yl)-3-azabicyclo[4.1.0]heptan-3-yl)methyl)-1-(((S)-oxetan-2-yl)methyl)-1H-benzo[d]imidazole-6-carboxylic acid (1-11-B): white solid. LCMS (ESI, m/z): [M+H]⁺=569.3. ¹H NMR (400 MHZ, CD₃OD): δ 8.36 (d, J=5.6 Hz, 1H), 8.30 (s, 1H), 8.01-7.98 (m, 1H), 7.68-7.57 (m, 4H), 6.68 (d, J=5.6 Hz, 1H), 5.56 (s, 2H), 5.27-5.20 (m, 1H), 4.92-4.88 (m, 1H), 4.73-4.60 (m, 2H), 4.51-4.46 (m, 1H), 4.01-3.98 (m, 1H), 3.84-3.80 (m, 1H), 3.14-3.10 (m, 1H), 2.98-2.95 (m, 1H), 2.83-2.74 (m, 2H), 2.57-2.49 (m, 2H), 2.27-2.20 (m, 1H), 1.98-1.85 (m, 2H), 1.46-1.42 (m, 1H), 1.14-1.12 (m, 1H).

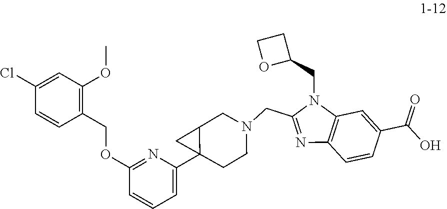 [SMILES: COc1cc(Cl)ccc1COc1cccc(C23CCN(Cc4nc5ccc(C(=O)O)cc5n4C[C@@H]4CCO4)CC2C3)n1]

2-((6-(6-((4-chloro-2-methoxybenzyl)oxy)pyridin-2-yl)-3-azabicyclo[4.1.0]heptan-3-yl)methyl)-1-(((S)-oxetan-2-yl)methyl)-1H-benzo[d]imidazole-6-carboxylic acid (1-12): LCMS (ESI, m/z): [M+H]⁺=589.2. ¹H NMR (400 MHZ, CD₃OD): δ 8.31 (s, 1H), 8.00-7.97 (m, 1H), 7.68-7.65 (m, 1H), 7.57-7.54 (m, 1H), 7.30 (d, J=8.0 Hz, 1H), 7.00 (d, J=2.0 Hz, 1H), 6.92-6.89 (m, 2H), 6.57 (d, J=8.4 Hz, 1H), 5.35 (s, 2H), 5.28-5.20 (m, 1H), 4.88-4.85 (m, 1H), 4.73-4.68 (m, 1H), 4.64-4.57 (m, 1H), 4.50-4.40 (m, 1H), 4.03-3.83 (m, 5H), 3.02-2.92 (m, 1H), 2.85-2.70 (m, 2H), 2.62-2.56 (m, 1H), 2.54-2.43 (m, 3H), 2.11-2.05 (m, 1H), 1.83-1.79 (m, 1H), 1.24-1.26 (m, 1H), 0.98-0.95 (m, 1H).

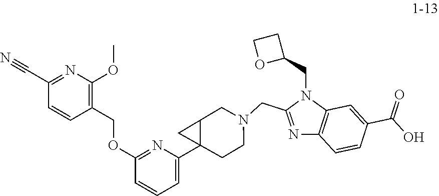 [SMILES: COc1nc(C#N)ccc1COc1cccc(C23CCN(Cc4nc5ccc(C(=O)O)cc5n4C[C@@H]4CCO4)CC2C3)n1]

2-((6-(6-((6-cyano-2-methoxypyridin-3-yl)methoxy)pyridin-2-yl)-3-azabicyclo[4.1.0]heptan-3-yl)methyl)-1-(((S)-oxetan-2-yl)methyl)-1H-benzo[d]imidazole-6-carboxylic acid (1-13): LCMS (ESI, m/z): [M+H]⁺==581.3. ¹H NMR (400 MHZ, DMSO-d₆): δ 12.78 (s, 1H), 8.25 (s, 1H), 7.86-7.79 (m, 2H), 7.66-7.62 (m, 3H), 6.95 (d, J=7.6 Hz, 1H), 6.69 (d, J=8.0 Hz, 1H), 5.33-5.30 (m, 2H), 5.08-5.04 (m, 1H), 4.80-4.74 (m, 1H), 4.65-4.60 (m, 1H), 4.50-4.43 (m, 1H), 4.40-4.30 (m, 1H), 3.96 (s, 3H), 3.93-3.84 (m, 1H), 3.78-3.68 (m, 1H), 2.86-2.78 (m, 1H), 2.72-2.64 (m, 2H), 2.51-2.37 (m, 3H), 2.30-2.26 (m, 1H), 1.96-1.91 (m, 1H), 1.67-1.62 (m, 1H), 1.07-1.02 (m, 1H), 0.92-0.89 (m, 1H).

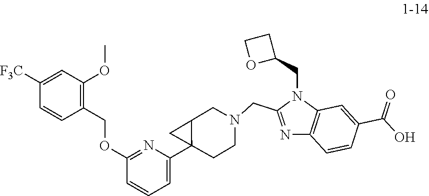 [SMILES: COc1cc(C(F)(F)F)ccc1COc1cccc(C23CCN(Cc4nc5ccc(C(=O)O)cc5n4C[C@@H]4CCO4)CC2C3)n1]

2-((6-(6-((2-methoxy-4-(trifluoromethyl)benzyl)oxy)pyridin-2-yl)-3-azabicyclo[4.1.0]heptan-3-yl)methyl)-1-(((S)-oxetan-2-yl)methyl)-1H-benzo[d]imidazole-6-carboxylic acid (1-14): The title compound was synthesized by using 1-14a as the starting material of which the synthesis was shown below. LCMS (ESI, m/z): [M+H]⁺=623.3. ¹H NMR (400 MHZ, CD₃OD): δ 8.32 (s, 1H), 8.00-7.98 (d, J=8.8 Hz, 1H), 7.68-7.65 (m, 1H), 7.59-7.55 (m, 1H), 7.50 (d, J=8.4 Hz, 1H), 7.22-7.20 (m, 2H), 6.91 (d, J=7.6 Hz, 1H), 6.62 (d, J=8.0 Hz, 1H), 5.41 (s, 2H), 5.25-5.21 (m, 1H), 4.89-4.85 (m, 1H), 4.72-4.67 (m, 1H), 4.63-4.57 (m, 1H), 4.49-4.39 (m, 1H), 4.02-3.83 (m, 5H), 3.00-2.69 (m, 3H), 2.61-2.42 (m, 4H), 2.09-2.05 (m, 1H), 1.78-1.71 (m, 1H), 1.19-1.16 (m, 1H), 0.96-0.93 (m, 1H).

The synthesis of 2-bromo-6-((2-methoxy-4-(trifluoromethyl)benzyl)oxy)pyridine (1-14a):

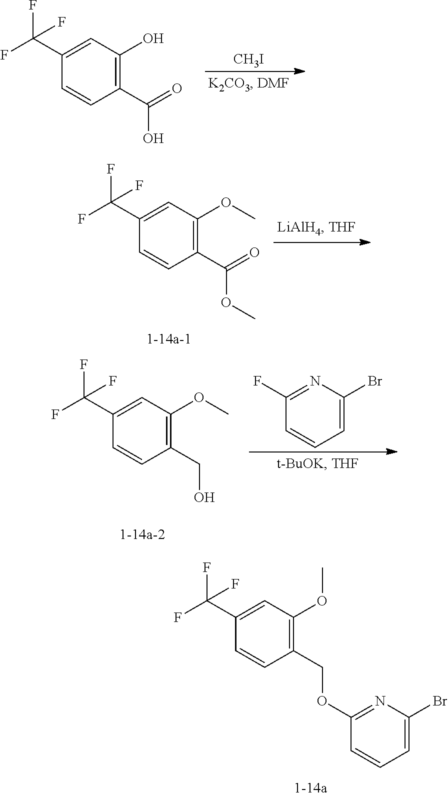

Step 1. To a solution of 2-hydroxy-4-(trifluoromethyl)benzoic acid (3.0 g, 14.56 mmol) in DMF (15.0 mL) was added K₂CO₃ (4.0 g, 29.11 mmol) and CH₃I (4.5 g, 32.02 mmol) at room temperature. The resulting mixture was stirred at room temperature for 16 h. After the reaction was completed, the resulting mixture was diluted with water and extracted with ethyl acetate. The combined organic layer was washed with brine, dried over anhydrous sodium sulfate and filtered. The filtrate was concentrated under vacuum to afford 1-14a-1 (3.0 g, crude) as a light yellow oil. LCMS (ESI, m/z): [M+H]⁺=235.0.

Step 2. To a solution of LiAlH₄ (769.8 mg, 20.28 mmol) in THF (25.0 mL) was added a solution of 1-14a-1 (1.9 g, crude) in THF (10.0 mL) at 0° C. under N₂. The resulting mixture was stirred at room temperature for 2 h under N₂. After the reaction was completed, the resulting mixture was quenched with H₂O and then extracted with ethyl acetate. The combined organic layer was washed with brine, dried over anhydrous sodium sulfate and filtered. The filtrate was concentrated under vacuum to afford 1-14a-2 (1.6 g, crude) as a light yellow oil.

Step 3. To a mixture of 2-bromo-6-fluoropyridine (1.2 g, 6.82 mmol) and 1-14a-2 (1.7 g, 8.18 mmol) in THF (20.0 mL) was added a solution of t-BuOK (1.4 g, 12.27 mmol) in THF (10 mL) at 0° C. under N₂. The resulting mixture was stirred at room temperature for 2 h. After the reaction was completed, the resulting mixture was quenched with NH₄Cl (aq) and then extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (74/26, v/v) to afford 1-14a (2.3 g, 93%) as a colorless oil. LCMS (ESI, m/z): [M+H]⁺=362.0.

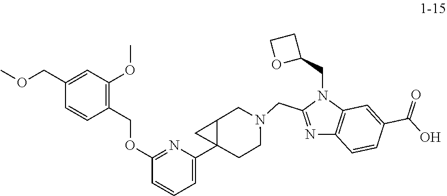 [SMILES: COCc1ccc(COc2cccc(C34CCN(Cc5nc6ccc(C(=O)O)cc6n5C[C@@H]5CCO5)CC3C4)n2)c(OC)c1]

2-((6-(6-((2-methoxy-4-(methoxymethyl)benzyl)oxy)pyridin-2-yl)-3-azabicyclo[4.1.0]heptan-3-yl)methyl)-1-(((S)-oxetan-2-yl)methyl)-1H-benzo[d]imidazole-6-carboxylic acid (1-15): LCMS (ESI, m/z): [M+H]⁺=599.3. ¹H NMR (400 MHZ, CD₃OD): δ 8.31 (s, 1H), 7.99 (d, J=8.0 Hz, 1H), 7.67-7.65 (m, 1H), 7.57-7.53 (m, 1H), 7.31 (d, J=7.6 Hz, 1H), 6.97 (s, 1H), 6.90-6.86 (m, 2H), 6.56 (d, J=8.0 Hz, 1H), 5.34 (s, 2H), 5.26-5.21 (m, 1H), 4.72-4.68 (m, 1H), 4.63-4.57 (m, 1H), 4.49-4.39 (m, 3H), 4.03-3.82 (m, 5H), 3.36 (s, 3H), 3.01-2.91 (m, 1H), 2.83-2.73 (m, 2H), 2.64-2.56 (m, 1H), 2.54-2.43 (m, 3H), 2.12-2.07 (m, 1H), 1.83-1.78 (m, 1H), 1.28-1.22 (m, 1H), 0.98-0.94 (m, 1H).

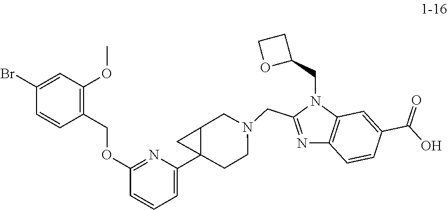 [SMILES: COc1cc(Br)ccc1COc1cccc(C23CCN(Cc4nc5ccc(C(=O)O)cc5n4C[C@@H]4CCO4)CC2C3)n1]

2-((6-(6-((4-bromo-2-methoxybenzyl)oxy)pyridin-2-yl)-3-azabicyclo[4.1.0]heptan-3-yl)methyl)-1-(((S)-oxetan-2-yl)methyl)-1H-benzo[d]imidazole-6-carboxylic acid (1-16): The title compound was synthesized by using 1-16c as the starting material of which the synthesis was shown below. white solid. LCMS (ESI, m/z): [M+H]⁺==633.3. ¹H NMR (400 MHz, DMSO-d₆): δ 8.24 (s, 1H), 7.81 (d, J=8.0 Hz, 1H), 7.64-7.58 (m, 2H), 7.27 (d, J=8.0 Hz, 1H), 7.21 (d, J=1.6 Hz, 1H), 7.13-7.11 (m, 1H), 6.92 (d, J=7.2 Hz, 1H), 6.60 (d, J=8.0 Hz, 1H), 5.24 (s, 2H), 5.09-5.03 (m, 1H), 4.80-4.75 (m, 1H), 4.65-4.60 (m, 1H), 4.49-4.43 (m, 1H), 4.40-4.31 (m, 1H), 3.93-3.88 (m, 1H), 3.83 (s, 3H), 3.78-3.69 (m, 1H), 2.88-2.84 (m, 1H), 2.79-2.68 (m, 2H), 2.53-2.50 (m, 1H), 2.44-2.31 (m, 3H), 1.99-1.94 (m, 1H), 1.76-1.72 (m, 1H), 1.17-1.13 (m, 1H), 0.94-0.91 (m, 1H).

The synthesis of 6-(6-((4-bromo-2-methoxybenzyl)oxy)pyridin-2-yl)-3-azabicyclo[4.1.0]heptane (1-16c):

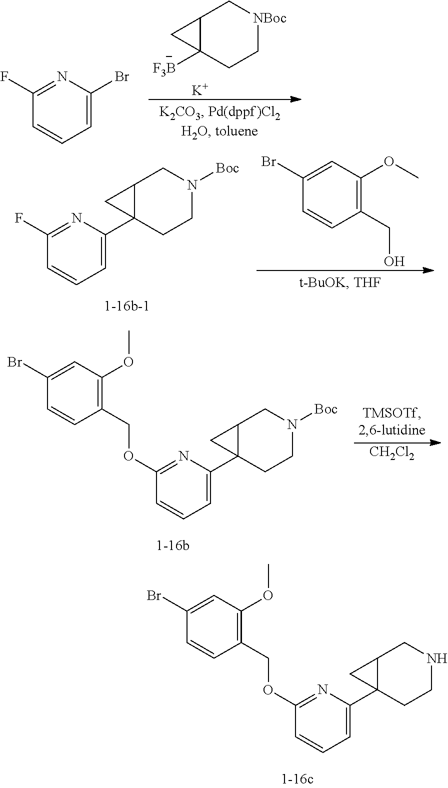

Step 1. To a solution of 2-bromo-6-fluoropyridine (300.0 mg, 1.71 mmol) in H₂O (3.0 mL) and toluene (15.0 mL) was added potassium (3-(tert-butoxycarbonyl)-3-azabicyclo[4.1.0]heptan-6-yl)trifluoroborate (537.5 mg, 1.77 mmol), Pd(dppf)Cl₂ (249.5 mg, 0.34 mmol) and K₂CO₃ (360.5 mg, 2.61 mmol) at room temperature under N₂. The resulting mixture was stirred at 100° C. for 16 h under N₂. After the reaction was completed, the resulting mixture was filtered. The filtrate was concentrated under reduced pressure. The residue was purified by flash column chromatography with petroleum ether/ethyl acetate (90/10, v/v) to afford 1-16b-1 (300.0 mg, 60%) as a white solid. LCMS (ESI, m/z): [M+H]⁺=293.2.

Step 2. To a solution of 1-16b-1 (200.0 mg, 0.68 mmol) in THF (10.0 mL) was added (4-bromo-2-methoxyphenyl) methanol (280.0 mg, 1.29 mmol) and potassium tert-butoxide (160.0 mg, 1.43 mmol) at room temperature. The resulting mixture was stirred at 40° C. for 1 h. After the reaction was completed, the mixture was quenched by the addition of NH₄Cl (aq.). and then extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (90/10, v/v) to afford 1-16b (266.0 mg, 79%) as a yellow solid. LCMS (ESI, m/z): [M+H]⁺=489.1.

Step 3. To a solution of 1-16b (226.0 mg, 0.46 mmol) in CH₂Cl₂ (10.0 mL) was added TMSOTf (206.0 mg, 0.93 mmol) and 2,6-lutidine (50.0 mg, 0.47 mmol) at room temperature. The resulting mixture was stirred at room temperature for 16 h. After the reaction was completed, the resulting mixture was diluted with water and extracted with CH₂Cl₂. The combined organic layer was washed with brine, dried over anhydrous sodium sulfate and filtered. The filtrate was concentrated under vacuum to afford 1-16c (200.0 mg, crude) as a white solid. LCMS (ESI, m/z): [M+H]⁺=389.1.

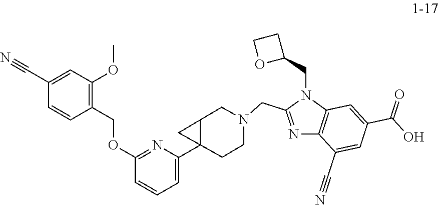 [SMILES: COc1cc(C#N)ccc1COc1cccc(C23CCN(Cc4nc5c(C#N)cc(C(=O)O)cc5n4C[C@@H]4CCO4)CC2C3)n1]

4-cyano-2-((6-(6-((4-cyano-2-methoxybenzyl)oxy)pyridin-2-yl)-3-azabicyclo[4.1.0]heptan-3-yl)methyl)-1-(((S)-oxetan-2-yl)methyl)-1H-benzo[d]imidazole-6-carboxylic acid (1-17): The title compound was synthesized by using 1-17e as the starting material of which the synthesis was shown below. LCMS (ESI, m/z): [M+H]⁺=605.3. ¹H NMR (400 MHZ, CD₃OD): δ 8.57-8.56 (m, 1H), 8.31 (d, J=1.6 Hz, 1H), 7.61-7.57 (m, 1H), 7.48 (d, J=7.6 Hz, 1H), 7.34 (s, 1H), 7.29 (d, J=7.6 Hz, 1H), 6.92 (d, J=7.6 Hz, 1H), 6.63 (d, J=8.0 Hz, 1H), 5.46-5.37 (m, 2H), 5.26-5.19 (m, 1H), 4.95-4.91 (m, 1H), 4.77-4.72 (m, 1H), 4.64-4.58 (m, 1H), 4.51-4.41 (m, 1H), 4.10-3.90 (m, 5H), 3.06-2.95 (m, 1H), 2.88-2.73 (m, 2H), 2.63-2.55 (m, 1H), 2.53-2.43 (m, 3H), 2.11-2.06 (m, 1H), 1.78-1.73 (m, 1H), 1.17-1.14 (m, 1H), 0.97-0.93 (m, 1H).

The synthesis of methyl(S)-2-(chloromethyl)-4-cyano-1-(oxetan-2-ylmethyl)-1H-benzo[d]imidazole-6-carboxylate (1-17e):

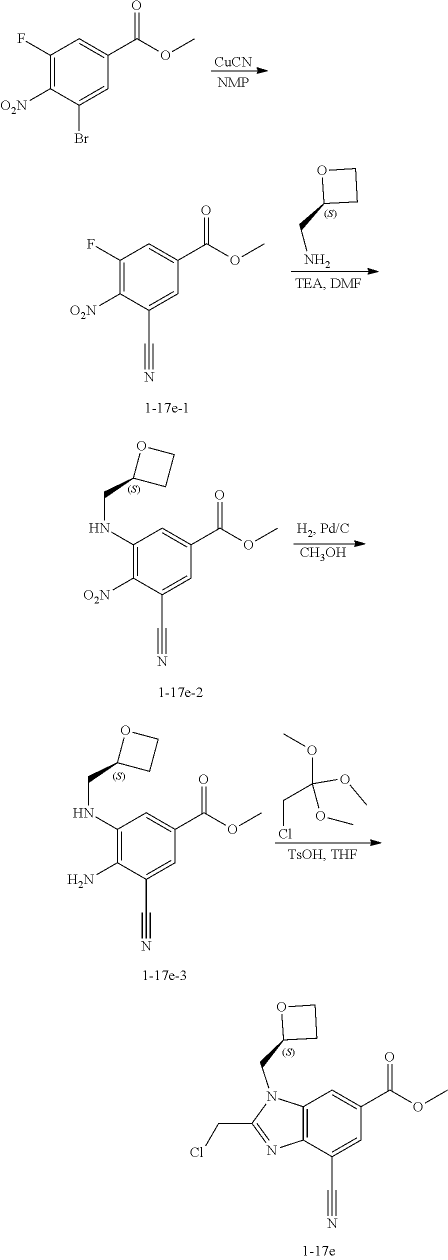

Step 1. To a solution of methyl 3-bromo-5-fluoro-4-nitrobenzoate (1.0 g, 3.60 mmol) in NMP (15.0 mL) was added CuCN (483.2 mg, 5.40 mmol) at room temperature. The resulting mixture was stirred at 150° C. for 7 h under N₂. After the reaction was completed, the mixture was cooled to room temperature and diluted with water. The mixture was extracted with ethyl acetate. The combined organic layer was washed with brine, dried over anhydrous sodium sulfate and filtered. The filtrate was concentrated under vacuum. The residue was purified by flash column chromatography with ethyl acetate/petroleum ether (25/75, v/v) to afford 1-17e-1 (304.7 mg, 38%) as a yellow green solid.

Step 2. To a solution of 1-17e-1 (304.7 mg, 1.36 mmol) in DMF (10.0 mL) was added(S)-oxetan-2-ylmethanamine (130.3 mg, 1.50 mmol) and TEA (412.7 mg, 4.08 mmol) at room temperature. The resulting mixture was stirred at room temperature for 4 h. After the reaction was completed, the mixture was diluted with water and extracted with ethyl acetate. The combined organic layer was washed with brine, dried over anhydrous sodium sulfate and filtered. The filtrate was concentrated under vacuum to afford 1-17e-2 (360.4 mg, crude) as a yellow solid. LCMS (ESI, m/z): [M+H]⁺=292.1.

Step 3. To a solution of 1-17e-2 (360.4 mg, 1.24 mmol) in CH₃OH (10.0 mL) was added Pd/C (90.0 mg, 10%) at room temperature under N₂. The resulting mixture was stirred at room temperature for 4 h under H₂. After the reaction was completed, the mixture was filtered. The filtrate was concentrated under vacuum. The residue was purified by flash column chromatography with ethyl acetate/petroleum ether (70/30, v/v) to afford 1-17e-3 (81.2 mg, 25%) as a yellow green solid. LCMS (ESI, m/z): [M+H]⁺=262.1.

Step 4. To a solution of 1-17e-3 (81.2 mg, 0.31 mmol) in THF (4.0 mL) was added 2-chloro-1,1,1-trimethoxyethane (72.1 mg, 0.47 mmol) and TsOH·H₂O (18.2 mg, 0.11 mmol) at room temperature. The resulting mixture was stirred at 40° C. for 16 h. After the reaction was completed, the mixture was concentrated under vacuum. The residue was purified by flash column chromatography with ethyl acetate/petroleum ether (55/45, v/v) to afford 1-17e (55.6 mg, 56%) as an off-white solid. LCMS (ESI, m/z): [M+H]⁺==320.1.

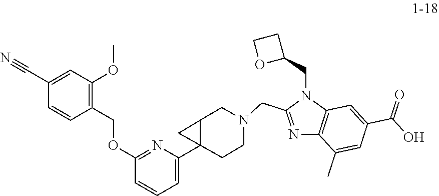 [SMILES: COc1cc(C#N)ccc1COc1cccc(C23CCN(Cc4nc5c(C)cc(C(=O)O)cc5n4C[C@@H]4CCO4)CC2C3)n1]

2-((6-(6-((4-cyano-2-methoxybenzyl)oxy)pyridin-2-yl)-3-azabicyclo[4.1.0]heptan-3-yl)methyl)-4-methyl-1-(((S)-oxetan-2-yl)methyl)-1H-benzo[d]imidazole-6-carboxylic acid (1-18): The title compound was synthesized by using 1-18e as the starting material of which the synthesis was shown below. white solid. LCMS (ESI, m/z): [M+H]⁺=594.3. ¹H NMR (400 MHZ, DMSO-d₆): δ 8.04 (s, 1H), 7.64-7.60 (m, 2H), 7.52-7.39 (m, 3H), 6.93 (d, J=7.6 Hz, 1H), 6.65 (d, J=8.0 Hz, 1H), 5.38-5.30 (m, 2H), 5.06-5.02 (m, 1H), 4.77-4.72 (m, 1H), 4.62-4.58 (m, 1H), 4.48-4.42 (m, 1H), 4.38-4.29 (m, 1H), 3.91-3.70 (m, 5H), 2.85-2.77 (m, 1H), 2.71-2.65 (m, 2H), 2.54 (s, 3H), 2.38-2.36 (m, 3H), 2.30-2.24 (m, 1H), 1.96-1.91 (m, 1H), 1.72-1.67 (m, 1H), 1.10-1.06 (m, 1H), 0.92-0.88 (m, 1H).

The synthesis of methyl(S)-2-(chloromethyl)-4-methyl-1-(oxetan-2-ylmethyl)-1H-benzo[d]imidazole-6-carboxylate (1-18e):

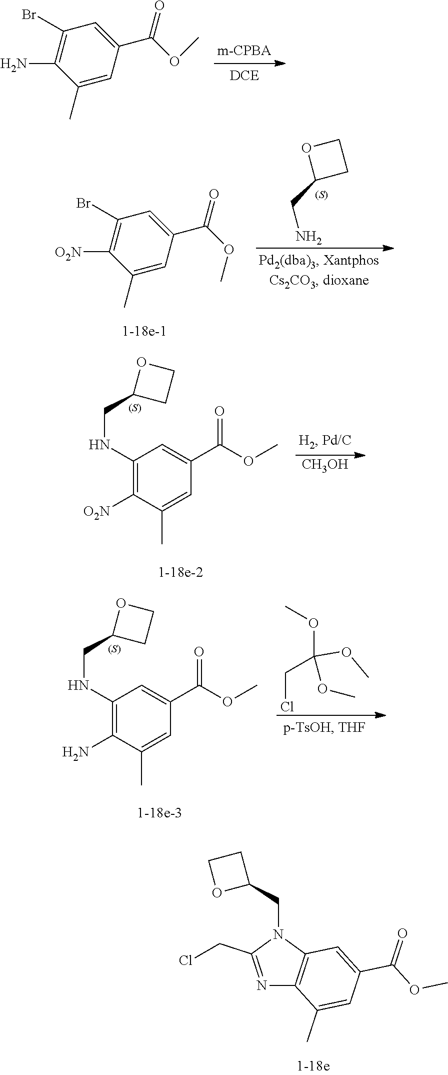

Step 1. To a solution of methyl 4-amino-3-bromo-5-methylbenzoate (2.0 g, 8.19 mmol) in DCE (35.0 mL) was added m-CPBA (7.1 g, 40.97 mmol) at room temperature. The resulting mixture was stirred at room temperature for 16 h. After the reaction was completed, the resulting mixture was filtered. The filtrate diluted with saturated Na₂CO₃ solution. The mixture was extracted with CH₂Cl₂. The combined organic layer was washed with brine, dried over anhydrous sodium sulfate and filtered. The filtrate was concentrated under vacuum. The residue was purified by flash column chromatography with ethyl acetate/petroleum ether (25/75, v/v) to afford 1-18e-1 (1.6 g, 71%) as a white solid. LCMS (ESI, m/z): [M+H]⁺=274.0.

Step 2. To a solution of 1-18e-1 (1.5 g, 5.47 mmol) in 1,4-dioxane (20.0 mL) was added 1-[(2S)-oxetan-2-yl]methanamine (0.5 g, 5.47 mmol), Pd₂(dba)₃ (0.5 g, 0.55 mmol), XantPhos (0.6 g, 1.09 mmol) and Cs₂CO₃ (3.5 g, 10.95 mmol) at room temperature under N₂. The resulting mixture was stirred at 100° C. for 16 h under N₂. After the reaction was completed, the resulting mixture was cooled to room temperature and filtered. The filtrate was concentrated under reduced pressure. The residue was purified by flash column chromatography with petroleum ether/ethyl acetate (78/22, v/v) to afford 1-18e-2 (1.2 g, 78%) as a light yellow solid. LCMS (ESI, m/z): [M+H]⁺=281.2.

Step 3. To a solution of 1-18e-2 (1.2 g, 4.28 mmol) in CH₃OH (15.0 mL) was added Pd/C (80.0 mg, 10%) at room temperature under N₂. The resulting mixture was stirred at room temperature for 4 h under H₂. After the reaction was completed, the mixture was filtered. The filtrate was concentrated under vacuum. The residue was purified by flash column chromatography with ethyl acetate/petroleum ether (79/21, v/v) to afford 1-18e-3 (1.0 g, 93%) as a light yellow solid. LCMS (ESI, m/z): [M+H]⁺=251.3.

Step 4. To a solution of 1-18e-3 (1.0 g, 3.99 mmol) in THF (15.0 mL) was added 2-chloro-1,1,1-trimethoxyethane (0.7 g, 4.79 mmol) and TsOH·H₂O (0.3 g, 1.99 mmol) at room temperature. The resulting mixture was stirred at 40° C. for 16 h. After the reaction was completed, the resulting mixture was concentrated under vacuum. The residue was purified by flash column chromatography with ethyl acetate/petroleum ether (60/40, v/v) to afford 1-18e (0.6 g, 49%) as an off-white solid. LCMS (ESI, m/z): [M+H]⁺=309.1.

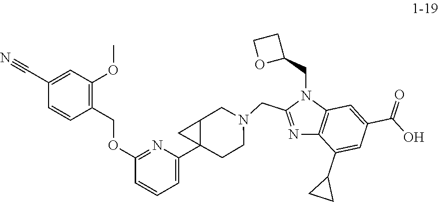 [SMILES: COc1cc(C#N)ccc1COc1cccc(C23CCN(Cc4nc5c(C6CC6)cc(C(=O)O)cc5n4C[C@@H]4CCO4)CC2C3)n1]

2-((6-(6-((4-cyano-2-methoxybenzyl)oxy)pyridin-2-yl)-3-azabicyclo[4.1.0]heptan-3-yl)methyl)-4-cyclopropyl-1-(((S)-oxetan-2-yl)methyl)-1H-benzo[d]imidazole-6-carboxylic acid (1-19): The title compound was synthesized by using 1-19e as the starting material of which the synthesis was shown below. LCMS (ESI, m/z): [M+H]⁺=620.4. ¹H NMR (400 MHZ, DMSO-d₆): δ 7.99 (s, 1H), 7.64-7.60 (s, 1H), 7.51 (s, 1H), 7.48-7.46 (m, 1H), 7.42-7.39 (m, 1H), 7.33 (s, 1H), 6.93 (d, J=7.6 Hz, 1H), 6.65 (d, J=8.0 Hz, 1H), 5.39-5.31 (m, 2H), 5.09-5.01 (m, 1H), 4.77-4.71 (m, 1H), 4.62-4.58 (m, 1H), 4.46-4.43 (m, 1H), 4.39-4.30 (m, 1H), 3.93-3.89 (m, 4H), 3.84-3.70 (m, 1H), 2.86-2.79 (m, 2H), 2.66-2.63 (m, 1H), 2.42-2.27 (m, 3H), 1.97-1.91 (m, 1H), 1.74-1.67 (m, 1H), 1.11-1.04 (m, 3H), 0.99-0.95 (m, 2H), 0.93-0.90 (m, 1H).

methyl(S)-2-(chloromethyl)-4-cyclopropyl-1-(oxetan-2-ylmethyl)-1H-benzo[d]imidazole-6-carboxylate (1-19e): The title compound was synthesized following the procedure of compound 1-17e by using 1-19e-1 as the starting material of which the synthesis was shown below. LCMS (ESI, m/z): [M+H]⁺=335.1.

The synthesis of methyl 3-cyclopropyl-5-fluoro-4-nitrobenzoate (1-19e-1):

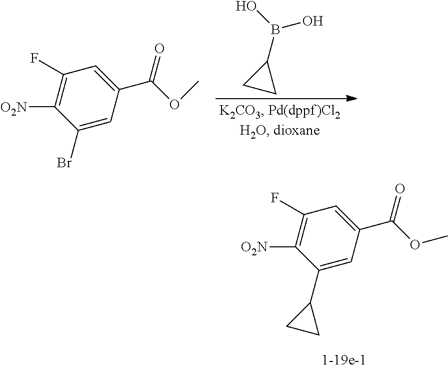

To a solution of methyl 3-bromo-5-fluoro-4-nitrobenzoate (500.0 mg, 1.80 mmol) in H₂O (3.0 mL) and dioxane (15.0 mL) was added cyclopropylboronic acid (775.0 mg, 9.02 mmol), Pd(dppf)Cl₂ (755.0 mg, 1.03 mmol) and K₂CO₃ (395.0 mg, 2.86 mmol) at room temperature under N₂. The resulting mixture was stirred at 100° C. for 16 h under N₂. After the reaction was completed, the resulting mixture was cooled to room temperature and filtered. The filtrate was concentrated under reduced pressure. The residue was purified by flash column chromatography with CH₂Cl₂/petroleum ether (10/90, v/v) to afford 1-19e-1 (160.0 mg, 37%) as a colorless oil.

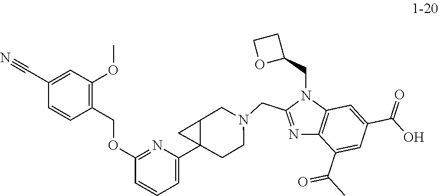 [SMILES: COc1cc(C#N)ccc1COc1cccc(C23CCN(Cc4nc5c(C(C)=O)cc(C(=O)O)cc5n4C[C@@H]4CCO4)CC2C3)n1]

4-acetyl-2-((6-(6-((4-cyano-2-methoxybenzyl)oxy)pyridin-2-yl)-3-azabicyclo[4.1.0]heptan-3-yl)methyl)-1-(((S)-oxetan-2-yl)methyl)-1H-benzo[d]imidazole-6-carboxylic acid (1-20): The title compound was synthesized by using 1-20e as the starting material of which the synthesis was shown below. LCMS (ESI, m/z): [M+H]⁺=622.3. ¹H NMR (400 MHZ, CD₃OD): δ 8.46-8.44 (m, 2H), 7.59-7.55 (m, 1H), 7.45 (d, J=7.6 Hz, 1H), 7.31 (s, 1H), 7.27-7.25 (m, 1H), 6.91 (d, J=7.2 Hz, 1H), 6.62 (d, J=8.0 Hz, 1H), 5.43-5.35 (m, 2H), 5.22-5.15 (m, 1H), 4.85-4.82 (m, 1H), 4.71-4.66 (m, 1H), 4.61-4.54 (m, 1H), 4.46-4.36 (m, 1H), 4.15-3.99 (m, 2H), 3.90 (s, 3H), 3.18-3.07 (m, 1H), 2.93-2.86 (m, 4H), 2.78-2.68 (m, 1H), 2.63-2.40 (m, 4H), 2.13-2.07 (m, 1H), 1.78-1.73 (m, 1H), 1.17-1.14 (m, 1H), 0.97-0.94 (m, 1H).

methyl(S)-4-acetyl-2-(chloromethyl)-1-(oxetan-2-ylmethyl)-1H-benzo[d]imidazole-6-carboxylate (1-20e): The title compound was synthesized following the procedure of compound 1-17e by using 1-20e-1 as the starting material of which the synthesis was shown below. LCMS (ESI, m/z): [M+H]⁺=337.1.

The synthesis of methyl 3-acetyl-5-fluoro-4-nitrobenzoate (1-20e-1): To a solution of methyl 3-bromo-5-fluoro-4-nitrobenzoate (500.0 mg, 1.80 mmol) in THF (10.0 mL) was added tributyl(1-ethoxyvinyl) stannane (974.2 mg, 2.70 mmol) and Pd(PPh₃)₂Cl₂ (126.2 mg, 0.18 mmol) at room temperature under N₂. The resulting mixture was stirred at 70° C. for 4 h under N₂. The mixture was diluted with water and extracted with ethyl acetate. The combined organic layer was washed with brine, dried over anhydrous sodium sulfate and filtered. The filtrate was concentrated under reduced pressure. The residue was dissolved in CH₂Cl₂ (10.0 mL). Then HCl/dioxane (5.0 mL, 4.0 mol/L) was added to the mixture at room temperature. The resulting mixture was stirred at room temperature for additional 1 h. After the reaction was completed, the mixture was concentrated under vacuum. The residue was purified by flash column chromatography with ethyl acetate/petroleum ether (25/75, v/v) to afford methyl 3-acetyl-5-fluoro-4-nitrobenzoate (395.8 mg, 91%) as a yellow solid.

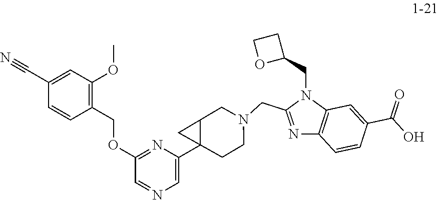 [SMILES: COc1cc(C#N)ccc1COc1cncc(C23CCN(Cc4nc5ccc(C(=O)O)cc5n4C[C@@H]4CCO4)CC2C3)n1]

2-((6-(6-((4-cyano-2-methoxybenzyl)oxy) pyrazin-2-yl)-3-azabicyclo[4.1.0]heptan-3-yl)methyl)-1-(((S)-oxetan-2-yl)methyl)-1H-benzo[d]imidazole-6-carboxylic acid (1-21): LCMS (ESI, m/z): [M+H]⁺=581.4. ¹H NMR (400 MHZ, DMSO-d₆): δ 12.54 (s, 1H), 8.26-8.23 (m, 2H), 8.14 (s, 1H), 7.81 (d, J=8.4 Hz, 1H), 7.65-7.62 (m, 1H), 7.53-7.51 (m, 2H), 7.43-7.41 (d, J=8.0 Hz, 1H), 5.41-5.34 (m, 2H), 5.08-5.04 (m, 1H), 4.80-4.75 (m, 1H), 4.65-4.61 (m, 1H), 4.50-4.43 (m, 1H), 4.40-4.30 (m, 1H), 3.94-3.85 (m, 4H), 3.79-3.70 (m, 1H), 2.88-2.63 (m, 3H), 2.58-2.53 (m, 1H), 2.43-2.27 (m, 3H), 2.05-1.96 (m, 1H), 1.75-1.70 (m, 1H), 1.19-1.14 (m, 1H), 1.03-0.99 (m, 1H).

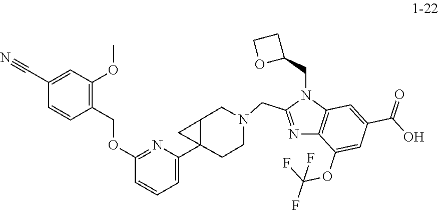 [SMILES: COc1cc(C#N)ccc1COc1cccc(C23CCN(Cc4nc5c(OC(F)(F)F)cc(C(=O)O)cc5n4C[C@@H]4CCO4)CC2C3)n1]

2-((6-(6-((4-cyano-2-methoxybenzyl)oxy)pyridin-2-yl)-3-azabicyclo[4.1.0]heptan-3-yl)methyl)-1-(((S)-oxetan-2-yl)methyl)-4-(trifluoromethoxy)-1H-benzo[d]imidazole-6-carboxylic acid (1-22): The title compound was synthesized by using 1-22e as the starting material of which the synthesis was shown below. LCMS (ESI, m/z): [M+H]⁺=664.3. ¹H NMR (400 MHZ, CD₃OD): δ 8.33 (s, 1H), 7.88 (s, 1H), 7.61-7.57 (m, 1H), 7.48 (d, J=7.6 Hz, 1H), 7.34 (s, 1H), 7.29 (d, J=8.0 Hz, 1H), 6.92 (d, J=7.2 Hz, 1H), 6.63 (d, J=8.0 Hz, 1H), 5.42-5.37 (m, 2H), 5.25-5.22 (m, 1H), 4.94-4.91 (m, 1H), 4.75-4.71 (m, 1H), 4.63-4.60 (m, 1H), 4.50-4.43 (m, 1H), 4.08-3.88 (m, 5H), 3.04-2.94 (m, 1H), 2.87-2.71 (m, 2H), 2.62-2.55 (m, 1H), 2.54-2.45 (m, 3H), 2.11-2.06 (m, 1H), 1.78-1.72 (m, 1H), 1.17-1.14 (m, 1H), 0.97-0.93 (m, 1H).

methyl(S)-2-(chloromethyl)-1-(oxetan-2-ylmethyl)-4-(trifluoromethoxy)-1H-benzo[d]imidazole-6-carboxylate (1-22e): The title compound was synthesized following the procedure of compound 1-18e by using 1-22e-1 as the starting material of which the synthesis was shown below. LCMS (ESI, m/z): [M+H]⁺=379.1.

The synthesis of methyl 3-bromo-4-nitro-5-(trifluoromethoxy)benzoate (1-22e-1)

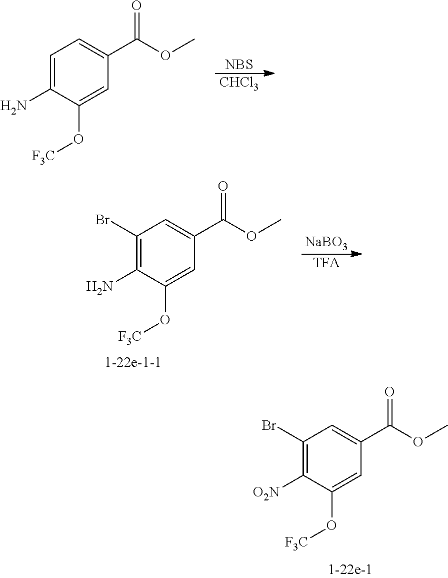

Step 1. To a solution of methyl 4-amino-3-(trifluoromethoxy)benzoate (2.0 g, 8.50 mmol) in CHCl₃ (30.0 mL) was added NBS (1.7 g, 9.36 mmol) at room temperature. The resulting mixture was stirred at room temperature for 2 h. After the reaction was completed, the reaction mixture was quenched with saturated Na₂SO₃ (aq.). The resulting mixture was extracted with ethyl acetate. The combined organic layer was washed with brine, dried over anhydrous sodium sulfate and filtered. The filtrate was concentrated under vacuum. The residue was purified by flash column chromatography with ethyl acetate/petroleum ether (20/80, v/v) to afford 1-22e-1-1 (2.6 g, 97%) as an orange solid. LCMS (ESI, m/z): [M+H]⁺==314.0.

Step 2. To a solution of 1-22e-1-1 (1.5 g, 4.78 mmol) in TFA (10.0 mL) was added NaBO₃ (2.0 g, 23.88 mmol) at room temperature. The resulting mixture was stirred at 80° C. for 3 h. After the reaction was completed, the mixture was cooled to room temperature and diluted with water. The resulting mixture was extracted with ethyl acetate. The combined organic layer was washed with brine, dried over anhydrous sodium sulfate and filtered. The filtrate was concentrated under vacuum. The residue was purified by flash column chromatography with CH₂Cl₂/petroleum ether (30/70, v/v) to afford 1-22e-1 (991.6 mg, 60%) as a light yellow solid. LCMS (ESI, m/z): [M+H]⁺=344.0.

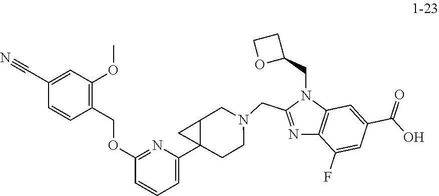 [SMILES: COc1cc(C#N)ccc1COc1cccc(C23CCN(Cc4nc5c(F)cc(C(=O)O)cc5n4C[C@@H]4CCO4)CC2C3)n1]

2-((6-(6-((4-cyano-2-methoxybenzyl)oxy)pyridin-2-yl)-3-azabicyclo[4.1.0]heptan-3-yl)methyl)-4-fluoro-1-(((S)-oxetan-2-yl)methyl)-1H-benzo[d]imidazole-6-carboxylic acid (1-23): LCMS (ESI, m/z): [M+H]⁺=598.3. ¹H NMR (400 MHZ, DMSO-d₆): δ 12.84 (s, 1H), 8.15 (s, 1H), 7.65-7.61 (m, 1H), 7.53-7.46 (m, 3H), 7.40 (d, J=7.6 Hz, 1H), 6.94 (d, J=7.6 Hz, 1H), 6.66 (d, J=8.0 Hz, 1H), 5.39-5.31 (m, 2H), 5.08-5.04 (m, 1H), 4.84-4.78 (m, 1H), 4.68-4.64 (m, 1H), 4.49-4.43 (m, 1H), 4.40-4.30 (m, 1H), 3.95-3.86 (m, 4H), 3.80-3.71 (m, 1H), 2.84-2.63 (m, 3H), 2.40-2.28 (m, 3H), 2.00-1.94 (m, 1H), 1.71-1.68 (m, 1H), 1.11-1.07 (m, 1H), 0.92-0.88 (m, 1H).

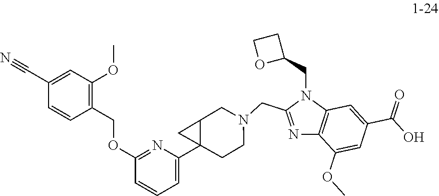 [SMILES: COc1cc(C#N)ccc1COc1cccc(C23CCN(Cc4nc5c(OC)cc(C(=O)O)cc5n4C[C@@H]4CCO4)CC2C3)n1]

2-((6-(6-((4-cyano-2-methoxybenzyl)oxy)pyridin-2-yl)-3-azabicyclo[4.1.0]heptan-3-yl)methyl)-4-methoxy-1-(((S)-oxetan-2-yl)methyl)-1H-benzo[d]imidazole-6-carboxylic acid (1-24): The title compound was synthesized by using 1-24e as the starting material of which the synthesis was shown below. LCMS (ESI, m/z): [M+H]⁺=610.3. ¹H NMR (400 MHz, DMSO-d₆): δ 7.85 (s, 1H), 7.64-7.59 (m, 1H), 7.51-7.46 (m, 2H), 7.41-7.39 (m, 1H), 7.29 (s, 1H), 6.93 (d, J=7.2 Hz, 1H), 6.65 (d, J=8.0 Hz, 1H), 5.38-5.30 (m, 2H), 5.07-5.01 (m, 1H), 4.74-4.69 (m, 1H), 4.60-4.57 (m, 1H), 4.45-4.40 (m, 1H), 4.37-4.24 (m, 1H), 3.95 (s, 3H), 3.93-3.67 (m, 5H), 2.84-2.64 (m, 3H), 2.38-2.26 (m, 3H), 1.96-1.91 (m, 1H), 1.70-1.67 (m, 1H), 1.09-1.06 (m, 1H), 0.92-0.89 (m, 1H).

methyl(S)-2-(chloromethyl)-4-methoxy-1-(oxetan-2-ylmethyl)-1H-benzo[d]imidazole-6-carboxylate (1-24e): The title compound was synthesized following the procedure of compound 1-18e by using 1-24e-1-2 as the starting material of which the synthesis was shown below.

The synthesis of methyl 4-amino-3-bromo-5-methoxybenzoate (1-24e-1-2):

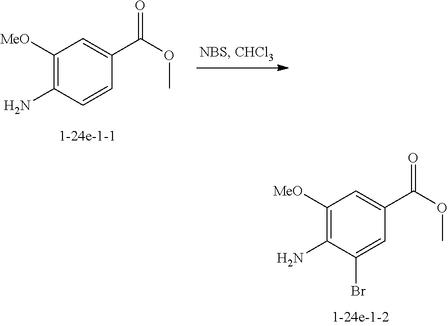

To a solution of methyl 4-amino-3-methoxybenzoate (5.0 g, 27.60 mmol) in CHCl₃ (50.0 mL) was added NBS (4.9 g, 27.60 mmol) at room temperature. The resulting mixture was stirred at room temperature for 2 h. After the reaction was completed, the resulting mixture was quenched with Na₂S₂O₃ (aq) and then extracted with CH₂Cl₂. The combined organic layers were washed with brine, dried over anhydrous sodium sulfate and filtered. The filtrate was concentrated under vacuum to afford 1-24e-1-2 (7.0 g, crude) as a brown solid. LCMS (ESI, m/z): [M+H]⁺=260.0.

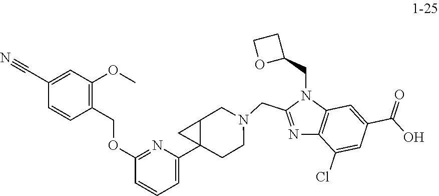 [SMILES: COc1cc(C#N)ccc1COc1cccc(C23CCN(Cc4nc5c(Cl)cc(C(=O)O)cc5n4C[C@@H]4CCO4)CC2C3)n1]

4-chloro-2-((6-(6-((4-cyano-2-methoxybenzyl)oxy)pyridin-2-yl)-3-azabicyclo[4.1.0]heptan-3-yl)methyl)-1-(((S)-oxetan-2-yl)methyl)-1H-benzo[d]imidazole-6-carboxylic acid (1-25): The title compound was synthesized by using 1-25e as the starting material of which the synthesis was shown below. LCMS (ESI, m/z): [M+H]⁺=614.2. ¹H NMR (400 MHZ, DMSO-d₆): δ 8.23 (s, 1H), 7.80 (s, 1H), 7.64-7.60 (m, 1H), 7.51 (s, 1H), 7.48-7.46 (m, 1H), 7.40 (d, J=8.0 Hz, 1H), 6.93 (d, J=8.0 Hz, 1H), 6.65 (d, J=8.4 Hz, 1H), 5.38-5.31 (m, 2H), 5.10-5.04 (m, 1H), 4.84-4.78 (m, 1H), 4.68-4.64 (m, 1H), 4.49-4.43 (m, 1H), 4.39-4.30 (m, 1H), 3.95-3.72 (m, 5H), 2.90-2.62 (m, 3H), 2.42-2.29 (m, 3H), 2.01-1.94 (m, 1H), 1.71-1.67 (m, 1H), 1.10-1.07 (m, 1H), 0.91-0.89 (m, 1H).

methyl(S)-4-chloro-2-(chloromethyl)-1-(oxetan-2-ylmethyl)-1H-benzo[d]imidazole-6-carboxylate (1-25e): The title compound was synthesized following the procedure of compound 1-17e by using methyl 3-chloro-5-fluoro-4-nitrobenzoate (1-25e-1) as the starting material of which the synthesis was shown below.

The synthesis of 3-chloro-5-fluoro-4-nitrobenzoate (1-25e-1):

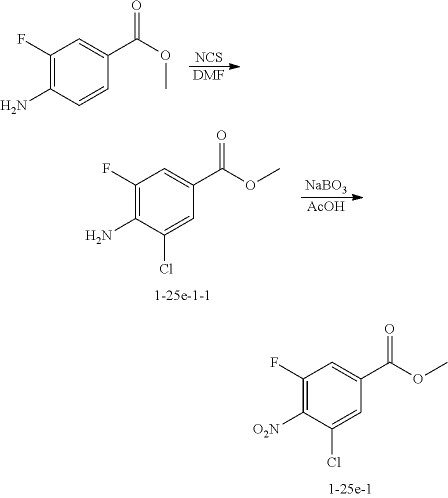

Step 1. To a solution of methyl 4-amino-3-fluorobenzoate (2.0 g, 11.82 mmol) in DMF (15.0 mL) was added NCS (1.9 g, 14.19 mmol) at room temperature. The resulting mixture was stirred at room temperature for 16 h. After the reaction was completed, the resulting mixture was diluted with water and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (37/63, v/v) to afford 1-25e-1-1 (2.1 g, 87%) as a white solid. LCMS (ESI, m/z): [M+H]⁺==204.0.

Step 2. To a solution of 1-25e-1-1 (1.9 g, 9.33 mmol) in AcOH (25.0 mL) was added dropwise a solution of NaBO₃ (3.8 g, 46.66 mmol) in AcOH (10.0 mL) at room temperature. The resulting mixture was stirred at 60° C. for 16 h. After the reaction was completed, the resulting mixture was diluted with ice water and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (87/13, v/v) to afford 1-25e-1 (1.5 g, 68%) as a light yellow solid. LCMS (ESI, m/z): [M+H]⁺=234.0.

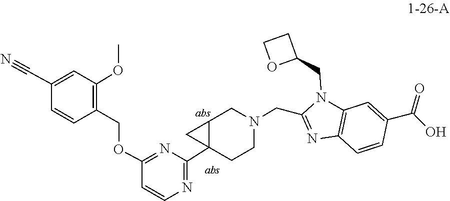 [SMILES: COc1cc(C#N)ccc1COc1ccnc(C23CCN(Cc4nc5ccc(C(=O)O)cc5n4C[C@@H]4CCO4)CC2C3)n1]

2-((6-(4-((4-cyano-2-methoxybenzyl)oxy)pyrimidin-2-yl)-3-azabicyclo[4.1.0]heptan-3-yl)methyl)-1-(((S)-oxetan-2-yl)methyl)-1H-benzo[d]imidazole-6-carboxylic acid (1-26-A): The title compound was synthesized following the procedure of compound 1-1-A by using 1-26a as the starting material of which the synthesis was shown below. LCMS (ESI, m/z): [M+H]⁺=581.3. ¹H NMR (400 MHZ, CD₃OD): δ 8.33-8.31 (m, 2H), 8.01-7.99 (m, 1H), 7.66 (d, J=8.4 Hz, 1H), 7.49 (d, J=8.0 Hz, 1H), 7.36-7.30 (m, 2H), 6.67 (d, J=6.0 Hz, 1H), 5.52-5.45 (m, 2H), 5.27-5.21 (m, 1H), 4.89-4.83 (m, 1H), 4.73-4.69 (m, 1H), 4.64-4.58 (m, 1H), 4.45-4.39 (m, 1H), 3.96-3.87 (m, 5H), 3.13-3.07 (m, 1H), 2.90-2.87 (m, 1H), 2.81-2.71 (m, 2H), 2.58-2.44 (m, 2H), 2.31-2.24 (m, 1H), 1.99-1.92 (m, 1H), 1.86-1.81 (m, 1H), 1.41-1.38 (m, 1H), 1.12-1.08 (m, 1H).

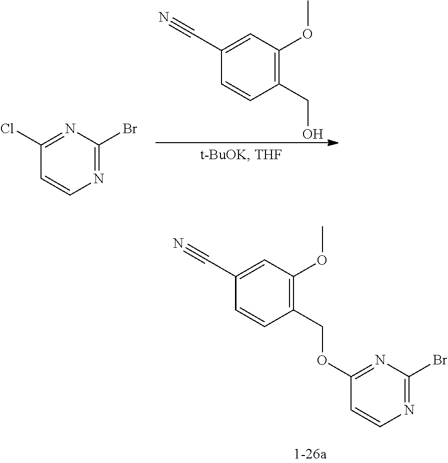 [SMILES: Clc1ccnc(Br)n1 ; COc1cc(C#N)ccc1CO ; COc1cc(C#N)ccc1COc1ccnc(Br)n1]

The synthesis of 4-(((2-bromopyrimidin-4-yl)oxy)methyl)-3-methoxybenzonitrile (1-26a): To a solution of 2-bromo-4-chloropyrimidine (652.0 mg, 3.37 mmol) in THF (50.0 mL) was added 4-(hydroxymethyl)-3-methoxybenzonitrile (1.5 g, 9.19 mmol) and t-BuOK (1.2 g, 11.03 mmol) at 0° C. The resulting mixture was stirred at 0° C. for 1 h. After the reaction was completed, the resulting mixture was quenched with NH₄Cl (aq) and then extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (74/26, v/v) and then purified by Prep-Achiral SFC with the following conditions (Column: DAICEL DCpak P4VP, 3×25 cm, 5 μm; Mobile Phase A: CO₂, Mobile Phase B: MEOH (0.1% 2M NH₃-MEOH); Flow rate: 60 mL/min; Gradient: isocratic 16% B; Column Temperature (° C.): 35; Back Pressure (bar): 100; Wave Length: 254 nm) to afford 1-26a (650.0 mg, 22%) as a white solid. LCMS (ESI, m/z): [M+H]⁺=320.0.

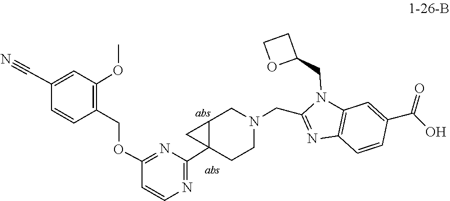 [SMILES: COc1cc(C#N)ccc1COc1ccnc(C23CCN(Cc4nc5ccc(C(=O)O)cc5n4C[C@@H]4CCO4)CC2C3)n1]

2-((6-(4-((4-cyano-2-methoxybenzyl)oxy)pyrimidin-2-yl)-3-azabicyclo[4.1.0]heptan-3-yl)methyl)-1-(((S)-oxetan-2-yl)methyl)-1H-benzo[d]imidazole-6-carboxylic acid (1-26-B): The title compound was synthesized following the procedure of compound 1-1-A by using 4-(((2-bromopyrimidin-4-yl)oxy)methyl)-3-methoxybenzonitrile (1-26a) as the starting material. LCMS (ESI, m/z): [M+H]⁺=581.3. ¹H NMR (400 MHZ, CD₃OD): δ 8.34-8.31 (m, 2H), 8.00-7.97 (m, 1H), 7.68 (d, J=8.4 Hz, 1H), 7.49 (d, J=7.6 Hz, 1H), 7.36-7.31 (m, 2H), 6.67 (d, J=6.0 Hz, 1H), 5.53-5.45 (m, 2H), 5.26-5.20 (m, 1H), 4.92-4.85 (m, 1H), 4.73-4.69 (m, 1H), 4.66-4.60 (m, 1H), 4.51-4.46 (m, 1H), 4.04-3.83 (m, 5H), 3.14-3.08 (m, 1H), 2.98-2.96 (m, 1H), 2.83-2.75 (m, 2H), 2.57-2.48 (m, 2H), 2.32-2.25 (m, 1H), 1.98-1.84 (m, 2H), 1.44-1.41 (m, 1H), 1.13-1.10 (m, 1H).

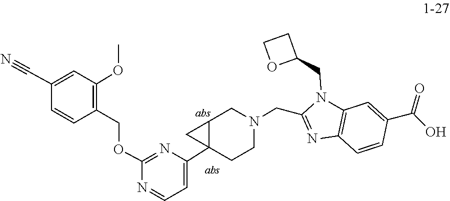 [SMILES: COc1cc(C#N)ccc1COc1nccc(C23CCN(Cc4nc5ccc(C(=O)O)cc5n4C[C@@H]4CCO4)CC2C3)n1]

2-((6-(2-((4-cyano-2-methoxybenzyl)oxy)pyrimidin-4-yl)-3-azabicyclo[4.1.0]heptan-3-yl)methyl)-1-(((S)-oxetan-2-yl)methyl)-1H-benzo[d]imidazole-6-carboxylic acid (1-27): The title compound was synthesized following the procedure of compound 1-1 by using 1-27a as the starting material of which the synthesis was shown below. LCMS (ESI, m/z): [M+H]⁺=581.3. ¹H NMR (400 MHZ, DMSO-d₆): δ 8.44 (d, J=4.0 Hz, 1H), 8.23 (s, 1H), 7.82 (d, J=8.4 Hz, 1H), 7.61-7.42 (m, 4H), 7.09 (d, J=5.2 Hz, 1H), 5.42-5.38 (m, 2H), 5.08-5.04 (m, 1H), 4.79-4.73 (m, 1H), 4.63-4.60 (m, 1H), 4.47-4.43 (m, 1H), 4.39-4.32 (m, 1H), 3.93-3.69 (m, 5H), 2.90-2.75 (m, 2H), 2.68-2.60 (m, 2H), 2.44-2.40 (m, 2H), 2.24-2.20 (s, 1H), 1.93-1.82 (m, 2H), 1.29-1.25 (m, 1H), 1.10-1.08 (m, 1H).

The synthesis of 4-(((4-chloropyrimidin-2-yl)oxy)methyl)-3-methoxybenzonitrile (1-27a):

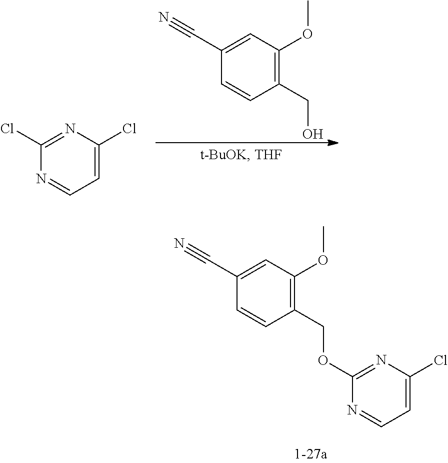 [SMILES: Clc1ccnc(Cl)n1 ; COc1cc(C#N)ccc1CO ; COc1cc(C#N)ccc1COc1nccc(Cl)n1]

To a solution of 2,4-dichloropyrimidine (1.0 g, 4.22 mmol) in THF (15.0 mL) was added 4-(hydroxymethyl)-3-methoxybenzonitrile (0.7 g, 4.22 mmol) and t-BuOK (0.4 g, 3.38 mmol) at 0° C. The resulting mixture was stirred at 0° C. for 2 h. After the reaction was completed, the resulting mixture was quenched with NH₄Cl (aq) and then extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (83/17, v/v) to afford 1-27a (0.6 g, 45%) as a light yellow solid. LCMS (ESI, m/z): [M+H]⁺=276.0.

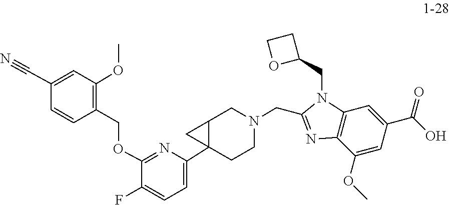 [SMILES: COc1cc(C#N)ccc1COc1nc(C23CCN(Cc4nc5c(OC)cc(C(=O)O)cc5n4C[C@@H]4CCO4)CC2C3)ccc1F]

2-((6-(6-((4-cyano-2-methoxybenzyl)oxy)-5-fluoropyridin-2-yl)-3-azabicyclo[4.1.0]heptan-3-yl)methyl)-4-methoxy-1-(((S)-oxetan-2-yl)methyl)-1H-benzo[d]imidazole-6-carboxylic acid (1-28): The title compound was synthesized following the procedure of compound 1-1 by using 4-(((6-bromo-3-fluoropyridin-2-yl)oxy)methyl)-3-methoxybenzonitrile (1-28a) as the starting material of which the synthesis was shown below, and using TMSOTf+2,6-lutidine for BOC-deprotection as shown for compound 1-16b. LCMS (ESI, m/z): [M+H]⁺=628.3. ¹H NMR (400 MHZ, DMSO-d₆): δ 12.89 (s, 1H), 7.88 (s, 1H), 7.60-7.41 (m, 4H), 7.26 (s, 1H), 6.93-6.91 (m, 1H), 5.42-5.30 (m, 2H), 5.05-5.01 (m, 1H), 4.76-4.70 (m, 1H), 4.60-4.57 (m, 1H), 4.48-4.41 (m, 1H), 4.36-4.27 (m, 1H), 3.95 (s, 3H), 3.88 (s, 3H), 3.85-3.66 (m, 2H), 2.89-2.65 (m, 3H), 2.49-2.28 (m, 4H), 1.96-1.91 (m, 1H), 1.64-1.61 (m, 1H), 1.04-1.02 (m, 1H), 0.91-0.88 (m, 1H).

The synthesis of 4-(((6-bromo-3-fluoropyridin-2-yl)oxy)methyl)-3-methoxybenzonitrile (1-28a):

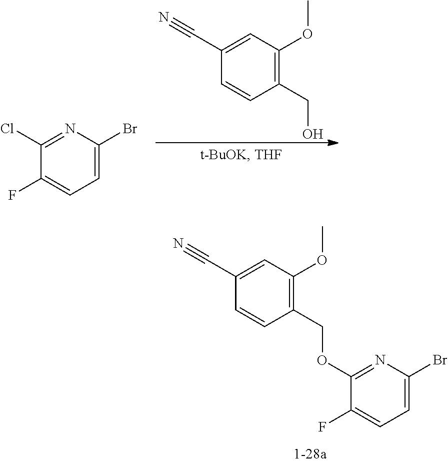 [SMILES: Fc1ccc(Br)nc1Cl ; COc1cc(C#N)ccc1CO ; COc1cc(C#N)ccc1COc1nc(Br)ccc1F]

To a solution of 6-bromo-2-chloro-3-fluoropyridine (900.0 mg, 4.28 mmol) in THF (30.0 mL) was added 4-(hydroxymethyl)-3-methoxybenzonitrile (720.0 mg, 4.41 mmol) and 1-BuOK (575.9 mg, 5.13 mmol) at room temperature. The resulting mixture was stirred at room temperature for 1 h. After the reaction was completed, the mixture was quenched with NH₄Cl (aq.) and then extracted with ethyl acetate. The combined organic layer was washed with brine, dried over anhydrous sodium sulfate and filtered. The filtrate was concentrated under vacuum.

The residue was purified by flash column chromatography with petroleum ether/ethyl acetate (85/15, v/v) to afford 1-28a (360.0 mg, 25%) as a white solid. LCMS (ESI, m/z): [M+H]⁺=337.0.

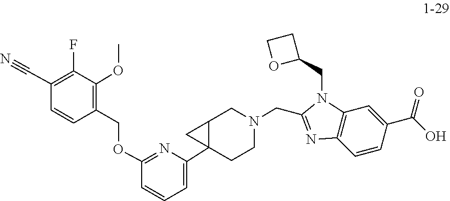 [SMILES: COc1c(COc2cccc(C34CCN(Cc5nc6ccc(C(=O)O)cc6n5C[C@@H]5CCO5)CC3C4)n2)ccc(C#N)c1F]

2-((6-(6-((4-cyano-3-fluoro-2-methoxybenzyl)oxy)pyridin-2-yl)-3-azabicyclo[4.1.0]heptan-3-yl)methyl)-1-(((S)-oxetan-2-yl)methyl)-1H-benzo[d]imidazole-6-carboxylic acid (1-29): LCMS (ESI, m/z): [M+H]⁺=598.3. ¹H NMR (400 MHZ, CD₃OD): δ 8.34-8.32 (m, 1H), 8.00-7.97 (m, 1H), 7.70-7.67 (m, 1H), 7.62-7.58 (m, 1H), 7.41-7.37 (m, 1H), 7.32 (d, J=8.4 Hz, 1H), 6.94 (d, J=7.6 Hz, 1H), 6.64 (d, J=8.0 Hz, 1H), 5.50-5.41 (m, 2H), 5.25-5.22 (m, 1H), 4.92-4.89 (m, 1H), 4.73-4.68 (m, 1H), 4.64-4.58 (m, 1H), 4.50-4.40 (m, 1H), 4.06-3.86 (m, 5H), 3.04-2.94 (m, 1H), 2.88-2.70 (m, 2H), 2.63-2.56 (m, 1H), 2.54-2.44 (m, 3H), 2.13-2.05 (m, 1H), 1.80-1.73 (m, 1H), 1.19-1.16 (m, 1H), 0.99-0.97 (m, 1H).

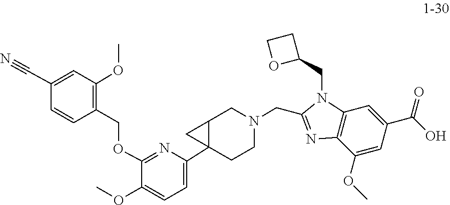 [SMILES: COc1cc(C#N)ccc1COc1nc(C23CCN(Cc4nc5c(OC)cc(C(=O)O)cc5n4C[C@@H]4CCO4)CC2C3)ccc1OC]

2-((6-(6-((4-cyano-2-methoxybenzyl)oxy)-5-methoxypyridin-2-yl)-3-azabicyclo[4.1.0]heptan-3-yl)methyl)-4-methoxy-1-(((S)-oxetan-2-yl)methyl)-1H-benzo[d]imidazole-6-carboxylic acid (1-30): The title compound was synthesized following the procedure of compound 1-28 by using 6-bromo-2-fluoro-3-methoxypyridine as the starting material and using TFA+DCM for the BOC-deprotection as shown for compound 1-1b. LCMS (ESI, m/z): [M+H]⁺=640.3. ¹H NMR (400 MHZ, DMSO-d₆): δ 7.86 (s, 1H), 7.51 (s, 1H), 7.45-7.39 (m, 2H), 7.27 (s, 1H), 7.22 (d, J=8.0 Hz, 1H), 6.84 (d, J=8.0 Hz, 1H), 5.35-5.30 (m, 2H), 5.07-5.00 (m, 1H), 4.72-4.69 (m, 1H), 4.59-4.56 (m, 1H), 4.46-4.43 (m, 1H), 4.32-4.27 (m, 1H), 3.95 (s, 3H), 3.88 (s, 3H), 3.83-3.65 (m, 5H), 2.84-2.62 (m, 3H), 2.39-2.22 (m, 4H), 1.92-1.89 (m, 1H), 1.56-1.53 (m, 1H), 1.00-0.97 (m, 1H), 0.82-0.79 (m, 1H).

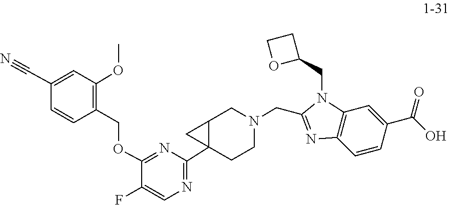 [SMILES: COc1cc(C#N)ccc1COc1nc(C23CCN(Cc4nc5ccc(C(=O)O)cc5n4C[C@@H]4CCO4)CC2C3)ncc1F]

2-((6-(4-((4-cyano-2-methoxybenzyl)oxy)-5-fluoropyrimidin-2-yl)-3-azabicyclo[4.1.0]heptan-3-yl)methyl)-1-(((S)-oxetan-2-yl)methyl)-1H-benzo[d]imidazole-6-carboxylic acid (1-31): The title compound was synthesized following the procedure of compound 1-28 by using 2-chloro-5-fluoro-4-(methylsulfonyl)pyrimidine as the starting material. LCMS (ESI, m/z): [M+H]⁺=599.3. ¹H NMR (400 MHZ, CD₃OD): δ 8.34 (s, 1H), 8.29 (d, J=2.8 Hz, 1H), 8.01-7.98 (m, 1H), 7.70-7.67 (m, 1H), 7.54 (d, J=7.6 Hz, 1H), 7.39-7.33 (m, 2H), 5.60-5.53 (m, 2H), 5.28-5.24 (m, 1H), 4.75-4.69 (m, 1H), 4.67-4.62 (m, 1H), 4.52-4.41 (m, 1H), 4.03-3.82 (m, 5H), 3.13-3.07 (m, 1H), 2.98-2.88 (m, 1H), 2.84-2.73 (m, 2H), 2.59-2.47 (m, 2H), 2.33-2.23 (m, 1H), 2.00-1.92 (m, 1H), 1.87-1.79 (m, 1H), 1.43-1.38 (m, 1H), 1.12-1.08 (m, 1H).

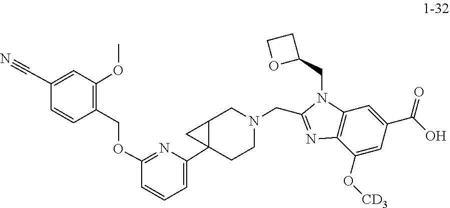 [SMILES: [2H]C([2H])([2H])Oc1cc(C(=O)O)cc2c1nc(CN1CCC3(c4cccc(OCc5ccc(C#N)cc5OC)n4)CC3C1)n2C[C@@H]1CCO1]

2-((6-(6-((4-cyano-2-methoxybenzyl)oxy)pyridin-2-yl)-3-azabicyclo[4.1.0]heptan-3-yl)methyl)-4-(methoxy-d3)-1-(((S)-oxetan-2-yl)methyl)-1H-benzo[d]imidazole-6-carboxylic acid (1-32): The title compound was synthesized by using 1-32e as the starting material. LCMS (ESI, m/z): [M+H]⁺=613.2. ¹H NMR (400 MHZ, CD₃OD): δ 7.92-7.90 (m, 1H), 7.60-7.56 (m, 1H), 7.49-7.46 (m, 2H), 7.39-7.27 (m, 2H), 6.91 (d, J=7.2 Hz, 1H), 6.62 (d, J=8.0 Hz, 1H), 5.45-5.37 (m, 2H), 5.24-5.21 (m, 1H), 4.82-4.80 (m, 1H), 4.71-4.57 (m, 2H), 4.48-4.39 (m, 1H), 3.98-3.83 (m, 5H), 3.01-2.89 (m, 1H), 2.83-2.70 (m, 2H), 2.49-2.41 (m, 4H), 2.09-2.04 (m, 1H), 1.77-1.70 (m, 1H), 1.15-1.12 (m, 1H), 0.96-0.93 (m, 1H).

methyl(S)-2-(chloromethyl)-4-(methoxy-d3)-1-(oxetan-2-ylmethyl)-1H-benzo[d]imidazole-6-carboxylate (1-32e): The title compound was synthesized following the procedure of compound 1-24e by using 1-32e-1-1 as the starting material of which the synthesis was shown below. LCMS (ESI, m/z): [M+H]⁺=328.0.

The synthesis of methyl 4-amino-3-(methoxy-d3)benzoate (1-32e-1-1):

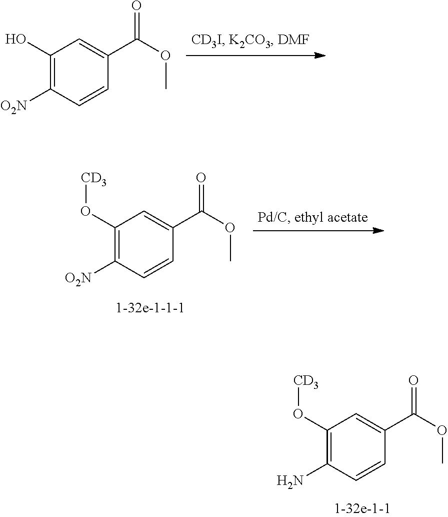 [SMILES: COC(=O)c1ccc([N+](=O)[O-])c(O)c1 ; [2H]C([2H])([2H])Oc1cc(C(=O)OC)ccc1[N+](=O)[O-] ; [2H]C([2H])([2H])Oc1cc(C(=O)OC)ccc1N]

Step 1. To a solution of methyl 3-hydroxy-4-nitrobenzoate (5.0 g, 25.36 mmol) in DMF (50.0 mL) was added K₂CO₃ (5.3 g, 38.04 mmol) and CD₃I (5.5 g, 38.04 mmol) at room temperature. The resulting mixture was stirred at 40° C. for 16 h. After the reaction was completed, the resulting mixture was diluted with water and extracted with ethyl acetate. The combined organic layer was washed with brine, dried over anhydrous sodium sulfate and filtered. The filtrate was concentrated under reduced pressure to afford 1-32e-1-1-1 (5.2 g, crude) as a yellow solid.

Step 2. To a solution of 1-32e-1-0 (5.2 g, crude) in ethyl acetate (40.0 mL) was added Pd/C (1.0 g, 10%) at room temperature under N₂. The resulting mixture was stirred at room temperature for 16 h under H₂. After the reaction was completed, the resulting mixture was filtered. The filtrate was concentrated under vacuum. The residue was purified by flash column chromatography with petroleum ether/ethyl acetate (27/73, v/v) to afford 1-32e-1-1 (4.4 g, 98%) as a light yellow solid. LCMS (ESI, m/z): [M+H]⁺=185.0.

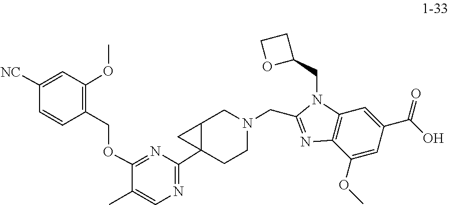 [SMILES: COc1cc(C#N)ccc1COc1nc(C23CCN(Cc4nc5c(OC)cc(C(=O)O)cc5n4C[C@@H]4CCO4)CC2C3)ncc1C]

2-((6-(4-((4-cyano-2-methoxybenzyl)oxy)-5-methylpyrimidin-2-yl)-3-azabicyclo[4.1.0]heptan-3-yl)methyl)-4-methoxy-1-(((S)-oxetan-2-yl)methyl)-1H-benzo[d]imidazole-6-carboxylic acid (1-33): The title compound was synthesized following the procedure of compound 1-28 by using 2,4-dichloro-5-methylpyrimidine as the starting material. LCMS (ESI, m/z): [M+H]⁺=625.3. ¹H NMR (400 MHZ, DMSO-d₆): δ 8.25 (s, 1H), 7.86 (s, 1H), 7.53-7.41 (m, 3H), 7.28 (s, 1H), 5.45-5.41 (m, 2H), 5.13-5.05 (m, 1H), 4.75-4.71 (m, 1H), 4.61-4.57 (m, 1H), 4.48-4.44 (m, 1H), 4.36-4.29 (m, 1H), 3.95 (s, 3H), 3.89 (s, 3H), 3.85-3.64 (m, 1H), 3.05-3.02 (m, 1H), 2.90-2.79 (m, 1H), 2.64-2.60 (m, 2H), 2.43-2.33 (m, 2H), 2.14-2.05 (m, 4H), 1.79-1.70 (m, 2H), 1.29-1.24 (m, 1H), 0.97-0.90 (m, 1H).

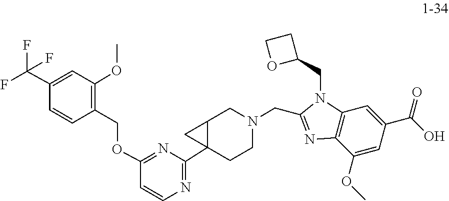 [SMILES: COc1cc(C(F)(F)F)ccc1COc1ccnc(C23CCN(Cc4nc5c(OC)cc(C(=O)O)cc5n4C[C@@H]4CCO4)CC2C3)n1]

4-methoxy-2-((6-(4-((2-methoxy-4-(trifluoromethyl)benzyl)oxy)pyrimidin-2-yl)-3-azabicyclo[4.1.0]heptan-3-yl)methyl)-1-(((S)-oxetan-2-yl)methyl)-1H-benzo[d]imidazole-6-carboxylic acid (1-34): The title compound was synthesized following the procedure of compound 1-28 by using 1-14a-2, 2-chloro-4-(methylsulfonyl)pyrimidine and 1-24e as the starting material. LCMS (ESI, m/z): [M+H]⁺==654.2. ¹H NMR (400 MHZ, DMSO-d₆): δ 8.40 (s, 1H), 7.84 (s, 1H), 7.55-7.53 (m, 1H), 7.32-7.28 (m, 3H), 6.74 (d, J=5.6 Hz, 1H), 5.42-5.39 (m, 2H), 5.07-5.01 (m, 1H), 4.74-4.69 (m, 1H), 4.60-4.56 (m, 1H), 4.47-4.44 (m, 1H), 4.38-4.27 (m, 1H), 3.94 (s, 3H), 3.90 (s, 3H), 3.85-3.64 (m, 2H), 3.10-3.07 (m, 1H), 2.89-2.81 (m, 1H), 2.67-2.60 (m, 2H), 2.46-2.37 (m, 1H), 2.14-2.05 (m, 1H), 1.85-1.76 (m, 2H), 1.37-1.32 (m, 1H), 1.02-0.98 (m, 1H).

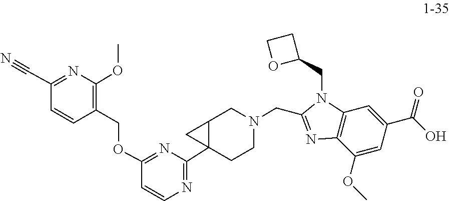 [SMILES: COc1nc(C#N)ccc1COc1ccnc(C23CCN(Cc4nc5c(OC)cc(C(=O)O)cc5n4C[C@@H]4CCO4)CC2C3)n1]

2-((6-(4-((6-cyano-2-methoxypyridin-3-yl)methoxy)pyrimidin-2-yl)-3-azabicyclo[4.1.0]heptan-3-yl)methyl)-4-methoxy-1-(((S)-oxetan-2-yl)methyl)-1H-benzo[d]imidazole-6-carboxylic acid (1-35): The title compound was synthesized following the procedure of compound 1-34. LCMS (ESI, m/z): [M+H]⁺=612.3. ¹H NMR (400 MHZ, Methanol-d₄): δ 8.36 (d, J=8.0 Hz, 1H), 7.95 (s, 1H), 7.85 (d, J=7.2 Hz, 1H), 7.47-7.44 (m, 2H), 6.71 (d, J=5.6 Hz, 1H), 5.50-5.42 (m, 2H), 5.25-5.21 (m, 1H), 4.86-4.82 (m, 1H), 4.73-4.67 (m, 1H), 4.65-4.59 (m, 1H), 4.50-4.39 (m, 1H), 4.05-4.03 (m, 6H), 4.00-3.82 (m, 2H), 3.12-3.06 (m, 1H), 2.96-2.88 (m, 1H), 2.83-2.74 (m, 2H), 2.56-2.50 (m, 2H), 2.28-2.25 (m, 1H), 1.99-1.92 (m, 1H), 1.87-1.83 (m, 1H), 1.42-1.38 (m, 1H), 1.13-1.10 (m, 1H).

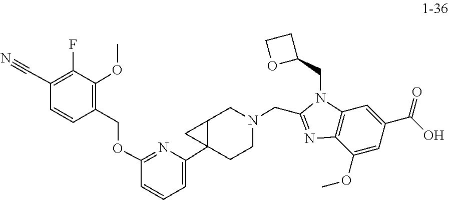 [SMILES: COc1c(COc2cccc(C34CCN(Cc5nc6c(OC)cc(C(=O)O)cc6n5C[C@@H]5CCO5)CC3C4)n2)ccc(C#N)c1F]

2-((6-(6-((4-cyano-3-fluoro-2-methoxybenzyl)oxy)pyridin-2-yl)-3-azabicyclo[4.1.0]heptan-3-yl)methyl)-4-methoxy-1-(((S)-oxetan-2-yl)methyl)-1H-benzo[d]imidazole-6-carboxylic acid (1-36): The title compound was synthesized following the procedure of compound 1-29 by using 1-24e as the starting material. LCMS (ESI, m/z): [M+H]⁺=628.3. ¹H NMR (400 MHZ, DMSO-d₆): δ 7.78 (s, 1H), 7.64-7.57 (m, 2H), 7.34-7.30 (m, 2H), 6.94 (d, J=7.2 Hz, 1H), 6.65 (d, J=8.4 Hz, 1H), 5.43-5.36 (m, 2H), 5.05-4.98 (m, 1H), 4.70-4.65 (m, 1H), 4.57-4.53 (m, 1H), 4.44-4.39 (m, 1H), 4.33-4.27 (m, 1H), 3.95-3.92 (m, 6H), 3.85-3.65 (m, 3H), 2.81-2.62 (m, 3H), 2.40-2.31 (m, 3H), 1.97-1.91 (m, 1H), 1.70-1.66 (m, 1H), 1.10-1.07 (m, 1H), 0.94-0.89 (m, 1H).

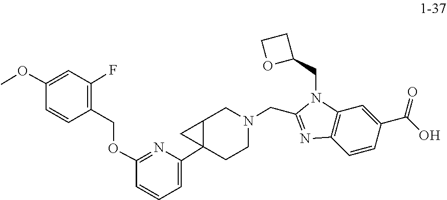 [SMILES: COc1ccc(COc2cccc(C34CCN(Cc5nc6ccc(C(=O)O)cc6n5C[C@@H]5CCO5)CC3C4)n2)c(F)c1]

2-((6-(6-((2-fluoro-4-methoxybenzyl)oxy)pyridin-2-yl)-3-azabicyclo[4.1.0]heptan-3-yl)methyl)-1-(((S)-oxetan-2-yl)methyl)-1H-benzo[d]imidazole-6-carboxylic acid (1-37): LCMS (ESI, m/z): [M+H]⁺=573.3. ¹H NMR (400 MHZ, CD₃OD): δ 8.32 (s, 1H), 8.00-7.98 (m, 1H), 7.69-7.67 (m, 1H), 7.57-7.53 (m, 1H), 7.40-7.35 (m, 1H), 6.91 (d, J=7.6 Hz, 1H), 6.73-6.68 (m, 2H), 6.54 (d, J=8.0 Hz, 1H), 5.31 (s, 2H), 5.28-5.23 (m, 1H), 4.75-4.70 (m, 1H), 4.62-4.60 (m, 1H), 4.51-4.40 (m, 1H), 4.06-3.86 (m, 2H), 3.78 (s, 3H), 3.06-2.96 (m, 1H), 2.89-2.81 (m, 1H), 2.78-2.72 (m, 1H), 2.67-2.61 (m, 1H), 2.55-2.45 (m, 3H), 2.16-2.10 (m, 1H), 1.89-1.86 (m, 1H), 1.31-1.27 (m, 1H), 1.02-0.99 (m, 1H).

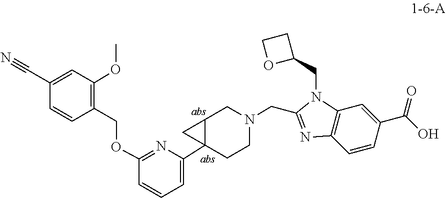 [SMILES: COc1cc(C#N)ccc1COc1cccc(C23CCN(Cc4nc5ccc(C(=O)O)cc5n4C[C@@H]4CCO4)CC2C3)n1]

2-((6-(6-((4-cyano-2-methoxybenzyl)oxy)pyridin-2-yl)-3-azabicyclo[4.1.0]heptan-3-yl)methyl)-1-(((S)-oxetan-2-yl)methyl)-1H-benzo[d]imidazole-6-carboxylic acid (1-6-A): LCMS (ESI, m/z): [M+H]⁺=580.2. ¹H NMR (400 MHZ, Methanol-d₄): δ 8.32 (s, 1H), 8.00-7.97 (m, 1H), 7.67 (d, J=8.4 Hz, 1H), 7.60-7.56 (m, 1H), 7.48 (d, J=7.6 Hz, 1H), 7.33 (d, J=3.6 Hz, 1H), 7.32-7.26 (m, 1H), 6.91 (d, J=7.6 Hz, 1H), 6.63 (d, J=8.0 Hz, 1H), 5.46-5.37 (m, 2H), 5.26-5.20 (m, 1H), 4.73-4.69 (m, 1H), 4.64-4.59 (m, 1H), 4.50-4.45 (m, 1H), 4.01 (d, J=13.6 Hz, 1H), 3.93 (s, 3H), 3.85 (d, J=13.6 Hz, 1H), 2.91-2.90 (m, 1H), 2.85-2.81 (m, 1H), 2.79-2.72 (m, 1H), 2.62-2.49 (m, 2H), 2.45-2.42 (m, 2H), 2.10-2.03 (m, 1H), 1.78-1.72 (m, 1H), 1.16-1.13 (m, 1H), 0.97-0.94 (m, 1H).

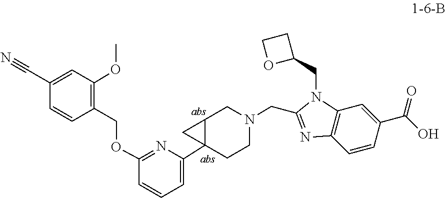 [SMILES: COc1cc(C#N)ccc1COc1cccc(C23CCN(Cc4nc5ccc(C(=O)O)cc5n4C[C@@H]4CCO4)CC2C3)n1]

2-((6-(6-((4-cyano-2-methoxybenzyl)oxy)pyridin-2-yl)-3-azabicyclo[4.1.0]heptan-3-yl)methyl)-1-(((S)-oxetan-2-yl)methyl)-1H-benzo[d]imidazole-6-carboxylic acid (1-6-B): LCMS (ESI, m/z): [M+H]⁺=580.3. ¹H NMR (400 MHZ, Methanol-d₄): δ 8.31 (s, 1H), 7.99 (d, J=8.4 Hz, 1H), 7.67 (d, J=8.4 Hz, 1H), 7.60-7.56 (m, 1H), 7.48 (d, J=8.0 Hz, 1H), 7.34-7.27 (m, 2H), 6.91 (d, J=7.2 Hz, 1H), 6.63 (d, J=8.0 Hz, 1H), 5.45-5.38 (m, 2H), 5.27-5.21 (m, 1H), 4.94-4.91 (m, 1H), 4.72-4.68 (m, 1H), 4.63-4.58 (m, 1H), 4.46-4.40 (m, 1H), 3.97-3.87 (m, 5H), 2.98-2.94 (m, 1H), 2.79-2.71 (m, 2H), 2.61-2.42 (m, 4H), 2.10-2.03 (m, 1H), 1.76-1.70 (m, 1H), 1.15-1.12 (m, 1H), 0.95-0.93 (m, 1H).

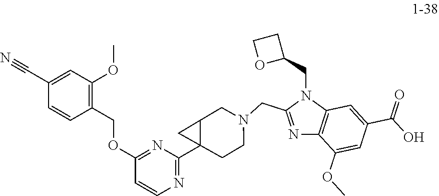 [SMILES: COc1cc(C#N)ccc1COc1ccnc(C23CCN(Cc4nc5c(OC)cc(C(=O)O)cc5n4C[C@@H]4CCO4)CC2C3)n1]

2-((6-(4-((4-cyano-2-methoxybenzyl)oxy)pyrimidin-2-yl)-3-azabicyclo[4.1.0]heptan-3-yl)methyl)-4-methoxy-1-(((S)-oxetan-2-yl)methyl)-1H-benzo[d]imidazole-6-carboxylic acid (1-38): The title compound was synthesized following the procedure of compound 1-26-A by using 1-24e as the starting material. LCMS (ESI, m/z): [M+H]⁺=611.3. ¹H NMR (400 MHz, CD₃OD): δ 8.34 (d, J=4.0 Hz, 1H), 7.94 (s, 1H), 7.50-7.46 (m, 2H), 7.36-7.30 (m, 2H), 6.67 (d, J=6.0 Hz, 1H), 5.53-5.45 (m, 2H), 5.26-5.19 (m, 1H), 4.88-4.86 (m, 1H), 4.72-4.59 (m, 2H), 4.50-4.39 (m, 1H), 4.05 (s, 3H), 4.00-3.82 (m, 5H), 3.13-3.07 (m, 1H), 2.96-2.87 (m, 1H), 2.82-2.72 (m, 2H), 2.58-2.44 (m, 2H), 2.32-2.23 (m, 1H), 2.00-1.90 (m, 1H), 1.89-1.82 (m, 1H), 1.43-1.38 (m, 1H), 1.12-1.08 (m, 1H).

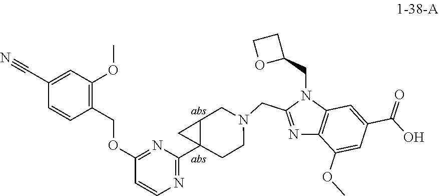 [SMILES: COc1cc(C#N)ccc1COc1ccnc(C23CCN(Cc4nc5c(OC)cc(C(=O)O)cc5n4C[C@@H]4CCO4)CC2C3)n1]

2-((6-(4-((4-cyano-2-methoxybenzyl)oxy)pyrimidin-2-yl)-3-azabicyclo[4.1.0]heptan-3-yl)methyl)-4-methoxy-1-(((S)-oxetan-2-yl)methyl)-1H-benzo[d]imidazole-6-carboxylic acid (1-38-A): LCMS (ESI, m/z): [M+H]⁺=611.3. ¹H NMR (400 MHZ, DMSO-d₆): δ 8.41 (d, J=4.0 Hz, 1H), 7.87 (s, 1H), 7.53-7.49 (m, 2H), 7.44-7.42 (m, 1H), 7.27 (s, 1H), 6.75 (d, J=6.0 Hz, 1H), 5.45-5.38 (m, 2H), 5.09-5.05 (m, 1H), 4.76-4.70 (m, 1H), 4.63-4.58 (m, 1H), 4.49-4.43 (m, 1H), 4.32-4.28 (m, 1H), 3.96 (s, 3H), 3.88 (s, 3H), 3.83-3.74 (m, 2H), 3.09-3.05 (m, 1H), 2.84-2.81 (m, 1H), 2.66-2.62 (m, 2H), 2.55-2.51 (m, 1H), 2.41-2.34 (m, 1H), 2.15-2.08 (m, 1H), 1.85-1.73 (m, 2H), 1.34-1.24 (m, 1H), 1.03-1.00 (m, 1H).

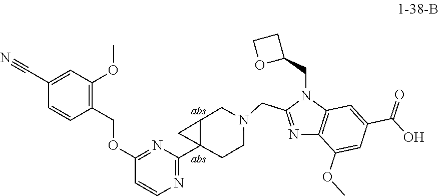 [SMILES: COc1cc(C#N)ccc1COc1ccnc(C23CCN(Cc4nc5c(OC)cc(C(=O)O)cc5n4C[C@@H]4CCO4)CC2C3)n1]

2-((6-(4-((4-cyano-2-methoxybenzyl)oxy)pyrimidin-2-yl)-3-azabicyclo[4.1.0]heptan-3-yl)methyl)-4-methoxy-1-(((S)-oxetan-2-yl)methyl)-1H-benzo[d]imidazole-6-carboxylic acid (1-38-B): LCMS (ESI, m/z): [M+H]⁺=611.3. ¹H NMR (400 MHZ, DMSO-d₆): δ 8.41 (d, J=4.0 Hz, 1H), 7.86 (s, 1H), 7.54-7.49 (m, 2H), 7.44-7.42 (m, 1H), 7.27 (s, 1H), 6.75 (d, J=6.0 Hz, 1H), 5.45-5.38 (m, 2H), 5.05-5.01 (m, 1H), 4.76-4.71 (m, 1H), 4.61-4.57 (m, 1H), 4.50-4.44 (m, 1H), 4.39-4.32 (m, 1H), 3.95 (s, 3H), 3.89-3.86 (m, 4H), 3.69-3.65 (m, 1H), 3.09-3.06 (m, 1H), 2.92-2.89 (m, 1H), 2.69-2.62 (m, 2H), 2.44-2.33 (m, 2H), 2.10-2.05 (m, 1H), 1.82-1.78 (m, 2H), 1.36-1.34 (m, 1H), 1.04-1.02 (m, 1H).

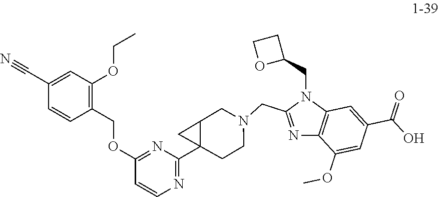 [SMILES: CCOc1cc(C#N)ccc1COc1ccnc(C23CCN(Cc4nc5c(OC)cc(C(=O)O)cc5n4C[C@@H]4CCO4)CC2C3)n1]

2-((6-(4-((4-cyano-2-ethoxybenzyl)oxy)pyrimidin-2-yl)-3-azabicyclo[4.1.0]heptan-3-yl)methyl)-4-methoxy-1-(((S)-oxetan-2-yl)methyl)-1H-benzo[d]imidazole-6-carboxylic acid (1-39): The title compound was synthesized following the procedure of compound 1-34 by using 3-ethoxy-4-(hydroxymethyl)benzonitrile as the starting material. LCMS (ESI, m/z): [M+H]⁺=625.3. ¹H NMR (400 MHZ, Methanol-d₄): δ 8.34 (d, J=8.0 Hz, 1H), 7.95-7.94 (m, 1H), 7.48-7.46 (m, 2H), 7.33-7.28 (m, 2H), 6.67 (d, J=6.0 Hz, 1H), 5.56-5.48 (m, 2H), 5.26-5.19 (m, 1H), 4.84-4.82 (m, 1H), 4.72-4.58 (m, 2H), 4.49-4.39 (m, 1H), 4.16-4.10 (m, 2H), 4.05 (s, 3H), 3.99-3.82 (m, 2H), 3.12-3.06 (m, 1H), 2.95-2.87 (m, 1H), 2.83-2.71 (m, 2H), 2.55-2.44 (m, 2H), 2.32-2.23 (m, 1H), 2.00-1.92 (m, 1H), 1.85-1.81 (m, 1H), 1.43-1.37 (m, 4H), 1.11-1.08 (m, 1H).

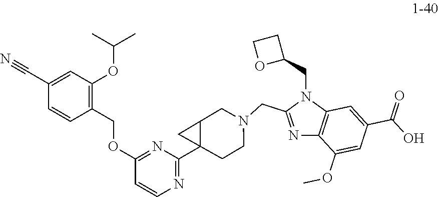 [SMILES: COc1cc(C(=O)O)cc2c1nc(CN1CCC3(c4nccc(OCc5ccc(C#N)cc5OC(C)C)n4)CC3C1)n2C[C@@H]1CCO1]

2-((6-(4-((4-cyano-2-isopropoxybenzyl)oxy)pyrimidin-2-yl)-3-azabicyclo[4.1.0]heptan-3-yl)methyl)-4-methoxy-1-(((S)-oxetan-2-yl)methyl)-1H-benzo[d]imidazole-6-carboxylic acid (1-40): The title compound was synthesized following the procedure of compound 1-34 by using 4-(hydroxymethyl)-3-isopropoxybenzonitrile as the starting material. LCMS (ESI, m/z): [M+H]⁺=639.3. ¹H NMR (400 MHZ, Methanol-d₄): δ 8.34 (d, J=8.0 Hz, 1H), 7.87 (s, 1H), 7.48-7.45 (m, 2H), 7.36 (s, 1H), 7.27 (d, J=7.6 Hz, 1H), 6.66 (d, J=5.6 Hz, 1H), 5.54-5.46 (m, 2H), 5.25-5.22 (m, 1H), 4.74-4.67 (m, 2H), 4.62-4.58 (m, 1H), 4.49-4.39 (m, 1H), 4.05 (s, 3H), 3.96-3.81 (m, 2H), 3.12-3.06 (m, 1H), 2.93-2.85 (m, 1H), 2.78-2.71 (m, 2H), 2.56-2.45 (m, 2H), 2.27-2.18 (m, 1H), 1.98-1.91 (m, 1H), 1.89-1.81 (m, 1H), 1.43-1.38 (m, 1H), 1.33-1.30 (m, 6H), 1.12-1.08 (m, 1H).

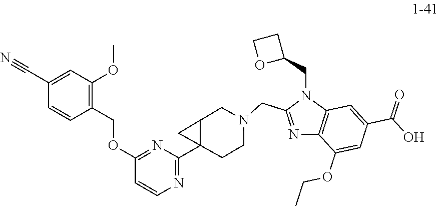 [SMILES: CCOc1cc(C(=O)O)cc2c1nc(CN1CCC3(c4nccc(OCc5ccc(C#N)cc5OC)n4)CC3C1)n2C[C@@H]1CCO1]

2-((6-(4-((4-cyano-2-methoxybenzyl)oxy)pyrimidin-2-yl)-3-azabicyclo[4.1.0]heptan-3-yl)methyl)-4-ethoxy-1-(((S)-oxetan-2-yl)methyl)-1H-benzo[d]imidazole-6-carboxylic acid (1-41): The title compound was synthesized following the procedure of compound 1-34 by using methyl (S)-2-(chloromethyl)-4-ethoxy-1-(oxetan-2-ylmethyl)-1H-benzo[d]imidazole-6-carboxylate (1-41e) (compound 1-41e was synthesized following the procedure of compound 1-32e) as the starting material. LCMS (ESI, m/z): [M+H]⁺=625.4. ¹H NMR (400 MHZ, DMSO-d₆): δ 8.40 (s, 1H), 7.85 (s, 1H), 7.53-7.49 (m, 2H), 7.44-7.42 (m, 1H), 7.26 (s, 1H), 6.74 (d, J=6.0 Hz, 1H), 5.48-5.42 (m, 2H), 5.05-5.01 (m, 1H), 4.76-4.71 (m, 1H), 4.62-4.58 (m, 1H), 4.46-4.43 (m, 1H), 4.38-4.23 (m, 3H), 3.88 (s, 3H), 3.79-3.64 (m, 2H), 3.10-3.06 (m, 1H), 2.92-2.81 (m, 1H), 2.67-2.63 (m, 2H), 2.44-2.37 (m, 1H), 2.12-2.08 (m, 1H), 1.87-1.76 (m, 2H), 1.44-1.41 (m, 3H), 1.34-1.32 (m, 1H), 1.02-0.98 (m, 1H).

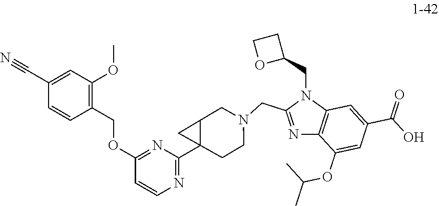 [SMILES: COc1cc(C#N)ccc1COc1ccnc(C23CCN(Cc4nc5c(OC(C)C)cc(C(=O)O)cc5n4C[C@@H]4CCO4)CC2C3)n1]

2-((6-(4-((4-cyano-2-methoxybenzyl)oxy)pyrimidin-2-yl)-3-azabicyclo[4.1.0]heptan-3-yl)methyl)-4-isopropoxy-1-(((S)-oxetan-2-yl)methyl)-1H-benzo[d]imidazole-6-carboxylic acid (1-42): The title compound was synthesized following the procedure of compound 1-34 by using 1-42e as the starting material of which the synthesis was shown below. LCMS (ESI, m/z): [M+H]⁺=639.3. ¹H NMR (400 MHZ, DMSO-d₆): δ 8.40 (d, J=4.0 Hz, 1H), 7.84 (s, 1H), 7.53-7.49 (m, 2H), 7.43 (d, J=7.6 Hz, 1H), 7.25 (s, 1H), 6.75 (d, J=5.6 Hz, 1H), 5.42-5.40 (m, 2H), 5.06-5.00 (m, 2H), 4.74-4.70 (m, 1H), 4.63-4.58 (m, 1H), 4.50-4.44 (m, 1H), 4.41-4.30 (m, 1H), 3.90-3.66 (m, 5H), 3.10-3.07 (m, 1H), 2.93-2.81 (m, 1H), 2.70-2.62 (m, 2H), 2.45-2.38 (m, 1H), 2.15-2.05 (m, 1H), 1.86-1.76 (m, 2H), 1.35-1.32 (m, 6H), 1.04-1.01 (m, 1H).

methyl(S)-2-(chloromethyl)-4-isopropoxy-1-(oxetan-2-ylmethyl)-1H-benzo[d]imidazole-6-carboxylate (1-42e): The title compound was synthesized following the procedure of compound 1-24e by using 1-42e-1-1 as the starting material. LCMS (ESI, m/z): [M+H]⁺=353.1.

The synthesis of methyl 4-amino-3-isopropoxybenzoate (1-42e-1-1):

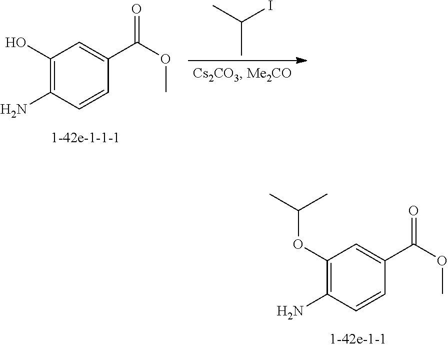

To a mixture of methyl 4-amino-3-hydroxybenzoate (1.5 g, 8.97 mmol) and Cs₂CO₃ (5.8 g, 17.95 mmol) in Me₂CO (50.0 mL) was added 2-iodopropane (1.5 g, 8.97 mmol) at room temperature. The resulting mixture was stirred at 70° C. for 5 h. After the reaction was completed, the resulting mixture was cooled to room temperature and filtered. The filtrate was concentrated under reduced pressure. The residue was purified by flash column chromatography with ethyl acetate/petroleum ether (76/24, v/v) to afford methyl 4-amino-3-isopropoxybenzoate (1.3 g, 69%) as a white solid.

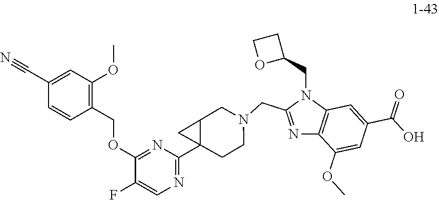 [SMILES: COc1cc(C#N)ccc1COc1nc(C23CCN(Cc4nc5c(OC)cc(C(=O)O)cc5n4C[C@@H]4CCO4)CC2C3)ncc1F]

2-((6-(4-((4-cyano-2-methoxybenzyl)oxy)-5-fluoropyrimidin-2-yl)-3-azabicyclo[4.1.0]heptan-3-yl)methyl)-4-methoxy-1-(((S)-oxetan-2-yl)methyl)-1H-benzo[d]imidazole-6-carboxylic acid (1-43): The title compound was synthesize following the procedure of compound 1-31 by using compound 1-24e as the starting material. LCMS (ESI, m/z): [M+H]⁺==629.3. ¹H NMR (300 MHZ, DMSO-d₆): δ 8.49 (s, 1H), 7.86 (d, J=1.2 Hz, 1H), 7.55-7.52 (m, 2H), 7.46-7.43 (m, 1H), 7.28 (s, 1H), 5.57-5.51 (m, 2H), 5.09-5.03 (m, 1H), 4.77-4.72 (m, 1H), 4.62-4.57 (m, 1H), 4.47-4.43 (m, 1H), 4.39-4.34 (m, 1H), 3.96 (s, 3H), 3.89 (s, 3H), 3.79-3.77 (m, 1H), 3.73-3.65 (m, 1H), 3.03-2.99 (m, 1H), 2.91-2.80 (m, 1H), 2.68-2.63 (m, 2H), 2.54-2.51 (m, 1H), 2.43-2.34 (m, 1H), 2.15-2.09 (m, 1H), 1.85-1.71 (m, 2H), 1.33-1.25 (m, 1H), 1.03-1.00 (m, 1H).

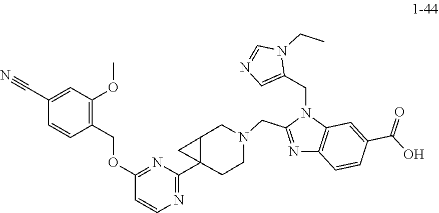 [SMILES: CCn1cncc1Cn1c(CN2CCC3(c4nccc(OCc5ccc(C#N)cc5OC)n4)CC3C2)nc2ccc(C(=O)O)cc21]

2-((6-(6-((4-cyano-2-methoxybenzyl)oxy)pyridin-2-yl)-3-azabicyclo[4.1.0]heptan-3-yl)methyl)-1-((1-ethyl-1H-imidazol-5-yl)methyl)-1H-benzo[d]imidazole-6-carboxylic acid (1-44): The title compound was synthesized with 1-44e as the starting material of which the synthesis was shown below. LCMS (ESI, m/z): [M+H]⁺=618.3. ¹H NMR (300 MHZ, CD₃OD): δ 8.13 (s, 1H), 8.03-7.99 (m, 1H), 7.74-7.68 (m, 2H), 7.60-7.55 (m, 1H), 7.47 (d, J=7.5 Hz, 1H), 7.34 (d, J=1.2 Hz, 1H), 7.30-7.27 (m, 1H), 6.83 (d, J=7.5 Hz, 1H), 6.63 (d, J=8.1 Hz, 1H), 6.52 (s, 1H), 5.75 (s, 2H), 5.39 (s, 2H), 4.09-3.97 (m, 2H), 3.94 (s, 3H), 3.81 (s, 2H), 2.96-2.90 (m, 1H), 2.72-2.69 (m, 1H), 2.31-2.26 (m, 3H), 1.93-1.87 (m, 1H), 1.73-1.66 (m, 1H), 1.24-1.19 (m, 3H), 1.06-1.01 (m, 1H), 0.72-0.69 (m, 1H). The synthesis of methyl 2-(chloromethyl)-1-((1-ethyl-1H-imidazol-5-yl)methyl)-1H-benzo[d]imidazole-6-carboxylate (1-44e):

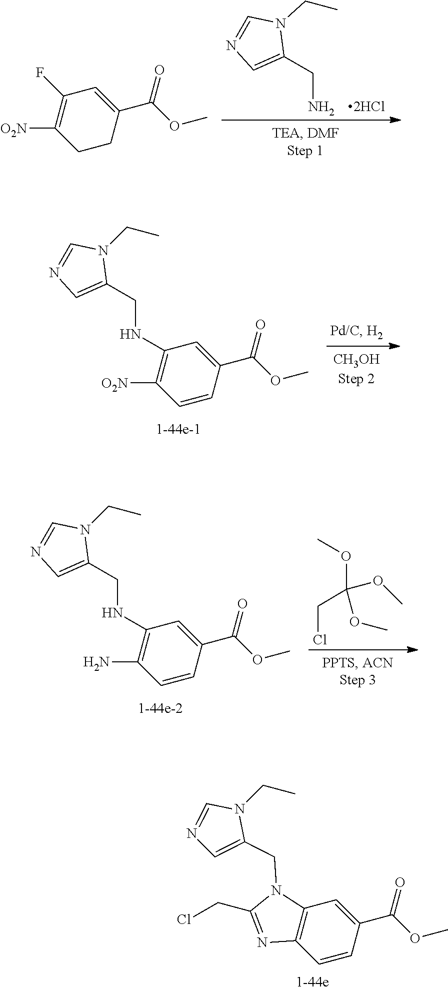

Step 1. To a solution of methyl 3-fluoro-4-nitrobenzoate (500.0 mg, 2.51 mmol) in DMF (10.0 m) was added 1-(3-ethylimidazol-4-yl) methanamine dihydrochloride (547.1 mg, 2.76 mmol) and TEA (762.2 mg, 7.53 mmol) at room temperature. The resulting mixture was stirred at room temperature for 16 h. After the reaction was completed, the resulting mixture was diluted with water and extracted with ethyl acetate. The combined organic layer was washed with brine, dried over anhydrous sodium sulfate and filtered. The filtrate was concentrated under vacuum. The residue was by flash column chromatography with CH₂C₁₂/CH₃OH (93/7, v/v) to afford methyl 3-(((1-ethyl-1H-imidazol-5-yl)methyl)amino)-4-nitrobenzoate (1-44e-1) (691.0 mg, 90%) as an orange solid. LCMS (ESI, m/z): [M+H]⁺=305.0.

Step 2. To a solution of compound 1-44e-1 (691.0 mg, 2.27 mmol) in methanol (10.0 mL) was added Pd/C (200.0 mg, 10%) at room temperature under N₂. The resulting mixture was stirred at room temperature for 2 h under H₂. After the reaction was completed, the resulting mixture was filtered. The filtrate was concentrated under vacuum to afford methyl 4-amino-3-(((1-ethyl-1H-imidazol-5-yl)methyl)amino) benzoate (1-44e-2) (622.0 mg, crude) as a yellow green solid. LCMS (ESI, m/z): [M+H]⁺=275.0.

Step 3. To a solution of compound 1-44e-2 (622.0 mg, crude) in ACN (10.0 mL) was added 2-chloro-1,1,1-trimethoxyethane (701.0 mg, 4.53 mmol) and PPTS (569.8 mg, 2.27 mmol) at room temperature. The resulting mixture was stirred at room temperature for 16 h. After the reaction was completed, the resulting mixture was concentrated under vacuum. The mixture was purified by flash column chromatography with CH₂Cl₂/CH₃OH (93/7, v/v) to afford 1-44e (594.7 mg, 79%) as a yellow solid. LCMS (ESI, m/z): [M+H]⁺=333.0.

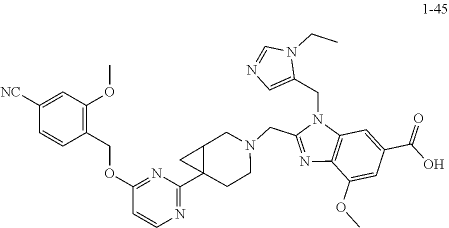 [SMILES: CCn1cncc1Cn1c(CN2CCC3(c4nccc(OCc5ccc(C#N)cc5OC)n4)CC3C2)nc2c(OC)cc(C(=O)O)cc21]

2-((6-(4-((4-cyano-2-methoxybenzyl)oxy)pyrimidin-2-yl)-3-azabicyclo[4.1.0]heptan-3-yl)methyl)-1-((1-ethyl-1H-imidazol-5-yl)methyl)-4-methoxy-1H-benzo[d]imidazole-6-carboxylic acid (1-45): the title compound was synthesized following the procedure of compound 1-34 by using methyl 2-(chloromethyl)-1-((1-ethyl-1H-imidazol-5-yl)methyl)-4-methoxy-1H-benzo[d]imidazole-6-carboxylate (1-45e) (1-45e was synthesized following the compound 1-18e by using methyl 3-bromo-5-methoxy-4-nitrobenzoate as the starting material). LCMS (ESI, m/z): [M+H]⁺=649.3. ¹H NMR (400 MHZ, DMSO-d₆): δ 8.38 (d, J=4.0 Hz, 1H), 7.67 (s, 1H), 7.63 (s, 1H), 7.53-7.48 (m, 2H), 7.44-7.41 (m, 1H), 7.29 (s, 1H), 6.73 (d, J=5.6 Hz, 1H), 6.32 (s, 1H), 5.65 (s, 2H), 5.40 (s, 2H), 3.99-3.93 (m, 5H), 3.88 (s, 3H), 3.77-3.74 (m, 1H), 3.68-3.64 (m, 1H), 3.01-2.97 (m, 1H), 2.87-2.84 (m, 1H), 2.58-2.54 (m, 1H), 2.46-2.43 (m, 1H), 2.04-1.98 (m, 1H), 1.73-1.60 (m, 2H), 1.24-1.14 (m, 4H), 0.72-0.70 (m, 1H).

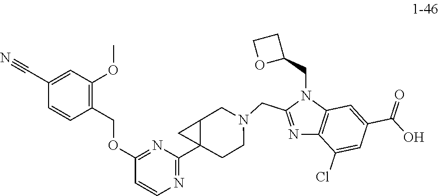 [SMILES: COc1cc(C#N)ccc1COc1ccnc(C23CCN(Cc4nc5c(Cl)cc(C(=O)O)cc5n4C[C@@H]4CCO4)CC2C3)n1]

4-chloro-2-((6-(4-((4-cyano-2-methoxybenzyl)oxy)pyrimidin-2-yl)-3-azabicyclo[4.1.0]heptan-3-yl)methyl)-1-(((S)-oxetan-2-yl)methyl)-1H-benzo[d]imidazole-6-carboxylic acid (1-46): the title compound was synthesized following the procedure of compound 1-34 by using 1-25e as the starting material. LCMS (ESI, m/z): [M+H]⁺=615.2. ¹H NMR (400 MHZ, Methanol-d₄): δ 8.34 (d, J=5.6 Hz, 1H), 8.27 (s, 1H), 7.97-7.96 (m, 1H), 7.49 (d, J=7.6 Hz, 1H), 7.36-7.30 (m, 2H), 6.68 (d, J=5.6 Hz, 1H), 5.53-5.45 (m, 2H), 5.27-5.19 (m, 1H), 4.93-4.90 (m, 1H), 4.76-4.70 (m, 1H), 4.66-4.59 (m, 1H), 4.52-4.40 (m, 1H), 4.07-3.87 (m, 5H), 3.14-3.08 (m, 1H), 2.99-2.73 (m, 3H), 2.61-2.44 (m, 2H), 2.38-2.29 (m, 1H), 2.01-1.92 (m, 1H), 1.88-1.83 (m, 1H), 1.45-1.40 (m, 1H), 1.12-1.08 (m, 1H).

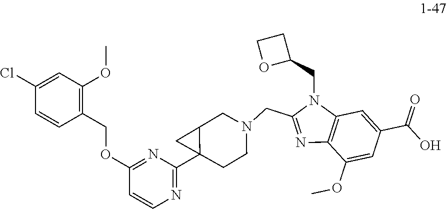 [SMILES: COc1cc(Cl)ccc1COc1ccnc(C23CCN(Cc4nc5c(OC)cc(C(=O)O)cc5n4C[C@@H]4CCO4)CC2C3)n1]

2-((6-(4-((4-chloro-2-methoxybenzyl)oxy)pyrimidin-2-yl)-3-azabicyclo[4.1.0]heptan-3-yl)methyl)-4-methoxy-1-(((S)-oxetan-2-yl)methyl)-1H-benzo[d]imidazole-6-carboxylic acid (1-47): the title compound was synthesized following the procedure of compound 1-34. LCMS (ESI, m/z): [M+H]⁺=620.4. ¹H NMR (400 MHZ, DMSO-d₆): δ 8.37 (d, J=6.0 Hz, 1H), 7.86 (s, 1H), 7.38-7.35 (m, 1H), 7.28 (s, 1H), 7.12 (d, J=1.8 Hz, 1H), 7.03-6.99 (m, 1H), 6.69 (d, J=5.7 Hz, 1H), 5.33 (s, 2H), 5.08-5.03 (m, 1H), 4.77-4.70 (m, 1H), 4.62-4.57 (m, 1H), 4.48-4.45 (m, 1H), 4.40-4.28 (m, 1H), 3.96 (s, 3H), 3.86-3.66 (m, 5H), 3.13-3.08 (m, 1H), 2.93-2.82 (m, 1H), 2.69-2.65 (m, 2H), 2.44-2.35 (m, 1H), 2.12-2.08 (m, 1H), 1.89-1.81 (m, 2H), 1.41-1.37 (m, 1H), 1.05-1.03 (m, 1H).

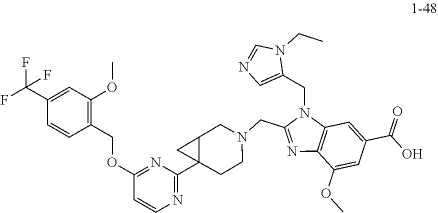 [SMILES: CCn1cncc1Cn1c(CN2CCC3(c4nccc(OCc5ccc(C(F)(F)F)cc5OC)n4)CC3C2)nc2c(OC)cc(C(=O)O)cc21]

1-((1-ethyl-1H-imidazol-5-yl)methyl)-4-methoxy-2-((6-(4-((2-methoxy-4-(trifluoromethyl)benzyl)oxy)pyrimidin-2-yl)-3-azabicyclo[4.1.0]heptan-3-yl)methyl)-1H-benzo[d]imidazole-6-carboxylic acid (1-48): the title compound was synthesized following the procedure of compound 1-34 by using compound 1-45e as the starting material. LCMS (ESI, m/z): [M+H]⁺=692.4. ¹H NMR (400 MHZ, DMSO-d₆): δ 8.39 (d, J=5.6 Hz, 1H), 7.65-7.61 (m, 2H), 7.53 (d, J=8.0 Hz, 1H), 7.32-7.30 (m, 3H), 6.72 (d, J=5.6 Hz, 1H), 6.31 (s, 1H), 5.63 (s, 2H), 5.42-4.38 (m, 2H), 3.99-3.93 (m, 5H), 3.90 (s, 3H), 3.76-3.73 (m, 1H), 3.67-3.63 (m, 1H), 3.02-2.98 (m, 1H), 2.87-2.84 (m, 1H), 2.56-2.54 (m, 1H), 2.44-2.42 (m, 1H), 2.02-1.97 (m, 1H), 1.71-1.64 (m, 2H), 1.24-1.19 (m, 1H), 1.18-1.14 (m, 3H), 0.72-0.69 (s, 1H).

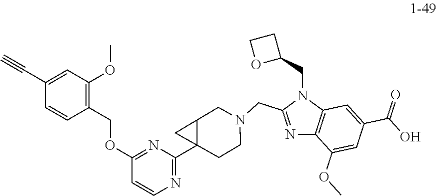 [SMILES: C#Cc1ccc(COc2ccnc(C34CCN(Cc5nc6c(OC)cc(C(=O)O)cc6n5C[C@@H]5CCO5)CC3C4)n2)c(OC)c1]

2-((6-(4-((4-ethynyl-2-methoxybenzyl)oxy)pyrimidin-2-yl)-3-azabicyclo[4.1.0]heptan-3-yl)methyl)-4-methoxy-1-(((S)-oxetan-2-yl)methyl)-1H-benzo[d]imidazole-6-carboxylic acid (1-49): the title compound was synthesized following the procedure of compound 1-34 by using compound 1-24e and (2-methoxy-4-((trimethylsilyl) ethynyl)phenyl) methanol as the starting material, and the protecting group (TMS group) was simultaneously removed at final hydrolysis step. LCMS (ESI, m/z): [M+H]⁺=610.3. ¹H NMR (400 MHZ, Methanol-d₄): δ 8.30 (d, J=4.0 Hz, 1H), 7.89 (d, J=0.8 Hz, 1H), 7.48 (s, 1H), 7.30 (d, J=8.0 Hz, 1H), 7.07-7.04 (m, 2H), 6.61 (d, J=6.0 Hz, 1H), 5.47-5.39 (m, 2H), 5.27-5.21 (m, 1H), 4.71-4.67 (m, 1H), 4.63-4.58 (m, 1H), 4.49-4.39 (m, 1H), 4.05 (s, 3H), 3.98-3.81 (m, 5H), 3.49 (s, 1H), 3.13-3.07 (m, 1H), 2.94-2.86 (m, 1H), 2.85-2.71 (m, 2H), 2.56-2.44 (m, 2H), 2.32-2.25 (m, 1H), 2.01-1.94 (m, 1H), 1.92-1.85 (m, 1H), 1.47-1.42 (m, 1H), 1.12-1.09 (m, 1H).

### Example 2. Synthesis of Compound 2

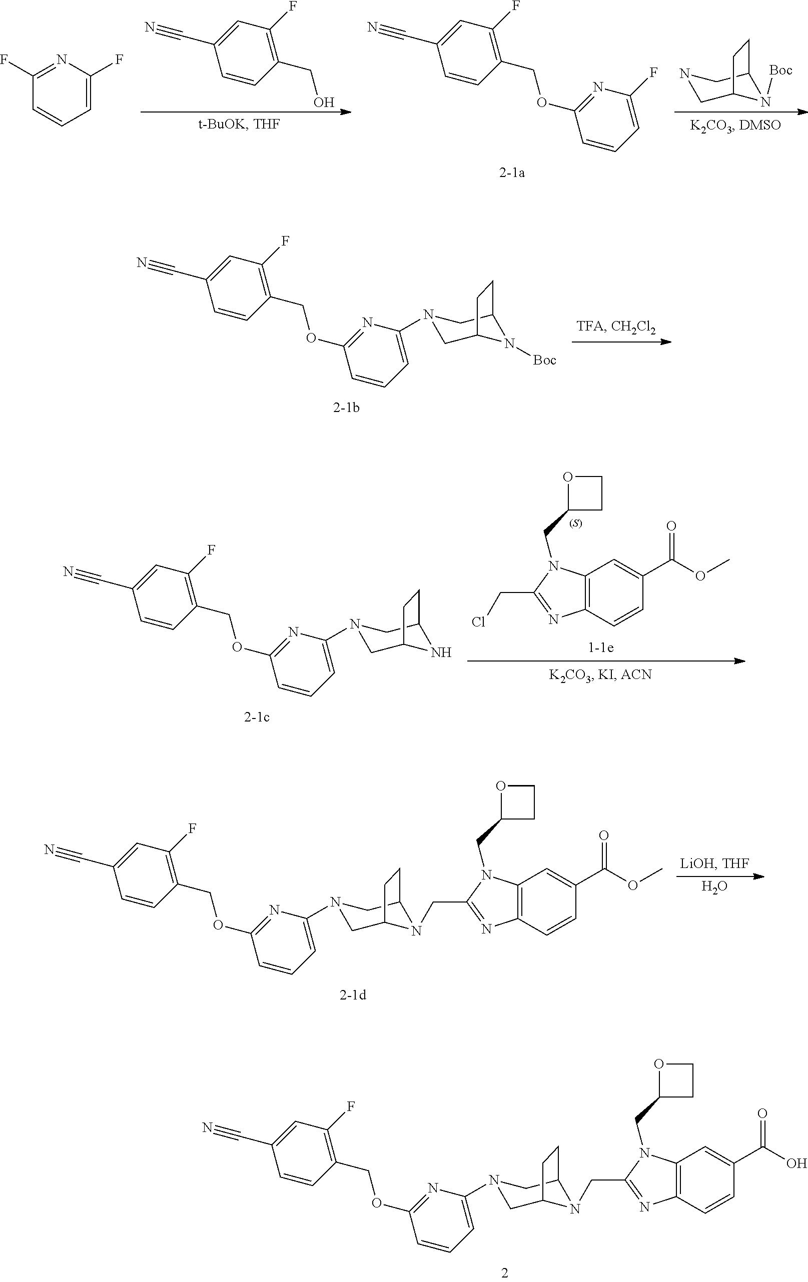

3-fluoro-4-{[(6-fluoropyridin-2-yl)oxy]methyl}benzonitrile (2-1a): To a solution of 2,6-difluoropyridine (5.0 g, 43.45 mmol) in THF (100.0 mL) was added 3-fluoro-4-(hydroxymethyl)benzonitrile (7.9 g, 52.14 mmol) and t-BuOK (8.8 g, 78.25 mmol) at room temperature. The reaction mixture was stirred at room temperature for 30 min. After the reaction was completed, the mixture was quenched with saturated NH₄Cl (aq.) and then extracted with ethyl acetate. The combined organic layer was washed with brine, dried over anhydrous sodium sulfate and filtered. The filtrate was concentrated under vacuum. The residue was purified by flash column chromatography with ethyl acetate/petroleum ether (10/90, v/v) to afford 3-fluoro-4-{[(6-fluoropyridin-2-yl)oxy]methyl}benzonitrile (10.0 g, 93%) a white solid. LCMS (ESI, m/z): [M+H]⁺=247.1.

Tert-butyl-3-(6-((4-cyano-2-fluorobenzyl)oxy)pyridin-2-yl)-3,8-diazabicyclo[3.2.1]octane-8-carboxylate (2-1b): To a mixture of 3-fluoro-4-{[(6-fluoropyridin-2-yl)oxy]methyl}benzonitrile (750.0 mg, 3.05 mmol) in DMSO (10.0 mL) was added tert-butyl 3,8-diazabicyclo[3.2.1]octane-8-carboxylate (711.3 mg, 3.35 mmol) and K₂CO₃ (1500.0 mg, 10.85 mmol) at room temperature. The resulting mixture was stirred at 120° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (50/50, v/v) to afford tert-butyl-3-(6-((4-cyano-2-fluorobenzyl)oxy)pyridin-2-yl)-3,8-diazabicyclo[3.2.1]octane-8-carboxylate (340.0 mg, 25%) as a white solid. LCMS (ESI, m/z): [M+H]⁺=439.2.

4-(((6-(−3,8-diazabicyclo[3.2.1]octan-3-yl)pyridin-2-yl)oxy)methyl)-3-fluorobenzonitrile (2-1c): To a mixture of tert-butyl-3-(6-((4-cyano-2-fluorobenzyl)oxy)pyridin-2-yl)-3,8-diazabicyclo[3.2.1]octane-8-carboxylate (340.0 mg, 0.78 mmol) in CH₂Cl₂ (5.0 mL) was added TFA (5.0 mL) at room temperature. The resulting mixture was stirred at room temperature for 30 min. After the reaction was completed, the mixture was concentrated under reduced pressure. The pH value of the residue was adjusted to 7.0 with saturated NaHCO₃(aq.). The mixture was extracted with ethyl acetate. The combined organic layer was washed with brine, dried over anhydrous sodium sulfate and filtered. The filtrate was concentrated under reduced pressure to afford 4-(((6-(−3,8-diazabicyclo[3.2.1]octan-3-yl)pyridin-2-yl)oxy)methyl)-3-fluorobenzonitrile (262.0 mg, crude) as a yellow solid. LCMS (ESI, m/z): [M+H]⁺=339.2.

Methyl 2-((−3-(6-((4-cyano-2-fluorobenzyl)oxy)pyridin-2-yl)-3,8-diazabicyclo[3.2.1]octan-8-yl)methyl)-1-(((S)-oxetan-2-yl)methyl)-1H-benzo[d]imidazole-6-carboxylate (2-1d): To a mixture of 4-(((6-(−3,8-diazabicyclo[3.2.1]octan-3-yl)pyridin-2-yl)oxy)methyl)-3-fluorobenzonitrile (123.0 mg, 0.36 mmol) in ACN (5.0 mL) was added methyl(S)-2-(chloromethyl)-1-(oxetan-2-ylmethyl)-1H-benzo[d]imidazole-6-carboxylate (107.1 mg, 0.36 mmol), K₂CO₃ (100.5 mg, 0.73 mmol) and KI (30.2 mg, 0.18 mmol) at room temperature. The resulting mixture was stirred at room temperature for 16 h. After the reaction was completed, the resulting mixture was filtered. The filtrate was concentrated under vacuum. The residue was purified by flash column chromatography with CH₂Cl₂/MeOH (90/10, v/v) to afford methyl 2-((−3-(6-((4-cyano-2-fluorobenzyl)oxy)pyridin-2-yl)-3,8-diazabicyclo[3.2.1]octan-8-yl)methyl)-1-(((S)-oxetan-2-yl)methyl)-1H-benzo[d]imidazole-6-carboxylate (170.0 mg, 78%) as a yellow solid. LCMS (ESI, m/z): [M+H]⁺=597.2.

2-((−3-(6-((4-cyano-2-fluorobenzyl)oxy)pyridin-2-yl)-3,8-diazabicyclo[3.2.1]octan-8-yl)methyl)-1-(((S)-oxetan-2-yl)methyl)-1H-benzo[d]imidazole-6-carboxylic acid (2): To a mixture of methyl 2-((−3-(6-((4-cyano-2-fluorobenzyl)oxy)pyridin-2-yl)-3,8-diazabicyclo[3.2.1]octan-8-yl)methyl)-1-(((S)-oxetan-2-yl)methyl)-1H-benzo[d]imidazole-6-carboxylate (140.0 mg, 0.24 mmol) in THF (6.0 mL) and water (4.0 mL) was added LiOH (56.2 mg, 2.35 mmol) at room temperature. The resulting mixture was stirred at room temperature for 16 h. After the reaction was completed, the pH value of the residue was adjusted to 5.0 with saturated HCl (aq.). The mixture was extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Shield RP18 OBD Column, 30×150 mm, 5 μm; Mobile Phase A: Water (10 mmol/L NH₄HCO₃), Mobile Phase B: ACN; Flow rate: 60 mL/min; Gradient: 30% B to 45% B in 8 min; Wave Length: 254 nm) to afford 2-((−3-(6-((4-cyano-2-fluorobenzyl)oxy)pyridin-2-yl)-3,8-diazabicyclo[3.2.1]octan-8-yl)methyl)-1-(((S)-oxetan-2-yl)methyl)-1H-benzo[d]imidazole-6-carboxylic acid (27.3 mg, 19%) as a white solid. LCMS (ESI, m/z): [M+H]⁺=583.4. ¹H NMR (400 MHZ, DMSO-d₆): δ 8.25 (s, 1H), 7.88-7.80 (m, 2H), 7.71-7.61 (m, 3H), 7.46-7.42 (m, 1H), 6.19 (d, J=8.4 Hz, 1H), 6.10 (d, J=7.6 Hz, 1H), 5.39 (s, 2H), 5.18-5.14 (m, 1H), 4.89-4.84 (m, 1H), 4.74-4.70 (m, 1H), 4.51-4.46 (m, 1H), 4.41-4.36 (m, 1H), 3.96-3.92 (m, 1H), 3.87-3.83 (m, 1H), 3.77-3.70 (m, 2H), 2.91-2.83 (m, 2H), 2.50-2.44 (m, 1H), 2.03-1.99 (m, 2H), 1.56-1.53 (m, 2H).

Following the synthetic route described above in Example 2 and substituting the appropriate reagents, starting materials and purification methods known to those skilled in the art, the compounds listed below were synthesized.

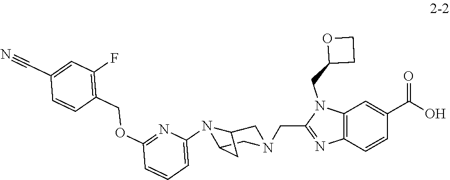 [SMILES: N#Cc1ccc(COc2cccc(N3C4CC3CN(Cc3nc5ccc(C(=O)O)cc5n3C[C@@H]3CCO3)C4)n2)c(F)c1]

2-((6-(6-((4-cyano-2-fluorobenzyl)oxy)pyridin-2-yl)-3,6-diazabicyclo[3.1.1]heptan-3-yl)methyl)-1-(((S)-oxetan-2-yl)methyl)-1H-benzo[d]imidazole-6-carboxylic acid (2-2): white solid. LCMS (ESI, m/z): [M+H]⁺=569.4. ¹H NMR (400 MHZ, CD₃OD): δ 8.13 (s, 1H), 8.00-7.98 (m, 1H), 7.67 (d, J=8.4 Hz, 1H), 7.53-7.42 (m, 2H), 7.32-7.29 (m, 1H), 7.03 (d, J=8.0 Hz, 1H), 6.31 (d, J=8.0 Hz, 1H), 6.17 (d, J=7.6 Hz, 1H), 5.22-5.17 (m, 2H), 4.91-4.86 (m, 1H), 4.57-4.51 (m, 1H), 4.34-4.21 (m, 3H), 4.05-3.99 (m, 2H), 3.97-3.92 (m, 1H), 3.84-3.79 (m, 1H), 3.34-3.32 (m, 1H), 3.11-3.02 (m, 1H), 2.68-2.58 (m, 4H), 2.35-2.21 (m, 1H), 1.53-1.50 (m, 1H).

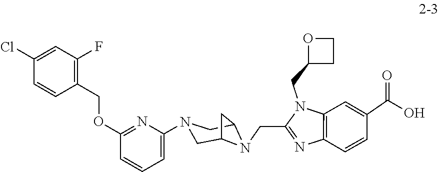 [SMILES: O=C(O)c1ccc2nc(CN3C4CC3CN(c3cccc(OCc5ccc(Cl)cc5F)n3)C4)n(C[C@@H]3CCO3)c2c1]

2-((−3-(6-((4-chloro-2-fluorobenzyl)oxy)pyridin-2-yl)-3,6-diazabicyclo[3.1.1]heptan-6-yl)methyl)-1-(((S)-oxetan-2-yl)methyl)-1H-benzo[d]imidazole-6-carboxylic acid (2-3): white solid. LCMS (ESI, m/z): [M+H]⁺=578.4. ¹H NMR (400 MHZ, DMSO-d₆): δ 8.22 (s, 1H), 7.80 (d, J=8.4 Hz, 1H), 7.62-7.59 (m, 2H), 7.53-7.45 (m, 2H), 7.33-7.31 (m, 1H), 6.23 (d, J=8.0 Hz, 1H), 6.12 (d, J=7.6 Hz, 1H), 5.42 (s, 2H), 5.11-5.05 (m, 1H), 4.78-4.72 (m, 1H), 4.65-4.60 (m, 1H), 4.46-4.40 (m, 1H), 4.31-4.26 (m, 1H), 3.92-3.83 (m, 3H), 3.75-3.69 (m, 2H), 3.52-3.35 (m, 2H), 2.72-2.64 (m, 1H), 2.43-2.35 (m, 1H), 1.56-1.53 (m, 1H).

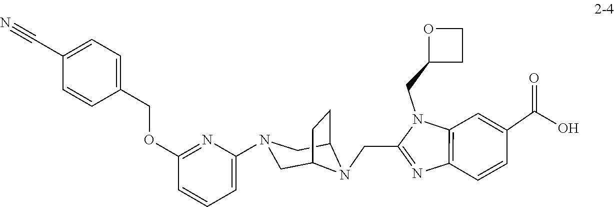 [SMILES: N#Cc1ccc(COc2cccc(N3CC4CCC(C3)N4Cc3nc4ccc(C(=O)O)cc4n3C[C@@H]3CCO3)n2)cc1]

2-[(3-{6-[(4-cyanophenyl)methoxy]pyridin-2-yl}-3,8-diazabicyclo[3.2.1]octan-8-yl)methyl]-3-[(2S)-oxetan-2-ylmethyl]-1,3-benzodiazole-5-carboxylic acid (2-4): white solid. LCMS (ESI, m/z): [M+H]⁺=565.4. ¹H NMR (400 MHZ, DMSO-d₆): δ 12.74 (s, 1H), 8.29 (s, 1H), 7.82-7.80 (m, 3H), 7.66 (d, J=8.4 Hz, 1H), 7.59-7.57 (m, 2H), 7.46-7.42 (m, 1H), 6.18-6.10 (m, 2H), 5.36 (s, 2H), 5.16-5.12 (m, 1H), 4.91-4.85 (m, 1H), 4.75-4.72 (m, 1H), 4.51-4.46 (m, 1H), 4.41-4.36 (m, 1H), 3.97-3.84 (m, 2H), 3.76-3.70 (m, 2H), 3.38-3.31 (m, 1H), 2.89-2.82 (m, 2H), 2.71-2.66 (m, 1H), 2.47-2.43 (m, 1H), 2.03-1.97 (m, 2H), 1.56-1.53 (m, 2H).

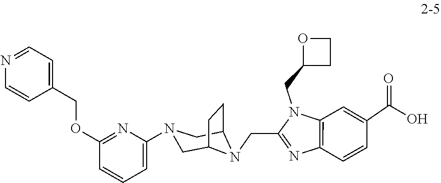 [SMILES: O=C(O)c1ccc2nc(CN3C4CCC3CN(c3cccc(OCc5ccncc5)n3)C4)n(C[C@@H]3CCO3)c2c1]

3-[(2S)-oxetan-2-ylmethyl]-2-({3-[6-(pyridin-4-ylmethoxy)pyridin-2-yl]-3,8-diazabicyclo[3.2.1]octan-8-yl}methyl)-1,3-benzodiazole-5-carboxylic acid (2-5): white solid. LCMS (ESI, m/z): [M+H]⁺=541.3. ¹H NMR (400 MHZ, DMSO-d₆): δ 12.80 (s, 1H), 8.53-8.51 (m, 2H), 8.27 (s, 1H), 7.82 (d, J=8.4 Hz, 1H), 7.65 (d, J=8.4 Hz, 1H), 7.46-7.42 (m, 1H), 7.36 (d, J=5.2 Hz, 2H), 6.19-6.12 (m, 2H), 5.32 (s, 2H), 5.18-5.12 (m, 1H), 4.90-4.84 (m, 1H), 4.75-4.70 (m, 1H), 4.51-4.46 (m, 1H), 4.41-4.36 (m, 1H), 3.96-3.92 (m, 1H), 3.86-3.83 (m, 1H), 3.75-3.69 (m, 2H), 3.37-3.31 (m, 2H), 2.89-2.80 (m, 2H), 2.70-2.66 (m, 1H), 2.49-2.40 (m, 1H), 2.02-1.98 (m, 2H), 1.55-1.52 (m, 2H).

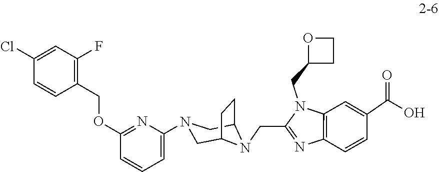 [SMILES: O=C(O)c1ccc2nc(CN3C4CCC3CN(c3cccc(OCc5ccc(Cl)cc5F)n3)C4)n(C[C@@H]3CCO3)c2c1]

2-[(3-{6-[(4-chloro-2-fluorophenyl)methoxy]pyridin-2-yl}-3,8-diazabicyclo[3.2.1]octan-8-yl)methyl]-3-[(2S)-oxetan-2-ylmethyl]-1,3-benzodiazole-5-carboxylic acid (Compound 2-6): white solid. LCMS (ESI, m/z): [M+H]⁺=592.3. ¹H NMR (400 MHZ, Methanol-d₄): δ 8.37 (s, 1H), 8.01-7.99 (m, 1H), 7.71 (d, J=8.8 Hz, 1H), 7.48-7.40 (m, 2H), 7.21-7.17 (m, 2H), 6.16 (d, J=8.0 Hz, 1H), 6.10 (d, J=8.0 Hz, 1H), 5.36-5.32 (m, 3H), 4.97-4.95 (m, 1H), 4.84-4.80 (m, 1H), 4.66-4.63 (m, 1H), 4.50-4.48 (m, 1H), 4.12-4.07 (m, 2H), 3.87-3.83 (m, 2H), 3.48-3.41 (m, 2H), 3.05-2.98 (m, 2H), 2.87-2.74 (m, 1H), 2.60-2.50 (m, 1H), 2.20-2.16 (m, 2H), 1.78-1.75 (m, 2H).

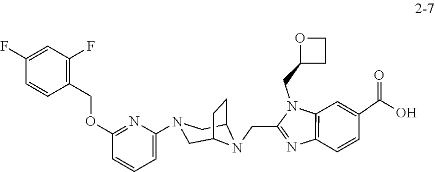 [SMILES: O=C(O)c1ccc2nc(CN3C4CCC3CN(c3cccc(OCc5ccc(F)cc5F)n3)C4)n(C[C@@H]3CCO3)c2c1]

2-((−3-(6-((2,4-difluorobenzyl)oxy)pyridin-2-yl)-3,8-diazabicyclo[3.2.1]octan-8-yl)methyl)-1-(((S)-oxetan-2-yl)methyl)-1H-benzo[d]imidazole-6-carboxylic acid (2-7): white solid. LCMS (ESI, m/z): [M+H]⁺=576.3. ¹H NMR (400 MHZ, CD₃OD): δ 8.34 (s, 1H), 8.02-7.99 (m, 1H), 7.68 (d, J=8.4 Hz, 1H), 7.49-7.43 (m, 1H), 7.41-7.37 (m, 1H), 6.97-6.90 (m, 2H), 6.14 (d, J=8.0 Hz, 1H), 6.06 (d, J=7.6 Hz, 1H), 5.31-5.29 (m, 3H), 4.94-4.90 (m, 1H), 4.82-4.78 (m, 1H), 4.63-4.60 (m, 1H), 4.48-4.45 (m, 1H), 4.05-3.99 (m, 2H), 3.89-3.82 (m, 2H), 3.43-3.32 (m, 2H), 3.03-2.96 (m, 2H), 2.81-2.70 (m, 1H), 2.58-2.47 (m, 1H), 2.17-2.13 (m, 2H), 1.76-1.73 (m, 2H).

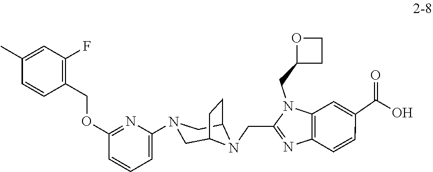 [SMILES: Cc1ccc(COc2cccc(N3CC4CCC(C3)N4Cc3nc4ccc(C(=O)O)cc4n3C[C@@H]3CCO3)n2)c(F)c1]

2-[(3-{6-[(2-fluoro-4-methylphenyl)methoxy]pyridin-2-yl}-3,8-diazabicyclo[3.2.1]octan-8-yl)methyl]-3-[(2S)-oxetan-2-ylmethyl]-1,3-benzodiazole-5-carboxylic acid (2-8): white solid. LCMS (ESI, m/z): [M+H]⁺=572.4. ¹H-NMR (400 MHZ, CD₃OD): δ 8.34 (s, 1H), 8.01-7.99 (m, 1H), 7.69 (d, J=8.8 Hz, 1H), 7.42-7.38 (m, 1H), 7.34-7.30 (m, 1H), 6.96-6.89 (m, 2H), 6.14 (d, J=8.0 Hz, 1H), 6.06 (d, J=8.0 Hz, 1H), 5.39-5.32 (m, 3H), 4.67-4.61 (m, 2H), 4.49-4.47 (m, 1H), 4.09-4.01 (m, 2H), 3.87-3.83 (m, 2H), 3.44-3.32 (m, 2H), 3.04-2.97 (m, 2H), 2.86-2.75 (m, 1H), 2.62-2.52 (m, 1H), 2.33 (s, 3H), 2.23-2.12 (m, 2H), 1.77-1.71 (m, 2H).

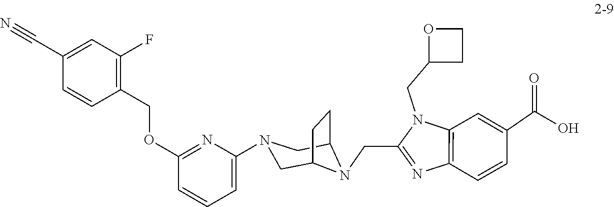 [SMILES: N#Cc1ccc(COc2cccc(N3CC4CCC(C3)N4Cc3nc4ccc(C(=O)O)cc4n3CC3CCO3)n2)c(F)c1]

2-((−3-(6-((4-cyano-2-fluorobenzyl)oxy)pyridin-2-yl)-3,8-diazabicyclo[3.2.1]octan-8-yl)methyl)-1-(oxetan-2-ylmethyl)-1H-benzo[d]imidazole-6-carboxylic acid (2-9): white solid. LCMS (ESI, m/z): [M+H]⁺=583.3. ¹H NMR (400 MHZ, DMSO-d₆): δ 8.28 (s, 1H), 7.88-7.81 (m, 2H), 7.71-7.63 (m, 3H), 7.46-7.42 (m, 1H), 6.19 (d, J=8.0 Hz, 1H), 6.10 (d, J=8.0 Hz, 1H), 5.39 (s, 2H), 5.16-5.14 (m, 1H), 4.87-4.85 (m, 1H), 4.74-4.71 (m, 1H), 4.49-4.38 (m, 2H), 3.96-3.93 (m, 1H), 3.87-3.83 (m, 1H), 3.76-3.70 (m, 2H), 3.38-3.33 (m, 2H), 2.91-2.83 (m, 2H), 2.77-2.67 (m, 1H), 2.50-2.45 (m, 1H), 2.03-1.99 (m, 2H), 1.56-1.53 (m, 2H).

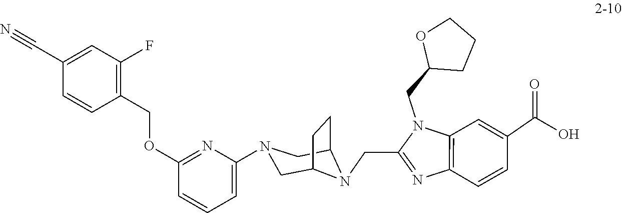 [SMILES: N#Cc1ccc(COc2cccc(N3CC4CCC(C3)N4Cc3nc4ccc(C(=O)O)cc4n3C[C@@H]3CCCO3)n2)c(F)c1]

2-((−3-(6-((4-cyano-2-fluorobenzyl)oxy)pyridin-2-yl)-3,8-diazabicyclo[3.2.1]octan-8-yl)methyl)-1-(((S)-tetrahydrofuran-2-yl)methyl)-1H-benzo[d]imidazole-6-carboxylic acid (2-10): white solid. LCMS (ESI, m/z): [M+H]⁺=597.2. ¹H NMR (400 MHZ, CD₃OD): δ 8.30 (s, 1H), 7.99 (d, J=8.0 Hz, 1H), 7.67-7.62 (m, 2H), 7.57-7.53 (m, 2H), 7.45-7.41 (m, 1H), 6.17-6.12 (m, 2H), 5.44 (s, 2H), 4.69-4.67 (m, 2H), 4.45-4.42 (m, 1H), 4.08-4.05 (m, 1H), 3.98-3.90 (m, 2H), 3.84-3.75 (m, 3H), 3.40-3.35 (m, 1H), 3.00-2.89 (m, 2H), 2.18-2.11 (m, 3H), 1.96-1.91 (m, 2H), 1.76-1.68 (m, 3H).

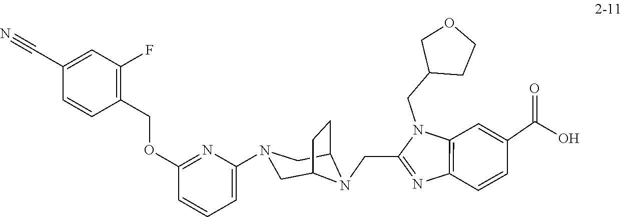 [SMILES: N#Cc1ccc(COc2cccc(N3CC4CCC(C3)N4Cc3nc4ccc(C(=O)O)cc4n3CC3CCOC3)n2)c(F)c1]

2-[(3-{6-[(4-cyano-2-fluorophenyl)methoxy]pyridin-2-yl}-3,8-diazabicyclo[3.2.1]octan-8-yl)methyl]-3-(oxolan-3-ylmethyl)-1,3-benzodiazole-5-carboxylic acid (2-11): white solid. LCMS (ESI, m/z): [M+H]⁺=597.2. ¹H NMR (400 MHZ, DMSO-d₆): δ 12.83 (s, 1H), 8.22 (s, 1H), 7.88-7.82 (m, 2H), 7.70-7.63 (m, 3H), 7.46-7.42 (m, 1H), 6.18 (d, J=8.0 Hz, 1H), 6.10 (d, J=7.6 Hz, 1H), 5.39 (s, 2H), 4.50-4.48 (m, 2H), 3.92-3.84 (m, 3H), 3.75-3.72 (m, 2H), 3.66-3.55 (m, 3H), 3.41 (s, 2H), 3.11-2.97 (m, 1H), 2.86-2.83 (m, 2H), 2.04-2.01 (m, 2H), 1.97-1.89 (m, 1H), 1.77-1.71 (m, 1H), 1.57-1.52 (m, 2H).

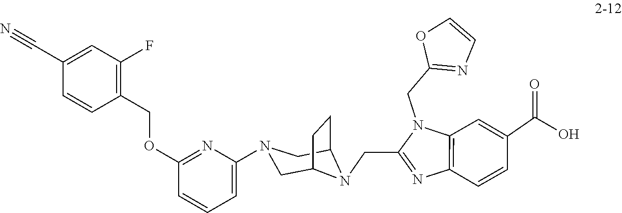 [SMILES: N#Cc1ccc(COc2cccc(N3CC4CCC(C3)N4Cc3nc4ccc(C(=O)O)cc4n3Cc3ncco3)n2)c(F)c1]

2-((−3-(6-((4-cyano-2-fluorobenzyl)oxy)pyridin-2-yl)-3,8-diazabicyclo[3.2.1]octan-8-yl)methyl)-1-(oxazol-2-ylmethyl)-1H-benzo[d]imidazole-6-carboxylic acid (2-12): yellow solid. LCMS (ESI, m/z): [M+H]⁺=594.3. ¹H NMR (400 MHZ, CD₃OD): δ 8.23 (s, 1H), 8.03 (d, J=8.4 Hz, 1H), 7.85 (d, J=7.2 Hz, 1H), 7.68-7.61 (m, 2H), 7.57-7.53 (m, 2H), 7.42-7.38 (m, 1H), 7.07 (s, 1H), 6.16-6.10 (m, 2H), 5.99 (s, 2H), 5.43 (s, 2H), 4.00 (s, 2H), 3.72-3.69 (m, 2H), 3.30-3.26 (m, 2H), 2.78-2.75 (m, 2H), 2.08-1.98 (m, 2H), 1.70-1.63 (m, 2H).

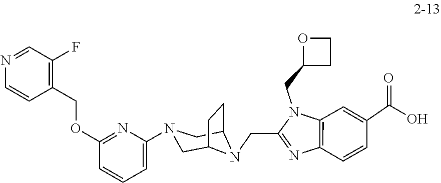 [SMILES: O=C(O)c1ccc2nc(CN3C4CCC3CN(c3cccc(OCc5ccncc5F)n3)C4)n(C[C@@H]3CCO3)c2c1]

2-[(3-{6-[(3-fluoropyridin-4-yl)methoxy]pyridin-2-yl}-3,8-diazabicyclo[3.2.1]octan-8-yl)methyl]-3-[(2S)-oxetan-2-ylmethyl]-1,3-benzodiazole-5-carboxylic acid (2-13): white solid. LCMS (ESI, m/z): [M+H]⁺=559.2. ¹H NMR (400 MHZ, DMSO-d₆): δ 8.55 (d, J=1.6 Hz, 1H), 8.40 (d, J=4.4 Hz, 1H), 8.25 (s, 1H), 7.82 (d, J=8.4 Hz, 1H), 7.62 (d, J=8.0 Hz, 1H), 7.48-7.43 (m, 2H), 6.20-6.12 (m, 2H), 5.40 (s, 2H), 5.18-5.12 (m, 1H), 4.89-4.83 (m, 1H), 4.73-4.70 (m, 1H), 4.51-4.46 (m, 1H), 4.41-4.36 (m, 1H), 3.95-3.83 (m, 2H), 3.75-3.68 (m, 2H), 3.38-3.33 (m, 2H), 2.90-2.81 (m, 2H), 2.70-2.65 (m, 1H), 2.52-2.50 (m, 1H), 2.05-1.98 (m, 2H), 1.55-1.49 (m, 2H).

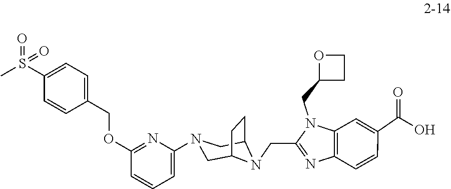 [SMILES: CS(=O)(=O)c1ccc(COc2cccc(N3CC4CCC(C3)N4Cc3nc4ccc(C(=O)O)cc4n3C[C@@H]3CCO3)n2)cc1]

2-((−3-(6-((4-(methylsulfonyl)benzyl)oxy)pyridin-2-yl)-3,8-diazabicyclo[3.2.1]octan-8-yl)methyl)-1-(((S)-oxetan-2-yl)methyl)-1H-benzo[d]imidazole-6-carboxylic acid (2-14): white solid. LCMS (ESI, m/z): [M+H]⁺=618.2. ¹H NMR (400 MHZ, DMSO-d₆): δ 8.27 (s, 1H), 7.91-7.81 (m, 3H), 7.66-7.62 (m, 3H), 7.46-7.42 (m, 1H), 6.18-6.10 (m, 2H), 5.38 (s, 2H), 5.16-5.14 (m, 1H), 4.90-4.85 (m, 1H), 4.74-4.71 (m, 1H), 4.51-4.45 (m, 1H), 4.41-4.36 (m, 1H), 3.93-3.88 (m, 2H), 3.76-3.70 (m, 2H), 3.38-3.30 (m, 2H), 3.16 (s, 3H), 2.90-2.81 (m, 2H), 2.72-2.64 (m, 1H), 2.46-2.40 (m, 1H), 2.04-1.97 (m, 2H), 1.55-1.53 (m, 2H).

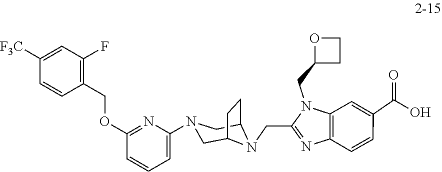 [SMILES: O=C(O)c1ccc2nc(CN3C4CCC3CN(c3cccc(OCc5ccc(C(F)(F)F)cc5F)n3)C4)n(C[C@@H]3CCO3)c2c1]

2-((−3-(6-((2-fluoro-4-(trifluoromethyl)benzyl)oxy)pyridin-2-yl)-3,8-diazabicyclo[3.2.1]octan-8-yl)methyl)-1-(((S)-oxetan-2-yl)methyl)-1H-benzo[d]imidazole-6-carboxylic acid (2-15): white solid. LCMS (ESI, m/z): [M+H]⁺=626.3. ¹H NMR (400 MHZ, DMSO-d₆): δ 12.70 (s, 1H), 8.27 (s, 1H), 7.83-7.81 (m, 1H), 7.70-7.58 (m, 4H), 7.46-7.42 (m, 1H), 6.18 (d, J=8.0 Hz, 1H), 6.10 (d, J=7.6 Hz, 1H), 5.40 (s, 2H), 5.16-5.14 (m, 1H), 4.90-4.85 (m, 1H), 4.75-4.71 (m, 1H), 4.51-4.46 (m, 1H), 4.39-4.37 (m, 1H), 3.96-3.84 (m, 2H), 3.77-3.71 (m, 2H), 3.38-3.33 (m, 2H), 2.91-2.83 (m, 2H), 2.70-2.66 (m, 1H), 2.45-2.42 (m, 1H), 2.03-1.96 (m, 2H), 1.56-1.53 (m, 2H).

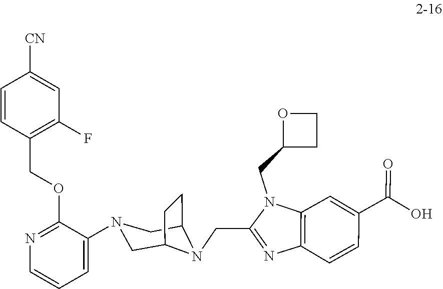 [SMILES: N#Cc1ccc(COc2ncccc2N2CC3CCC(C2)N3Cc2nc3ccc(C(=O)O)cc3n2C[C@@H]2CCO2)c(F)c1]

2-[(3-{2-[(4-cyano-2-fluorophenyl)methoxy]pyridin-3-yl}-3,8-diazabicyclo[3.2.1]octan-8-yl)methyl]-3-[(2S)-oxetan-2-ylmethyl]-1,3-benzodiazole-5-carboxylic acid (2-16): The title compound was synthesized by using 2-16b as the starting material of which the synthesis was shown below. Yellow solid. LCMS (ESI, m/z): [M+H]⁺=583.4. ¹H NMR (400 MHZ, DMSO-d₆): δ 8.28 (s, 1H), 7.93-7.91 (m, 1H), 7.82-7.80 (m, 1H), 7.76-7.69 (m, 3H), 7.64 (d, J=8.4 Hz, 1H), 7.14 (d, J=6.8 Hz, 1H), 6.94-6.91 (m, 1H), 5.50 (s, 2H), 5.19-5.15 (m, 1H), 4.87-4.85 (m, 1H), 4.77-4.72 (m, 1H), 4.50-4.48 (m, 1H), 4.39-4.37 (m, 1H), 3.91-3.80 (m, 2H), 3.27-3.21 (m, 3H), 2.81-2.72 (m, 3H), 2.48-2.45 (m, 1H), 2.02-1.94 (m, 2H), 1.82-1.79 (m, 2H).

The synthesis of tert-butyl(1R,5S)-3-(2-((4-cyano-2-fluorobenzyl)oxy)pyridin-3-yl)-3,8-diazabicyclo[3.2.1]octane-8-carboxylate (2-16b):

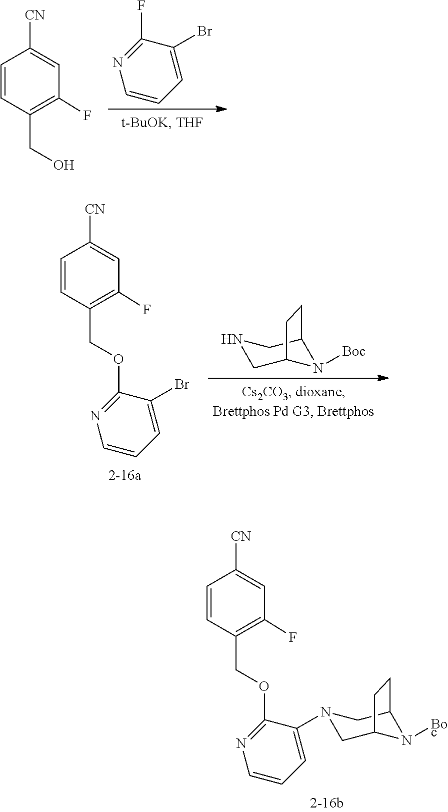

Step 1. To a solution of 3-bromo-2-fluoropyridine (2.0 g, 11.36 mmol) in THF (50.0 mL) was added 3-fluoro-4-(hydroxymethyl)benzonitrile (2.1 g, 13.64 mmol) and t-BuOK (2.3 g, 20.46 mmol) at room temperature. The reaction mixture was stirred at room temperature for 30 min. After the reaction was completed, the mixture was quenched with saturated NH₄Cl (aq.) and then extracted with ethyl acetate. The combined organic layer was washed with brine, dried over anhydrous sodium sulfate and filtered. The filtrate was concentrated under vacuum. The residue was purified by flash column chromatography with ethyl acetate/petroleum ether (20/80, v/v) to afford 2-16a (2.3 g, 53%) as a yellow solid. LCMS (ESI, m/z): [M+H]⁺=307.1.

Step 2. To a mixture of 2-16a (1.2 g, 3.91 mmol) in dioxane (50.0 mL) was added tert-butyl 3,8-diazabicyclo[3.2.1]octane-8-carboxylate (0.9 g, 4.30 mmol), Cs₂CO₃ (3.8 g, 11.72 mmol), Brettphos (0.4 g, 0.78 mmol) and BrettPhos Pd G3 (0.4 g, 0.39 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (50/50, v/v) to afford 2-16b (110.0 mg, 6%) as a white solid. LCMS (ESI, m/z): [M+H]⁺=439.2.

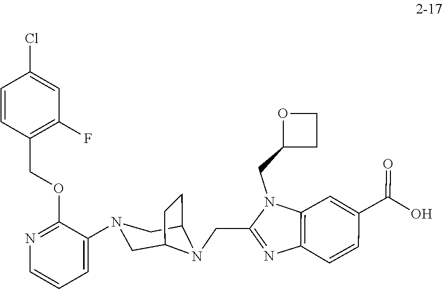 [SMILES: O=C(O)c1ccc2nc(CN3C4CCC3CN(c3cccnc3OCc3ccc(Cl)cc3F)C4)n(C[C@@H]3CCO3)c2c1]

2-[(3-{2-[(4-chloro-2-fluorophenyl)methoxy]pyridin-3-yl}-3,8-diazabicyclo[3.2.1]octan-8-yl)methyl]-3-[(2S)-oxetan-2-ylmethyl]-1,3-benzodiazole-5-carboxylic acid (2-17): The title compound was synthesized by following the procedure of 2-16. white solid. LCMS (ESI, m/z): [M+H]⁺=592.2. ¹H NMR (400 MHZ, DMSO-d₆): δ 12.72 (s, 1H), 8.15 (s, 1H), 7.81 (d, J=8.4 Hz, 1H), 7.71-7.64 (m, 2H), 7.60-7.56 (m, 1H), 7.51-7.48 (m, 1H), 7.33 (d, J=8.0 Hz, 1H), 7.12 (d, J=7.2 Hz, 1H), 6.92-6.89 (m, 1H), 5.39 (s, 2H), 5.19-5.14 (m, 1H), 4.91-4.85 (m, 1H), 4.77-4.72 (m, 1H), 4.51-4.45 (m, 1H), 4.40-4.35 (m, 1H), 3.91-3.79 (m, 2H), 3.24-3.19 (m, 3H), 2.79-2.67 (m, 3H), 2.47-2.43 (m, 1H), 1.99-1.92 (m, 2H), 1.79-1.75 (m, 2H).

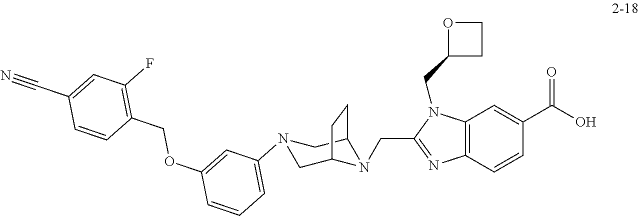 [SMILES: N#Cc1ccc(COc2cccc(N3CC4CCC(C3)N4Cc3nc4ccc(C(=O)O)cc4n3C[C@@H]3CCO3)c2)c(F)c1]

2-((−3-(3-((4-cyano-2-fluorobenzyl)oxy)phenyl)-3,8-diazabicyclo[3.2.1]octan-8-yl)methyl)-1-(((S)-oxetan-2-yl)methyl)-1H-benzo[d]imidazole-6-carboxylic acid (2-18): The title compound was synthesized by using 2-18b as the starting material of which the synthesis was shown below. white solid. LCMS (ESI, m/z): [M+H]⁺=582.3. ¹H NMR (400 MHz, CD₃OD): δ 8.34 (s, 1H), 8.01-7.99 (m, 1H), 7.75-7.66 (m, 2H), 7.60-7.57 (m, 2H), 7.12-7.08 (m, 1H), 6.50-6.40 (m, 3H), 5.33-5.28 (m, 1H), 5.18 (s, 2H), 4.94-4.88 (m, 1H), 4.84-4.79 (m, 1H), 4.64-4.60 (m, 1H), 4.48-4.45 (m, 1H), 4.11-4.02 (m, 2H), 3.45-3.33 (m, 4H), 2.95-2.89 (m, 2H), 2.85-2.79 (m, 1H), 2.58-2.47 (m, 1H), 2.21-2.17 (m, 2H), 1.88-1.85 (m, 2H).

The synthesis of tert-butyl(1R,5S)-3-(3-((4-cyano-2-fluorobenzyl)oxy)phenyl)-3,8-diazabicyclo[3.2.1]octane-8-carboxylate (2-18b):

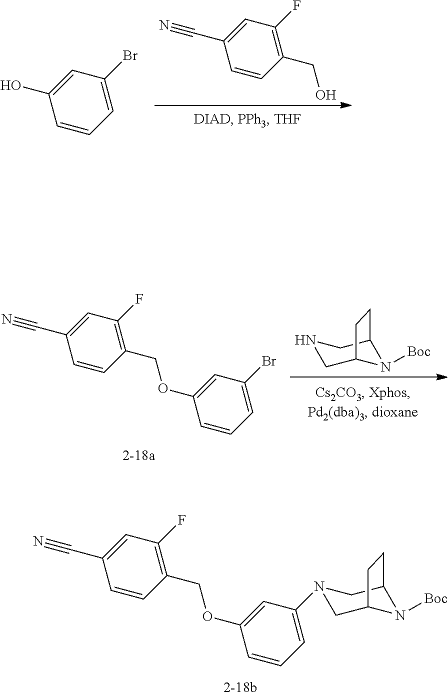 [SMILES: Oc1cccc(Br)c1 ; N#Cc1ccc(CO)c(F)c1 ; N#Cc1ccc(COc2cccc(Br)c2)c(F)c1 ; CC(C)(C)OC(=O)N1C2CCC1CNC2 ; CC(C)(C)OC(=O)N1C2CCC1CN(c1cccc(OCc3ccc(C#N)cc3F)c1)C2]

Step 1. To a mixture of PPh₃ (2.3 g, 8.67 mmol) and DIAD (1.7 g, 8.67 mmol) in THF (30.0 mL) was added 3-fluoro-4-(hydroxymethyl)benzonitrile (1.1 g, 5.98 mmol) at 0° C. under N₂. The resulting mixture was stirred at 0° C. for 10 min. Then 3-bromophenol (1.0 g, 5.78 mmol) was added to the mixture at 0° C. under N₂. The resulting mixture was stirred at 0° C. for 30 min. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with ethyl acetate/petroleum ether (10/90, v/v) to afford 4-((3-bromophenoxy)methyl)-3-fluorobenzonitrile (2-18a) (2.9 g, 68%) as a white solid. LCMS (ESI, m/z): [M+H]⁺=306.0.

Step 2. To a mixture of 2-18a (700.0 mg, 2.29 mmol) and tert-butyl 3,8-diazabicyclo[3.2.1]octane-8-carboxylate (582.5 mg, 2.74 mmol) in dioxane (20.0 mL) was added Cs₂CO₃ (2.2 g, 6.86 mmol), Xphos (218.0 mg, 0.46 mmol) and Pd₂(dba)₃ (209.4 mg, 0.23 mmol) at room temperature under N₂. The reaction mixture was stirred at 100° C. for 4 h. After the reaction was completed, the mixture was diluted with water and extracted with ethyl acetate. The combined organic layer was washed with brine, dried over anhydrous sodium sulfate and filtered. The filtrate was concentrated under vacuum. The residue was purified by flash column chromatography with ethyl acetate/petroleum ether (15/85, v/v) to afford tert-butyl-3-(3-((4-cyano-2-fluorobenzyl)oxy)phenyl)-3,8-diazabicyclo[3.2.1]octane-8-carboxylate (2-18b) (710.0 mg, 70%) as a brown solid. LCMS (ESI, m/z): [M+H]⁺=438.2.

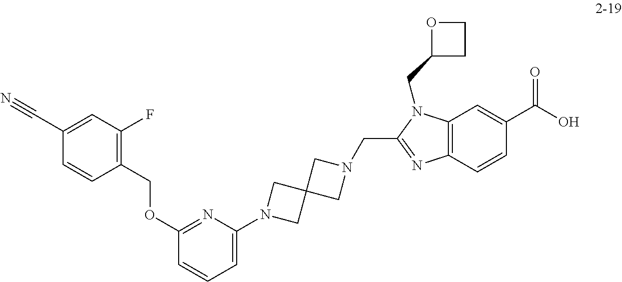 [SMILES: N#Cc1ccc(COc2cccc(N3CC4(CN(Cc5nc6ccc(C(=O)O)cc6n5C[C@@H]5CCO5)C4)C3)n2)c(F)c1]

(S)-2-((6-(6-((4-cyano-2-fluorobenzyl)oxy)pyridin-2-yl)-2,6-diazaspiro[3.3]heptan-2-yl)methyl)-1-(oxetan-2-ylmethyl)-1H-benzo[d]imidazole-6-carboxylic acid (2-19): white solid. LCMS (ESI, m/z): [M+H]⁺=569.3. ¹H NMR (400 MHZ, DMSO-d₆): δ 8.22 (s, 1H), 7.89-7.87 (m, 1H), 7.80 (d, J=8.4 Hz, 1H), 7.72-7.66 (m, 2H), 7.62 (d, J=8.4 Hz, 1H), 7.45-7.41 (m, 1H), 6.10 (d, J=8.0 Hz, 1H), 5.90 (d, J=8.0 Hz, 1H), 5.39 (s, 2H), 5.07-5.04 (m, 1H), 4.72-4.67 (m, 1H), 4.59-4.56 (m, 1H), 4.48-4.45 (m, 1H), 4.33-4.30 (m, 1H), 3.98-3.95 (m, 5H), 3.88-3.85 (m, 1H), 3.45-3.40 (m, 4H), 2.71-2.67 (m, 1H), 2.42-2.35 (m, 1H).

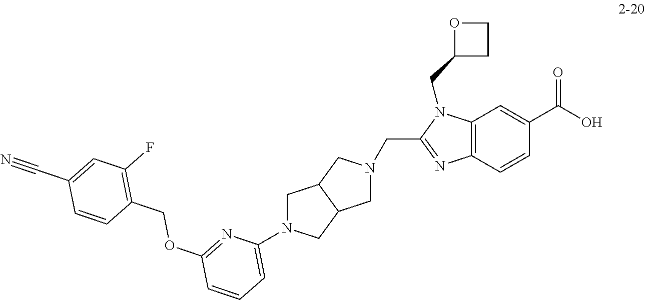 [SMILES: N#Cc1ccc(COc2cccc(N3CC4CN(Cc5nc6ccc(C(=O)O)cc6n5C[C@@H]5CCO5)CC4C3)n2)c(F)c1]

2-[(5-{6-[(4-cyano-2-fluorophenyl)methoxy]pyridin-2-yl}-hexahydropyrrolo[3,4-c]pyrrol-2-yl)methyl]-3-[(2S)-oxetan-2-ylmethyl]-1,3-benzodiazole-5-carboxylic acid (2-20): white solid. LCMS (ESI, m/z): [M+H]⁺=583.4. ¹H NMR (400 MHZ, DMSO-d₆): δ 8.23-8.19 (m, 1H), 7.88-7.85 (m, 1H), 7.80-7.78 (m, 1H), 7.70-7.61 (m, 3H), 7.46-7.42 (m, 1H), 6.08-6.02 (m, 2H), 5.43-5.38 (m, 2H), 5.01-4.95 (m, 1H), 4.71-4.65 (m, 1H), 4.52-4.48 (m, 1H), 4.30-4.20 (m, 2H), 4.11-4.08 (m, 1H), 3.78-3.75 (m, 1H), 3.58-3.48 (m, 2H), 3.24-3.17 (m, 3H), 2.93-2.88 (m, 2H), 2.66-2.58 (m, 3H), 2.42-2.36 (m, 1H), 2.30-2.21 (m, 1H).

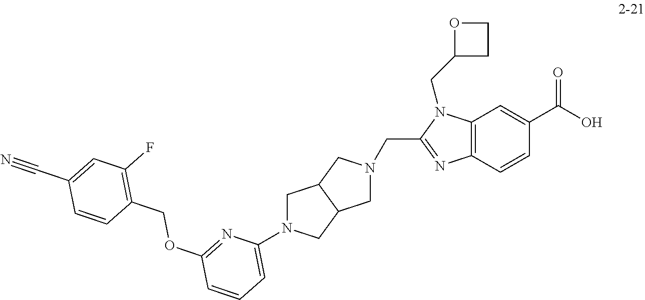 [SMILES: N#Cc1ccc(COc2cccc(N3CC4CN(Cc5nc6ccc(C(=O)O)cc6n5CC5CCO5)CC4C3)n2)c(F)c1]

2-[(5-{6-[(4-cyano-2-fluorophenyl)methoxy]pyridin-2-yl}-hexahydropyrrolo[3,4-c]pyrrol-2-yl)methyl]-3-(oxetan-2-ylmethyl)-1,3-benzodiazole-5-carboxylic acid (2-21): white solid. LCMS (ESI, m/z): [M+H]⁺=583.3. ¹H NMR (400 MHZ, DMSO-d₆): δ 8.23-8.17 (m, 1H), 7.88-7.85 (m, 1H), 7.80-7.78 (m, 1H), 7.68-7.62 (m, 3H), 7.45-7.42 (m, 1H), 6.08-6.02 (m, 2H), 5.43-5.41 (m, 2H), 5.01-4.95 (m, 1H), 4.71-4.65 (m, 1H), 4.53-4.48 (m, 1H), 4.29-4.23 (m, 2H), 4.11-4.08 (m, 1H), 3.79-3.75 (m, 1H), 3.55-3.47 (m, 2H), 3.24-3.17 (m, 2H), 2.92-2.87 (m, 2H), 2.66-2.60 (m, 3H), 2.42-2.34 (m, 1H), 2.30-2.21 (m, 1H).

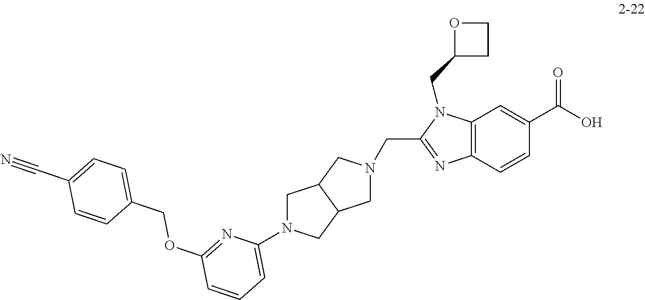 [SMILES: N#Cc1ccc(COc2cccc(N3CC4CN(Cc5nc6ccc(C(=O)O)cc6n5C[C@@H]5CCO5)CC4C3)n2)cc1]

2-((5-(6-((4-cyanobenzyl)oxy)pyridin-2-yl) hexahydropyrrolo[3,4-c]pyrrol-2 (1H)-yl)methyl)-1-(((S)-oxetan-2-yl)methyl)-1H-benzo[d]imidazole-6-carboxylic acid (2-22): white solid. LCMS (ESI, m/z): [M+H]⁺=565.3. ¹H NMR (400 MHZ, DMSO-d₆): δ 8.22 (s, 1H), 7.83-7.79 (m, 3H), 7.62-7.59 (m, 3H), 7.46-7.42 (m, 1H), 6.09-6.02 (m, 2H), 5.40-5.35 (m, 2H), 5.02-4.96 (m, 1H), 4.70-4.65 (m, 1H), 4.51-4.47 (m, 1H), 4.29-4.21 (m, 2H), 4.11-4.08 (m, 1H), 3.78-3.75 (m, 1H), 3.58-3.48 (m, 3H), 3.24-3.18 (m, 2H), 2.94-2.88 (m, 2H), 2.68-2.56 (m, 3H), 2.41-2.38 (m, 1H), 2.30-2.26 (m, 1H).

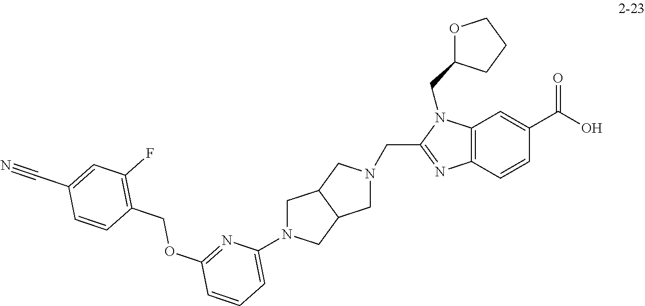 [SMILES: N#Cc1ccc(COc2cccc(N3CC4CN(Cc5nc6ccc(C(=O)O)cc6n5C[C@@H]5CCCO5)CC4C3)n2)c(F)c1]

2-[(5-{6-[(4-cyano-2-fluorophenyl)methoxy]pyridin-2-yl}-hexahydropyrrolo[3,4-c]pyrrol-2-yl)methyl]-3-[(2S)-oxolan-2-ylmethyl]-1,3-benzodiazole-5-carboxylic acid (2-23): white solid. LCMS (ESI, m/z): [M+H]⁺=597.3. ¹H NMR (400 MHz, DMSO-d₆): δ 12.72 (s, 1H), 8.17 (s, 1H), 7.87 (d, J=10.0 Hz, 1H), 7.80-7.77 (m, 1H), 7.68-7.61 (m, 3H), 7.44-7.40 (m, 1H), 6.06 (d, J=8.0 Hz, 1H), 5.99 (d, J=8.0 Hz, 1H), 5.44-5.37 (m, 2H), 4.40-4.33 (m, 2H), 4.16-4.12 (m, 2H), 3.72-3.68 (m, 2H), 3.60-3.56 (m, 1H), 3.53-3.48 (m, 1H), 3.41-3.37 (m, 1H), 3.21-3.18 (m, 1H), 3.13-3.10 (m, 1H), 2.94-2.88 (m, 2H), 2.73-2.70 (m, 1H), 2.63-2.54 (m, 2H), 1.82-1.73 (m, 1H), 1.70-1.62 (m, 1H), 1.44-1.35 (m, 2H).

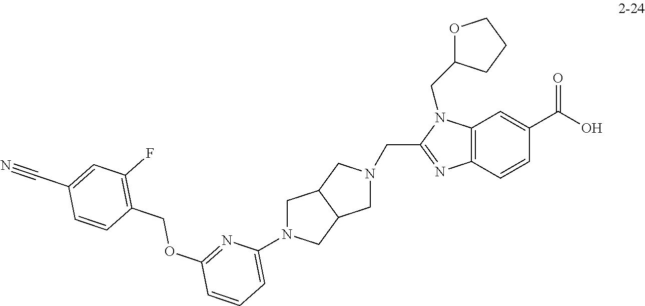 [SMILES: N#Cc1ccc(COc2cccc(N3CC4CN(Cc5nc6ccc(C(=O)O)cc6n5CC5CCCO5)CC4C3)n2)c(F)c1]

2-[(5-{6-[(4-cyano-2-fluorophenyl)methoxy]pyridin-2-yl}-hexahydropyrrolo[3,4-c]pyrrol-2-yl)methyl]-3-(oxolan-3-ylmethyl)-1,3-benzodiazole-5-carboxylic acid (2-24): white solid. LCMS (ESI, m/z): [M+H]⁺=597.3. ¹H NMR (400 MHZ, CD₃OD): δ 8.21 (s, 1H), 7.99-7.97 (m, 1H), 7.68-7.62 (m, 2H) 7.55-7.49 (m, 2H), 7.42-7.38 (m, 1H), 6.08 (d, J=7.6 Hz, 1H), 5.99 (d, J=8.0 Hz, 1H), 5.46-5.42 (m, 2H), 4.34-4.27 (m, 2H), 4.07-4.03 (m, 1H), 3.92-3.88 (m, 1H), 3.84-3.78 (m, 1H), 3.52-3.41 (m, 5H), 3.35-3.33 (m, 1H), 3.30-3.26 (m, 1H), 3.05-2.96 (m, 3H), 2.73-2.66 (m, 4H), 1.83-1.74 (m, 1H), 1.64-1.56 (m, 1H).

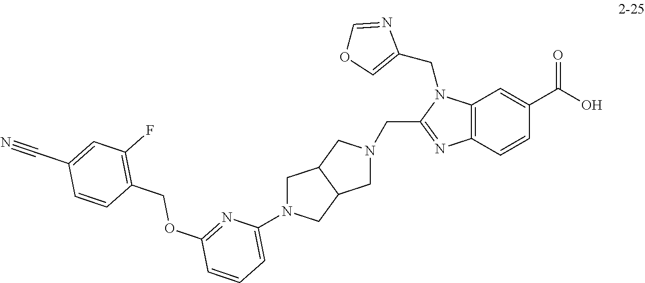 [SMILES: N#Cc1ccc(COc2cccc(N3CC4CN(Cc5nc6ccc(C(=O)O)cc6n5Cc5cocn5)CC4C3)n2)c(F)c1]

2-[(5-{6-[(4-cyano-2-fluorophenyl)methoxy]pyridin-2-yl}-hexahydropyrrolo[3,4-c]pyrrol-2-yl)methyl]-3-(1,3-oxazol-4-ylmethyl)-1,3-benzodiazole-5-carboxylic acid (2-25): white solid. LCMS (ESI, m/z): [M+H]⁺=594.2. ¹H NMR (400 MHZ, DMSO-d₆): δ 8.32-8.19 (m, 2H), 8.06 (s, 1H), 7.88-7.74 (m, 2H), 7.69-7.62 (m, 3H), 7.48-7.41 (m, 1H), 6.07 (d, J=7.6 Hz, 1H), 6.00 (d, J=8.0 Hz, 1H), 5.55 (s, 2H), 5.41 (s, 2H), 4.02 (s, 2H), 3.51-3.48 (m, 2H), 3.17-3.12 (m, 2H), 2.92-2.86 (m, 2H), 2.72-2.68 (m, 2H), 2.58-2.52 (m, 2H).

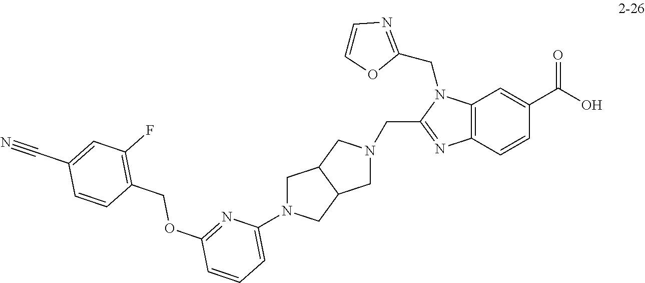 [SMILES: N#Cc1ccc(COc2cccc(N3CC4CN(Cc5nc6ccc(C(=O)O)cc6n5Cc5ncco5)CC4C3)n2)c(F)c1]

2-[(5-{6-[(4-cyano-2-fluorophenyl)methoxy]pyridin-2-yl}-hexahydropyrrolo[3,4-c]pyrrol-2-yl)methyl]-3-(1,3-oxazol-2-ylmethyl)-1,3-benzodiazole-5-carboxylic acid (2-26): white solid. LCMS (ESI, m/z): [M+H]⁺=594.3. ¹H NMR (400 MHZ, DMSO-d₆): δ 8.18-8.15 (m, 1H), 7.88-7.79 (m, 3H), 7.71-7.65 (m, 3H), 7.44-7.40 (m, 1H), 6.98 (s, 1H), 6.06 (d, J=7.6 Hz, 1H), 6.00 (d, J=8.0 Hz 1H), 5.84 (s, 2H), 5.41 (s, 2H), 3.95 (s, 2H), 3.51-3.46 (m, 2H), 3.05-3.02 (m, 2H), 2.82-2.77 (m, 2H), 2.68-2.62 (m, 2H), 2.50-2.47 (m, 2H).

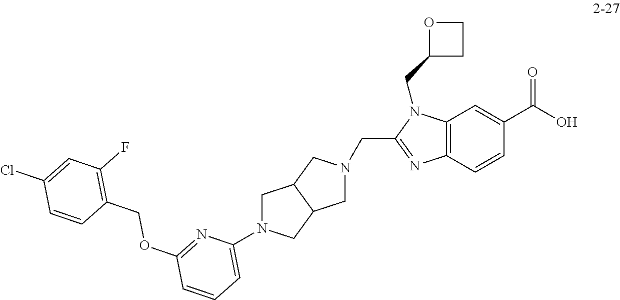 [SMILES: O=C(O)c1ccc2nc(CN3CC4CN(c5cccc(OCc6ccc(Cl)cc6F)n5)CC4C3)n(C[C@@H]3CCO3)c2c1]

2-((5-(6-((4-chloro-2-fluorobenzyl)oxy)pyridin-2-yl) hexahydropyrrolo[3,4-c]pyrrol-2 (1H)-yl)methyl)-1-(((S)-oxetan-2-yl)methyl)-1H-benzo[d]imidazole-6-carboxylic acid (2-27): white solid. LCMS (ESI, m/z): [M+H]⁺=592.3. ¹H NMR (400 MHZ, DMSO-d₆): δ 12.55 (s, 1H), 8.22 (d, J=1.5 Hz, 1H), 7.80-7.78 (m, 1H), 7.62 (d, J=8.4 Hz, 1H), 7.54-7.50 (m, 1H), 7.45-7.40 (m, 2H), 7.28-7.25 (m, 1H), 6.04-6.01 (m, 2H), 5.36-5.29 (m, 2H), 5.02-4.96 (m, 1H), 4.71-4.65 (m, 1H), 4.53-4.48 (m, 1H), 4.29-4.21 (m, 2H), 4.11-4.08 (m, 1H), 3.79-3.75 (m, 1H), 3.61-3.51 (m, 2H), 3.26-3.19 (m, 3H), 2.94-2.88 (m, 2H), 2.67-2.60 (m, 3H), 2.42-2.34 (m, 1H), 2.31-2.22 (m, 1H).

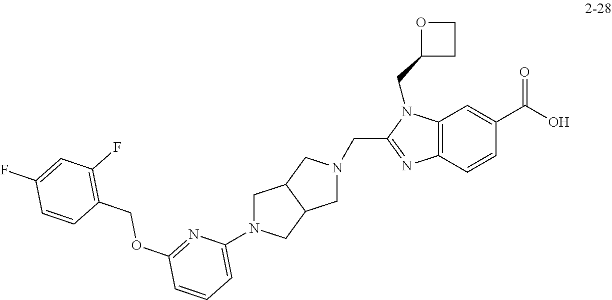 [SMILES: O=C(O)c1ccc2nc(CN3CC4CN(c5cccc(OCc6ccc(F)cc6F)n5)CC4C3)n(C[C@@H]3CCO3)c2c1]

2-[(5-{6-[(2,4-difluorophenyl)methoxy]pyridin-2-yl}-hexahydropyrrolo[3,4-c]pyrrol-2-yl)methyl]-3-[(2S)-oxetan-2-ylmethyl]-1,3-benzodiazole-5-carboxylic acid (2-28): white solid. LCMS (ESI, m/z): [M+H]⁺=576.3. ¹H NMR (400 MHz, DMSO-d₆): δ 8.20 (s, 1H), 7.80 (d, J=8.8 Hz, 1H), 7.59-7.55 (m, 2H), 7.43-7.39 (m, 1H), 7.27-7.22 (m, 1H), 7.09-7.06 (m, 1H), 6.03-6.00 (m, 2H), 5.35-5.26 (m, 2H), 5.03-4.97 (m, 1H), 4.70-4.63 (m, 1H), 4.52-4.48 (m 1H), 4.29-4.22 (m, 2H), 4.11-4.07 (m, 1H), 3.79-3.75 (m, 1H), 3.62-3.52 (m, 2H), 3.29-3.22 (m, 2H), 2.93-2.88 (m, 2H), 2.67-2.60 (m, 3H), 2.44-2.22 (m, 2H).

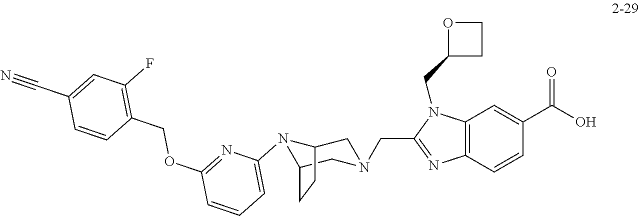 [SMILES: N#Cc1ccc(COc2cccc(N3C4CCC3CN(Cc3nc5ccc(C(=O)O)cc5n3C[C@@H]3CCO3)C4)n2)c(F)c1]

2-((−8-(6-((4-cyano-2-fluorobenzyl)oxy)pyridin-2-yl)-3,8-diazabicyclo[3.2.1]octan-3-yl)methyl)-1-(((S)-oxetan-2-yl)methyl)-1H-benzo[d]imidazole-6-carboxylic acid (2-29): white solid. LCMS (ESI, m/z): [M+H]⁺=583.3. ¹H NMR (400 MHZ, DMSO-d₆): δ 8.24 (s, 1H), 7.87-7.80 (m, 2H), 7.69-7.61 (m, 3H), 7.48-7.44 (m, 1H), 6.29 (d, J=8.0 Hz, 1H), 6.10 (d, J=8.0 Hz, 1H), 5.40 (s, 2H), 5.13-5.07 (m, 1H), 4.90-4.86 (m, 1H), 4.72-4.68 (m, 1H), 4.53-4.48 (m, 1H), 4.42-4.37 (m, 3H), 3.82-3.79 (m, 1H), 3.65-3.62 (m, 1H), 2.78-2.67 (m, 2H), 2.45-2.23 (m, 4H), 1.87-1.76 (m, 4H).

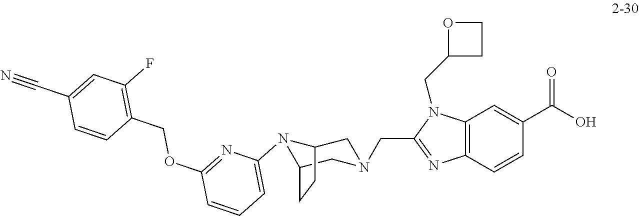 [SMILES: N#Cc1ccc(COc2cccc(N3C4CCC3CN(Cc3nc5ccc(C(=O)O)cc5n3CC3CCO3)C4)n2)c(F)c1]

2-((3-(6-((4-cyano-2-fluorobenzyl)oxy)pyridin-2-yl)-3,8-diazabicyclo[3.2.1]octan-8-yl)methyl)-1-((oxetan-2-yl)methyl)-1H-benzo[d]imidazole-6-carboxylic acid (2-30): white solid. LCMS (ESI, m/z): [M+H]⁺=583.4. ¹H NMR (400 MHZ, CD₃OD): δ 8.34 (d, J=0.4 Hz, 1H), 8.00-7.97 (m, 1H), 7.70-7.62 (m, 2H), 7.57-7.54 (m, 2H), 7.48-7.44 (m, 1H), 6.25 (d, J=8.0 Hz, 1H), 6.14 (d, J=8.0 Hz, 1H), 5.44 (s, 2H), 5.28-5.24 (m, 1H), 5.02-4.96 (m, 1H), 4.85-4.78 (m, 1H), 4.67-4.64 (m, 1H), 4.48-4.40 (m, 3H), 3.90-3.87 (m, 1H), 3.79-3.76 (m, 1H), 2.90-2.77 (m, 1H), 2.58-2.52 (m, 2H), 2.46-2.38 (m, 3H), 2.05-1.87 (m, 4H).

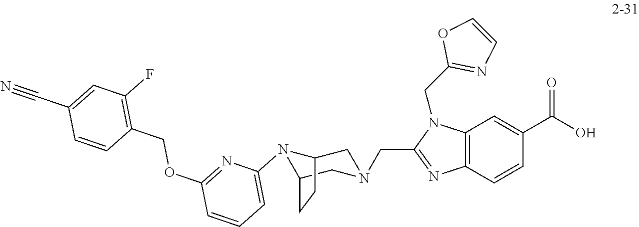 [SMILES: N#Cc1ccc(COc2cccc(N3C4CCC3CN(Cc3nc5ccc(C(=O)O)cc5n3Cc3ncco3)C4)n2)c(F)c1]

2-[(8-{6-[(4-cyano-2-fluorophenyl)methoxy]pyridin-2-yl}-3,8-diazabicyclo[3.2.1]octan-3-yl)methyl]-3-(1,3-oxazol-2-ylmethyl)-1,3-benzodiazole-5-carboxylic acid (2-31): white solid. LCMS (ESI, m/z): [M+H]⁺=594.3. ¹H NMR (400 MHZ, DMSO-d₆): δ 8.11 (s, 2H), 7.86-7.82 (m, 2H), 7.71-7.62 (m, 3H), 7.48-7.44 (m, 1H), 7.21 (s, 1H), 6.28 (d, J=8.0 Hz, 1H), 6.09 (d, J=7.6 Hz, 1H), 5.94 (s, 2H), 5.39 (s, 2H), 4.36 (s, 2H), 3.68 (s, 2H), 2.50-2.47 (m, 2H), 2.26-2.24 (m, 2H), 1.70-1.66 (m, 4H).

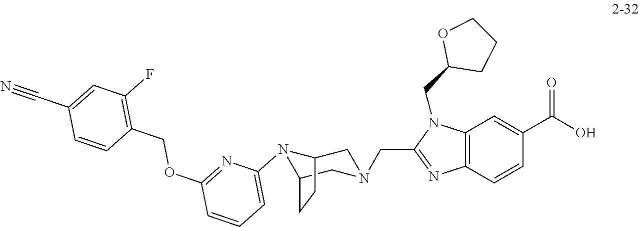 [SMILES: N#Cc1ccc(COc2cccc(N3C4CCC3CN(Cc3nc5ccc(C(=O)O)cc5n3C[C@@H]3CCCO3)C4)n2)c(F)c1]

2-((−8-(6-((4-cyano-2-fluorobenzyl)oxy)pyridin-2-yl)-3,8-diazabicyclo[3.2.1]octan-3-yl)methyl)-1-(((S)-tetrahydrofuran-2-yl)methyl)-1H-benzo[d]imidazole-6-carboxylic acid (2-32): white solid. LCMS (ESI, m/z): [M+H]⁺=597.2. ¹H NMR (400 MHZ, DMSO-d₆): δ 8.19 (s, 1H), 7.87-7.80 (m, 2H), 7.71-7.59 (m, 3H), 7.48-7.44 (m, 1H), 6.29 (d, J=8.0 Hz, 1H), 6.10 (d, J=8.0 Hz, 1H), 5.39 (s, 2H), 4.61-4.51 (m, 2H), 4.43-4.37 (m, 2H), 4.28-4.25 (m, 1H), 3.84-3.78 (m, 2H), 3.67-3.55 (m, 3H), 2.38-2.22 (m, 3H), 2.10-2.05 (m, 1H), 1.89-1.72 (m, 6H), 1.69-1.62 (m, 1H).

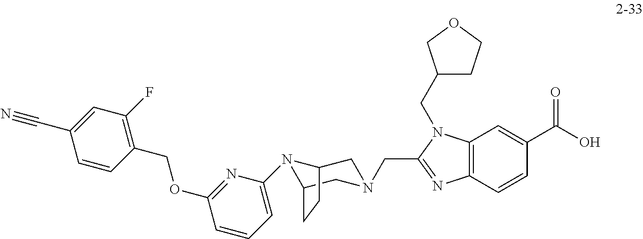 [SMILES: N#Cc1ccc(COc2cccc(N3C4CCC3CN(Cc3nc5ccc(C(=O)O)cc5n3CC3CCOC3)C4)n2)c(F)c1]

2-[(8-{6-[(4-cyano-2-fluorophenyl)methoxy]pyridin-2-yl}-3,8-diazabicyclo[3.2.1]octan-3-yl)methyl]-3-(oxolan-3-ylmethyl)-1,3-benzodiazole-5-carboxylic acid (2-33): white solid. LCMS (ESI, m/z): [M+H]⁺=597.3. ¹H NMR (400 MHZ, DMSO-d₆): δ 8.19 (s, 1H), 7.87-7.81 (m, 2H), 7.71-7.61 (m, 3H), 7.49-7.45 (m, 1H), 6.30 (d, J=8.0 Hz, 1H), 6.10 (d, J=8.0 Hz, 1H), 5.40 (s, 2H), 4.48-4.41 (m, 4H), 3.93-3.88 (m, 1H), 3.71-3.60 (m, 4H), 3.53-3.49 (m, 1H), 2.98-2.93 (m, 1H), 2.52-2.50 (m, 2H), 2.34-2.28 (m, 2H), 1.99-1.90 (m, 1H), 1.78-1.69 (m, 5H).

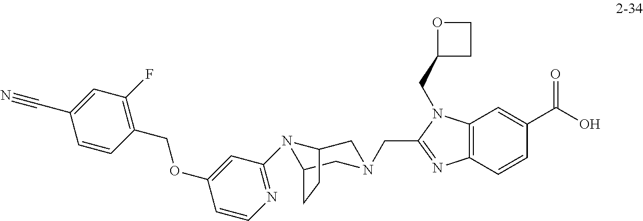 [SMILES: N#Cc1ccc(COc2ccnc(N3C4CCC3CN(Cc3nc5ccc(C(=O)O)cc5n3C[C@@H]3CCO3)C4)c2)c(F)c1]

2-((−8-(4-((4-cyano-2-fluorobenzyl)oxy)pyridin-2-yl)-3,8-diazabicyclo[3.2.1]octan-3-yl)methyl)-1-(((S)-oxetan-2-yl)methyl)-1H-benzo[d]imidazole-6-carboxylic acid (2-34): The title compound was synthesized via following the synthesis of 2-18 with 4-(((2-bromopyridin-4-yl)oxy)methyl)-3-fluorobenzonitrile as the starting material. white solid. LCMS (ESI, m/z): [M+H]⁺=583.3. ¹H NMR (400 MHZ, DMSO-d₆): δ 8.25 (s, 1H), 7.96-7.92 (m, 2H), 7.82-7.76 (m, 3H), 7.60 (d, J=8.4 Hz, 1H), 6.39-6.34 (m, 2H), 5.28 (s, 2H), 5.12-5.10 (m, 1H), 4.92-4.87 (m, 1H), 4.74-4.70 (m, 1H), 4.56-4.48 (m, 3H), 4.39-4.37 (m, 1H), 3.88-3.84 (m, 1H), 3.72-3.69 (m, 1H), 2.73-2.68 (m, 1H), 2.62-2.59 (m, 1H), 2.46-2.41 (m, 3H), 2.39-2.36 (m, 1H), 1.90-1.82 (m, 4H).

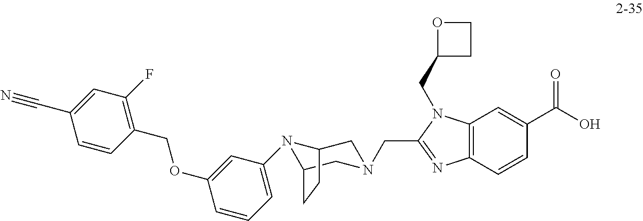 [SMILES: N#Cc1ccc(COc2cccc(N3C4CCC3CN(Cc3nc5ccc(C(=O)O)cc5n3C[C@@H]3CCO3)C4)c2)c(F)c1]

2-((−8-(3-((4-cyano-2-fluorobenzyl)oxy)phenyl)-3,8-diazabicyclo[3.2.1]octan-3-yl)methyl)-1-(((S)-oxetan-2-yl)methyl)-1H-benzo[d]imidazole-6-carboxylic acid (2-35): The title compound was synthesized via following the synthesis of 2-18. white solid. LCMS (ESI, m/z): [M+H]⁺=582.2. ¹H NMR (400 MHZ, DMSO-d₆): δ 8.24 (s, 1H), 7.93-7.90 (m, 1H), 7.82-7.74 (m, 3H), 7.58 (d, J=8.4 Hz, 1H), 7.11-7.07 (m, 1H), 6.47-6.45 (m, 2H), 6.35-6.32 (m, 1H), 5.20 (s, 2H), 5.14-5.08 (m, 1H), 4.92-4.85 (m, 1H), 4.72-4.69 (m, 1H), 4.53-4.47 (m, 1H), 4.40-4.35 (m, 1H), 4.26-4.21 (m, 2H), 3.84-3.81 (m, 1H), 3.68-3.65 (m, 1H), 2.75-2.70 (m, 1H), 2.55-2.50 (m, 1H), 2.45-2.40 (m, 2H), 2.38-2.33 (m, 1H), 1.90-1.77 (m, 3H).

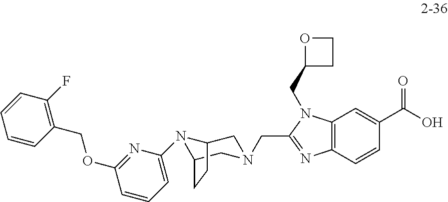 [SMILES: O=C(O)c1ccc2nc(CN3CC4CCC(C3)N4c3cccc(OCc4ccccc4F)n3)n(C[C@@H]3CCO3)c2c1]

2-((−8-(6-((2-fluorobenzyl)oxy)pyridin-2-yl)-3,8-diazabicyclo[3.2.1]octan-3-yl)methyl)-1-(((S)-oxetan-2-yl)methyl)-1H-benzo[d]imidazole-6-carboxylic acid (2-36): white solid. LCMS (ESI, m/z): [M+H]⁺=558.4. ¹H NMR (400 MHZ, DMSO-d₆): δ 8.26 (s, 1H), 7.82 (d, J=8.4 Hz, 1H), 7.60 (d, J=8.4 Hz, 1H), 7.50-7.42 (m, 2H), 7.38-7.34 (m, 1H), 7.22-7.17 (m, 2H), 6.28 (d, J=8.0 Hz, 1H), 6.05 (d, J=7.6 Hz, 1H), 5.31 (s, 2H), 5.12-5.08 (m, 1H), 4.89-4.86 (m, 1H), 4.73-4.69 (m, 1H), 4.51-4.37 (m, 4H), 3.85-3.81 (m, 1H), 3.68-3.65 (m, 1H), 2.77-2.73 (m, 1H), 2.59-2.57 (m, 1H), 2.44-2.38 (m, 3H), 2.34-2.31 (m, 1H), 1.89-1.76 (m, 4H).

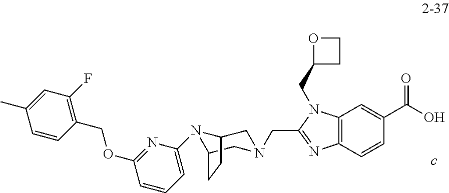 [SMILES: Cc1ccc(COc2cccc(N3C4CCC3CN(Cc3nc5ccc(C(=O)O)cc5n3C[C@@H]3CCO3)C4)n2)c(F)c1]

2-((−8-(6-((2-fluoro-4-methylbenzyl)oxy)pyridin-2-yl)-3,8-diazabicyclo[3.2.1]octan-3-yl)methyl)-1-(((S)-oxetan-2-yl)methyl)-1H-benzo[d]imidazole-6-carboxylic acid (2-37): white solid. LCMS (ESI, m/z): [M+H]⁺=572.3. ¹H NMR (400 MHZ, CD₃OD): δ 8.38 (s, 1H), 8.00-7.97 (m, 1H), 7.67 (d, J=8.4 Hz, 1H), 7.45-7.41 (m, 1H), 7.34-7.30 (m, 1H), 6.98-6.90 (m, 2H), 6.24 (d, J=8.0 Hz, 1H), 6.08 (d, J=7.6 Hz, 1H), 5.30-5.27 (m, 3H), 5.03-4.97 (m, 1H), 4.87-4.80 (m, 1H), 4.68-4.65 (m, 1H), 4.50-4.46 (m, 3H), 3.93-3.90 (m, 1H), 3.81-3.78 (m, 1H), 2.91-2.80 (m, 1H), 2.59-2.48 (m, 5H), 2.33 (s, 3H), 2.03-1.89 (m, 3H).

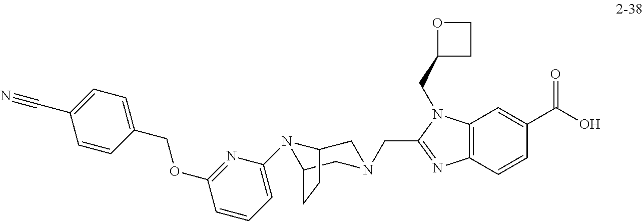 [SMILES: N#Cc1ccc(COc2cccc(N3C4CCC3CN(Cc3nc5ccc(C(=O)O)cc5n3C[C@@H]3CCO3)C4)n2)cc1]

2-((8-(6-((4-cyanobenzyl)oxy)pyridin-2-yl)-3,8-diazabicyclo[3.2.1]octan-3-yl)methyl)-1-(((S)-oxetan-2-yl)methyl)-1H-benzo[d]imidazole-6-carboxylic acid (2-38): white solid. LCMS (ESI, m/z): [M+H]⁺=565.4. ¹H NMR (400 MHZ, CD₃OD): δ 8.33 (s, 1H), 8.00-7.97 (m, 1H), 7.71-7.68 (m, 2H), 7.57 (d, J=6.8 Hz, 2H), 7.47-7.43 (m, 1H), 6.25-6.23 (m, 1H), 6.15-6.13 (m, 1H), 5.38 (s, 2H), 5.28-5.23 (m, 1H), 5.03-4.96 (m, 1H), 4.82-4.78 (m, 2H), 4.67-4.61 (m, 1H), 4.48-4.39 (m, 3H), 3.90-3.87 (m, 1H), 3.79-3.76 (m, 1H), 2.86-2.79 (m, 1H), 2.56-2.53 (m, 2H), 2.45-2.40 (m, 3H), 2.02-1.90 (m, 4H).

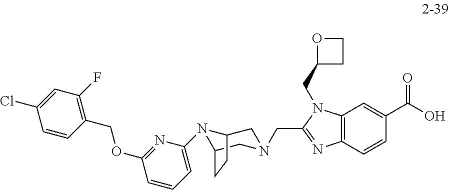 [SMILES: O=C(O)c1ccc2nc(CN3CC4CCC(C3)N4c3cccc(OCc4ccc(Cl)cc4F)n3)n(C[C@@H]3CCO3)c2c1]

2-[(8-{6-[(4-chloro-2-fluorophenyl)methoxy]pyridin-2-yl}-3,8-diazabicyclo[3.2.1]octan-3-yl)methyl]-3-[(2S)-oxetan-2-ylmethyl]-1,3-benzodiazole-5-carboxylic acid (2-39): white solid. LCMS (ESI, m/z): [M+H]⁺=592.3. ¹H NMR (400 MHZ, CD₃OD): δ 8.42 (s, 1H), 8.00-7.97 (m, 1H), 7.67 (d, J=8.4 Hz, 1H), 7.48-7.42 (m, 2H), 7.20 (d, J=8.4 Hz, 2H), 6.25 (d, J=8.0 Hz, 1H), 6.11 (d, J=8.0 Hz, 1H), 5.34 (s, 2H), 5.29-5.25 (m, 1H), 5.02-4.98 (m, 1H), 4.85-4.79 (m, 1H), 4.67-4.62 (m, 1H), 4.50-4.45 (m, 3H), 3.93-3.89 (m, 1H), 3.81-3.78 (m, 1H), 2.92-2.84 (m, 1H), 2.61-2.47 (m, 5H), 2.00-1.92 (m, 4H).

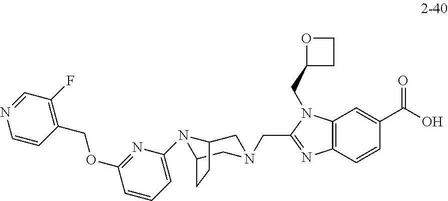 [SMILES: O=C(O)c1ccc2nc(CN3CC4CCC(C3)N4c3cccc(OCc4ccncc4F)n3)n(C[C@@H]3CCO3)c2c1]

2-((−8-(6-((3-fluoropyridin-4-yl)methoxy)pyridin-2-yl)-3,8-diazabicyclo[3.2.1]octan-3-yl)methyl)-1-(((S)-oxetan-2-yl)methyl)-1H-benzo[d]imidazole-6-carboxylic acid (2-40): white solid. LCMS (ESI, m/z): [M+H]⁺=559.3. ¹H-NMR (400 MHZ, CD₃OD): δ 8.42 (s, 1H), 8.36-8.34 (m, 2H), 8.00-7.97 (m, 1H), 7.67 (d, J=8.4 Hz, 1H), 7.53-7.45 (m, 2H), 6.26 (d, J=8.0 Hz, 1H), 6.18 (d, J=8.0 Hz, 1H), 5.46 (s, 2H), 5.27-5.25 (m, 1H), 4.97-4.95 (m, 1H), 4.85-4.78 (m, 1H), 4.69-4.65 (m, 1H), 4.47-4.37 (m, 3H), 3.90-3.86 (m, 1H), 3.79-3.75 (m, 1H), 2.87-2.77 (m, 1H), 2.63-2.47 (m, 2H), 2.45-2.37 (m, 3H), 2.03-1.87 (m, 4H).

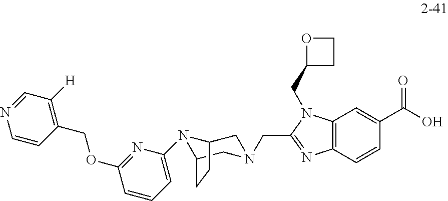 [SMILES: O=C(O)c1ccc2nc(CN3CC4CCC(C3)N4c3cccc(OCc4ccncc4)n3)n(C[C@@H]3CCO3)c2c1]

3-[(2S)-oxetan-2-ylmethyl]-2-({8-[6-(pyridin-4-ylmethoxy)pyridin-2-yl]-3,8-diazabicyclo[3.2.1]octan-3-yl}methyl)-1,3-benzodiazole-5-carboxylic acid (2-41): white solid. LCMS (ESI, m/z): [M+H]⁺=541.4. ¹H NMR (400 MHz, DMSO-d₆): δ 8.51 (s, 2H), 8.26 (s, 1H), 7.80 (d, J=8.4 Hz, 1H), 7.62 (d, J=8.4 Hz, 1H), 7.48-7.44 (m, 1H), 7.36 (d, J=5.2 Hz, 2H), 6.28 (d, J=8.0 Hz, 1H), 6.12 (d, J=7.6 Hz, 1H), 5.31 (s, 2H), 5.13-5.08 (m, 1H), 4.91-4.85 (m, 1H), 4.73-4.69 (m, 1H), 4.53-4.49 (m, 1H), 4.40-4.36 (m, 3H), 3.83-3.79 (m, 1H), 3.66-3.63 (m, 1H), 2.78-2.71 (m, 1H), 2.50-2.38 (m, 3H), 2.33-2.26 (m, 2H), 1.92-1.71 (m, 4H).

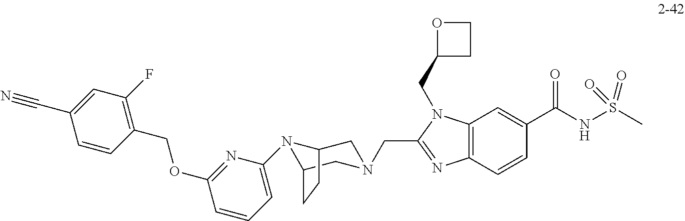 [SMILES: CS(=O)(=O)NC(=O)c1ccc2nc(CN3CC4CCC(C3)N4c3cccc(OCc4ccc(C#N)cc4F)n3)n(C[C@@H]3CCO3)c2c1]

2-((−8-(6-((4-cyano-2-fluorobenzyl)oxy)pyridin-2-yl)-3,8-diazabicyclo[3.2.1]octan-3-yl)methyl)-N-(methylsulfonyl)-1-(((S)-oxetan-2-yl)methyl)-1H-benzo[d]imidazole-6-carboxamide (2-42): To a solution of 2-[(8-{6-[(4-cyano-2-fluorophenyl)methoxy]pyridin-2-yl}-3,8-diazabicyclo[3.2.1]octan-3-yl)methyl]-3-[(2S)-oxetan-2-ylmethyl]-1,3-benzodiazole-5-carboxylic acid (2-29) (100.0 mg, crude) in CH₂Cl₂ (5.0 mL) was added 2-chloro-1-methylpyridin-1-ium iodide (52.6 mg, 0.21 mmol), methanesulfonamide (32.7 mg, 0.34 mmol), Et₃N (52.1 mg, 0.52 mmol) and DMAP (1.1 mg, 0.01 mmol) at room temperature. The resulting mixture was stirred at room temperature for 4 h. After the reaction was completed, the mixture was concentrated under reduced pressure. The residue was purified by reverse phase flash column chromatography with H₂O/ACN (90/10, v/v) and then purified by Prep-HPLC with the following conditions (Column: XSelect CSH Prep C18 OBD Column, 19×250 mm, 5 μm; Mobile Phase A: Water (10 mmol/L NH₄HCO₃), Mobile Phase B: MeOH-Preparative; Flow rate: 25 mL/min; Gradient: 52% B to 77% B in 10 min; Wave Length: 254 nm) to afford 2-((−8-(6-((4-cyano-2-fluorobenzyl)oxy)pyridin-2-yl)-3,8-diazabicyclo[3.2.1]octan-3-yl)methyl)-N-(methylsulfonyl)-1-(((S)-oxetan-2-yl)methyl)-1H-benzo[d]imidazole-6-carboxamide (6.3 mg, 5%) as a white solid. LCMS (ESI, m/z): [M+H]⁺=660.3. ¹H NMR (400 MHZ, DMSO-d₆): δ 12.13 (s, 1H), 8.24-8.20 (m, 1H), 7.87-7.81 (m, 2H), 7.71-7.58 (m, 3H), 7.48-7.44 (m, 1H), 6.30 (d, J=8.0 Hz, 1H), 6.10 (d, J=8.0 Hz, 1H), 5.40 (s, 2H), 5.16-5.10 (m, 1H), 4.90-4.84 (m, 1H), 4.70-4.65 (m, 1H), 4.54-4.49 (m, 1H), 4.43-4.38 (m, 3H), 3.84-3.80 (m, 1H), 3.66-3.63 (m, 1H), 3.32 (s, 3H), 2.79-2.71 (m, 1H), 2.57-2.50 (m, 1H), 2.41-2.38 (m, 2H), 2.33-2.27 (m, 2H), 1.91-1.79 (m, 4H).

### Example 3

2-(((1R,3s,5S)-8-(6-((4-cyano-2-fluorobenzyl)oxy)pyridin-2-yl)-8-azabicyclo[3.2.1]octan-3-yl)methyl)-1-(((S)-oxetan-2-yl)methyl)-1H-benzo[d]imidazole-6-carboxylic acid (Compound 3)

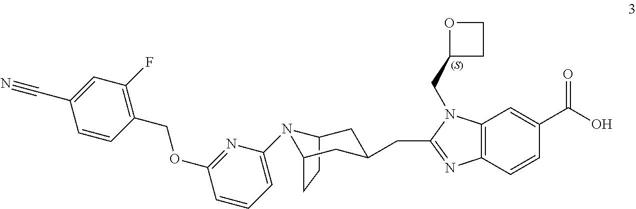

The title compound may be synthesized according to the synthetic route below.

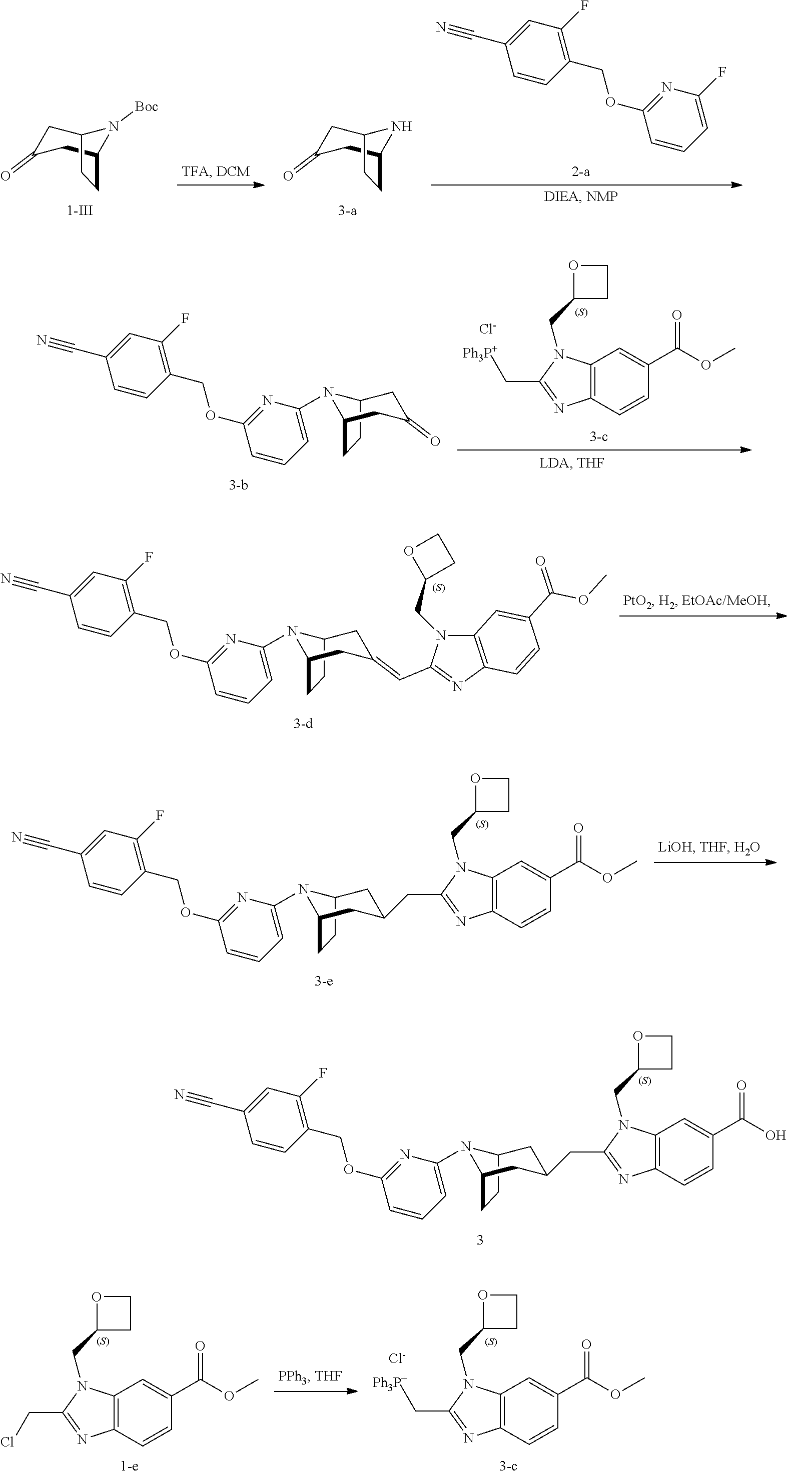

Following the synthetic route described above for Example 3 and substituting the appropriate reagents, starting materials and purification methods known to those skilled in the art, the compounds listed below may be synthesized.

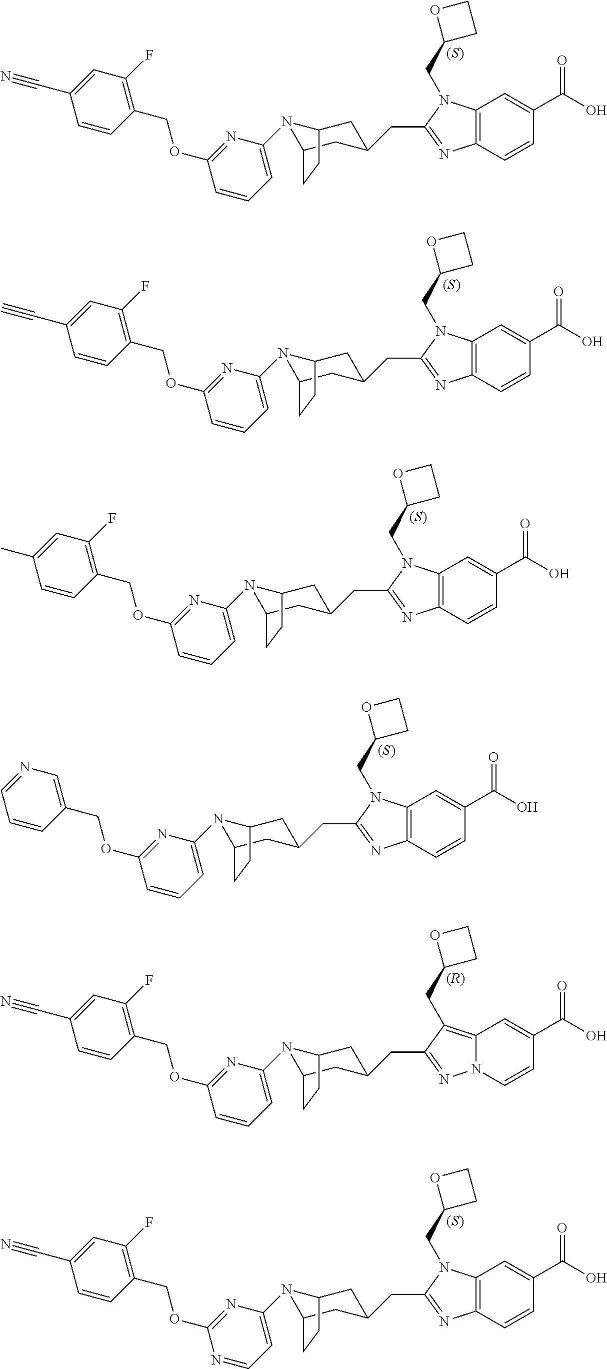

### Example 4

### (S)-2-((2-(6-((4-cyano-2-fluorobenzyl)oxy)pyridin-2-yl)-1,3-dioxan-5-yl)methyl)-1-(oxetan-2-ylmethyl)-1H-benzo[d]imidazole-6-carboxylic acid (Compound 4)

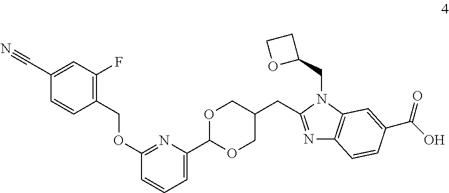 [SMILES: N#Cc1ccc(COc2cccc(C3OCC(Cc4nc5ccc(C(=O)O)cc5n4C[C@@H]4CCO4)CO3)n2)c(F)c1]

The title compound may be synthesized according to the synthetic route below.

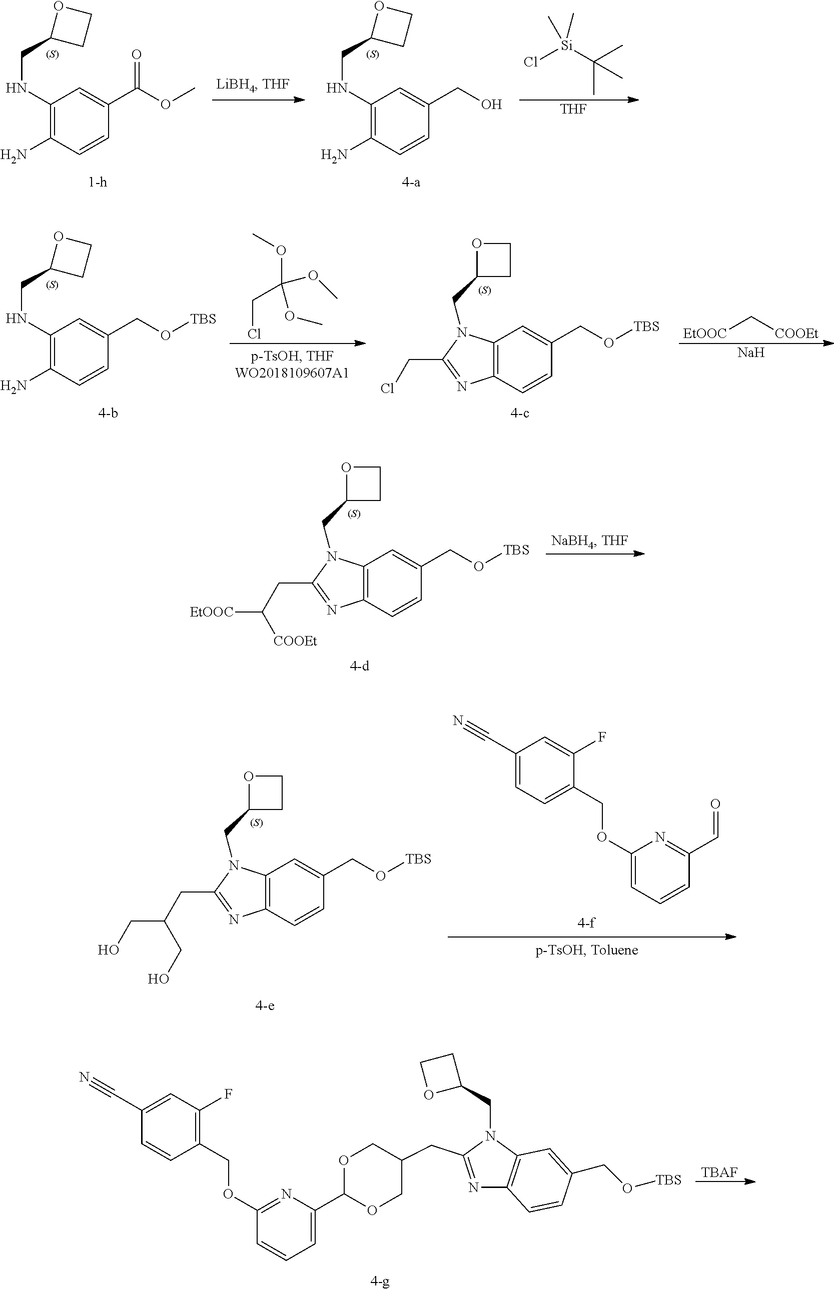

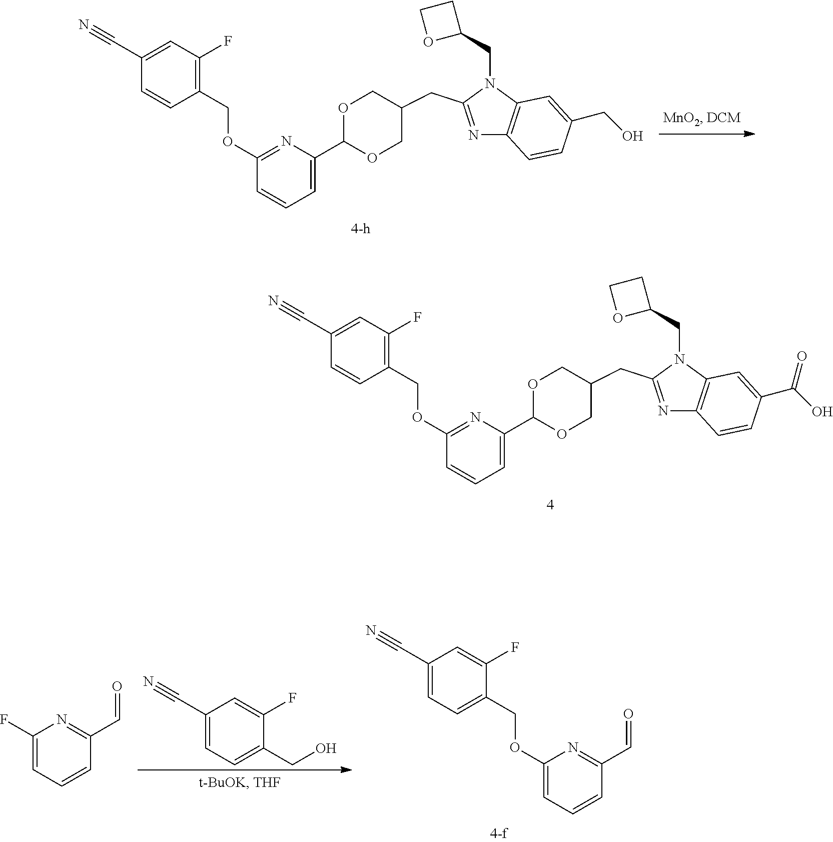

Following the synthetic route described above for Example 4 and substituting the appropriate reagents, starting materials and purification methods known to those skilled in the art, the compounds listed below may be synthesized.

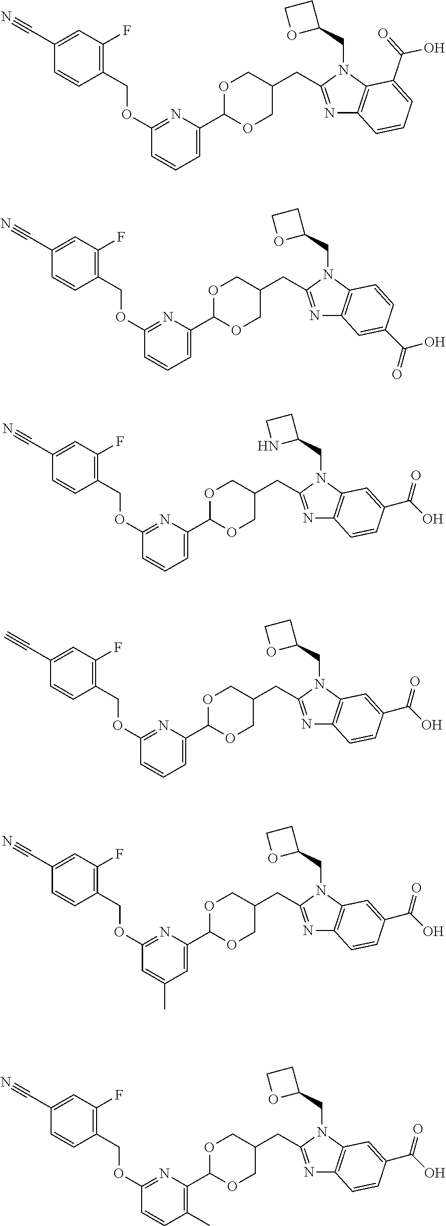 [SMILES: N#Cc1ccc(COc2cccc(C3OCC(Cc4nc5cccc(C(=O)O)c5n4C[C@@H]4CCO4)CO3)n2)c(F)c1 ; N#Cc1ccc(COc2cccc(C3OCC(Cc4nc5cc(C(=O)O)ccc5n4C[C@@H]4CCO4)CO3)n2)c(F)c1 ; N#Cc1ccc(COc2cccc(C3OCC(Cc4nc5ccc(C(=O)O)cc5n4C[C@@H]4CCN4)CO3)n2)c(F)c1 ; C#Cc1ccc(COc2cccc(C3OCC(Cc4nc5ccc(C(=O)O)cc5n4C[C@@H]4CCO4)CO3)n2)c(F)c1 ; Cc1cc(OCc2ccc(C#N)cc2F)nc(C2OCC(Cc3nc4ccc(C(=O)O)cc4n3C[C@@H]3CCO3)CO2)c1 ; Cc1ccc(OCc2ccc(C#N)cc2F)nc1C1OCC(Cc2nc3ccc(C(=O)O)cc3n2C[C@@H]2CCO2)CO1]

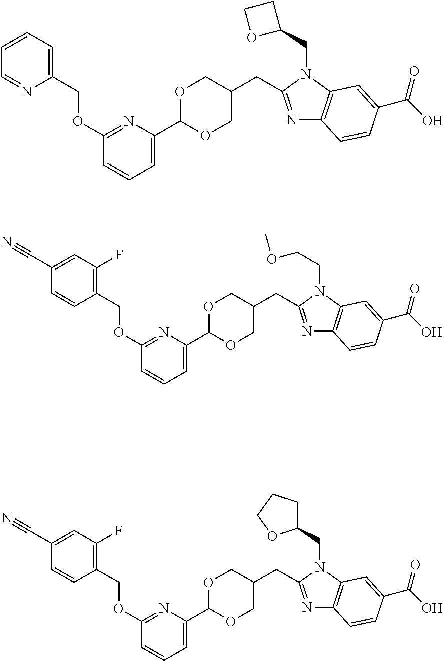 [SMILES: O=C(O)c1ccc2nc(CC3COC(c4cccc(OCc5ccccn5)n4)OC3)n(C[C@@H]3CCO3)c2c1 ; COCCn1c(CC2COC(c3cccc(OCc4ccc(C#N)cc4F)n3)OC2)nc2ccc(C(=O)O)cc21 ; N#Cc1ccc(COc2cccc(C3OCC(Cc4nc5ccc(C(=O)O)cc5n4C[C@@H]4CCCO4)CO3)n2)c(F)c1]

### Example 5

### (S)-2-((4-(3-(benzyloxy)phenyl) bicyclo[2.2.2]octan-1-yl)methyl)-1-(oxetan-2-ylmethyl)-1H-benzo[d]imidazole-6-carboxylic acid (Compound 5)

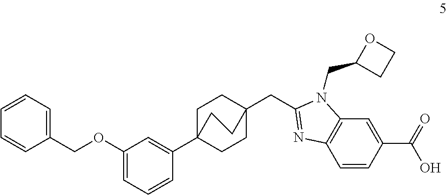 [SMILES: O=C(O)c1ccc2nc(CC34CCC(c5cccc(OCc6ccccc6)c5)(CC3)CC4)n(C[C@@H]3CCO3)c2c1]

The title compound may be synthesized according to the synthetic route below.

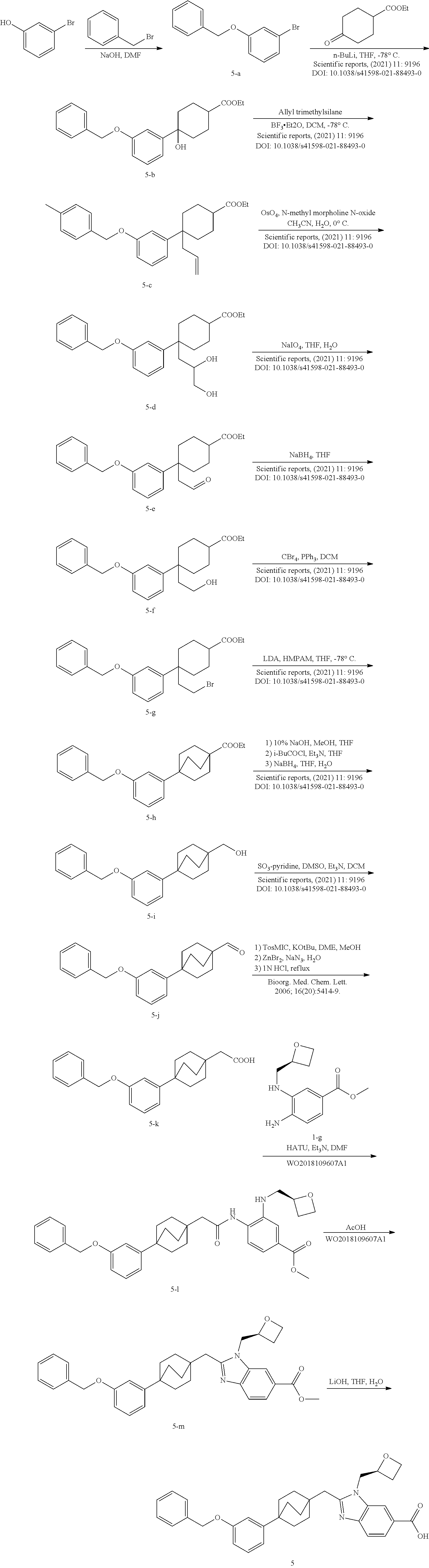

Following the synthetic route described above for Example 5 and substituting the appropriate reagents, starting materials and purification methods known to those skilled in the art, the compounds listed below may be synthesized.

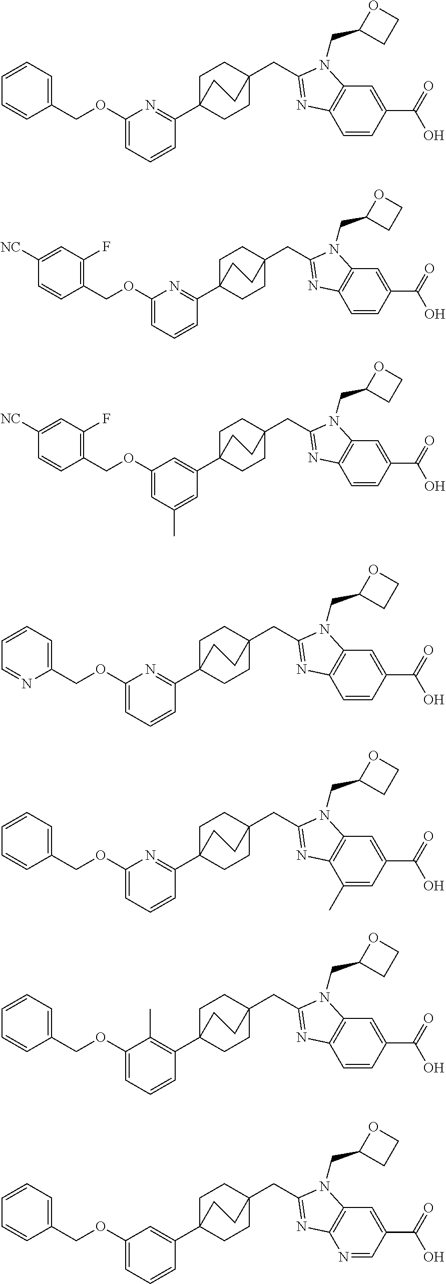

### Example 6. Synthesis of Compound 6-1

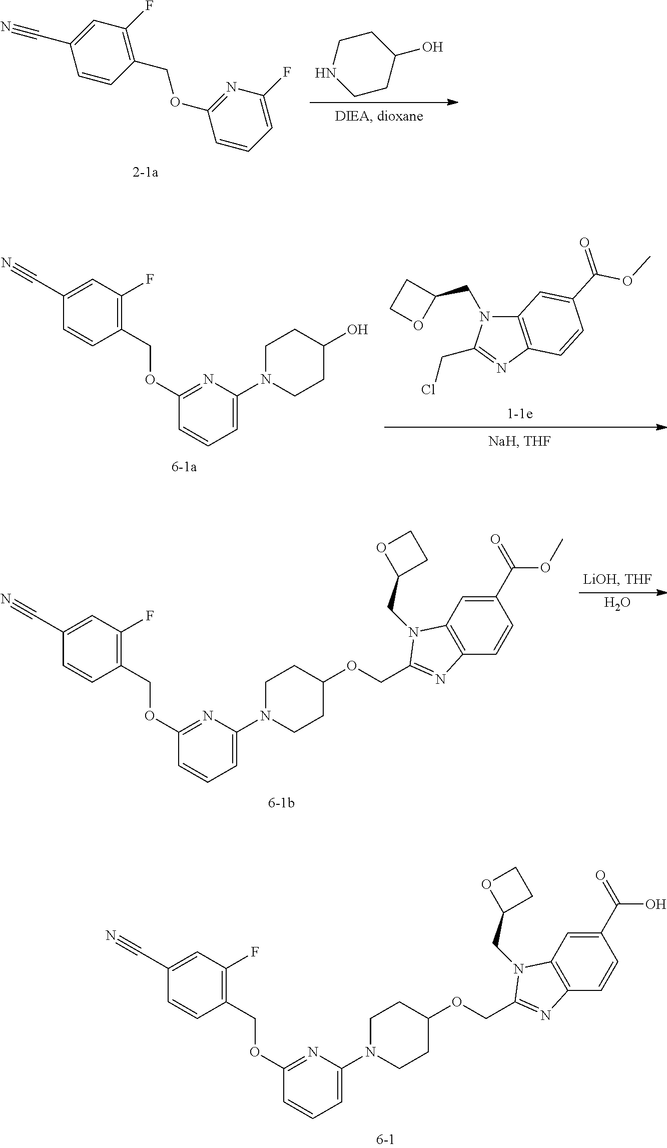

3-fluoro-4-({[6-(4-hydroxypiperidin-1-yl)pyridin-2-yl]oxy}methyl)benzonitrile (6-1a): To a solution of 3-fluoro-4-{[(6-fluoropyridin-2-yl)oxy]methyl}benzonitrile (2.0 g, 8.12 mmol) in dioxane (15.0 mL) was added piperidin-4-ol (986.0 mg, 9.75 mmol) and DIEA (2.1 g, 16.25 mmol) at room temperature. The resulting mixture was stirred at 100° C. for 16 h. After the reaction was completed, the resulting mixture was concentrated under vacuum. The residue was purified by flash chromatography with CH₂Cl₂/CH₃OH (94/6, v/v) to afford 3-fluoro-4-({[6-(4-hydroxypiperidin-1-yl)pyridin-2-yl]oxy}methyl)benzonitrile (500.0 mg, 19%) as a yellow solid. LCMS (ESI, m/z): [M+H]⁺=328.1.

Methyl 2-{[(1-{6-[(4-cyano-2-fluorophenyl)methoxy]pyridin-2-yl}piperidin-4-yl)oxy]methyl}-3-[(2S)-oxetan-2-ylmethyl]-1,3-benzodiazole-5-carboxylate (6-1b): To a solution of 3-fluoro-4-({[6-(4-hydroxypiperidin-1-yl)pyridin-2-yl]oxy}methyl)benzonitrile (500 mg, 1.53 mmol) in THF (20.0 mL) was added NaH (305.0 mg, 60% purity) at room temperature under N₂. The resulting mixture was stirred at 40° C. for 0.5 h under N₂. Then methyl 2-(chloromethyl)-3-[(2S)-oxetan-2-ylmethyl]-1,3-benzodiazole-5-carboxylate (900.4 mg, 3.06 mmol) was added to the mixture. The resulting mixture was stirred at 40° C. for 1 h.

After the reaction was completed, the resulting mixture was cooled to room temperature and quenched by the addition of NH₄Cl (aq) at room temperature. The resulting mixture was extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (90/10, v/v) to afford methyl 2-{[(1-{6-[(4-cyano-2-fluorophenyl)methoxy]pyridin-2-yl}piperidin-4-yl)oxy]methyl}-3-[(2S)-oxetan-2-ylmethyl]-1,3-benzodiazole-5-carboxylate (100.0 mg, 11%) as a yellow solid. LCMS (ESI, m/z): [M+H]⁺==586.2.

2-{[(1-{6-[(4-cyano-2-fluorophenyl)methoxy]pyridin-2-yl}piperidin-4-yl)oxy]methyl}-3-[(2S)-oxetan-2-ylmethyl]-1,3-benzodiazole-5-carboxylic acid (6-1): To a solution of methyl 2-{[(1-{6-[(4-cyano-2-fluorophenyl)methoxy]pyridin-2-yl}piperidin-4-yl)oxy]methyl}-3-[(2S)-oxetan-2-ylmethyl]-1,3-benzodiazole-5-carboxylate (50.0 mg, 0.09 mmol) in THF (3.0 mL) and H₂O (2.0 mL) was added LiOH (21.0 mg, 0.88 mmol) at room temperature. The resulting mixture was stirred at 30° C. for 16 h. After the reaction was completed, the pH value of the mixture was adjusted to 5.0 with CH₃COOH. The mixture was extracted with ethyl acetate. The combined organic layer was washed with brine, dried over anhydrous sodium sulfate and filtered. The filtrate was concentrated under vacuum. The residue was purified by Prep-HPLC with the following conditions (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 29% B to 39% B in 8 min; Wave Length: 254 nm) to afford 2-{[(1-{6-[(4-cyano-2-fluorophenyl)methoxy]pyridin-2-yl}piperidin-4-yl)oxy]methyl}-3-[(2S)-oxetan-2-ylmethyl]-1,3-benzodiazole-5-carboxylic acid (10.4 mg, 21%) as a white solid. LCMS (ESI, m/z): [M+H]⁺=572.2. ¹H NMR (400 MHZ, DMSO-d₆): δ 8.27 (s, 1H), 7.90-7.82 (m, 2H), 7.72-7.63 (m, 3H), 7.48-7.44 (m, 1H), 6.37 (d, J=8.0 Hz, 1H), 6.10 (d, J=7.6 Hz, 1H), 5.41 (s, 2H), 5.11-5.05 (m, 1H), 4.93-4.83 (m, 2H), 4.70-4.33 (m, 4H), 3.88-3.72 (m, 3H), 3.14-3.09 (m, 2H), 2.69-2.65 (m, 1H), 2.44-2.39 (m, 1H), 1.92-1.84 (m, 2H), 1.44-1.38 (m, 2H).

### Example 7. Synthesis of Compound 7-1

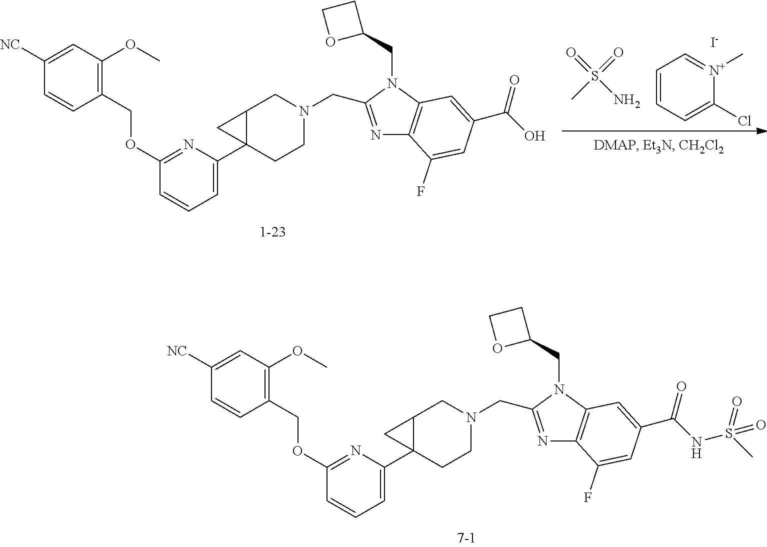

To a solution of 2-((6-(6-((4-cyano-2-methoxybenzyl)oxy)pyridin-2-yl)-3-azabicyclo[4.1.0]heptan-3-yl)methyl)-4-fluoro-1-(((S)-oxetan-2-yl)methyl)-1H-benzo[d]imidazole-6-carboxylic acid (1-23) (100.0 mg, 0.17 mmol) in CH₂Cl₂ (5.0 mL) was added methanesulfonamide (31.8 mg, 0.33 mmol), 2-chloro-1-methylpyridin-1-ium iodide (51.3 mg, 0.20 mmol), DMAP (1.0 mg, 0.01 mmol) and Et₃N (33.9 mg, 0.33 mmol) at room temperature. The resulting mixture was stirred at room temperature for 16 h. After the reaction was completed, the mixture was diluted with water and extracted with CH₂Cl₂. The combined organic layer was washed with water, dried over anhydrous sodium sulfate and filtered. The filtrate was concentrated under vacuum. The residue was purified by Prep-HPLC with the following conditions (Column: XBridge Shield RP18 OBD Column, 30×150 mm, 5 μm; Mobile Phase A: Water (10 mmol/L NH₄HCO₃), Mobile Phase B: ACN; Flow rate: 60 mL/min; Gradient: 35% B to 45% B in 10 min; Wave Length: 254 nm) to afford 2-((6-(6-((4-cyano-2-methoxybenzyl)oxy)pyridin-2-yl)-3-azabicyclo[4.1.0]heptan-3-yl)methyl)-4-fluoro-N-(methylsulfonyl)-1-(((S)-oxetan-2-yl)methyl)-1H-benzo[d]imidazole-6-carboxamide (7-1) (53.7 mg, 48%) as a white solid. LCMS (ESI, m/z): [M+H]⁺=675.3. ¹H NMR (400 MHZ, CD₃OD): δ 8.14 (s, 1H), 7.74-7.68 (m, 1H), 7.57-7.53 (m, 1H), 7.46 (d, J=7.6 Hz, 1H), 7.32 (s, 1H), 7.26 (d, J=7.6, 1H), 6.89 (d, J=7.6 Hz, 1H), 6.60 (d, J=8.0 Hz, 1H), 5.43-5.34 (m, 2H), 5.23-5.19 (m, 1H), 4.68-4.55 (m, 2H), 4.47-4.38 (m, 1H), 3.98-3.77 (m, 5H), 3.11 (s, 3H), 2.93-2.83 (m, 1H), 2.81-2.71 (m, 2H), 2.57-2.44 (m, 2H), 2.39-2.34 (m, 2H), 2.07-1.99 (m, 1H), 1.75-1.68 (m, 1H), 1.13-1.08 (m, 1H), 0.94-0.89 (m, 1H).

### Biological Example 1

### Assay 1. GLP1R CAMP Assay for Demonstrating Small Molecule Compound-Mediated GLP1R Activation

GLP1R-mediated agonist activity was determined with a cell-based functional assay utilizing an HTRF (Homogeneous Time-Resolved Fluorescene) cAMP detection kit that measures CAMP levels in the cell. Reagents and equipment used in the assay are listed below, followed by the protocol.

| Materials and Reagents | Vendor | Cat# |
|---|---|---|
| Fetal Bovine Serum, 500ml | Gibco | 10099141C |
| DMEM | Gibco | 11965-092 |
| Penicillin-Streptomycin, Liquid, | Gibco | 15140122 |
| 100ml (100X) | | |
| Hygromycin B Gold (solution) 5 g | Invivogen | ant-hg-5 |
| (1 × 50 ml, 100mg/ml) | | |
| BSA stabilizer 7.5%, 50ml | Perkin Elmer | CR84-100 |
| cAMP Kit | Perkin Elmer | TRF0263 |
| IBMX | Sigma | I5879 |
| HEPES, 1M, 100ml | Gibco | 15630080 |
| CulturPlate-384, White Opaque 384-well | Perkin Elmer | 6007680 |
| Microplate, Sterile and Tissue , | | |
| Culture Treated | | |
| includes lid, 50 plates/case | | |
| 96-well conical btm PP Plt nature | Thermo | 249944 |
| RNASE/Dnase- | Fisher | |
| free plate (120 plates/case) | | |
| Consumables and Instrument | Vendor | Cat# or |
| | | Model |
| En Vision | Perkin Elmer | 2105 |

### Protocol:

### 1. Cell Culture and Reagent Preparation

1) Cell Line: Flp-In-293-GLP1R (constructed by Pharmaron Beijing Co., Ltd)
2) Complete Medium: DMEM+10% FBS+1×Penicillin-Streptomycin+200 μg/mL HB
3) Assay Buffer: 1×HBSS+20 mM HEPES+0.1% BSA+500 μM IBMX

### 2. Agonist Activity Test

a) Seed Flpin-293-GLP1R cells at 20,000 cells/well into 384 well assay plate (6007680-50, PE) using complete medium.
b) Prepare 4×compound working solution with assay buffer.
c) Add 5 μL 4×compound working solution into cell plate, incubate 30 min at 37° C.
d) Dilute Eu-cAMP tracer (1/50) with lysis buffer and add 10 μl/well to assay plate.
e) Dilute Ulight-anti-cAMP (1/150) with lysis buffer and add 10 μl/well to assay plate.
f) Incubate 1 h at RT.
g) Read the plate at wavelengths of 665 nm and 615 nm on Envision 2105 plate reader.

### 3. Date Analysis

3.1% Activity is calculated as follow:

% Activity=100−(Signal_{cmpd}−Signal_{Ave_PC})/(Signal_{Ave_VC}−Signal_{Ave_PC})×100.
3.2 Calculate EC₅₀ and Plot effect-dose curve of cmpds:

Y=Bottom+(Top−Bottom)/(1+10\hat{ }((Log EC₅₀−X)*Hill Slope))
X: log of agonist concentration; Y: % Activity.

### Assay 2. GLP1R/CRE-Luc Assay for Demonstrating Small Molecule Compound-Mediated GLP1R Activation

GLP1R-mediated agonist activity was determined with a cell-based functional assay utilizing a britelite plus luciferase reporter gene assay system. The stable cell line overexpress GLP1R and target response element. The stimulation with GLP1R agonist results in the upregulation of intracellular cAMP, which regulate the activities of cAMP-response element binding protein (CREB) and CREB-Luc. Reagents and equipment used in the assay are listed below, followed by the protocol.

| Materials and Reagents | Vendor | Cat# |
|---|---|---|
| Fetal Bovine Serum, 500ml | AUSGeneX | FBS500-S |
| DMEM | Gibco | 10566-016 |
| HKE293-Human GLP1R/CRE-luc | Cobioer | CBP71117 |
| Penicillin-Streptomycin, Liquid, 100ml | Gibco | 15140122 |
| (100X) | | |
| G418 | Invitrogen | 10131-027 |
| britelite plus Luciferase Assay System | PE | 6066769 |
| CulturPlate-384, White Opaque 384-well | Perkin Elmer | 6007680 |
| Microplate, Sterile and Tissue Culture | | |
| Treated, includes lid, 50 plates/case | | |
| 96-well conical btm PP Plt nature | Thermo Fisher | 249944 |
| RNASE/Dnase-free plate (120 plates/case) | | |
| Consumables and Instrument | Vendor | Cat# or Model |
| En Vision | Perkin Elmer | 2105 |

### Protocol:

### 1. Cell Culture and Reagent Preparation

1) Cell Line: HKE293-Human GLP1R/CRE-luc
2) Complete Medium: DMEM+10% FBS+1×Penicillin-Streptomycin+400 μg/mL G418

### 2. Agonist Activity Test

a) Seed HKE293-Human GLP1R/CRE-luc cells at 20,000 cells/well into 384 well assay plate (6007680-50, PE) using complete medium and incubate overnight.
b) Prepare 6×compound working solution with Medium.
c) Add 5 μL 6×compound working solution into cell plate, incubate 5 h at 37° C.
d) Add 20 μl britelite plus Luciferase Assay Reagent into each well of 384-well assay plate.
e) Record the luminescence value on Envision plate reader.

### 3. Date Analysis

3.1% Activity is calculated as follow:

% Activity=(Signal_{cmpd}−Signal_{Ave_VC})/(Signal_{Ave_PC}−Signal_{Ave_VC})×100.
3.2 Calculate EC₅₀ and Plot effect-dose curve of cmpds:

Y=Bottom+(Top−Bottom)/(1+10\hat{ }((Log EC₅₀−X)*HillSlope))
X: log of agonist concentration; Y: % Activity.

### Biological Data

Activity data for GLP1R agonists are displayed in Table 2. A blank cell means there was no data for that Compound.

TABLE 2
Compound EC₅₀ values

| Compound | EC₅₀ (nM) | | Compound | EC₅₀ (nM) | |
|---|---|---|---|---|---|
| ID | Assay 1 | Assay 2 | ID | Assay 1 | Assay 2 |
| 1-1 | 0.029 | 80.8 | 2-41 | >4 | |
| 1-1-A | 0.028 | 48.72 | 2-42 | >4 | |
| 1-1-B | 0.035 | 84.91 | 2-4 | >4 | |
| 1-2-A | >4 | | 2-5 | >4 | |
| 1-2-B | >4 | | 2-6 | 0.643 | |
| 1-11 | 0.079 | | 2-7 | >4 | |
| 1-11-A | 0.995 | | 2-8 | >4 | |
| 1-11-B | 0.017 | | 2 | 1.216 | |
| 1-8 | 0.035 | 107.70 | 2-9 | >4 | |
| 1-5 | 0.030 | 100.40 | 2-10 | >4 | |
| 1-6 | 0.003 | | 2-11 | >4 | |
| 1-7 | 0.175 | | 2-12 | >4 | |
| 1-9-A | 1.007 | | 2-13 | >4 | |
| 1-9-B | 1.046 | | 2-14 | >4 | |
| 1-10 | 0.047 | 31.22 | 2-15 | >4 | |
| 1-10-A | 0.009 | 19.31 | 2-16 | >4 | |
| 1-10-B | 0.021 | 47.58 | 2-17 | >4 | |
| 6-1 | 0.888 | | 2-18 | 1.174 | |
| 2-29 | 0.287 | >1000 | 2-2 | >4 | |
| 2-30 | 0.375 | | 2-3 | >4 | |
| 2-31 | 1.499 | | 2-19 | >4 | |
| 2-32 | 3.252 | | 2-20 | 1.842 | |
| 2-33 | >4 | | 2-21 | >4 | |
| 2-34 | >4 | >1000 | 2-22 | 1.995 | |
| 2-35 | 2.788 | >1000 | 2-23 | >4 | |
| 2-36 | >4 | | 2-24 | 3.05 | |
| 2-37 | 0.929 | | 2-25 | 1.696 | |
| 2-38 | 0.458 | | 2-26 | >4 | |
| 2-39 | 0.346 | | 2-27 | >4 | |
| 2-40 | >4 | | 2-28 | >4 | |
| 1-26-A | | 102.3 | | | |
| 1-27 | | 11.68 | | | |
| 1-29 | | 168.2 | | | |
| 1-31 | | 132.0 | 1-12 | | 26.35 |
| 1-33 | | 105.4 | 1-13 | | 35.82 |
| 1-35 | | 2.68 | 1-14 | | 17.4 |
| 1-37 | | 496.5 | 1-15 | | 438.7 |
| 1-6-A | | 5.55 | 1-16 | | 24.83 |
| 1-6-B | | 4.98 | 1-17 | | 6.66 |
| 1-18 | | 13.4 | 1-19 | | 19.04 |
| 1-20 | | 9.67 | 1-21 | | 39.08 |
| 1-22 | | 14.43 | 1-23 | | 3.05 |
| 1-24 | | 0.37 | 1-25 | | 0.95 |
| 1-28 | | 0.46 | 1-26-B | | 7.54 |
| 1-30 | | 28.07 | 1-32 | | 0.49 |
| 1-34 | | 1.06 | 1-36 | | 9.38 |
| 7-1 | | 266.1 | 1-38 | | 0.79 |
| 1-38-A | | 16.0 | 1-38-B | | 0.28 |
| 1-39 | | 29.01 | 1-40 | | 22.06 |
| 1-41 | | 0.34 | 1-42 | | 6.5 |
| 1-43 | | 0.49 | 1-44 | | 0.30 |
| 1-45 | | 0.16 | 1-46 | | 2.60 |
| 1-47 | | 3.85 | 1-48 | | 0.33 |
| 1-49 | | 10.2 | | | |

The foregoing description is considered as illustrative only of the principles of the present disclosure. Further, since numerous modifications and changes will be readily apparent to those skilled in the art, it is not desired to limit the invention to the exact construction and process shown as described above. Accordingly, all suitable modifications and equivalents may be considered to fall within the scope of the present invention as defined by the claims that follow.

1. A compound of Formula I 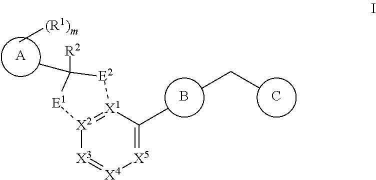
or a pharmaceutically acceptable salt thereof, wherein
ring A is a 5- or 6-membered aryl or heteroaryl group;
each R¹ is independently halogen, —OH, —CN, —C≡CH, —S(O)—C₁₋₃alkyl, —S(O)₂—C₁₋₃alkyl, —P(O)—(C₁₋₃alkyl)₂, —C₃₋₆cycloalkyl, a 3- to 6-membered heterocycloalkyl group, a 5- or 6-membered heteroaryl group, —C₁₋₃alkyl, —C₁₋₃alkylOC₁₋₃alkyl, or —OC₁₋₃alkyl, wherein said alkyl of —C₁₋₃alkyl, —C₁₋₃alkylOC₁₋₃alkyl, and —OC₁₋₃alkyl is substituted with 0 to 5 halogen atoms;
or two R¹ taken together with the carbon atom to which they are attached form a cycloalkyl or heterocyclyl;
m is 0, 1, 2, 3, or 4;
E¹ and E² are independently H, D, halogen, O, NH, or CH₂;
X¹ and X² are independently N or CR⁶, R⁶ is independently absent, H, halogen, —C₁₋₃alkyl or —CN;
X³, X⁴ and X⁵ are independently N or CR⁷, wherein R⁷ is independently H, halogen, —C₁₋₃alkyl, —OC₁₋₃alkyl or —CN, wherein said alkyl of —C₁₋₃alkyl and —OC₁₋₃alkyl is substituted with 0 to 3 halogen atoms;
--- denotes the presence or absence of a bond; provided that,
a) when the --- between E¹ and X² denotes the absence of a bond, then the --- between E² and X¹ denotes the presence of a bond, E¹ is H, D, or halogen, and X¹ is C,
b) when the --- between E² and X¹ denotes the absence of a bond, then the --- between E¹ and X² denotes the presence of a bond, E² is H, D, or halogen, and X² is C, and
c) when the --- between E¹ and X² and the --- between E² and X¹ both denote the presence of a bond, then E¹ and E² are independently O, NH or CH₂, and X¹ and X² are C;
R² is independently H, D, halogen, or —C₁₋₃alkyl;
ring B is a 6- to 8-membered cycloalkylene, cycloalkenylene, heterocycloalkylene or heterocycloalkenylene group substituted as valency allows with 0 to 3 substituents independently selected from 0 to 3 halogen atoms and 0 to 1 oxo (═O), and may be further substituted by 0, 1 or 2 substituent R, wherein each R is independently H, halogen, —CN or C₁₋₃ alkyl;
ring C is 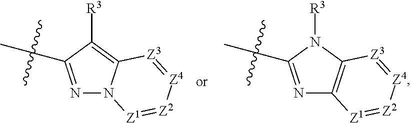
wherein Z¹, Z², Z³ and Z⁴ are independently N, CR⁴ or CR⁸, wherein R⁸ is independently H, —OH, CN, halogen, —C(O) C₁₋₃alkyl, —C(O) C₃-6cycloalkyl, —OC₁₋₃alkyl, —C₃₋₆cycloalkyl, or —C₁₋₃alkyl, wherein said alkyl and said cycloalkyl of —C(O)C₁₋₃alkyl, —C(O) C₃₋₆cycloalkyl, —OC₁₋₃alkyl, —C₃₋₆cycloalkyl, and —C₁₋₃alkyl are independently unsubstituted or substituted with one or more substituents selected from D, OH, NH₂, —CN, and halogen; provided that one of Z¹, Z², Z³ and Z⁴ is CR⁴;
R³ is —C₁₋₃alkyl, —C₀₋₃alkylene-C₃₋₆cycloalkyl, or —C₀₋₃alkylene-R⁵, wherein said alkyl may be substituted as valency allows with 0 to 3 substituents independently selected from 0 to 3 halogen atoms and 0 to 1 substituent selected from —C₀₋₃alkylene-CN, —C₀₋₁alkylene-OR⁹, and —N(R¹⁰)₂, and wherein said alkylene and cycloalkyl may be independently substituted as valency allows with 0 to 2 substituents independently selected from 0 to 2 halogen atoms and 0 to 1 substituent selected from —C₀₋₁alkylene-CN, —C₀₋₁alkylene-OR⁹, and —N(R¹⁰)₂;
R⁵ is a 5- or 6-membered heteroaryl group, or a 4- to 6-membered heterocycloalkyl group, wherein said heteroaryl and heterocycloalkyl may be substituted with 0 to 2 substituents as valency allows independently selected from:
0 to 1 oxo (═O),
0 to 1 —CN,
0 to 2 halogen atoms, and
0 to 2 substituents independently selected from —C₁₋₃alkyl, —OC₁₋₃alkyl and —C₁₋₃alkylene-O—C₁₋₃alkyl wherein the alkyl of —C₁₋₃alkyl and —OC₁₋₃alkyl may be substituted with 0 to 3 substituents as valency allows independently selected from 0 to 3 halogen atoms, 0 to 1 —CN, and 0 to 1 —OR⁹;
each R⁹ is independently H, or —C₁₋₃alkyl, wherein-C₁₋₃alkyl may be substituted with 0 to 3 halogen atoms;
each R¹⁰ is independently H, or —C₁₋₃alkyl; and
R⁴ is COOH or a carboxylic group surrogate, and particularly, the carboxylic group surrogate is:

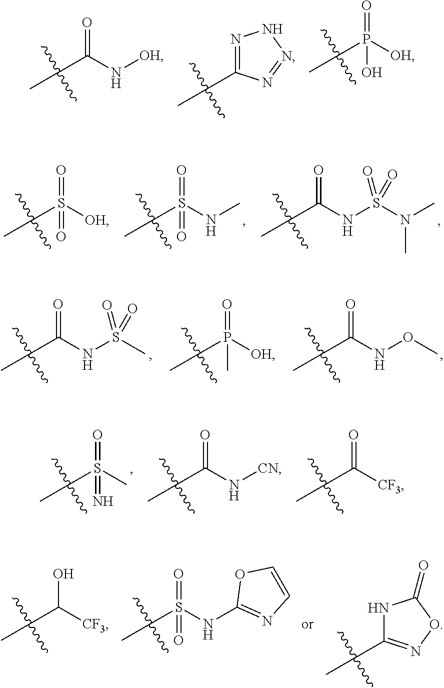

2. A compound of Formula I 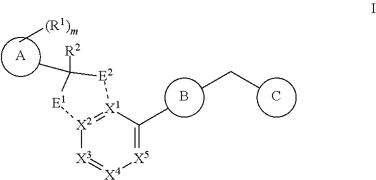
or a pharmaceutically acceptable salt thereof as claimed in claim 1, wherein
ring A is a 5- or 6-membered aryl or heteroaryl group;
each R¹ is independently halogen, —CN, —C≡CH, —S(O)—C₁₋₃alkyl, —S(O)₂—C₁₋₃alkyl, —P(O)—(C₁₋₃alkyl)₂, —C₃₋₆cycloalkyl, a 3- to 6-membered heterocycloalkyl group, a 5- or 6-membered heteroaryl group, —C₁₋₃alkyl, or —OC₁₋₃alkyl, wherein said alkyl of —C₁₋₃alkyl and —OC₁₋₃alkyl is substituted with 0 to 5 halogen atoms;
m is 0, 1, 2 or 3;
E¹ and E² are independently H, O, NH, or CH₂;
X¹ and X² are independently N or CR⁶, R⁶ is independently absent, H, halogen, —C₁₋₃alkyl or —CN;
X³, X⁴ and X⁵ are independently N or CR⁷, wherein R⁷ is independently H, halogen, —C₁₋₃alkyl, —OC₁₋₃alkyl or —CN, wherein said alkyl of —C₁₋₃alkyl and —OC₁₋₃alkyl is substituted with 0 to 3 halogen atoms;
--- denotes the presence or absence of a bond; provided that,
a) when the --- between E¹ and X² denotes the absence of a bond, then the --- between E² and X¹ denotes the presence of a bond, E¹ is H, and X¹ is C,
b) when the --- between E² and X¹ denotes the absence of a bond, then the --- between E¹ and X² denotes the presence of a bond, E² is H, and X² is C, and
c) when the --- between E¹ and X² and the --- between E² and X¹ both denote the presence of a bond, then E¹ and E² are independently O, NH or CH₂, and X¹ and X² are C;
R² is independently H or —C₁₋₃alkyl;
ring B is a 6- to 8-membered cycloalkylene, cycloalkenylene, heterocycloalkylene or heterocycloalkenylene group substituted as valency allows with 0 to 3 substituents independently selected from 0 to 3 halogen atoms and 0 to 1 oxo (═O), and may be further substituted by 0, 1 or 2 substituent R, wherein each R is independently H, halogen, —CN or C₁₋₃ alkyl;
ring C is 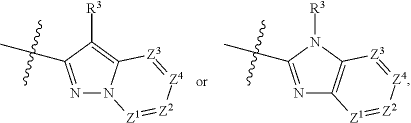
wherein Z¹, Z², Z³ and Z⁴ are independently N, CR⁴ or CR⁸, wherein R⁸ is independently H, CN, halogen or —C₁₋₃alkyl, provided that one of Z¹, Z², Z³ and Z⁴ is CR⁴;
R³ is —C₁₋₃alkyl, —C₀₋₃alkylene-C₃₋₆cycloalkyl, or —C₀₋₃alkylene-R⁵, wherein said alkyl may be substituted as valency allows with 0 to 3 substituents independently selected from 0 to 3 halogen atoms and 0 to 1 substituent selected from —C₀₋₁alkylene-CN, —C₀₋₁alkylene-OR⁹, and —N(R¹⁰)₂, and wherein said alkylene and cycloalkyl may be independently substituted as valency allows with 0 to 2 substituents independently selected from 0 to 2 halogen atoms and 0 to 1 substituent selected from —C₀₋₁alkylene-CN, —C₀₋₁alkylene-OR⁹, and —N(R¹⁰)₂;
R⁵ is a 5- or 6-membered heteroaryl group, or a 4- to 6-membered heterocycloalkyl group, wherein said heteroaryl and heterocycloalkyl may be substituted with 0 to 2 substituents as valency allows independently selected from:
0 to 1 oxo (═O),
0 to 1 —CN,
0 to 2 halogen atoms, and
0 to 2 substituents independently selected from —C₁₋₃alkyl, —OC₁₋₃alkyl and —C₁₋₃alkylene-O—C₁₋₃alkyl wherein the alkyl of —C₁₋₃alkyl and —OC₁₋₃alkyl may be substituted with 0 to 3 substituents as valency allows independently selected from 0 to 3 halogen atoms, 0 to 1 —CN, and 0 to 1 —OR⁹;
each R⁹ is independently H, or —C₁₋₃alkyl, wherein-C₁₋₃alkyl may be substituted with 0 to 3 halogen atoms;
each R¹⁰ is independently H, or —C₁₋₃alkyl; and
R⁴ is COOH or a carboxylic group surrogate, and particularly, the carboxylic group surrogate is:

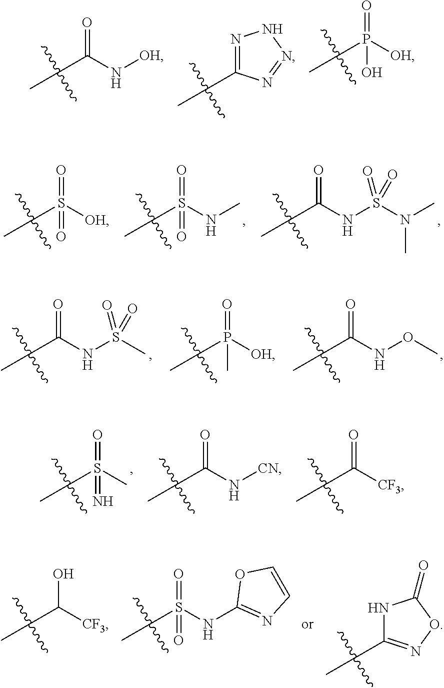

3. The compound of Formula I or a pharmaceutically acceptable salt thereof as claimed in claim 1, wherein ring A is phenyl, pyridinyl, or thiophenyl.

4. The compound of Formula I or a pharmaceutically acceptable salt thereof as claimed in claim 1, wherein each R¹ is independently halogen, —CN, —C≡CH, —C₁₋₃alkyl, or —OC₁₋₃alkyl, wherein said alkyl of —C₁₋₃alkyl and —OC₁₋₃alkyl is substituted with 0 to 5 halogen atoms.

5.-6. (canceled)

7. The compound of Formula I or a pharmaceutically acceptable salt thereof as claimed in claim 1, wherein each R¹ is independently halogen, —CN, —C₁₋₃alkyl, or —OC₁₋₃alkyl, wherein said alkyl of —C₁₋₃alkyl and —OC₁₋₃alkyl is substituted with 0 to 5 halogen atoms, and m is 1, 2 or 3, provided that one R¹ is —OC₁₋₃alkyl.

8. The compound of Formula I or a pharmaceutically acceptable salt thereof as claimed in claim 1, wherein R² is H or —CH₃.

9. The compound of Formula I or a pharmaceutically acceptable salt thereof as claimed in claim 1, wherein ring B is 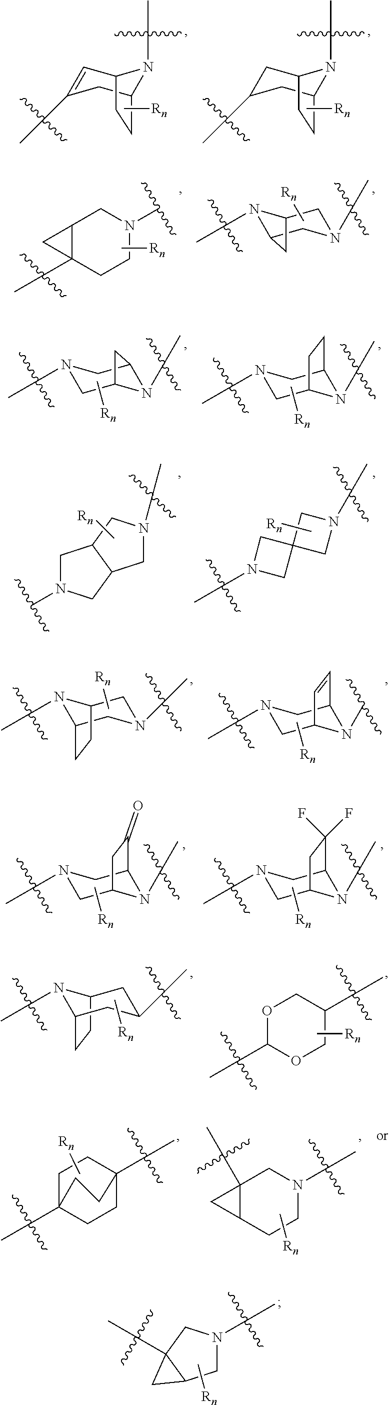
each R is independently H, halogen, —CN or —C₁₋₃alkyl; and
n is 0, 1 or 2.

10. (canceled)

11. The compound of Formula I or a pharmaceutically acceptable salt thereof as claimed in claim 1, wherein R³ is —CH₂—R⁵, and R⁵ is oxetan-2-yl, oxazol-2-yl, oxazol-5-yl, thiazol-2-yl, thiazol-5-yl, oxetan-3-yl, azetidin-2-yl, azetidin-3-yl, tetrahydrofuran-2-yl or tetrahydrofuran-3-yl; particularly, oxetan-2-yl, oxazol-2-yl, azetidin-2-yl or tetrahydrofuran-2-yl; or R³ is —CH₂CH₂OC₁₋₃alkyl, particularly, —CH₂CH₂OCH₃.

12. (canceled)

13. The compound of Formula I or a pharmaceutically acceptable salt thereof as claimed in claim 1, wherein the compound has the structure of formula Ia: 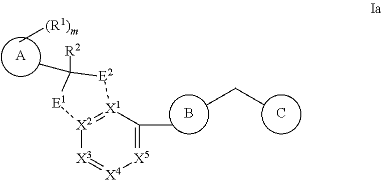
wherein
ring A is phenyl, pyridinyl, or thiophenyl;
each R¹ is independently F, Cl, —CN, —C≡CH, —CH₃, or —CF₃;
m is 0, 1 or 2;
E¹ and E² are independently H, O, NH, or CH₂;
X¹ and X² are independently N or CR⁶, R⁶ is independently absent, H, halogen, —C₁₋₃alkyl or —CN; particularly, R⁶ is independently absent, H, halogen or CH₃;
X³, X⁴ and X⁵ are independently N or CR⁷, wherein R⁷ is independently H, halogen, —C₁₋₃alkyl or —CN; particularly, R⁷ is independently H or CH₃;
--- denotes the presence or absence of a bond; provided that,
a) when the --- between E¹ and X² denotes the absence of a bond, then the --- between E² and X¹ denotes the presence of a bond, E¹ is H, and X¹ is C,
b) when the --- between E² and X¹ denotes the absence of a bond, then the --- between E¹ and X² denotes the presence of a bond, E² is H, and X² is C, and
c) when the --- between E¹ and X² and the --- between E² and X¹ both denote the presence of a bond, then E¹ and E² are independently O, NH or CH₂, and X¹ and X² are C;
R² is H or —CH₃;
ring B is 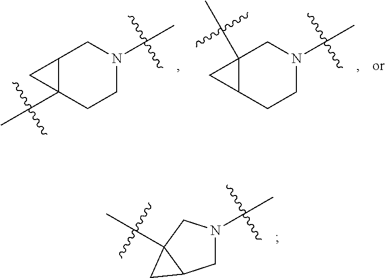 [SMILES: *N1CCC2(*)CC2C1 ; *N1CCC2CC2(*)C1 ; *N1CC2CC2(*)C1]
ring C is 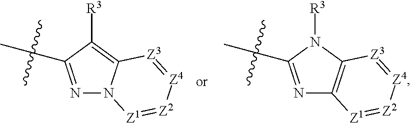
wherein Z¹, Z², Z³ and Z⁴ are independently N, CR⁴ or CR⁸, wherein R⁸ is independently H, halogen or —C₁₋₃alkyl, provided that one of Z¹, Z², Z³ and Z⁴ is CR⁴;
R³ is —CH₂CH₂OC₁₋₃alkyl, particularly, —CH₂CH₂OCH₃; or R³ is —CH₂—R⁵, and R⁵ is oxetan-2-yl, oxazol-2-yl, oxazol-5-yl, thiazol-2-yl, thiazol-5-yl, oxetan-3-yl, azetidin-2-yl, azetidin-3-yl, tetrahydrofuran-2-yl or tetrahydrofuran-3-yl, particularly, oxetan-2-yl, azetidin-2-yl or tetrahydrofuran-2-yl; and
R⁴ is COOH,

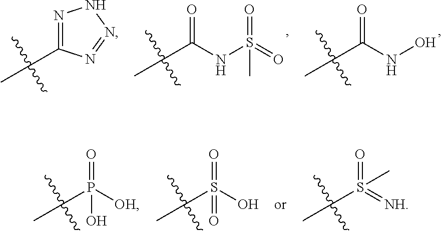

14. The compound of Formula I or a pharmaceutically acceptable salt thereof as claimed in claim 13, wherein the compound has the structure of formula Ia-1 or formula Ia-2: 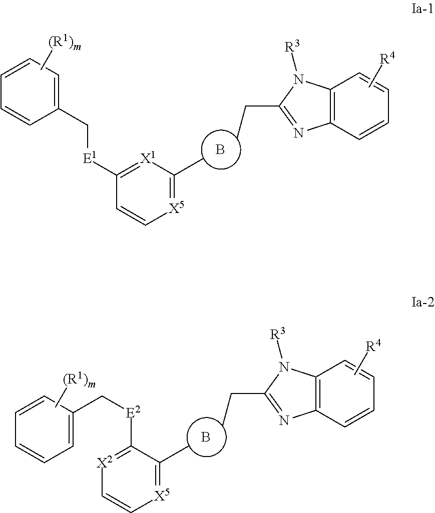
wherein
each R¹ is independently F, Cl, —CN, —C≡CH, or —CH₃;
m is 0, 1 or 2;
E¹ is O, NH, or CH₂;
E² is O, NH, or CH₂;
X¹, X² and X⁵ are independently CH, CCH₃, or N;
ring B is 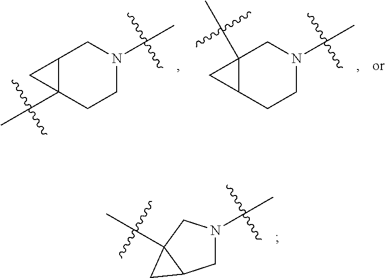 [SMILES: *N1CCC2(*)CC2C1 ; *N1CCC2CC2(*)C1 ; *N1CC2CC2(*)C1]
R³ is —CH₂CH₂OCH₃; or R³ is —CH₂—R⁵, and R⁵ is oxetan-2-yl, oxazol-2-yl, oxazol-5-yl, azetidin-2-yl or tetrahydrofuran-2-yl;
R⁴ is COOH, 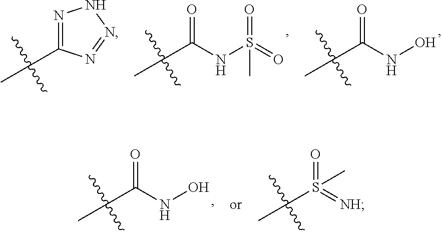
or
the compound has the structure of formula Ia-3: 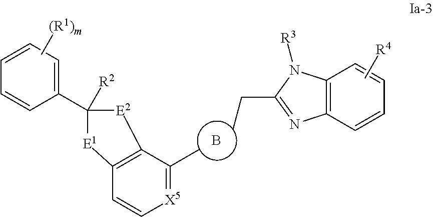
wherein
each R¹ is independently F, Cl, —CN, —C≡CH, or —CH₃;
m is 0, 1 or 2;
E¹ is O, NH, or CH₂;
E² is O, NH, or CH₂;
X⁵ is CH, CCH₃, or N;
R² is H or —CH₃;
ring B is 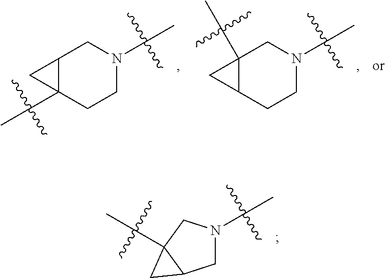 [SMILES: *N1CCC2(*)CC2C1 ; *N1CCC2CC2(*)C1 ; *N1CC2CC2(*)C1]
R³ is —CH₂CH₂OCH₃; or R³ is —CH₂—R⁵, and R⁵ is oxetan-2-yl, oxazol-2-yl, oxazol-5-yl, azetidin-2-yl or tetrahydrofuran-2-yl;
R⁴ is COOH, 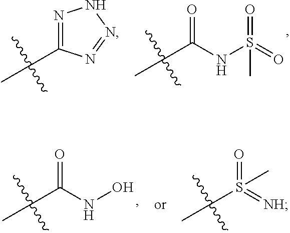
or
the compound has the structure of formula Ia-4: 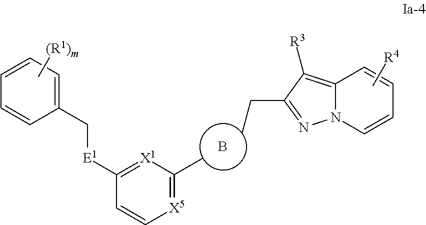
wherein
each R¹ is independently F, Cl, —CN, —C≡CH, or —CH₃;
m is 0, 1 or 2;
E¹ is O, NH, or CH₂;
X¹ and X⁵ are independently CH, CCH₃, or N;
ring B is 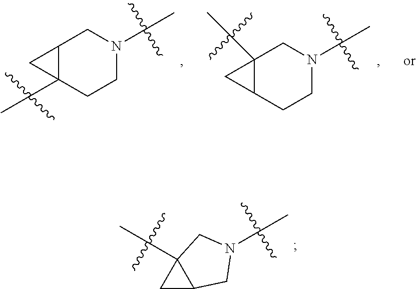 [SMILES: *N1CCC2(*)CC2C1 ; *N1CCC2CC2(*)C1 ; *N1CC2CC2(*)C1]
R³ is —CH₂CH₂OCH₃; or R³ is —CH₂—R⁵, and R⁵ is oxetan-2-yl, oxazol-2-yl, oxazol-5-yl, azetidin-2-yl or tetrahydrofuran-2-yl;
R⁴ is COOH,

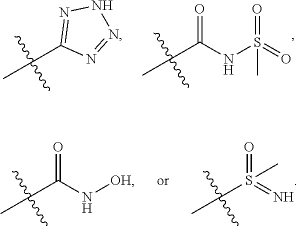

15.-16. (canceled)

17. The compound of Formula I or a pharmaceutically acceptable salt thereof as claimed in claim 1, wherein the compound has the structure of formula Ib-1 or Ib-2: 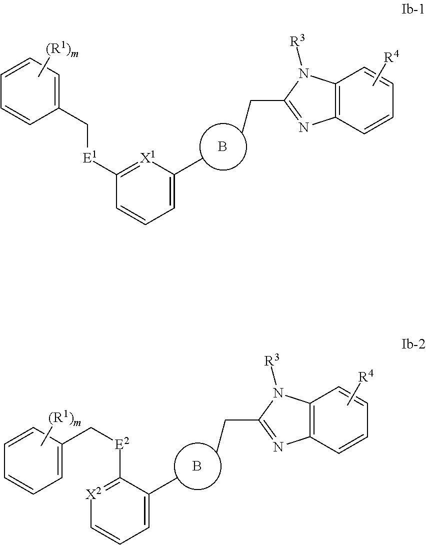
wherein
each R¹ is independently F, Cl, —CN, —C≡CH, or —CH₃;
m is 0, 1 or 2;
E¹ is O, NH, or CH₂;
E² is O, NH, or CH₂;
X¹ and X² is independently CH, CCH₃, or N;
ring B is 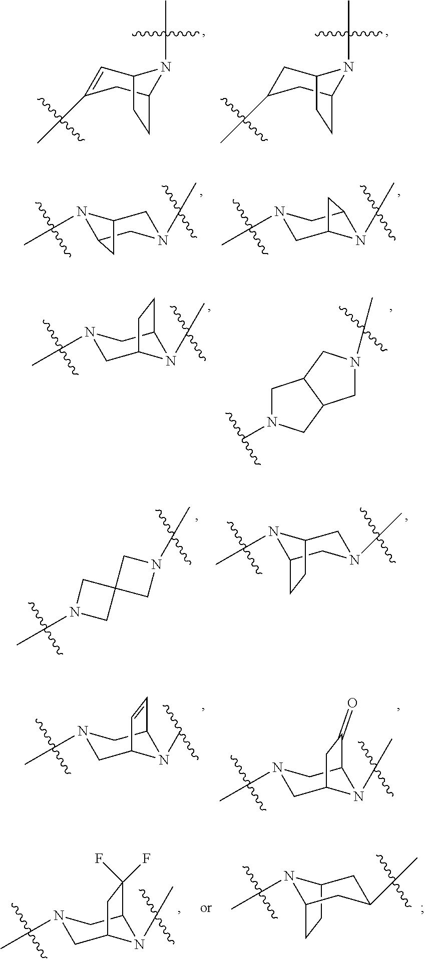
R³ is —CH₂CH₂OCH₃; or R³ is —CH₂—R⁵, and R⁵ is oxetan-2-yl, azetidin-2-yl or tetrahydrofuran-2-yl;
R⁴ is COOH, 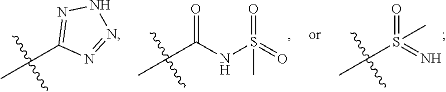
or
the compound has the structure of formula Ic: 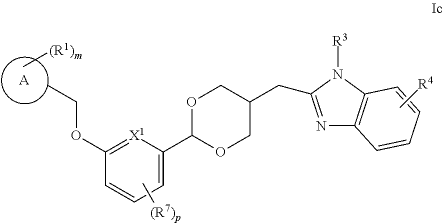
wherein
ring A is phenyl or pyridinyl;
each R¹ is independently F, Cl, —CN, —C≡CH, or —CH₃;
m is 0, 1 or 2;
X¹ is CH, CCH₃, or N;
R⁷ is independently-C₁₋₃alkyl; particularly, R⁷ is CH₃;
p is 0, 1 or 2;
R³ is —CH₂CH₂OCH₃; or R³ is —CH₂—R⁵, and R⁵ is oxetan-2-yl, azetidin-2-yl or tetrahydrofuran-2-yl;
R⁴ is COOH, 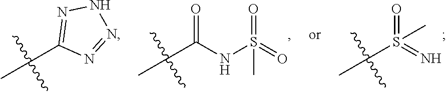
or
the compound has the structure of formula Id: 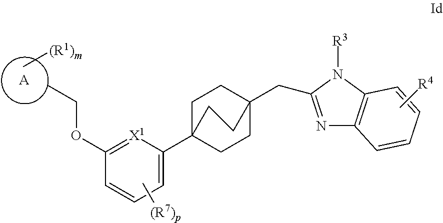
wherein
ring A is phenyl or pyridinyl;
each R¹ is independently F, Cl, —CN, —C≡CH, or —CH₃;
m is 0, 1 or 2;
X¹ is CH, CCH₃, or N;
R⁷ is independently-C₁₋₃alkyl; particularly, R⁷ is CH₃;
p is 0, 1 or 2;
R³ is —CH₂CH₂OCH₃; or R³ is —CH₂—R⁵, and R⁵ is oxetan-2-yl, azetidin-2-yl or tetrahydrofuran-2-yl;
R⁴ is COOH,

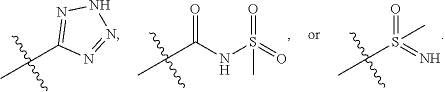

18.-19. (canceled)

20. The compound of Formula I or a pharmaceutically acceptable salt thereof as claimed in claim 1, wherein ring B is: 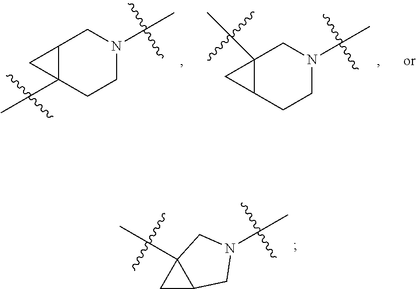 [SMILES: *N1CCC2(*)CC2C1 ; *N1CCC2CC2(*)C1 ; *N1CC2CC2(*)C1]
and/or
ring C is: 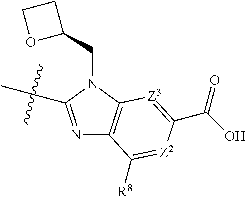
wherein
Z² and Z³ are independently N or CH₂;
and/or
R⁸ is independently H, CN, halogen, —C(O) C₁₋₃alkyl, —OC₁₋₃alkyl, —C₃₋₆cycloalkyl, or —C₁₋₃alkyl, wherein said alkyl and said cycloalkyl of —C(O) C₁₋₃alkyl, —OC₁₋₃alkyl, —C₃₋₆cycloalkyl, and —C₁₋₃alkyl are independently unsubstituted or substituted with one or more substituents selected from OH, NH₂, —CN, and halogen.

21.-22. (canceled)

23. The compound of Formula I or a pharmaceutically acceptable salt thereof as claimed in claim 1, wherein 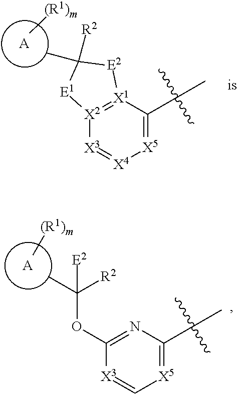
E² is independently H, D, or halogen.

24. The compound of Formula I or a pharmaceutically acceptable salt thereof as claimed in claim 1, wherein the compound has the structure of formula Ie: 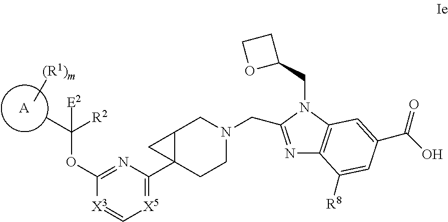
wherein
each R¹ is independently F, Cl, —CN, —C≡CH, —C₁₋₃alkyl, or —OC₁₋₃alkyl, wherein said alkyl of —C₁₋₃alkyl and —OC₁₋₃alkyl is substituted with 0 to 5 halogen atoms;
ring A is phenyl, or pyridinyl;
m is 1, 2 or 3;
E² is independently H, D, or halogen;
X³ and X⁵ are independently N or CR⁷, wherein R⁷ is independently H, halogen, —C₁₋₃alkyl, —OC₁₋₃alkyl or —CN, wherein said alkyl of —C₁₋₃alkyl and —OC₁₋₃alkyl is substituted with 0 to 3 halogen atoms.

25. The compound of Formula I or a pharmaceutically acceptable salt thereof as claimed in claim 1, wherein

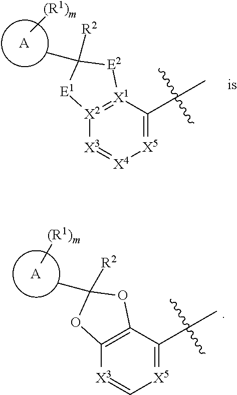

26. The compound of Formula I or a pharmaceutically acceptable salt thereof as claimed in claim 1, wherein the compound has the structure of formula If: 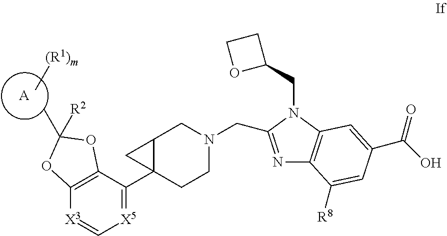
wherein
each R¹ is independently F, Cl, —CN, —C≡CH, —C₁₋₃alkyl, or —OC₁₋₃alkyl, wherein said alkyl of —C₁₋₃alkyl and —OC₁₋₃alkyl is substituted with 0 to 5 halogen atoms;
ring A is phenyl or pyridinyl;
m is 1, 2 or 3;
X³ and X⁵ are independently N or CR⁷, wherein R⁷ is independently H, halogen, —C₁₋₃alkyl, —OC₁₋₃alkyl or —CN, wherein said alkyl of —C₁₋₃alkyl and —OC₁₋₃alkyl is substituted with 0 to 3 halogen atoms.

27.-28. (canceled)

29. The compound of Formula I or a pharmaceutically acceptable salt thereof as claimed in claim 1, wherein the compound is selected from the group consisting of the following compounds:

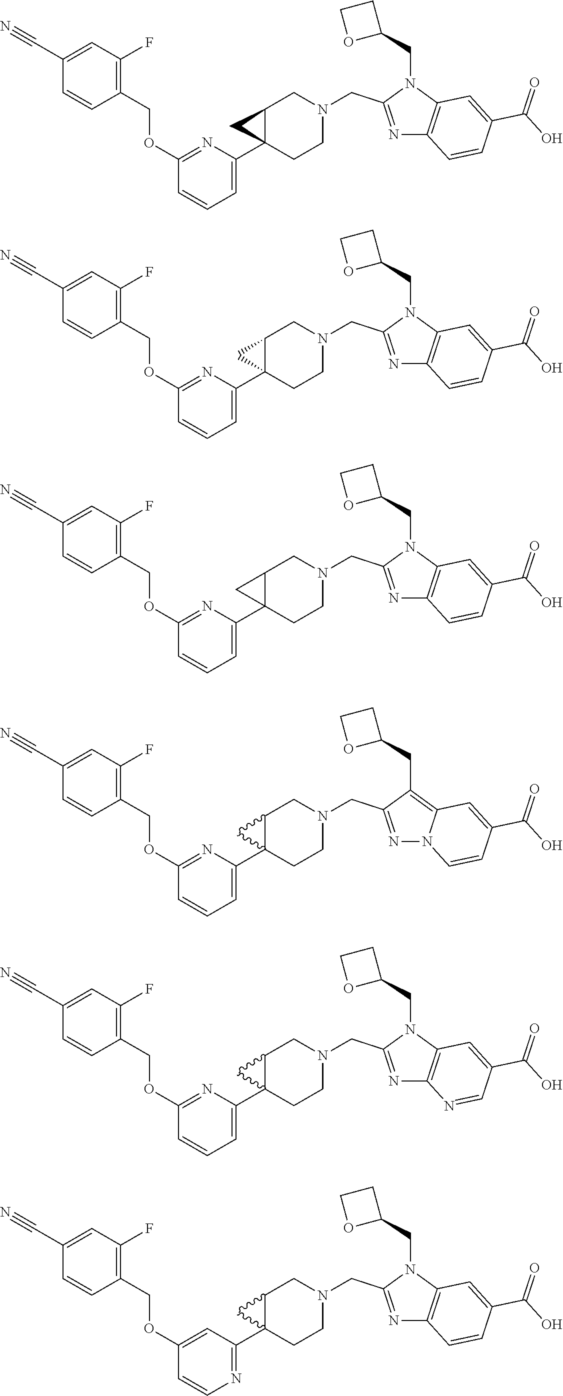

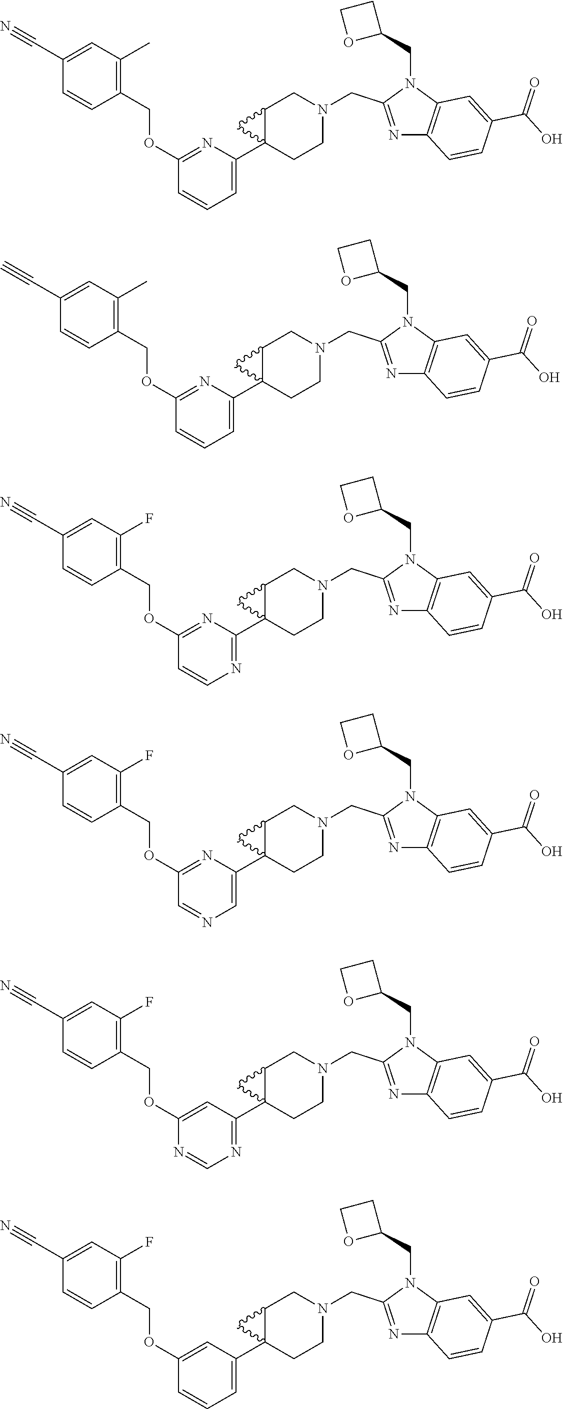

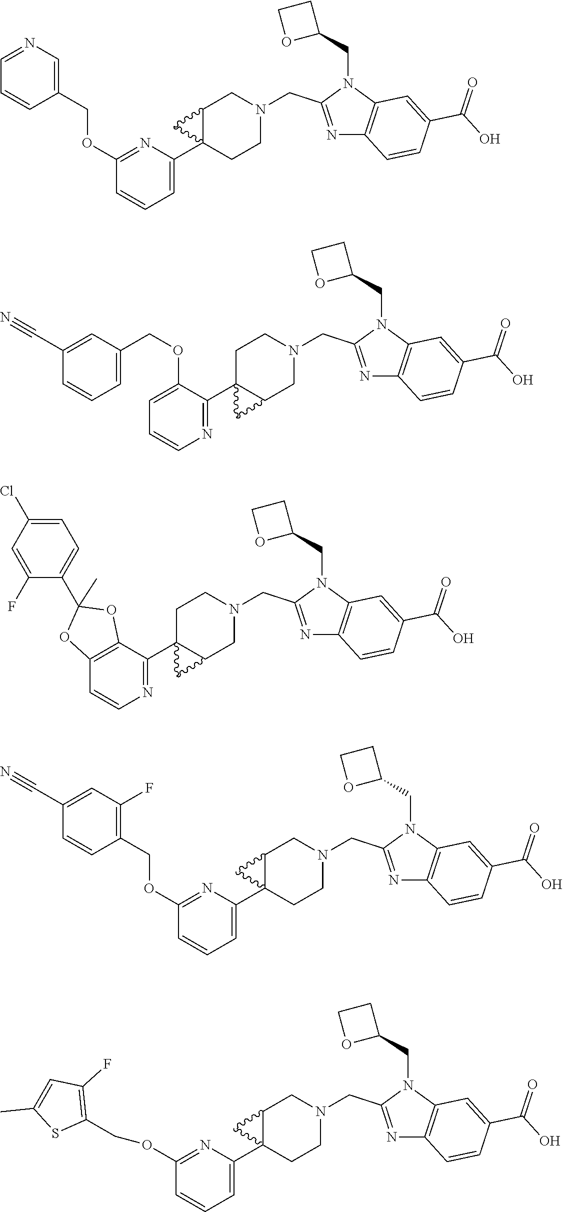

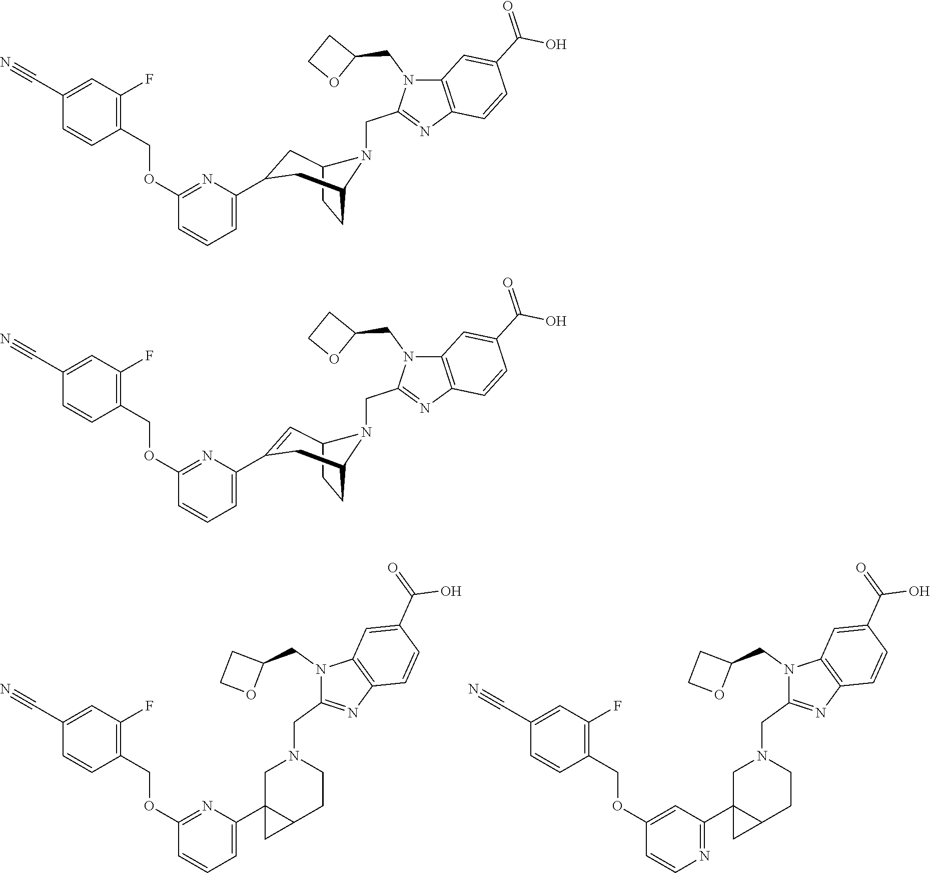

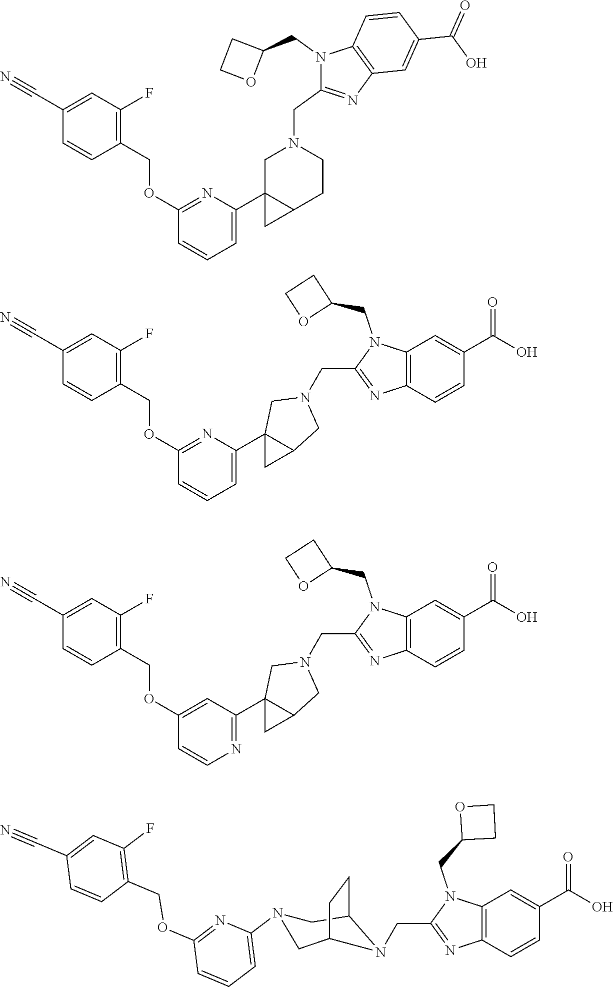 [SMILES: N#Cc1ccc(COc2cccc(C34CC3CCN(Cc3nc5cc(C(=O)O)ccc5n3C[C@@H]3CCO3)C4)n2)c(F)c1 ; N#Cc1ccc(COc2cccc(C34CC3CN(Cc3nc5ccc(C(=O)O)cc5n3C[C@@H]3CCO3)C4)n2)c(F)c1 ; N#Cc1ccc(COc2ccnc(C34CC3CN(Cc3nc5ccc(C(=O)O)cc5n3C[C@@H]3CCO3)C4)c2)c(F)c1 ; N#Cc1ccc(COc2cccc(N3CC4CCC(C3)N4Cc3nc4ccc(C(=O)O)cc4n3C[C@@H]3CCO3)n2)c(F)c1]

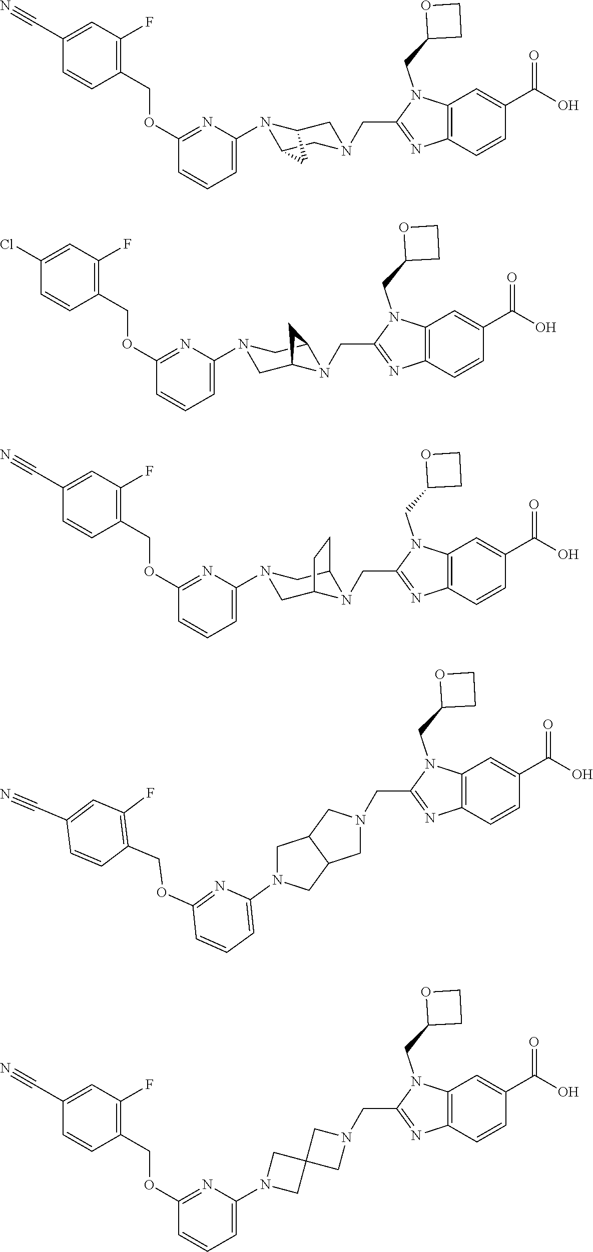 [SMILES: N#Cc1ccc(COc2cccc(N3[C@H]4C[C@H]3CN(Cc3nc5ccc(C(=O)O)cc5n3C[C@@H]3CCO3)C4)n2)c(F)c1 ; O=C(O)c1ccc2nc(CN3[C@@H]4C[C@@H]3CN(c3cccc(OCc5ccc(Cl)cc5F)n3)C4)n(C[C@@H]3CCO3)c2c1 ; N#Cc1ccc(COc2cccc(N3CC4CCC(C3)N4Cc3nc4ccc(C(=O)O)cc4n3C[C@H]3CCO3)n2)c(F)c1 ; N#Cc1ccc(COc2cccc(N3CC4CN(Cc5nc6ccc(C(=O)O)cc6n5C[C@@H]5CCO5)CC4C3)n2)c(F)c1 ; N#Cc1ccc(COc2cccc(N3CC4(CN(Cc5nc6ccc(C(=O)O)cc6n5C[C@@H]5CCO5)C4)C3)n2)c(F)c1]

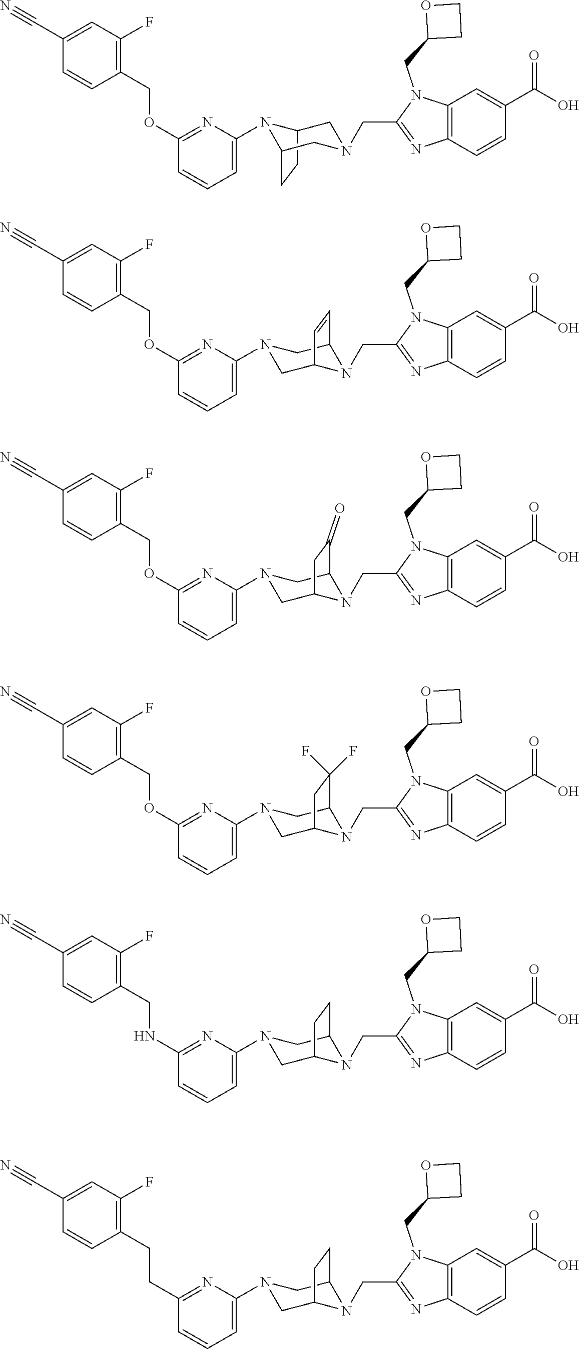 [SMILES: N#Cc1ccc(COc2cccc(N3C4CCC3CN(Cc3nc5ccc(C(=O)O)cc5n3C[C@@H]3CCO3)C4)n2)c(F)c1 ; N#Cc1ccc(COc2cccc(N3CC4C=CC(C3)N4Cc3nc4ccc(C(=O)O)cc4n3C[C@@H]3CCO3)n2)c(F)c1 ; N#Cc1ccc(COc2cccc(N3CC4CC(=O)C(C3)N4Cc3nc4ccc(C(=O)O)cc4n3C[C@@H]3CCO3)n2)c(F)c1 ; N#Cc1ccc(COc2cccc(N3CC4CC(F)(F)C(C3)N4Cc3nc4ccc(C(=O)O)cc4n3C[C@@H]3CCO3)n2)c(F)c1 ; N#Cc1ccc(CNc2cccc(N3CC4CCC(C3)N4Cc3nc4ccc(C(=O)O)cc4n3C[C@@H]3CCO3)n2)c(F)c1 ; N#Cc1ccc(CCc2cccc(N3CC4CCC(C3)N4Cc3nc4ccc(C(=O)O)cc4n3C[C@@H]3CCO3)n2)c(F)c1]

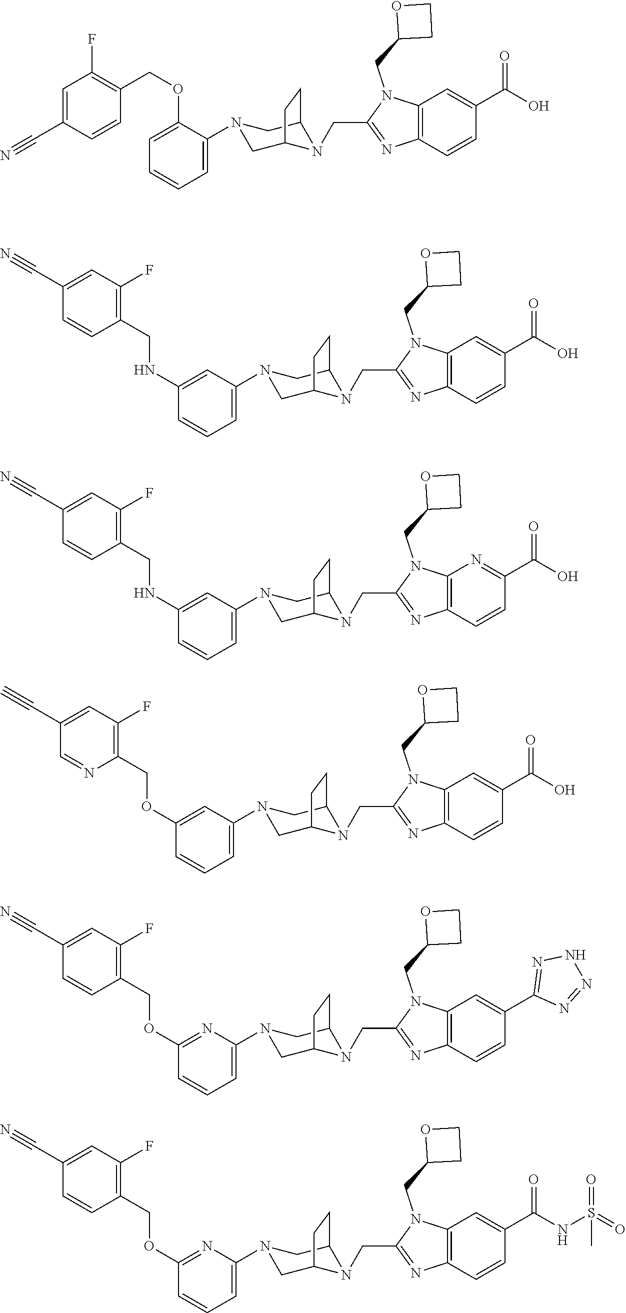 [SMILES: N#Cc1ccc(COc2ccccc2N2CC3CCC(C2)N3Cc2nc3ccc(C(=O)O)cc3n2C[C@@H]2CCO2)c(F)c1 ; N#Cc1ccc(CNc2cccc(N3CC4CCC(C3)N4Cc3nc4ccc(C(=O)O)cc4n3C[C@@H]3CCO3)c2)c(F)c1 ; N#Cc1ccc(CNc2cccc(N3CC4CCC(C3)N4Cc3nc4ccc(C(=O)O)nc4n3C[C@@H]3CCO3)c2)c(F)c1 ; C#Cc1cnc(COc2cccc(N3CC4CCC(C3)N4Cc3nc4ccc(C(=O)O)cc4n3C[C@@H]3CCO3)c2)c(F)c1 ; N#Cc1ccc(COc2cccc(N3CC4CCC(C3)N4Cc3nc4ccc(-c5nn[nH]n5)cc4n3C[C@@H]3CCO3)n2)c(F)c1 ; CS(=O)(=O)NC(=O)c1ccc2nc(CN3C4CCC3CN(c3cccc(OCc5ccc(C#N)cc5F)n3)C4)n(C[C@@H]3CCO3)c2c1]

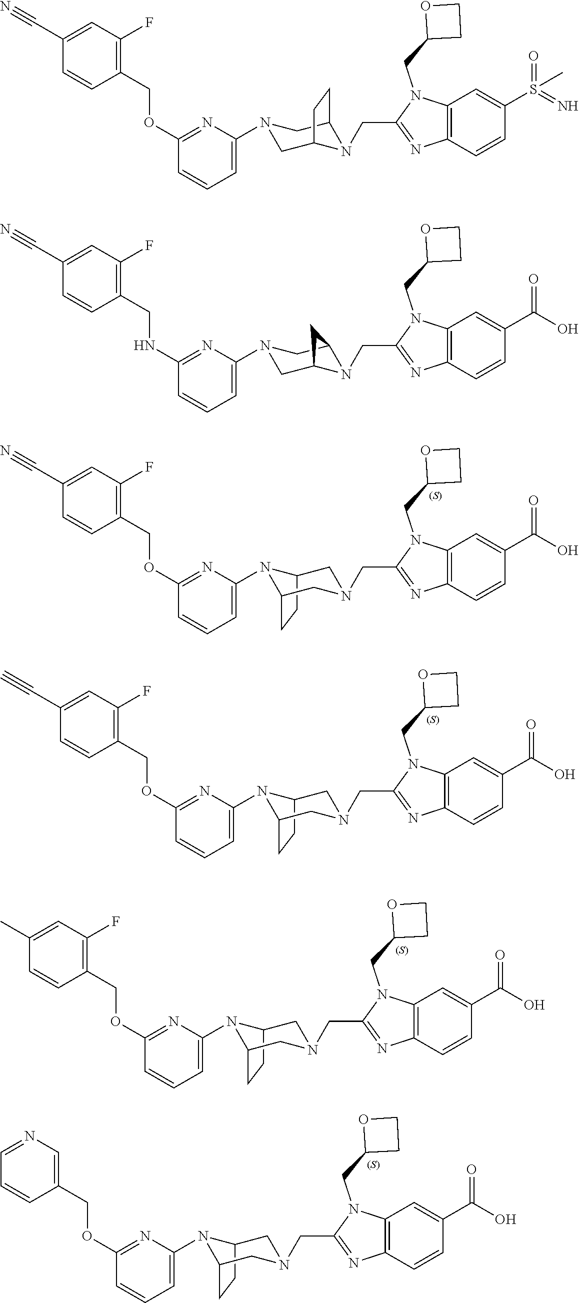

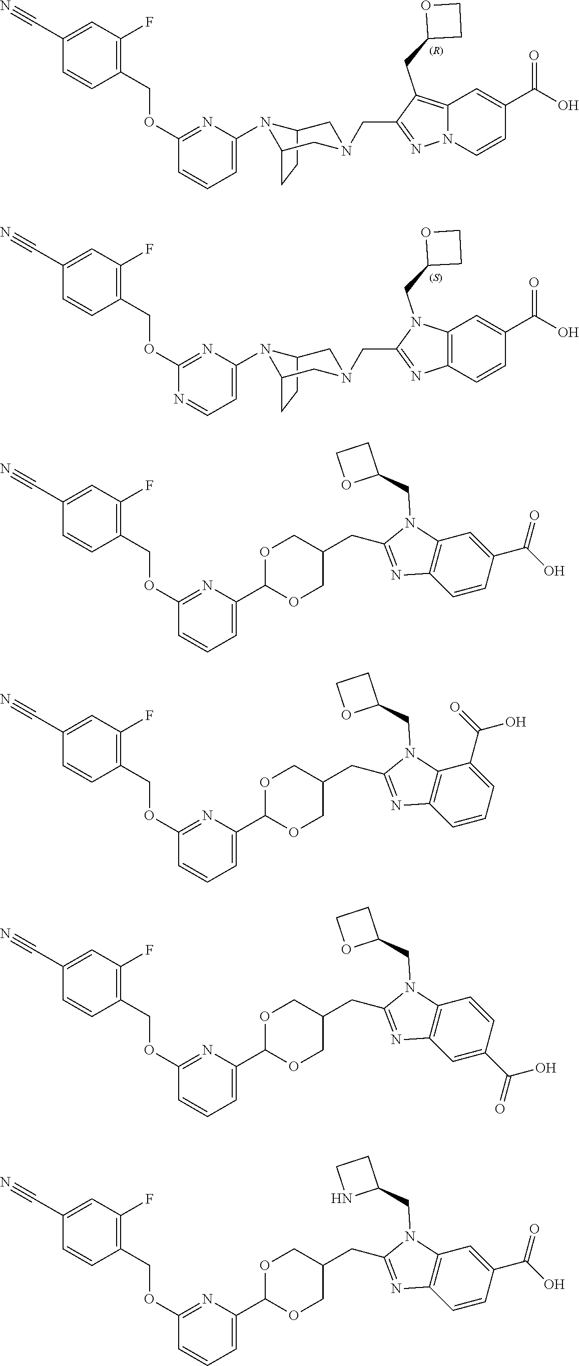

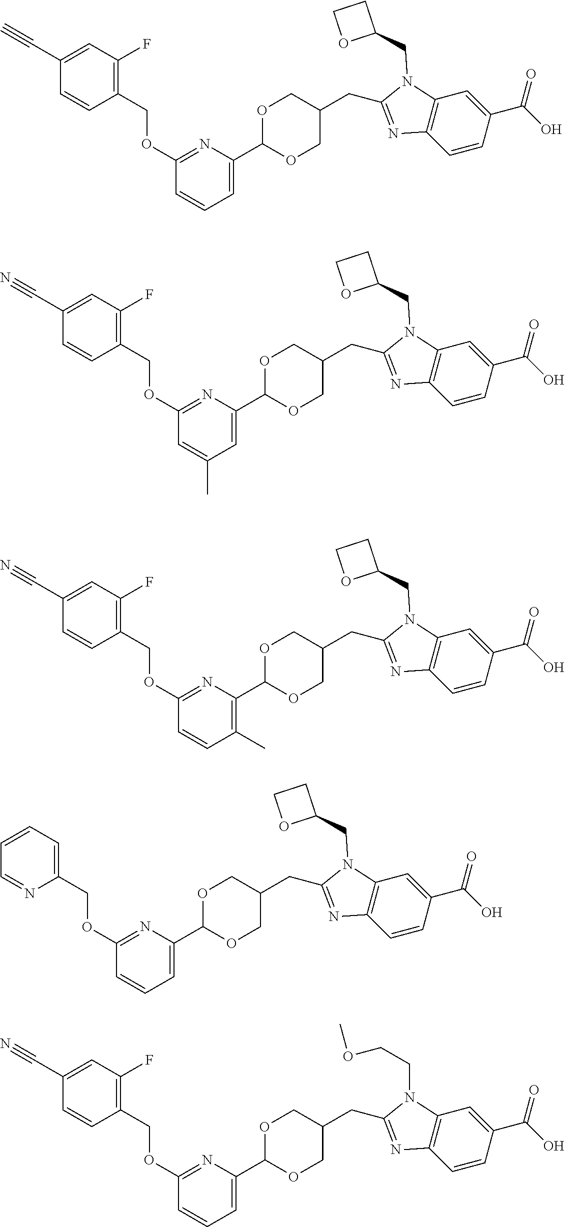 [SMILES: C#Cc1ccc(COc2cccc(C3OCC(Cc4nc5ccc(C(=O)O)cc5n4C[C@@H]4CCO4)CO3)n2)c(F)c1 ; Cc1cc(OCc2ccc(C#N)cc2F)nc(C2OCC(Cc3nc4ccc(C(=O)O)cc4n3C[C@@H]3CCO3)CO2)c1 ; Cc1ccc(OCc2ccc(C#N)cc2F)nc1C1OCC(Cc2nc3ccc(C(=O)O)cc3n2C[C@@H]2CCO2)CO1 ; O=C(O)c1ccc2nc(CC3COC(c4cccc(OCc5ccccn5)n4)OC3)n(C[C@@H]3CCO3)c2c1 ; COCCn1c(CC2COC(c3cccc(OCc4ccc(C#N)cc4F)n3)OC2)nc2ccc(C(=O)O)cc21]

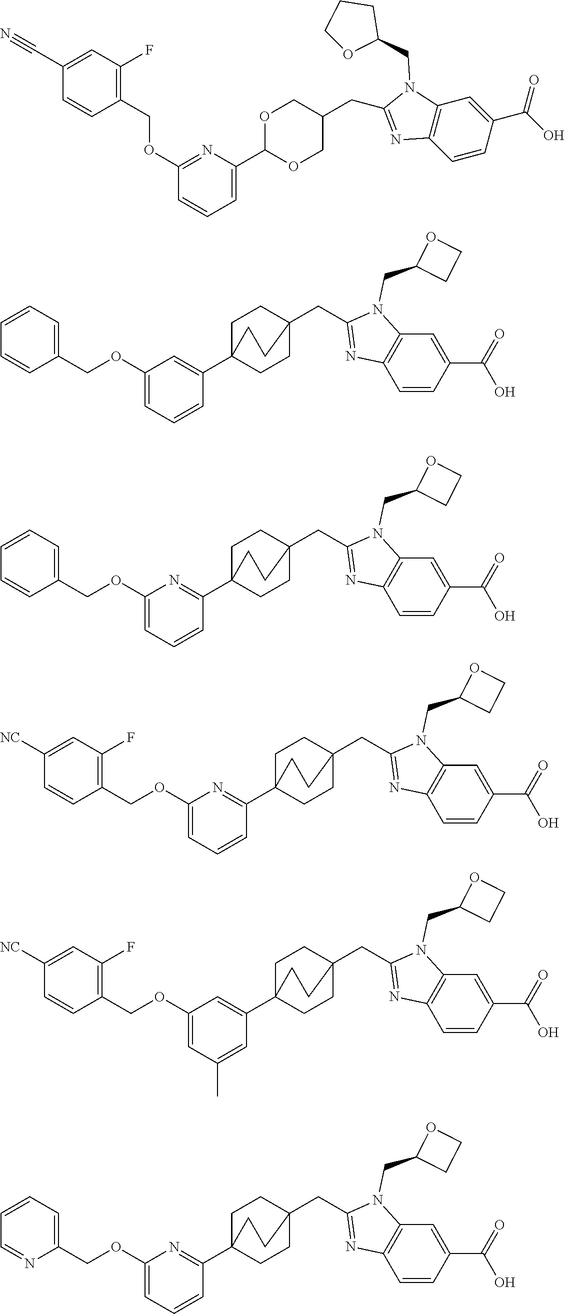 [SMILES: N#Cc1ccc(COc2cccc(C3OCC(Cc4nc5ccc(C(=O)O)cc5n4C[C@@H]4CCCO4)CO3)n2)c(F)c1 ; O=C(O)c1ccc2nc(CC34CCC(c5cccc(OCc6ccccc6)c5)(CC3)CC4)n(C[C@@H]3CCO3)c2c1 ; O=C(O)c1ccc2nc(CC34CCC(c5cccc(OCc6ccccc6)n5)(CC3)CC4)n(C[C@@H]3CCO3)c2c1 ; N#Cc1ccc(COc2cccc(C34CCC(Cc5nc6ccc(C(=O)O)cc6n5C[C@@H]5CCO5)(CC3)CC4)n2)c(F)c1 ; Cc1cc(OCc2ccc(C#N)cc2F)cc(C23CCC(Cc4nc5ccc(C(=O)O)cc5n4C[C@@H]4CCO4)(CC2)CC3)c1 ; O=C(O)c1ccc2nc(CC34CCC(c5cccc(OCc6ccccn6)n5)(CC3)CC4)n(C[C@@H]3CCO3)c2c1]

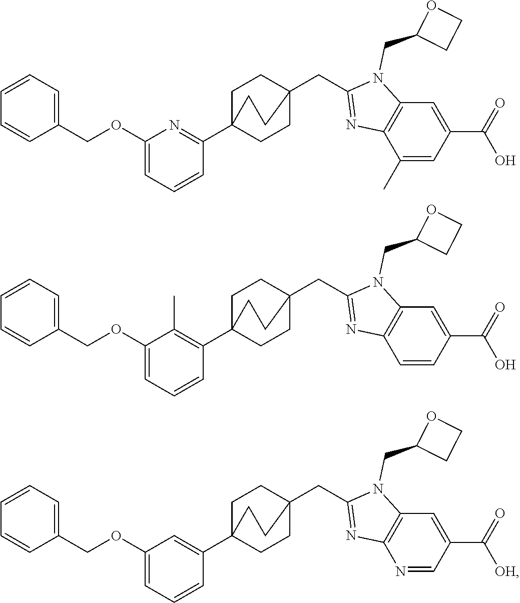 [SMILES: Cc1cc(C(=O)O)cc2c1nc(CC13CCC(c4cccc(OCc5ccccc5)n4)(CC1)CC3)n2C[C@@H]1CCO1 ; Cc1c(OCc2ccccc2)cccc1C12CCC(Cc3nc4ccc(C(=O)O)cc4n3C[C@@H]3CCO3)(CC1)CC2 ; O=C(O)c1cnc2nc(CC34CCC(c5cccc(OCc6ccccc6)c5)(CC3)CC4)n(C[C@@H]3CCO3)c2c1]

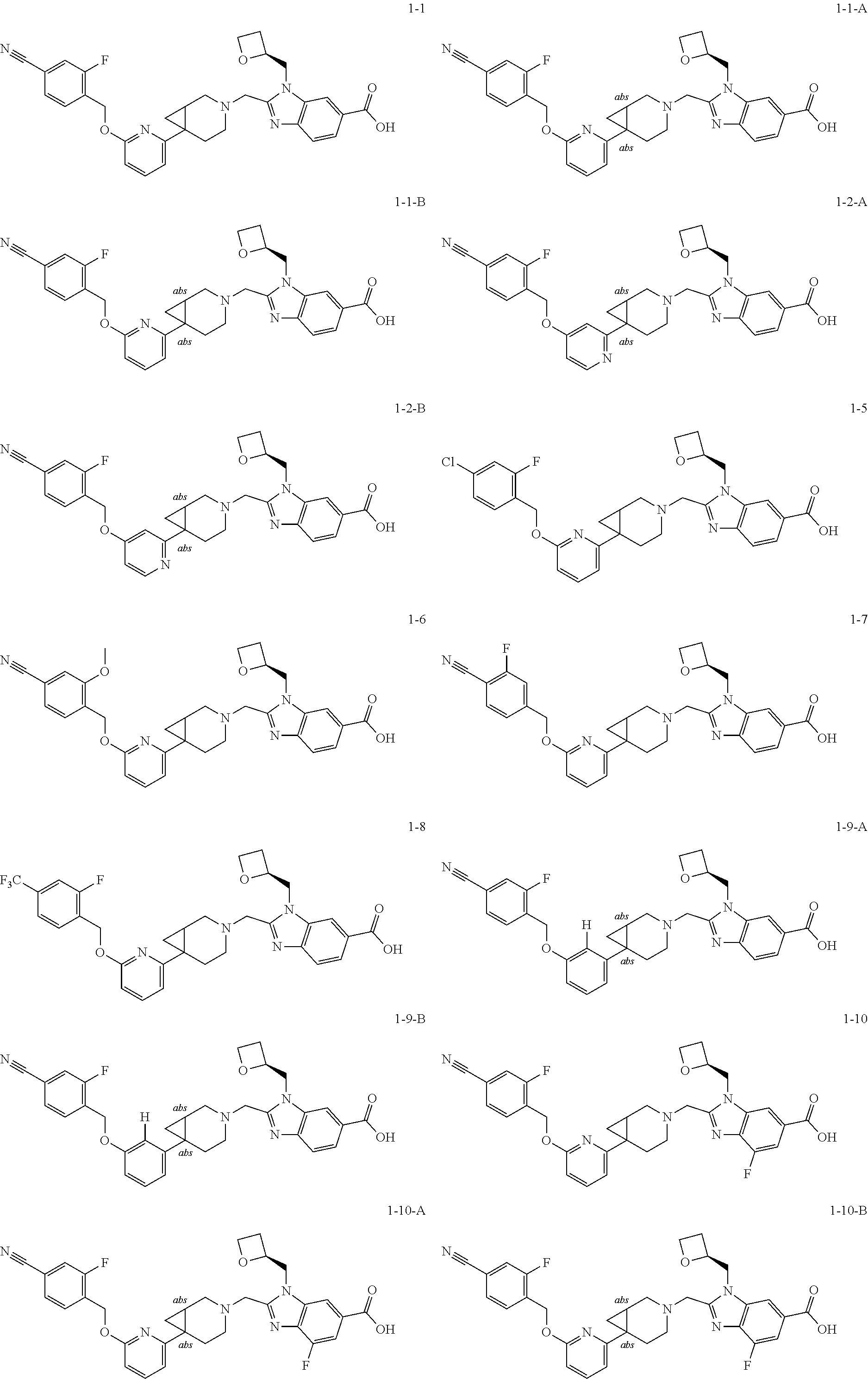

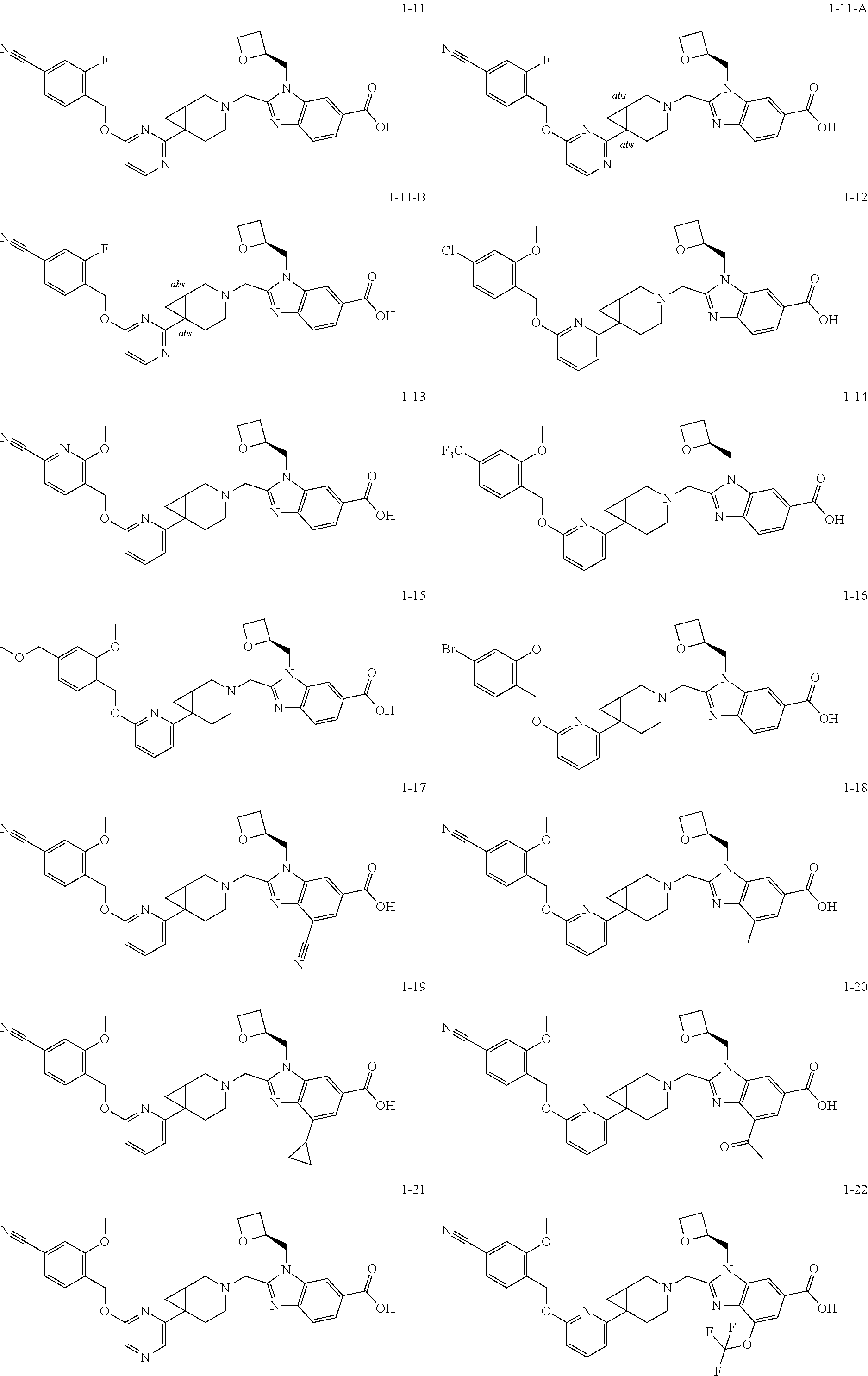

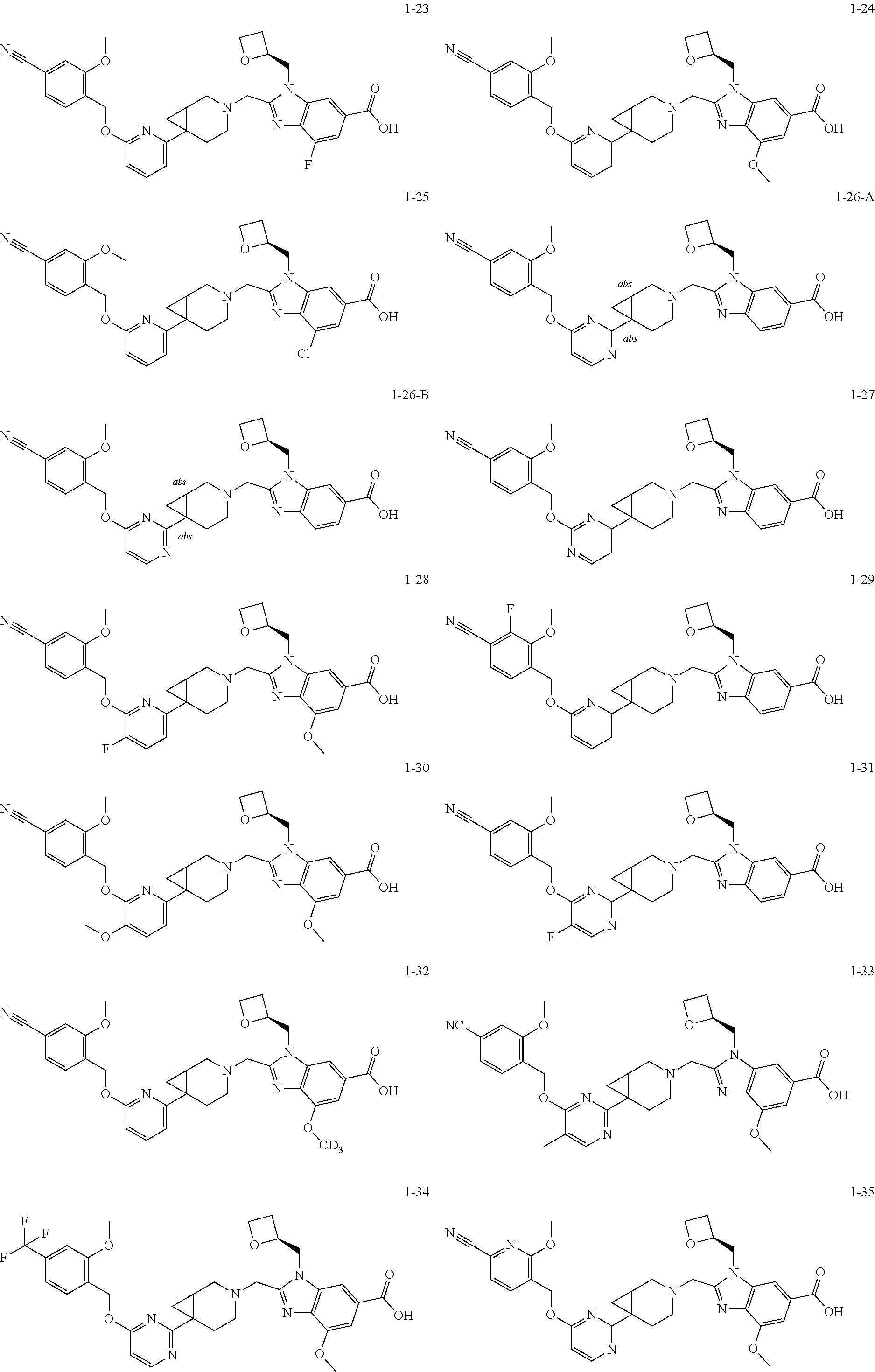

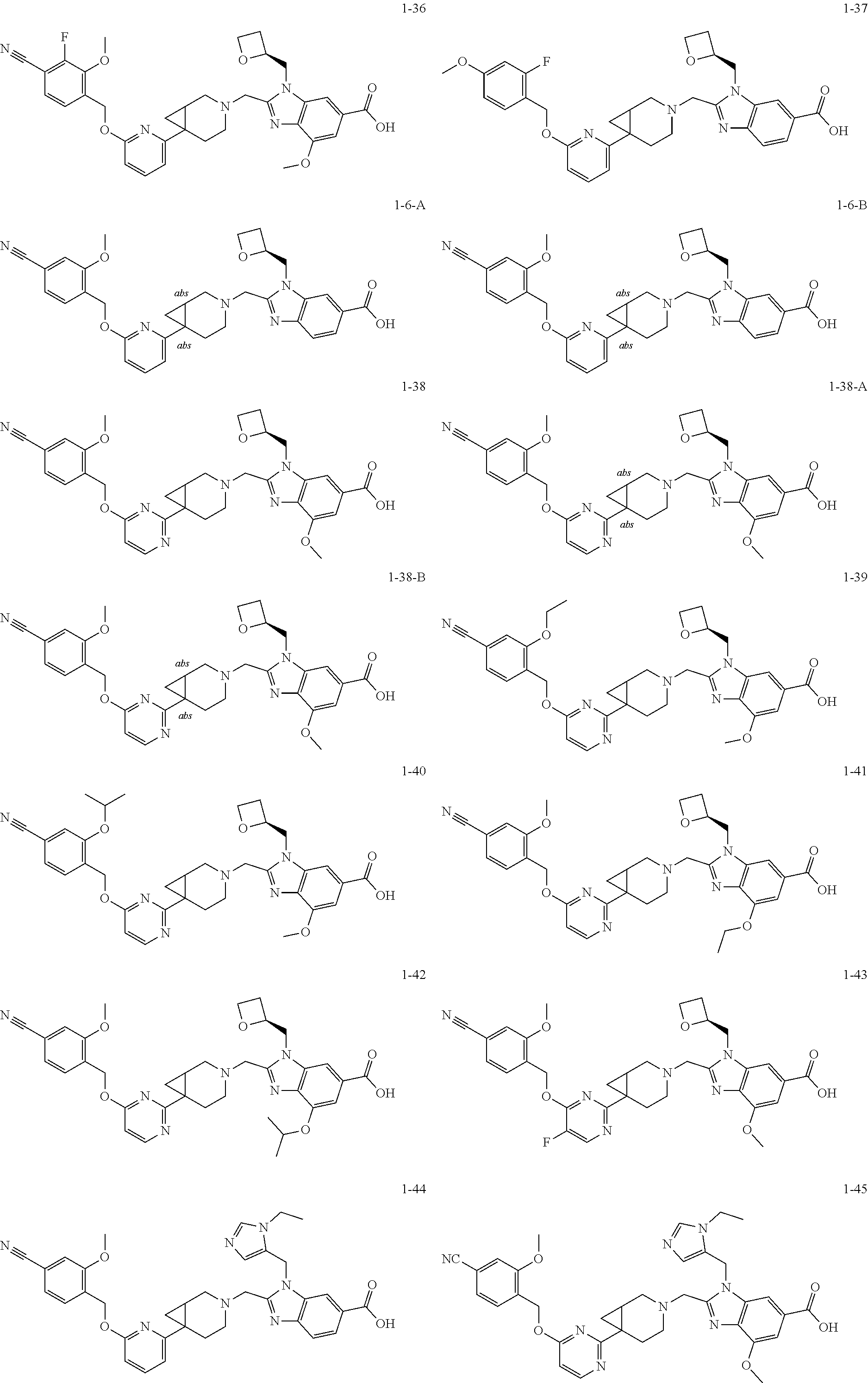

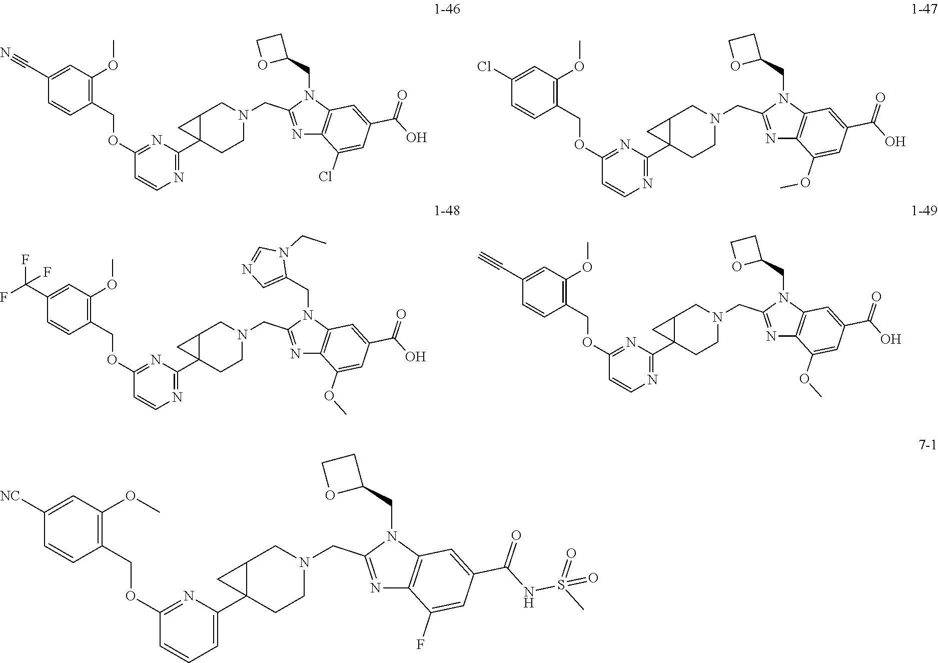 [SMILES: COc1cc(C#N)ccc1COc1ccnc(C23CCN(Cc4nc5c(Cl)cc(C(=O)O)cc5n4C[C@@H]4CCO4)CC2C3)n1 ; COc1cc(Cl)ccc1COc1ccnc(C23CCN(Cc4nc5c(OC)cc(C(=O)O)cc5n4C[C@@H]4CCO4)CC2C3)n1 ; CCn1cncc1Cn1c(CN2CCC3(c4nccc(OCc5ccc(C(F)(F)F)cc5OC)n4)CC3C2)nc2c(OC)cc(C(=O)O)cc21 ; C#Cc1ccc(COc2ccnc(C34CCN(Cc5nc6c(OC)cc(C(=O)O)cc6n5C[C@@H]5CCO5)CC3C4)n2)c(OC)c1 ; COc1cc(C#N)ccc1COc1cccc(C23CCN(Cc4nc5c(F)cc(C(=O)NS(C)(=O)=O)cc5n4C[C@@H]4CCO4)CC2C3)n1]

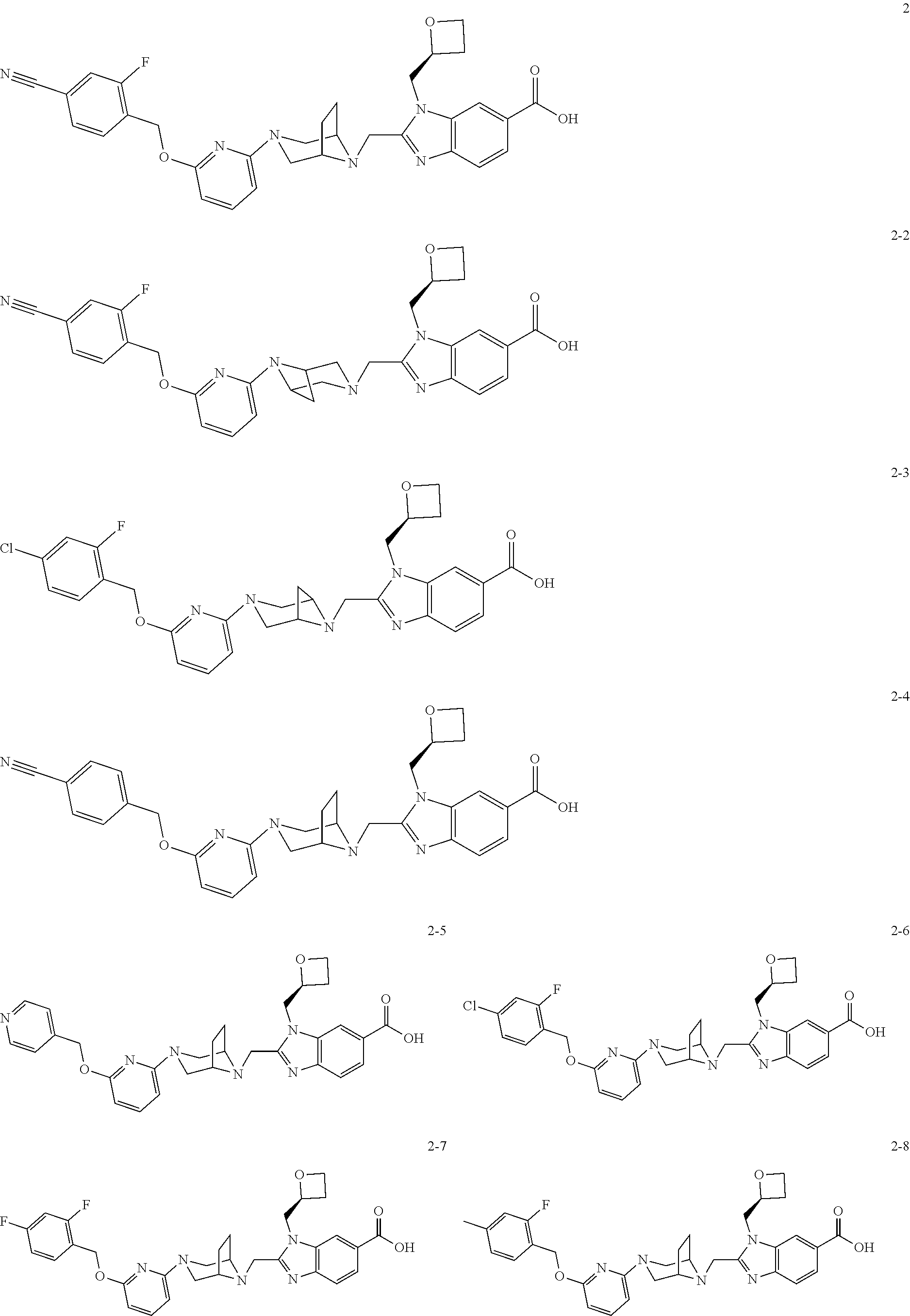

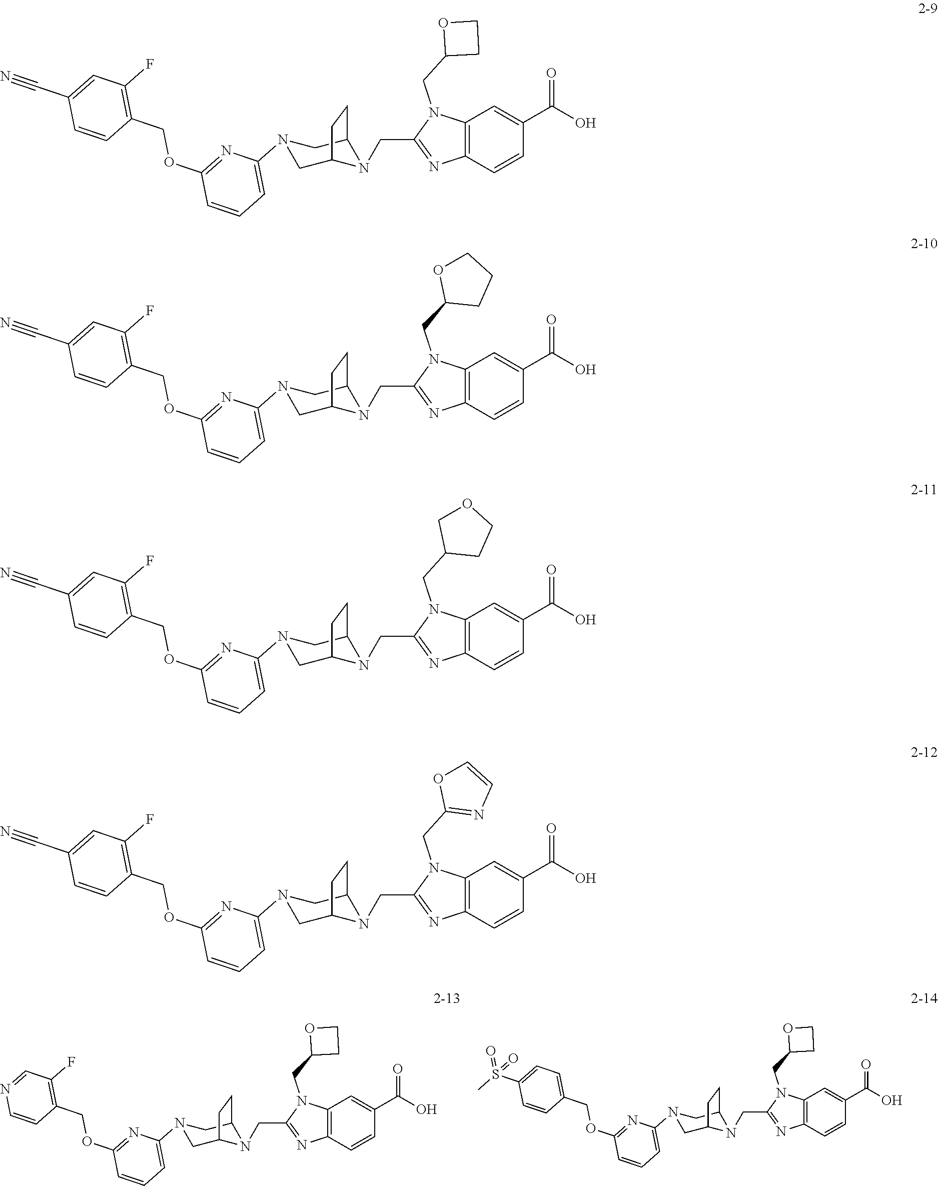 [SMILES: N#Cc1ccc(COc2cccc(N3CC4CCC(C3)N4Cc3nc4ccc(C(=O)O)cc4n3CC3CCO3)n2)c(F)c1 ; N#Cc1ccc(COc2cccc(N3CC4CCC(C3)N4Cc3nc4ccc(C(=O)O)cc4n3C[C@@H]3CCCO3)n2)c(F)c1 ; N#Cc1ccc(COc2cccc(N3CC4CCC(C3)N4Cc3nc4ccc(C(=O)O)cc4n3CC3CCOC3)n2)c(F)c1 ; N#Cc1ccc(COc2cccc(N3CC4CCC(C3)N4Cc3nc4ccc(C(=O)O)cc4n3Cc3ncco3)n2)c(F)c1 ; O=C(O)c1ccc2nc(CN3C4CCC3CN(c3cccc(OCc5ccncc5F)n3)C4)n(C[C@@H]3CCO3)c2c1 ; CS(=O)(=O)c1ccc(COc2cccc(N3CC4CCC(C3)N4Cc3nc4ccc(C(=O)O)cc4n3C[C@@H]3CCO3)n2)cc1]

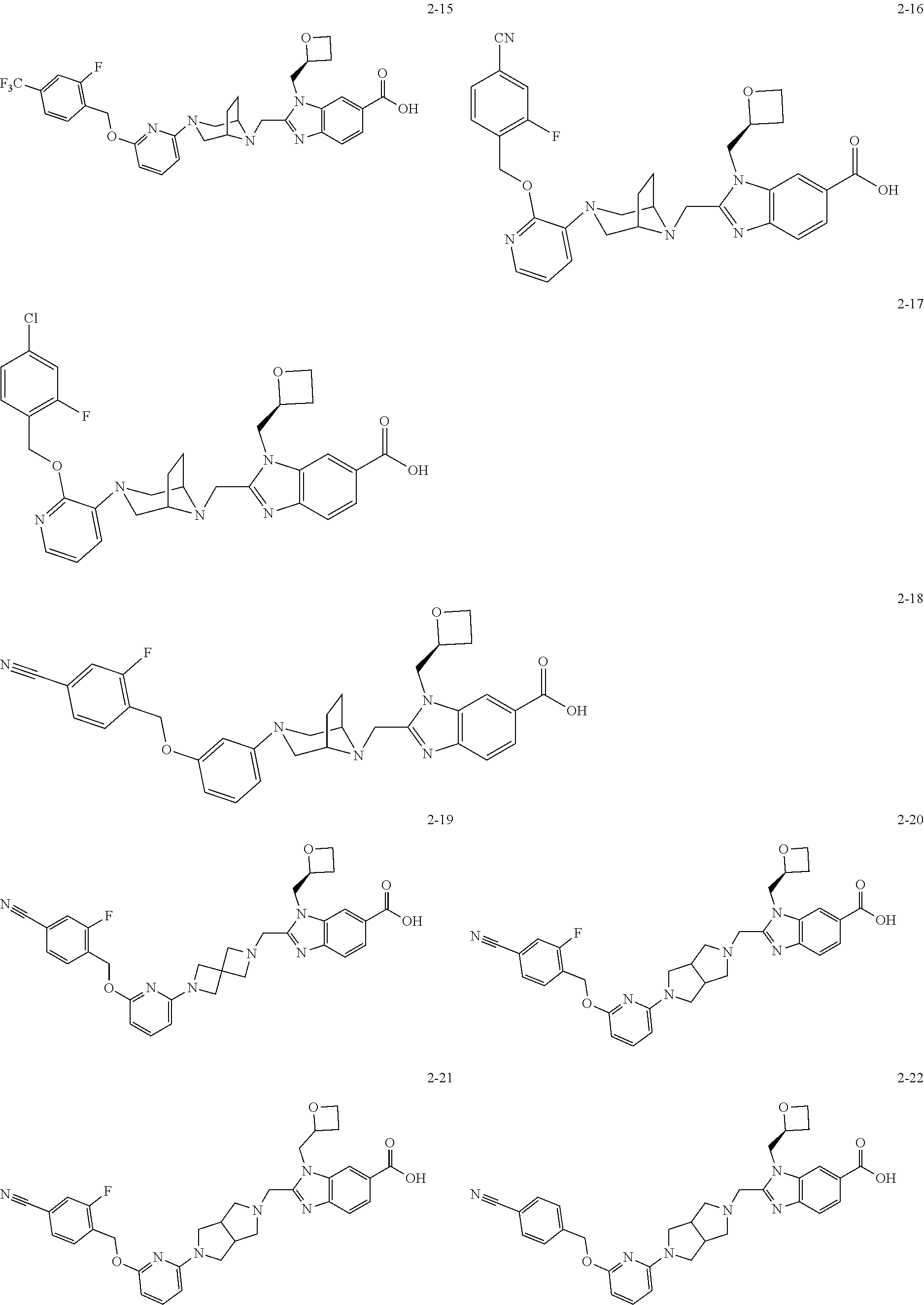

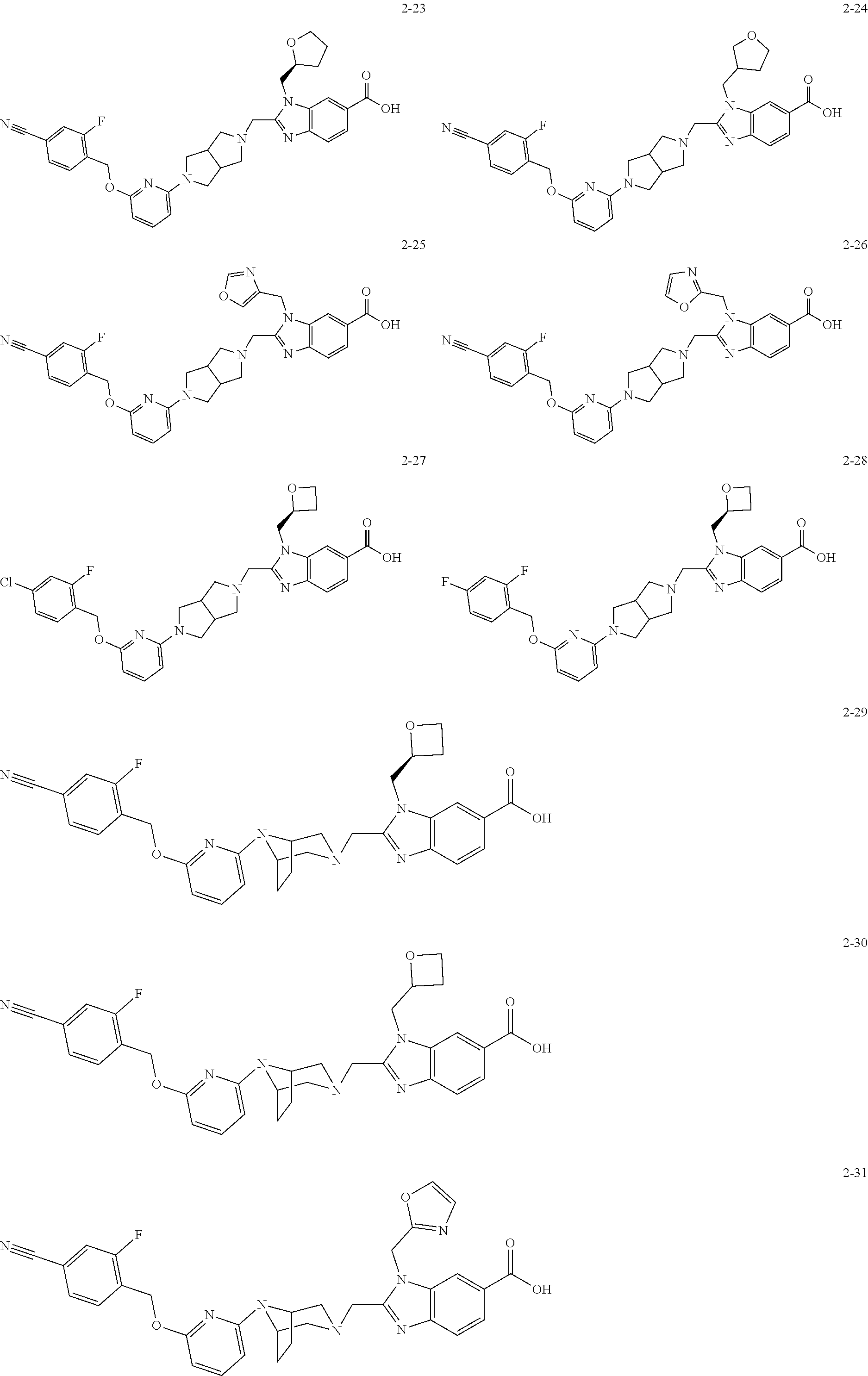

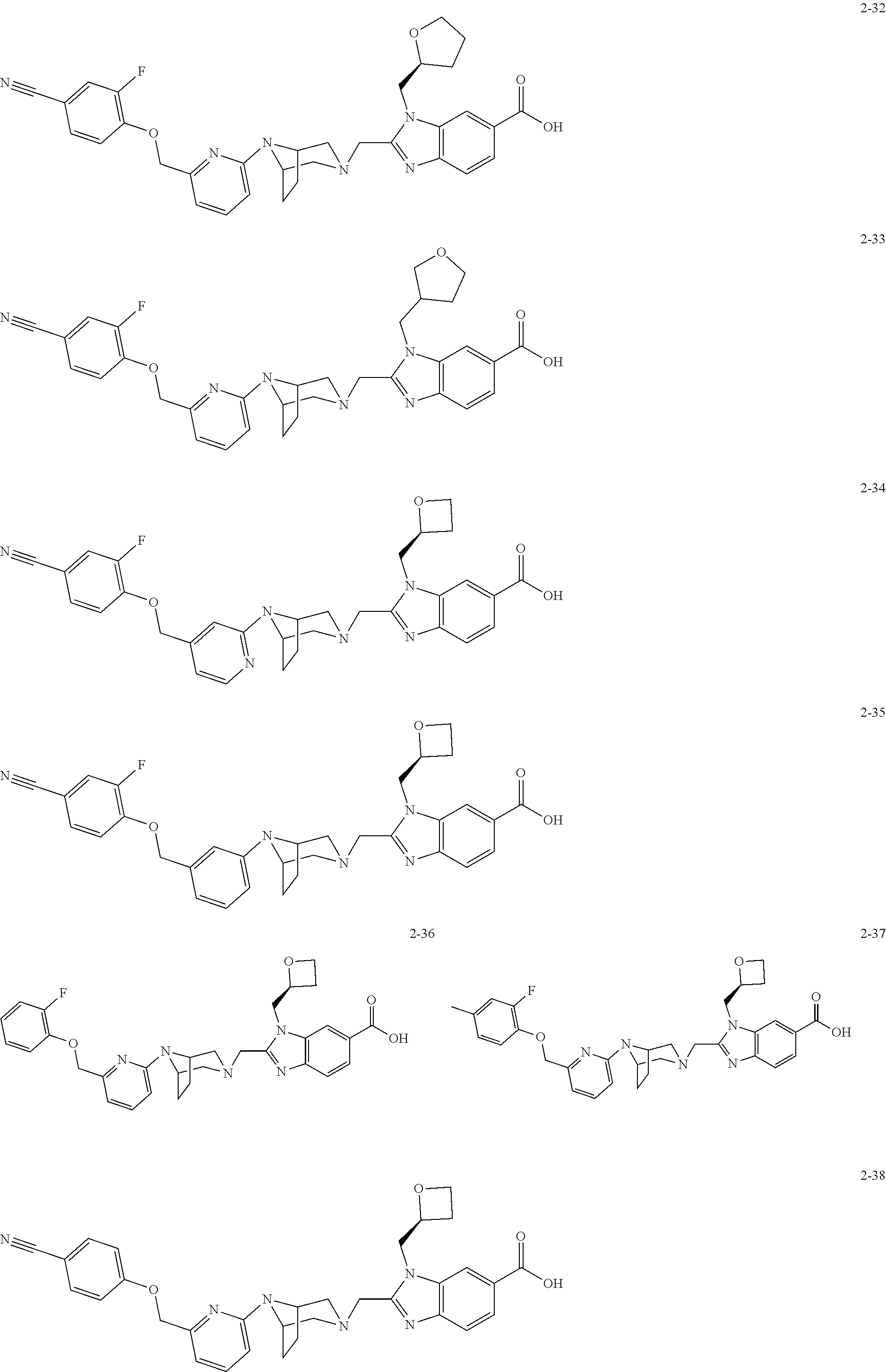

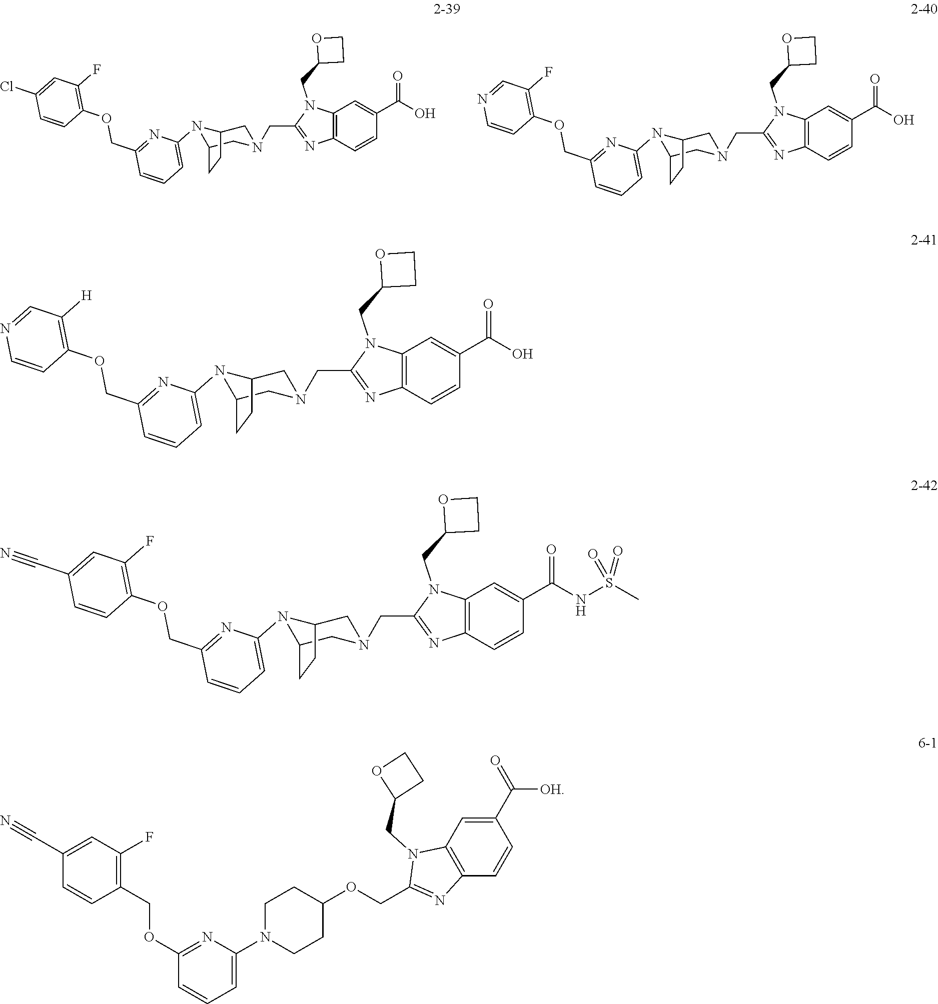 [SMILES: O=C(O)c1ccc2nc(CN3CC4CCC(C3)N4c3cccc(COc4ccc(Cl)cc4F)n3)n(C[C@@H]3CCO3)c2c1 ; O=C(O)c1ccc2nc(CN3CC4CCC(C3)N4c3cccc(COc4ccncc4F)n3)n(C[C@@H]3CCO3)c2c1 ; O=C(O)c1ccc2nc(CN3CC4CCC(C3)N4c3cccc(COc4ccncc4)n3)n(C[C@@H]3CCO3)c2c1 ; CS(=O)(=O)NC(=O)c1ccc2nc(CN3CC4CCC(C3)N4c3cccc(COc4ccc(C#N)cc4F)n3)n(C[C@@H]3CCO3)c2c1 ; N#Cc1ccc(COc2cccc(N3CCC(OCc4nc5ccc(C(=O)O)cc5n4C[C@@H]4CCO4)CC3)n2)c(F)c1]

30.-31. (canceled)

32. A pharmaceutical composition comprising the compound of Formula I or a pharmaceutically acceptable salt thereof as claimed in claim 1, and at least one pharmaceutically acceptable carrier.

33. A method of treating a GLP-1R-related disorder or condition in a subject in need thereof, comprising administering to the subject a therapeutically effective amount of a compound of Formula I or a pharmaceutically acceptable salt thereof as claimed in claim 1.

34. The method as claimed in claim 33, wherein the GLP-1R-related disorder or condition is selected from the group consisting of obesity, type 2 diabetes mellitus (T2DM), Non-alcoholic fatty liver disease (NAFLD), and non-alcoholic steatohepatitis (NASH).

35.-38. (canceled)
